(19) United States
(12) Patent Application Publication
(10) Pub. No.: US 2024/0150375 A1
(43) Pub. Date: May 9, 2024

(54) MACROCYCLIC COMPOUNDS AND USES THEREOF

(71) Applicant: Theseus Pharmaceuticals, Inc., Cambridge, MA (US)

(72) Inventors: Wei-Sheng HUANG, Cambridge, MA (US); William C. SHAKESPEARE, Cambridge, MA (US); Charles J. EYERMANN, Cambridge, MA (US); David C. DALGARNO, Cambridge, MA (US)

(73) Assignee: Theseus Pharmaceuticals, Inc., Cambridge, MA (US)

(21) Appl. No.: 18/506,625

(22) Filed: Nov. 10, 2023

Related U.S. Application Data
(63) Continuation of application No. PCT/US2022/028755, filed on May 11, 2022.
(60) Provisional application No. 63/187,041, filed on May 11, 2021.

(51) Int. Cl. C07D 498/22

(52) U.S. Cl. CPC C07D 498/22

(57) ABSTRACT

Described herein are macrocyclic compounds of Formula (I), which can inhibit kinases such as EGFR, including mutant forms such as T790M EGFR mutants. Also described herein are pharmaceutical compositions comprising a compound of Formula (I), or any pharmaceutically acceptable form thereof, processes for their preparation, and use in therapy for the prevention or treatment of cancer. In particular, compounds described herein can be effective for treating EGFR-driven cancers including non-small cell lung cancer (NSCLC).

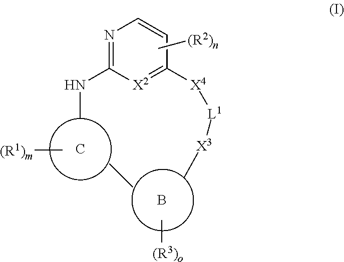

### CROSS-REFERENCE TO RELATED APPLICATIONS

The present application claims benefit of U.S. Provisional Application No. 63/187,041, filed May 11, 2021, which is hereby incorporated by reference in its entirety.

### FIELD OF THE INVENTION

Described herein are macrocyclic compounds that can be used as kinase inhibitors. In particular, compounds described herein can inhibit epidermal growth factor receptor (EGFR), including mutant forms of EGFR. Compounds described herein can be effective for treating various disorders that include cancers such as EGFR-driven cancers (e.g., non-small cell lung cancer (NSCLC) characterized by mutant EGFR).

### BACKGROUND

Signal transduction refers to the transmission of stimulatory or inhibitory signals into and within a cell leading, often via a cascade of signal transmission events, to a biological response within the cell. Defects in various components of signal transduction pathways have been found to account for a large number of diseases, including numerous forms of cancer, inflammatory disorders, metabolic disorders, vascular and neuronal diseases.

Signal transduction is often mediated by certain proteins called kinases. Kinases can generally be classified into protein kinases and lipid kinases, and certain kinases exhibit dual specificities. For example, epidermal growth factor receptor (EGFR) belongs to a family of receptor tyrosine kinases (RTKs) that include EGFR/ERBB1, HER2/ERBB2/NEU, HER3/ERBB3, and HER4/ERBB4. The binding of a ligand, such as epidermal growth factor (EGF), induces a conformational change in EGFR that facilitates receptor homo- or heterodimer formation, leading to activation of EGFR tyrosine kinase activity. Activated EGFR then phosphorylates its substrates, resulting in activation of multiple downstream pathways within the cell, including the PI3K-AKT-mTOR pathway, which is involved in cell survival, and the RAS-RAF-MEK-ERK pathway, which is involved in cell proliferation. (Chong et al. Nature Med. 2013; 19(11):1389-1400).

Certain cancers are characterized by mutations of EGFR, which results in increased cell proliferation. Tyrosine kinase inhibitor (TKI) therapies that inhibit EGFR can lead to clinical responses; however, mutations in EGFR can also confer resistance to such therapies.

New therapeutic methods therefore remain necessary for treating cancers associated with defective signal transduction pathways, including EGFR-driven cancers.

### SUMMARY OF THE INVENTION

Described herein are new compounds that can be effective inhibitors of EGFR. Such compounds can be useful for treating various diseases and disorders, including EGFR-driven cancers such as non-small cell lung cancer (NSCLC) characterized by mutant EGFR.

A first aspect of the invention relates to compounds of Formula (I):

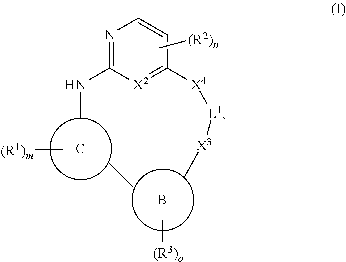

or a pharmaceutically acceptable salt thereof, wherein
X² is independently N or CR⁵; each of X³ and X⁴ is independently a covalent bond, O, S, NR⁶, C(O)NR⁶, NR⁶C(O), NR⁶C(O)NR⁶, or (C(R⁷)₂)_{q};
L¹ is independently a covalent bond, C₁₋₆ heteroalkylene, C₁₋₆ alkylene, C₂₋₆ alkenylene, C₂₋₆ alkynylene, C₃₋₆ cycloalkylene, 3- to 10-membered heterocyclylene, phenylene, naphthylene, or 5- to 10-membered heteroarylene;
each R¹ and R² is independently

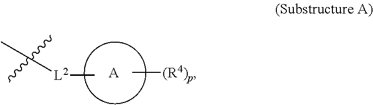

OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, or (CH₂)^{t}R¹²; or two R¹ or two R², together to which the atoms they are attached form a 5- to 10-membered ring.
L² is independently a covalent bond, O, NR^{L}, C(O), C(O)NR^{L}, NR^{L}C(O), CR^{L}₂;
R^{L} is independently H or C₁₋₆ alkyl;
A is independently phenyl, naphthyl, 5- to 13-membered heteroaryl, C₃-C₁₀ cycloaliphatic, or 3- to 10-membered heterocyclyl;
B is independently phenyl, naphthyl, 5- to 13-membered heteroaryl, C₃-C₁₀ cycloaliphatic, or 3- to 10-membered heterocyclyl;
C is independently 5- or 6-membered heteroaryl;
each R³ is independently OH, CN, halogen, C₁₋₆ aliphatic, C₁₋₆ alkoxy, NR⁸R⁹. C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, or (CH₂)ᵣR¹²;
each R⁴ is independently H, OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, NR¹¹(CH₂)ₛNR⁸R⁹, (CH₂)ₜNR⁸R⁹, (CH₂)ₜOH, (CH₂)—OCH₃, O(CH₂)ₜOH, O(CH₂)ₜOCH₃, O(CH₂)ᵣR¹², or (CH₂)ᵣR¹²; or R⁴ and R⁶ or R⁴ and R⁷, together with the atoms to which they are attached, form a 5- to 6-membered ring;
each R⁵ is independently H, OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, or (CH₂)ᵣR¹²;
each R⁶ is independently H, a N-protecting group, or C₁₋₆ alkyl; or R⁶ and R⁴, together with the atoms to which they are attached, form a 5- to 6-membered ring;
each R⁷ is independently H or C₁₋₆ alkyl; or two R⁷ on the same carbon combine to from an oxo (═O) group; or R⁷ and R⁴, together with the atoms to which they are attached, form a 5- to 6-membered ring;
each R⁸, R⁹, and R¹¹ is independently H or C₁₋₆ alkyl; or R⁸ and R⁹, together with the nitrogen atom to which they are attached, form a 3- to 10-membered heterocyclyl, or R⁸ and R¹¹, together with the atoms to which they are attached, form a 3- to 10-membered heterocyclyl;
each R¹⁰ is independently C₁₋₆ aliphatic, C₃-C₁₀ cycloaliphatic, 3- to 10-membered heterocyclyl, phenyl, naphthyl, or a 5- to 12-membered heteroaryl, or R¹⁰ and R¹¹, together with the atoms to which they are attached, form a 3- to 10-membered heterocyclyl;
each R¹² is independently C₃-C₁₀ cycloaliphatic, 3- to 10-membered heterocyclyl, phenyl, naphthyl, or a 5- to 12-membered heteroaryl;
each m, n, and o, is independently 0, 1, or 2;
each p is independently 0, 1, 2; 3, or 4;
each q is independently 1 or 2;
each r is independently an integer of 0-4;
each s is independently an integer of 2-6; and
each t is independently an integer of 1-6.

In embodiments, each R⁴ is independently H, OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, NR¹¹(CH₂)ₛNR⁸R⁹, (CH₂)ₜNR⁸R⁹, or (CH₂)ᵣR¹² or R⁴ and R⁶, or R⁴ and R⁷, together with the atoms to which they are attached, form a 5- to 6-membered ring.

In embodiments, each m, n, o, and p is independently 0, 1, or 2.

In embodiments, at least one m or n is not 0.

In embodiments, one of R¹ and R² is present and is Substructure A or halogen.

In embodiments, one of R¹ and R² is present and is Substructure A.

In embodiments, no more than one Substructure A is present.

In embodiments, C is 5- or 6-membered N-containing heteroaryl.

In embodiments, C is pyridyl, pyrimidyl, pyrazolyl, pyrrolyl, thiazolyl, oxazolyl, or imidazolyl.

In embodiments, a compound has a structure according to Formula (I-A)

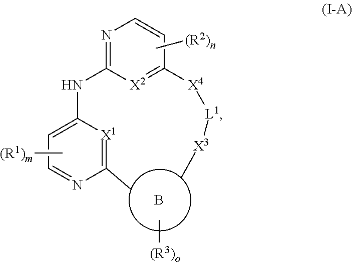

or a pharmaceutically acceptable salt thereof, wherein X¹ is N or CR⁵.

In embodiments, a compound has a structure according to Formula (I-B)

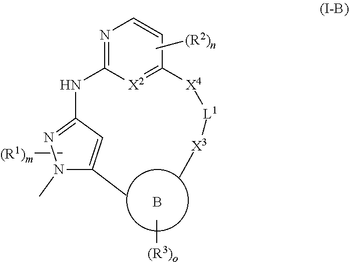

or a pharmaceutically acceptable salt thereof, wherein
m is 0 or 1.

In embodiments, a compound has a structure according to Formula (I-C)

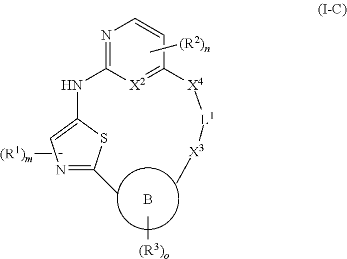

or a pharmaceutically acceptable salt thereof, wherein
m is 0 or 1.

In embodiments, a compound has a structure according to Formula (II),

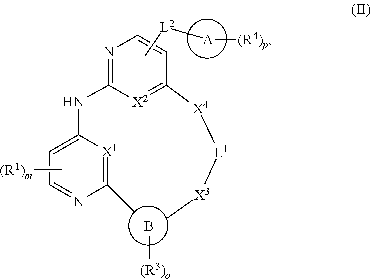

or a pharmaceutically acceptable salt thereof, wherein
each R¹ is independently OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, or R¹².

In embodiments, a compound has a structure according to Formula (II-A),

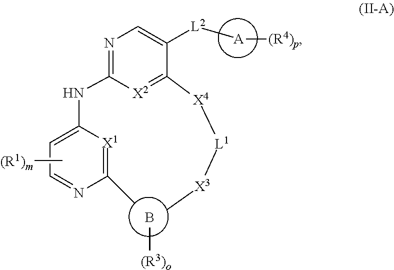

or a pharmaceutically acceptable salt thereof.

In embodiments, m is 0.

In embodiments, a compound has a structure according to Formula (III),

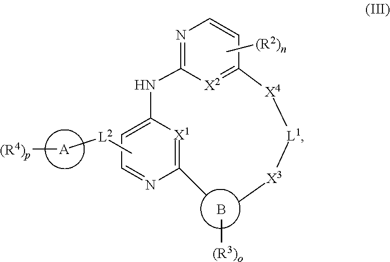

or a pharmaceutically acceptable salt thereof, wherein
each R² is independently OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, or R¹².

In embodiments, a compound has a structure according to Formula (III-A),

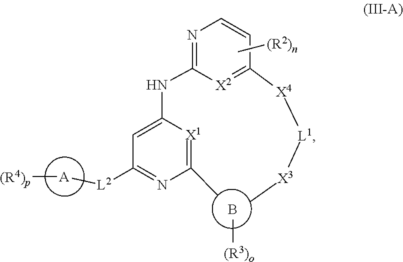

or a pharmaceutically acceptable salt thereof.

In embodiments, n is 0.

In embodiments, each of X¹ and X² is independently N or CH.

In embodiments, X¹ is N.

In embodiments, X² is CH.

In embodiments, X³ is O.

In embodiments, X⁴ is O.

In embodiments, each X³ and X⁴ is independently a covalent bond, O, S, NR⁶, C(O), CH₂, CHCH₃, or C(CH₃)₂.

In embodiments, X² is CH, X¹ is O, and X⁴ is O. In embodiments, X¹ is N.

In embodiments, L¹ is unsubstituted C₁₋₆ alkylene or C₁₋₆ alkylene comprising 1 or 2 oxo (═O) substituents.

In embodiments, L is unsubstituted C₁₋₆ heteroalkylene or C₁₋₆ heteroalkylene comprising 1 or 2 oxo (═O) substituents.

In embodiments, L¹ is an unsubstituted linear C₄₋₆ alkylene or an unsubstituted branched C₄₋₆ alkylene.

In embodiments, L¹ is

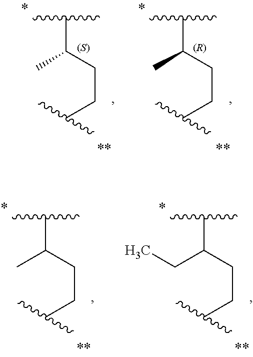

where * denotes the point of covalent attachment to X⁴, and ** denotes the point of covalent attachment to X³.

In embodiments, a C₁₋₆ heteroalkylene comprises 1, 2, or 3 heteroatoms that are independently oxygen or nitrogen.

In embodiments, a C₁₋₆ heteroalkylene is —O(CH₂)ᵤ—, —(CH₂)ᵤO—, —O(CH₂)ᵤO—, —OCH₂OCH₂C₂OCH₂—, —CH₂OCH₂CH₂O—, —OCH₂CH₂OCH₂—, —NH(CH₂)ᵤ—, —(CH₂)NH—, or —NH(CH₂)ᵤNH—, and wherein u is an integer of 1-4.

In embodiments, B is phenyl or 5- to 6-membered heteroaryl.

In embodiments, B is phenyl, pyridyl, pyrimidyl, pyrazolyl, pyrrolyl, thiazolyl, oxazolyl, or imidazolyl.

In embodiments, B is

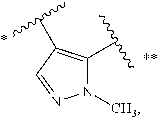 [SMILES: *c1cnn(C)c1*]

where * denotes the point of covalent attachment to C, and ** denotes the point of covalent attachment to X³.

In embodiments, R³ is methyl, halogen, or CN, and o is 0 or 1.

In embodiments, A is phenyl or 5- to 6-membered heteroaryl.

In embodiments, A is phenyl, pyridyl, pyrimidyl, pyrazolyl, pyrrolyl, thiazolyl, oxazolyl, or imidazolyl.

In embodiments, a compound has a structure according to Formula (IV),

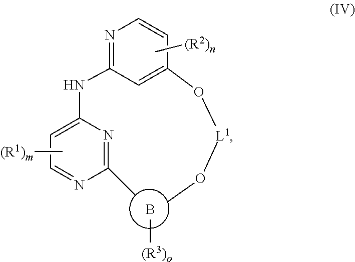

or a pharmaceutically acceptable salt thereof, wherein
L¹ is unsubstituted linear or branched C₂₋₆ alkylene;
B is phenyl or 5- to 6-membered heteroaryl;
R³ is methyl, halogen, or CN;
is 0 or 1; and
one of R¹ and R² is present as Substructure A.

In embodiments, a compound has a structure according to Formula (V),

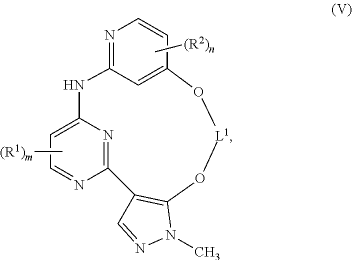

or a pharmaceutically acceptable salt thereof, wherein
L¹ is —(CH₂)₃— or —CH(CH₃)CH₂CH₂—.

In embodiments, a compound has a structure according to Formula (VI-1) or Formula (VI-2),

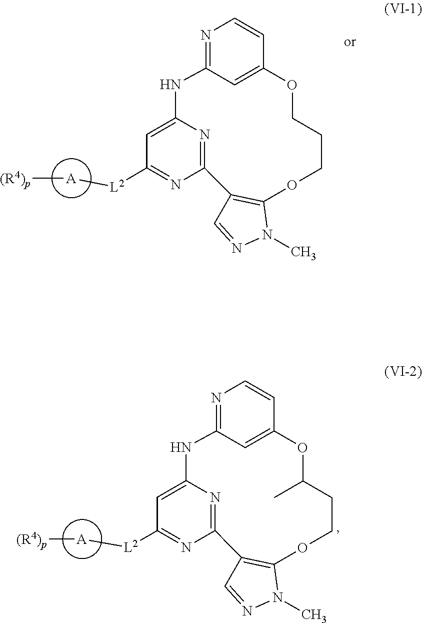

or a pharmaceutically acceptable salt thereof.

In embodiments, a compound has a structure according to Formula (VI-3) or Formula (VI-4),

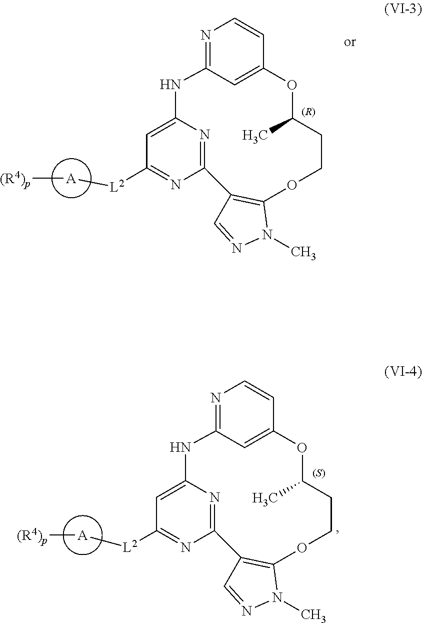

or a pharmaceutically acceptable salt thereof.

In embodiments, a compound has a structure according to Formula (VII-1) or Formula (VII-2),

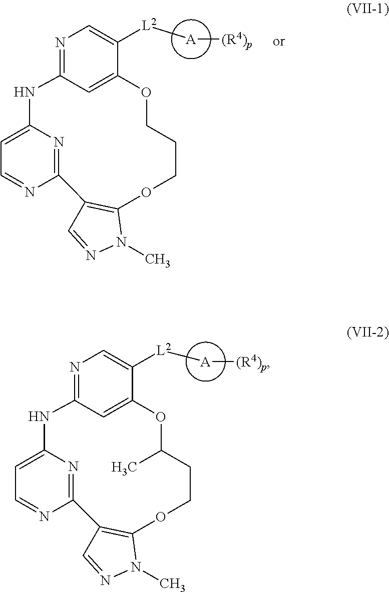

or a pharmaceutically acceptable salt thereof.

In embodiments, a compound has a structure according to Formula (VII-3) or Formula (VII-4),

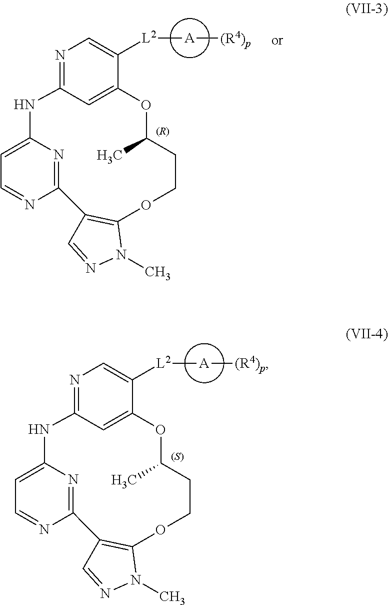

or a pharmaceutically acceptable salt thereof.

In embodiments, A is phenyl or 5- to 6-membered heteroaryl.

In embodiments, L² is a covalent bond.

In embodiments,

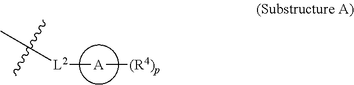

is selected from the group consisting of:

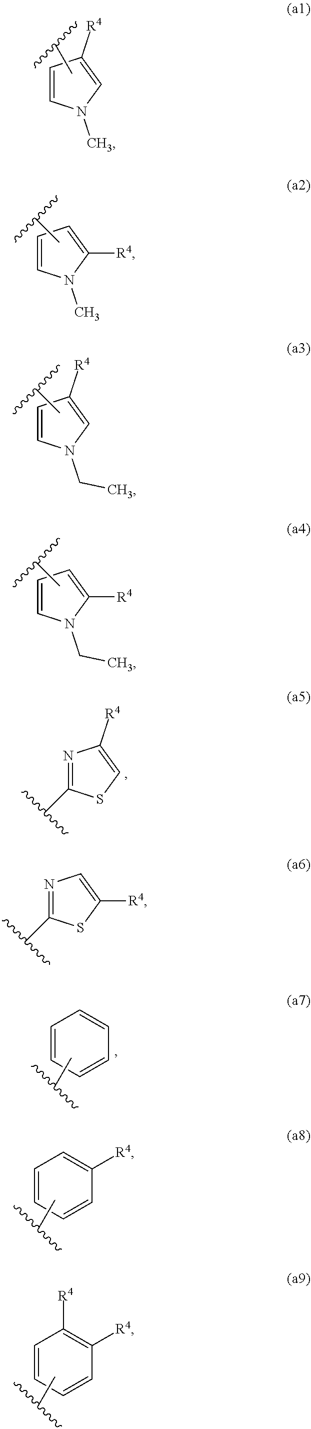

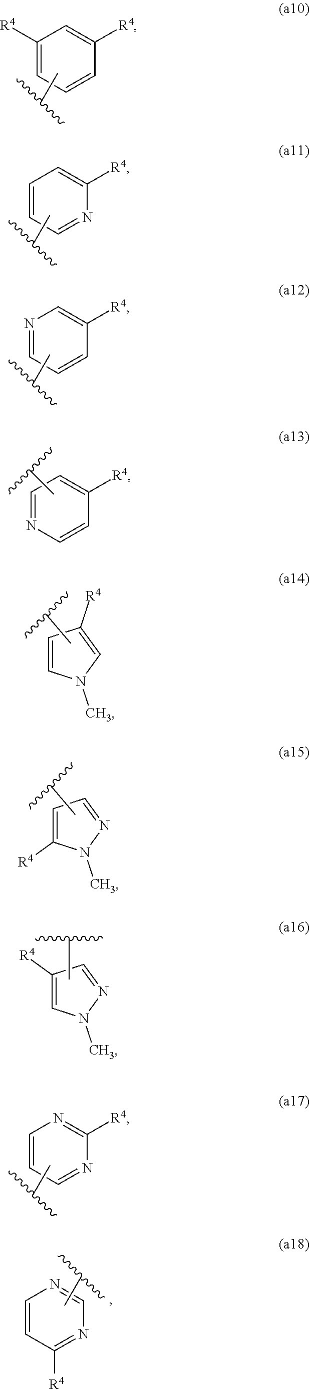

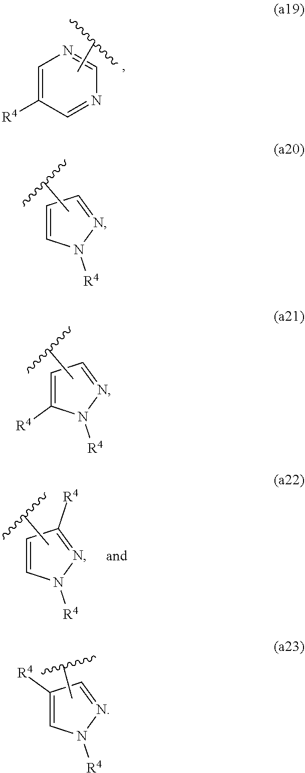

In embodiments, a compound has a structure according to Formula (VIII),

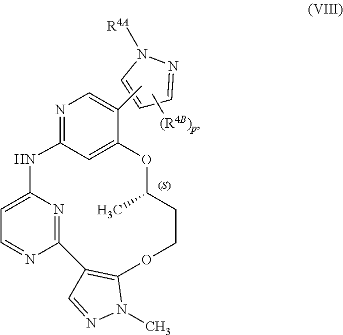

or a pharmaceutically acceptable salt thereof, wherein
R^{4A} is a first R⁴ group,
R^{4B} is a second R⁴ group, and
p is 0 or 1.

In embodiments, a compound has a structure according to Formula (IX),

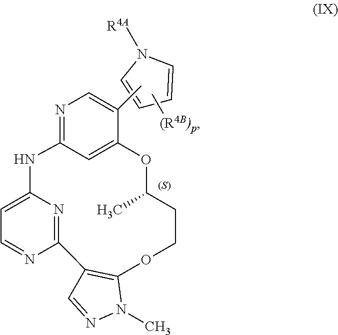

or a pharmaceutically acceptable salt thereof, wherein
R_{4A} is a first R⁴ group,
R^{4B} is a second R⁴ group, and
p is 0 or 1.

In embodiments, a compound has a structure according to Formula (X),

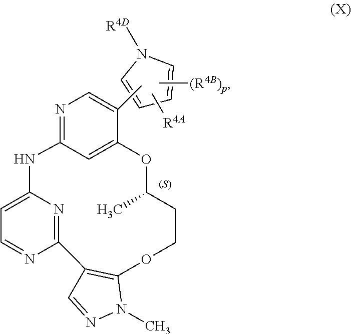

or a pharmaceutically acceptable salt thereof, wherein
R^{4A} is a first R⁴ group,
R^{4B} is a second R⁴ group,
p is 0 or 1, and
R^{4D} is a R⁴ group that is unsubstituted C₁₋₆ alkyl.

In embodiments, a compound has a structure according to Formula (XI),

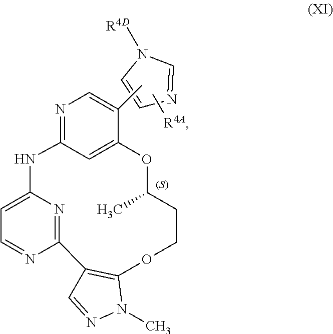

or a pharmaceutically acceptable salt thereof, wherein
R^{4A} is a first R⁴ group, and
R^{4D} is a R⁴ group that is unsubstituted C₁₋₆ alkyl.

In embodiments, a compound has a structure according to Formula (XII),

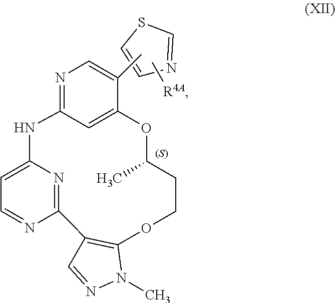

or a pharmaceutically acceptable salt thereof, wherein
R^{4A} is a first R⁴ group.

In embodiments, a compound has a structure according to Formula (XIII),

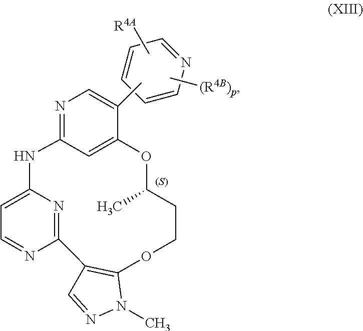

or a pharmaceutically acceptable salt thereof, wherein
R^{4A} is a first R⁴ group,
R^{4B} is a second R⁴ group, and
p is 0 or 1.

In embodiments, a compound has a structure according to Formula (XIV),

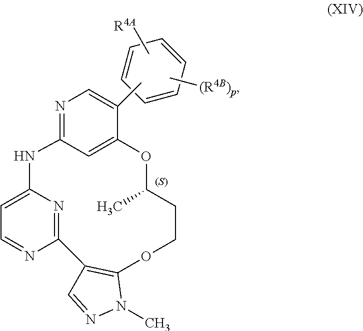

or a pharmaceutically acceptable salt thereof, wherein
R^{4A} is a first R⁴ group,
R^{4B} is a second R⁴ group, and
p is 0 or 1.

In embodiments, a compound has a structure according to Formula (XV),

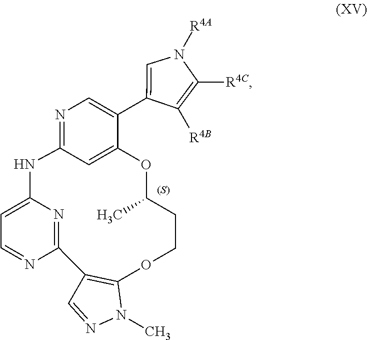

or a pharmaceutically acceptable salt thereof, wherein
R^{4A} is a first R⁴ group,
R^{4B} is a second R⁴ group, and
R^{4C} is a third R⁴ group.

In embodiments, a compound has a structure according to Formula (XVI),

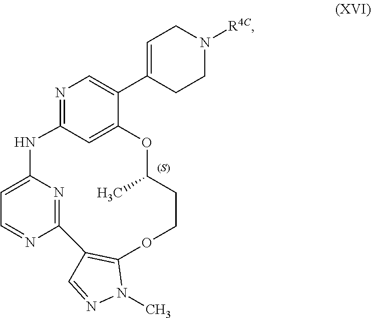

or a pharmaceutically acceptable salt thereof, wherein
R^{4C} is a first R⁴ group.

In embodiments, a compound has a structure according to Formula (XVII),

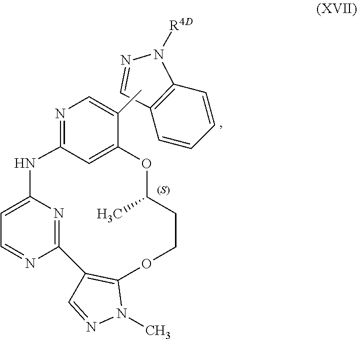

or a pharmaceutically acceptable salt thereof, wherein
R^{4D} is a R⁴ group that is unsubstituted C₁₋₆ alkyl.

In embodiments, a compound has a structure according to Formula (XVIII),

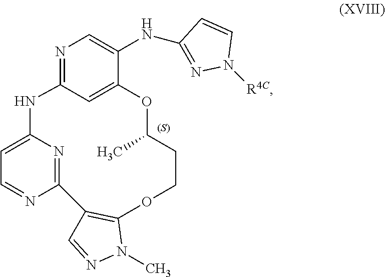

or a pharmaceutically acceptable salt thereof, wherein
R^{4C} is a first R⁴ group.

In embodiments, a compound has a structure according to Formula (XIX),

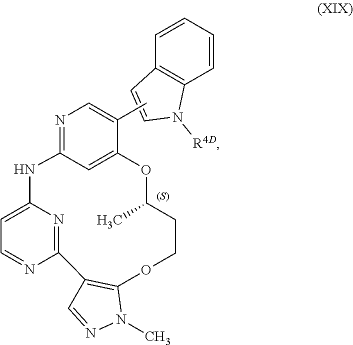

or a pharmaceutically acceptable salt thereof, wherein
R^{4D} is a R⁴ group that is unsubstituted C₁₋₆ alkyl.

In embodiments, a compound has a structure according to Formula (XX),

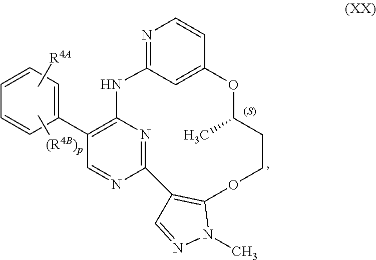

or a pharmaceutically acceptable salt thereof, wherein
R^{4A} is a first R⁴ group,
R^{4B} is a second R⁴ group, and
p is 0 or 1.

In embodiments, a compound has a structure according to Formula (XXI),

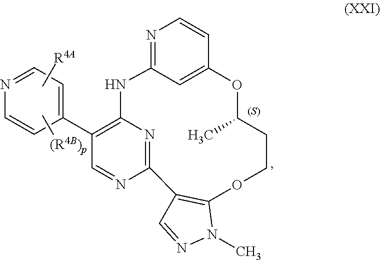

or a pharmaceutically acceptable salt thereof, wherein
R^{4A} is a first R⁴ group,
R^{4B} is a second R⁴ group, and
p is 0 or 1.

In embodiments, a compound has a structure according to Formula (XXII),

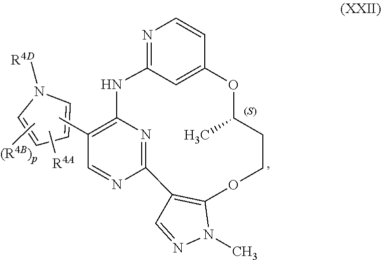

or a pharmaceutically acceptable salt thereof, wherein
R^{4A} is a first R⁴ group,
R^{4B} is a second R⁴ group,
p is 0 or 1, and
R^{4D} is a R⁴ group that is unsubstituted C₁₋₆ alkyl.

In embodiments, a compound has a structure according to Formula (XXIII),

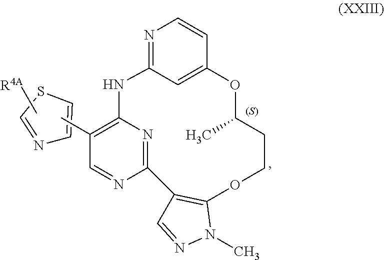

or a pharmaceutically acceptable salt thereof, wherein
R^{4A} is a first R⁴ group.

In embodiments, a compound described herein comprises one or more R⁴ groups selected from: —C≡N; —C≡CH; a saturated linear or branched C₁₋₆ aliphatic or C₁₋₆ alkoxy comprising 0-4 fluoro substituents; NR¹¹(CH₂)ₛNR⁸R⁹; (CH₂)ₜNR⁸R⁹; O(CH₂)ₜOCH₃; O(CH₂)R¹²; and (CH₂)ᵣR¹². In embodiments, R¹² is selected from the group consisting of: a C₃₋₆ cycloalkyl; a 3-9 membered heterocyclyl comprising 1-3 heteroatoms selected from O, N, and S; and 5- to 6-membered heteroaryl. In embodiments, R¹² is selected from cyclopropyl, cyclobutyl, cyclopentyl, cyclohexyl, oxetanyl, tetrahydrofiryl, tetrahydropyanyl, azetidine, pyrroldinyl, piperidinyl, piperazinyl, and morpholino. In embodiments, R¹² is substituted with 0-4 R¹⁴, wherein each R¹⁴ is independently selected from —CN, oxo (═O), halogen, —OH, —NH₂, monoalkylamino, dialkylamino, unsubstituted C₃₋₆ cycloalkyl, or unsubstituted 3- to 4-membered heterocyclyl. In embodiments, each R¹⁴ is independently selected from —CN, —F, —OH, —NH₂, —NHCH₃, —N(CH₃)₂, —NHCH₂CH₃, —N(CH₂CH₃)₂, —CH₃, —CH₂F, —CHF₂, —CF₃, —CH₂CH₃, —CH₂CH₂F, —CH₂CHF₂, —CH₂CF₃, —CH₂CH₂CH₃, —CH₂CH₂CH₂F, —CH₂CH₂CHF₂, —CH₂CH₂CF₃, —CH₂CH₂OCH₃, —COCH₃, —COCH₂CH₃, —CH₂COCH₃, —CH₂COCH₂CH₃, cyclopropyl, cyclobutyl, oxetanyl, and azetidinyl.

In embodiments, a compound described herein comprises:
an R⁴ group selected from: —CN, —CH₃, —CH₂F, —CHF₂, —CF₃, —CH₂CH₃, —CH₂CFH₂, —CH₂CHF₂, —CH₂CF₃, —CH(C₁₁₃)₂, —C(CH₃)₃, —C≡CH,

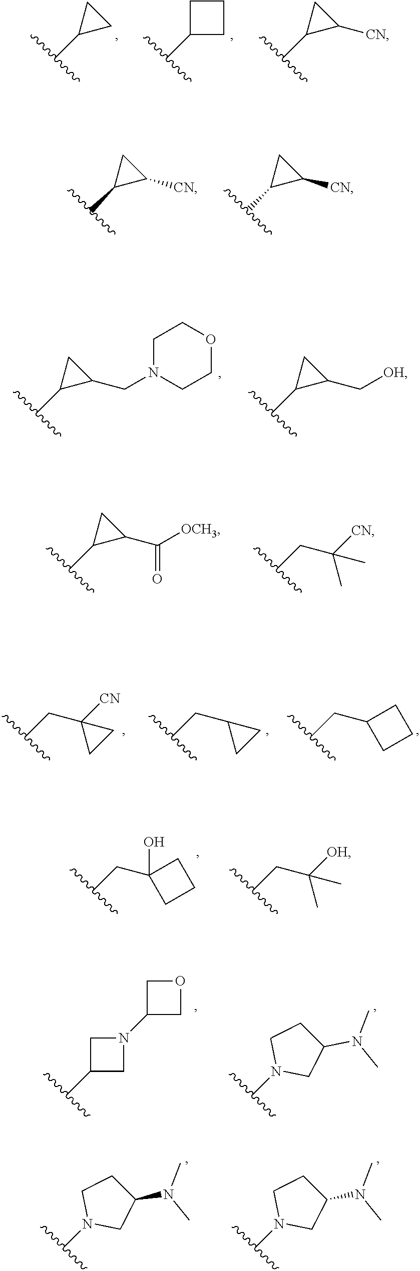

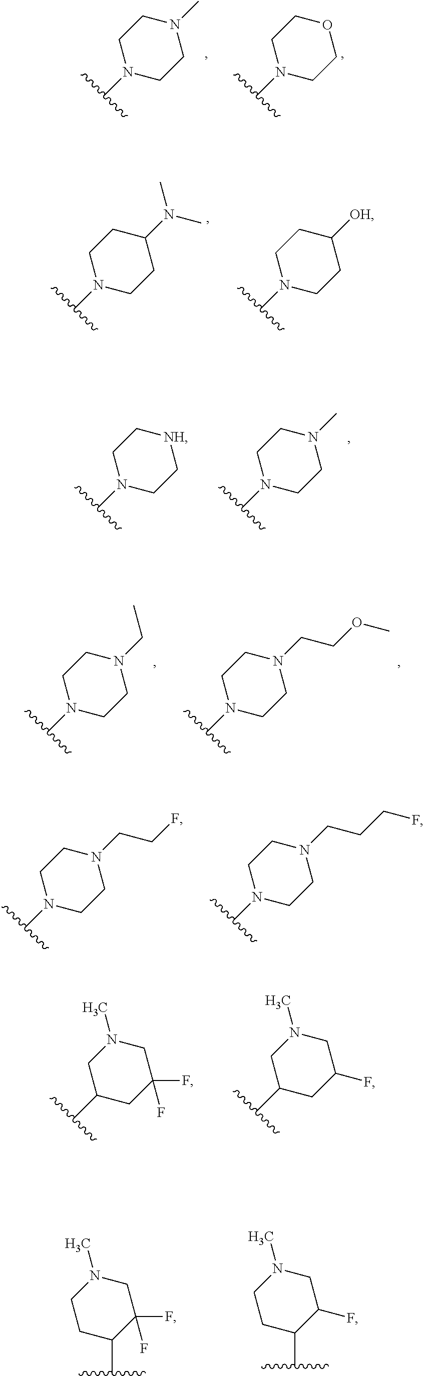

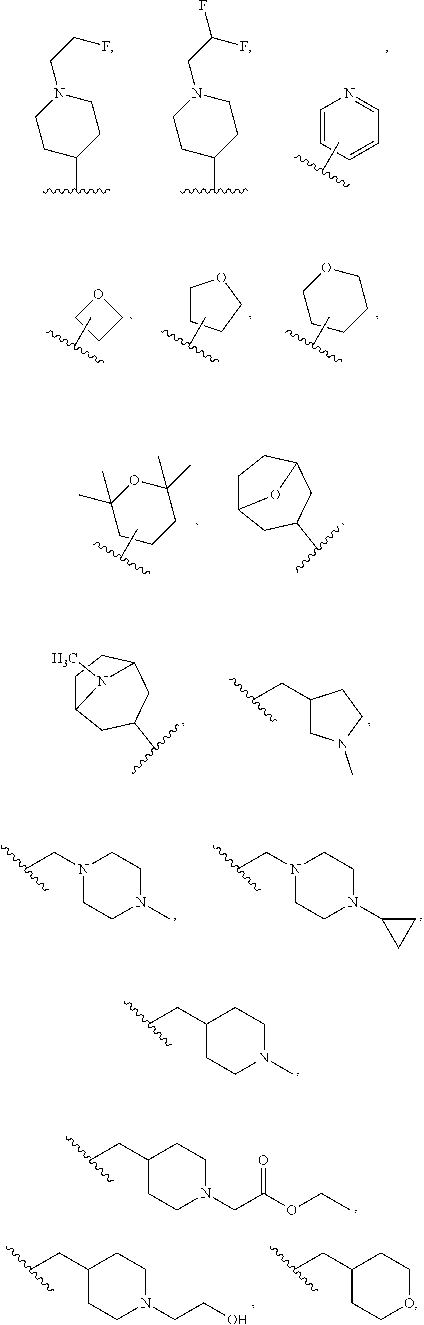

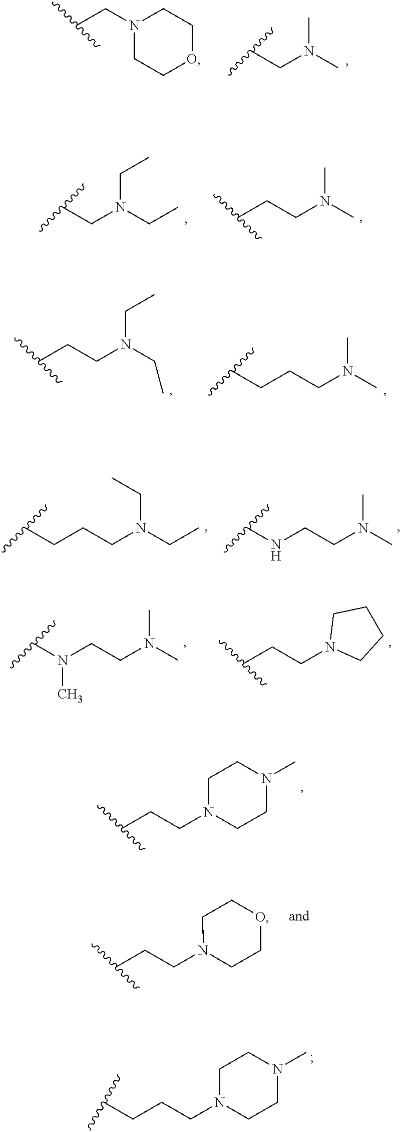

and/or
an R⁴ group selected from —CH₂OCH₃, —OCH₃, —OCH₂F, —OCHF₂, —OCF₃, —OCH₂CH₃, —OCH₂CH₂F, —OCH₂CHF₂, —OCH₂CF₃, —OCH₂CH₂CH₃, —O CH₂CH(CH₃)₂, —OCH₂CH₂OCH₃,

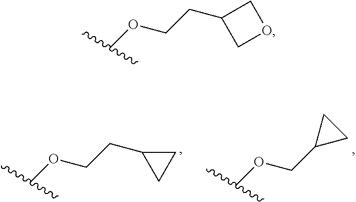 [SMILES: *OCCC1COC1 ; *OCCC1CC1 ; *OCC1CC1]

—CO₂CH₃, and CH₃.

In embodiments, R⁴ is selected from unsubstituted C₁₋₆ alkyl, CO₂(unsubstituted C₁₋₆ alkyl), O-(unsubstituted C₁₋₆ alkyl), O—(C₁₋₆ haloalkyl), NH(CH₂)ₛNMe₂, (CH₂)ₜNMe₂, or

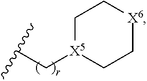

wherein
X⁵ is independently CH or N;
X⁶ is independently O, CHR¹³, or NR¹³;
R³ is independently H, C₁₋₆ alkyl, or C₃₋₆ cycloalkyl;
r is 0 or 1;
s is an integer of 2-4; and
t is an integer of 1-6.

In embodiments, one R⁴ is

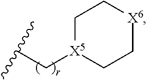

and, if present, a second R⁴ is selected from unsubstituted C₁₋₆ alkyl, CO₂(unsubstituted C₁₋₆ alkyl), O-(unsubstituted C₁₋₆ alkyl), O—(C₁₋₆ haloalkyl), NH(CH₂)ₛNMe₂, and (CH₂)NMe₂.

In embodiments,

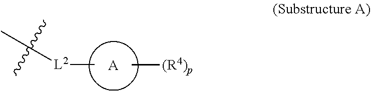

is

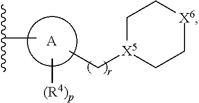

wherein
A is phenyl or 5- to 6-membered heteroaryl.
X⁵ is independently CH or N;
X⁶ is independently O, CHR¹³, or NR¹³;
R¹³ is independently H, unsubstituted C₁₋₆ alkyl, or unsubstituted C₃₋₆ cycloalkyl;
r is 0 or 1;
R⁴ is selected from unsubstituted C₁₋₆ alkyl, CO₂(unsubstituted C₁₋₆ alkyl), O-(unsubstituted C₁₋₆ alkyl), O—(C₁₋₆ haloalkyl), or NH(CH₂)ₛNMe₂;
p is 0 or 1; and
s is an integer of 2-6.

In embodiments,

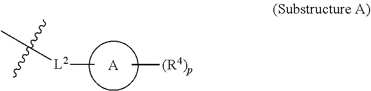

is

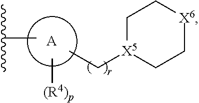

wherein X⁶ is O, NCH₃, or N(cyclopropyl).

In embodiments, r is 0.

In embodiments, r is 1.

In embodiments, a compound comprises a R⁴ group that is —CO₂CH₃, —OCH₂CF₃, —CH₃, —CH₂CH₃, —OCH₃, —OCH₂CH₃, —NHCH₂CH₂N(CH₃)₂, or —CH₂N(CH₃)₂.

In embodiments, A is phenyl, pyridyl, pyrimidyl, pyrazolyl, pyrrolyl, thiazolyl, oxazolyl, or imidazolyl.

In embodiments, each R^{4A}, R^{4B}, and R^{4C}, when present, is independently selected from: —C≡N; —C≡CH; a saturated linear or branched C₁₋₆ aliphatic or C₁₋₆ alkoxy comprising 0-4 fluoro substituents; NR¹¹(CH₂)ₛNR⁸R⁹; (CH₂)ₜNR⁸R⁹; O(CH₂)ₜOCH₃; O(CH₂)ᵣR¹²; and (CH₂)ᵣR¹².

In embodiments,
an R^{4A} and/or a R^{4C} group, when present, is selected
from: —CN, —CH₃, —CH₂F, —CHF₂,
—CF₃, —CH₂CH₃, —CH₂CFH₂, —CH₂CHF₂, —CH₂CF₃, —CH(CH₃)₂, —C(CH₃)₃, —C≡CH,

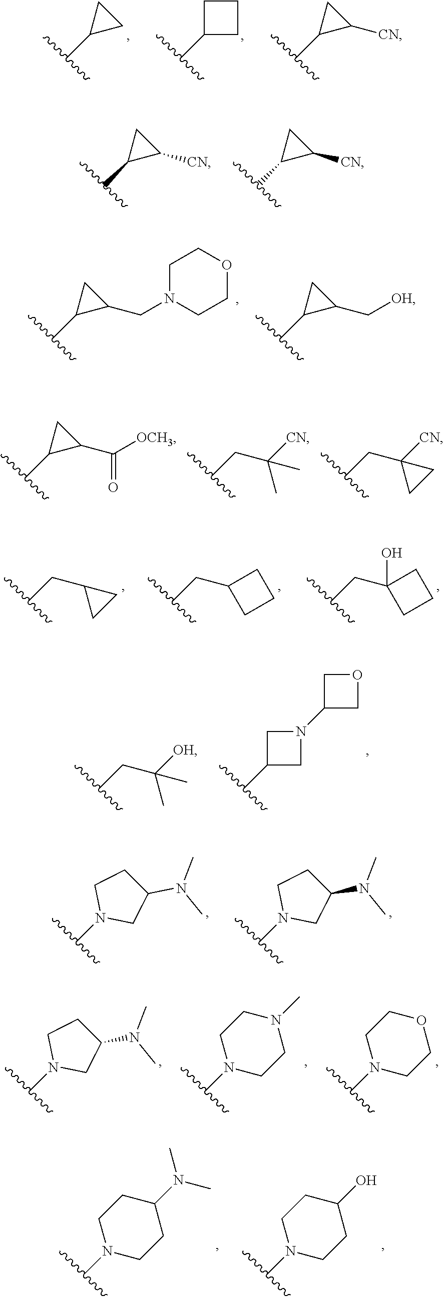

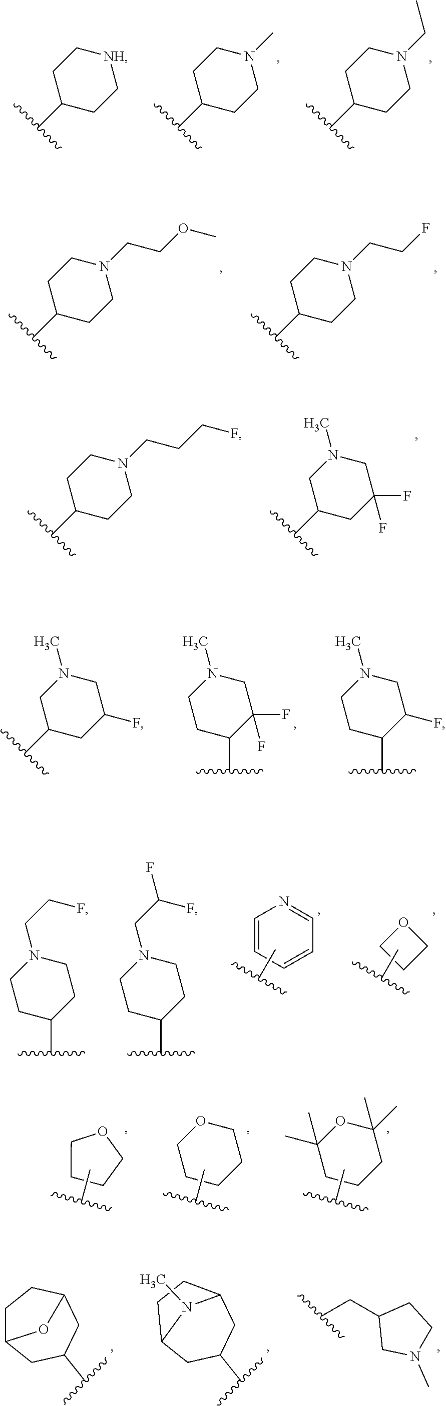

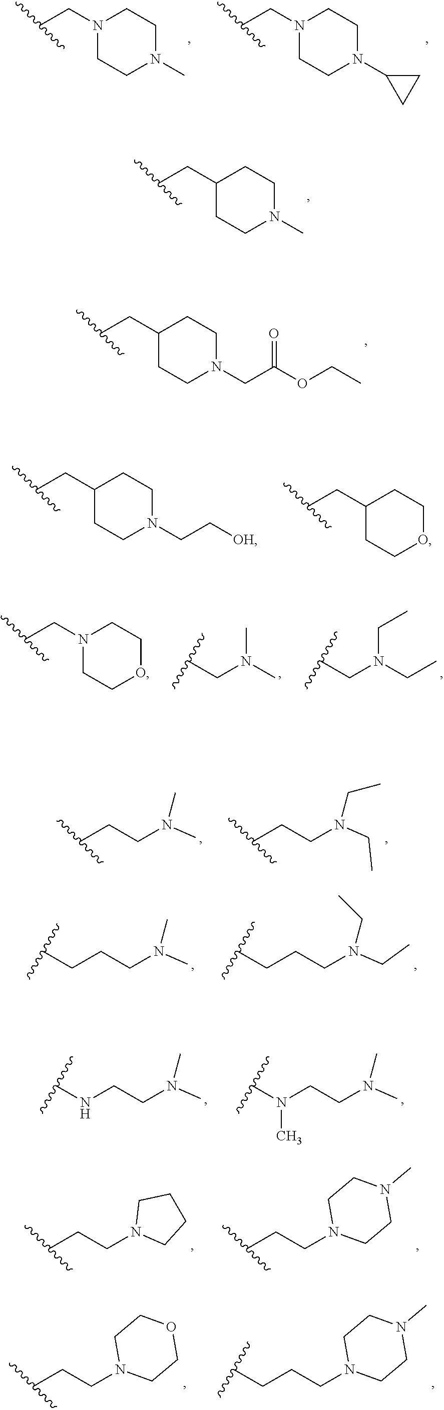

and/or
an R^{4B} group, when present, is selected from —CH₂OCH₃, —OCH₃, —OCH₂F, —OCHF₂, —OCF₃, —OCH₂CH₃, —OCH₂CH₂F, —OCH₂CHF₂, —OCH₂CF₃, —OCH₂CH₂CH₃, —OCH₂CH(CH₃)₂, —OCH₂CH₂OCH₃,

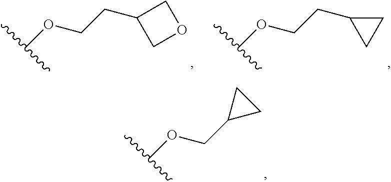

—CO₂CH₃, and CH₃.

In embodiments, a compound is any exemplary compound described herein, including any compound described in Table A (e.g., any one of Compounds (1)-(169)), or a pharmaceutically acceptable salt thereof.

In another aspect, the invention features a pharmaceutical composition comprising any compound described herein, or a pharmaceutically acceptable salt thereof.

In another aspect, the invention features a method of treating cancer comprising administering to a human in need thereof an effective amount of any compound described herein, or a pharmaceutically acceptable salt thereof, in a pharmaceutical composition.

In embodiments, a cancer is a lung cancer.

In embodiments, a cancer is non-small cell lung cancer.

In embodiments, a cancer (e.g., a lung cancer such as non-small cell lung cancer) is an EGFR-driven cancer.

In embodiments, a cancer (e.g., a lung cancer such as non-small cell lung cancer) is characterized by an EGFR mutation.

### DETAILED DESCRIPTION OF THE INVENTION

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, a bovine, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used in the description and the appended claims, the singular forms “a,” “an,” and “the” include plural referents unless the context clearly dictates otherwise. Thus, for example, reference to “a composition” includes mixtures of two or more such compositions.

Throughout the description and claims of this specification the word “comprise” and other forms of the word, such as “comprising” and “comprises,” means including but not limited to, and is not intended to exclude, for example, other additives, components, integers, or steps.

“Optional” or “optionally” means that the subsequently described event or circumstance may or may not occur, and that the description includes instances where the event or circumstance occurs and instances where it does not.

Improve, increase, or reduce: As used herein, the terms “improve,” “increase,” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable,” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio. Accordingly, pharmaceutically acceptable relates to substances that are not biologically or otherwise undesirable, i.e., the material can be administered to an individual along with the relevant active compound without causing clinically unacceptable biological effects or interacting in a deleterious manner with any of the other components of the pharmaceutical composition in which it is contained.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid, or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N(C₁₋₄-alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, sulfonate, and aryl sulfonate. Further pharmaceutically acceptable salts include salts formed from the quaternization of an amine using an appropriate electrophile, e.g., an alkyl halide, to form a quarternized alkylated amino salt.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Whenever a term (e.g., alkyl or aryl) or either of their prefix roots (e.g., alk- or ar-) appear in a name of a substituent the name is to be interpreted as including those limitations provided herein. For example, affixing the suffix “-ene” to a group indicates the group is a divalent moiety, e.g., arylene is the divalent moiety of aryl, heteroarylene is the divalent moiety of heteroaryl, cycloalkylene is the divalent moiety of cycloalkyl, heterocycloalkylene is the divalent moiety of heterocycloalkyl, or or heterocyclelene is the divalent moiety of heterocyclyl. Similarly, affixing the suffix “-oxy” to a group indicates the group is attached to the parent molecular structure through an oxygen atom (—O—).

Aliphatic: As used herein, the term aliphatic refers to hydrocarbons and includes both saturated and unsaturated hydrocarbons. An aliphatic may be linear, branched, or cyclic. For example, C₁-C₂₀ aliphatics can include C₁-C₂₀ alkyls (e.g., linear or branched C₁-C₂₀ saturated alkyls), C₂-C₂₀ alkenyls (e.g., linear or branched C₄-C₂₀ dienyls, linear, or branched C₂-C₂₀ trienyls, and the like), and C₂-C₂₀ alkynyls (e.g., linear or branched C₂-C₂₀ alkynyls). C₁-C₂₀ aliphatics can include C₃-C₂₀ cyclic aliphatics (e.g., C₃-C₂₀ cycloalkyls, C₄-C₂₀ cycloalkenyls, or C₃-C₂₀ cycloalkynyls). In certain embodiments, the aliphatic may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. An aliphatic group is unsubstituted or substituted with one or more substituent groups as described herein. For example, an aliphatic may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′), —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the aliphatic is unsubstituted. In some embodiments, the aliphatic does not include any heteroatoms.

Alkyl: As used herein, the term “alkyl” means acyclic linear and branched hydrocarbon groups, e.g. “C₁-C₂₀ alkyl” refers to alkyl groups having 1-20 carbons and “C₁-C₄ alkyl” refers to alkyl groups having 1-4 carbons. Alkyl groups include C₁-C₂₀ alkyl, C₁-C₁₅ alkyl, C₁-C₁₀ alkyl, C₁-C₄ alkyl, and C₁-C₃ alkyl). In embodiments, an alkyl group is C₁-C₄ alkyl. An alkyl group may be linear or branched. Examples of alkyl groups include, but are not limited to, methyl, ethyl, n-propyl, isopropyl, butyl, isobutyl, sec-butyl, tert-butyl, pentyl, isopentyl tert-pentylhexyl, isohexyl, etc. The term “lower alkyl” means an alkyl group straight chain or branched alkyl having 1 to 6 carbon atoms. Other alkyl groups will be readily apparent to those of skill in the art given the benefit of the present disclosure. An alkyl group may be unsubstituted or substituted with one or more substituent groups as described herein. For example, an alkyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, C₁-C₄ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the alkyl is substituted (e.g., with 1, 2, 3, 4, 5, or 6 substituent groups as described herein). In some embodiments, an alkyl group is substituted with a-OH group and may also be referred to herein as a “hydroxyalkyl” group, where the prefix denotes the —OH group and “alkyl” is as described herein. In some embodiments, an alkyl group is substituted with a-OR′ group.

Alkylene: The term “alkylene,” as used herein, represents a saturated divalent straight or branched chain hydrocarbon group and is exemplified by methylene, ethylene, isopropylene and the like. Likewise, the term “alkenylene” as used herein represents an unsaturated divalent straight or branched chain hydrocarbon group having one or more unsaturated carbon-carbon double bonds that may occur in any stable point along the chain, and the term “alkynylene” herein represents an unsaturated divalent straight or branched chain hydrocarbon group having one or more unsaturated carbon-carbon triple bonds that may occur in any stable point along the chain. In certain embodiments, an alkylene, alkenylene, or alkynylene group may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. For example, an alkylene, alkenylene, or alkynylene may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In certain embodiments, an alkylene, alkenylene, or alkynylene is unsubstituted. In certain embodiments, an alkylene, alkenylene, or alkynylene does not include any heteroatoms.

Alkenyl: As used herein, “alkenyl” means any linear or branched hydrocarbon chains having one or more unsaturated carbon-carbon double bonds that may occur in any stable point along the chain, e.g. “C₂-C₂₀ alkenyl” refers to an alkenyl group having 2-20 carbons. For example, an alkenyl group includes prop-2-enyl, but-2-enyl, but-3-enyl, 2-methylprop-2-enyl, hex-2-enyl, hex-5-enyl, 2,3-dimethylbut-2-enyl, and the like. In some embodiments, the alkenyl comprises 1, 2, or 3 carbon-carbon double bond. In some embodiments, the alkenyl comprises a single carbon-carbon double bond. In some embodiments, multiple double bonds (e.g., 2 or 3) are conjugated. An alkenyl group may be unsubstituted or substituted with one or more substituent groups as described herein. For example, an alkenyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHW, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀alkyl, or C₁-C₃ alkyl). In some embodiments, R′ independently is unsubstituted C₁-C₃ alkyl. In some embodiments, the alkenyl is unsubstituted. In some embodiments, the alkenyl is substituted (e.g., with 1, 2, 3, 4, 5, or 6 substituent groups as described herein). In some embodiments, an alkenyl group is substituted with a-OH group and may also be referred to herein as a “hydroxyalkenyl” group, where the prefix denotes the —OH group and “alkenyl” is as described herein.

Alkynyl: As used herein, “alkynyl” means any hydrocarbon chain of either linear or branched configuration, having one or more carbon-carbon triple bonds occurring in any stable point along the chain, e.g. “C₂-C₂₀ alkynyl” refers to an alkynyl group having 2-20 carbons. Examples of an alkynyl group include prop-2-ynyl, but-2-ynyl, but-3-ynyl, pent-2-ynyl, 3-methylpent-4-ynyl, hex-2-ynyl, hex-5-ynyl, etc. In some embodiments, an alkynyl comprises one carbon-carbon triple bond. An alkynyl group may be unsubstituted or substituted with one or more substituent groups as described herein. For example, an alkynyl group may be substituted with one or more (e.g., 1, 2, 3, 4, 5, or 6 independently selected substituents) of halogen, —COR′, —CO₂, —CO₂R, —CN, —OH, —OR′, —OCOR′, —OCOR′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁, alkyl, or C₁-C₃ alkyl). In some embodiments, R′ independently is unsubstituted C₁-C₃ alkyl. In some embodiments, the alkynyl is unsubstituted. In some embodiments, the alkynyl is substituted (e.g., with 1, 2, 3, 4, 5, or 6 substituent groups as described herein).

Alkoxy: The term “alkoxy” refers to the group —O-alkyl, including from 1 to 10 carbon atoms of a straight, branched, saturated cyclic configuration and combinations thereof, attached to the parent molecular structure through an oxygen. Examples include methoxy, ethoxy, propoxy, isopropoxy, butoxy, t-butoxy, pentoxy, cyclopropyloxy, cyclohexyloxy and the like. “Lower alkoxy” refers to alkoxy groups containing one to six carbons. In some embodiments, C₁₋₄ alkoxy is an alkoxy group which encompasses both straight and branched chain alkyls of from 1 to 4 carbon atoms. Unless stated otherwise in the specification, an alkoxy group can be optionally substituted by one or more substituents (e.g., as described herein for alkyl). The terms “alkenoxy” and “alkynoxy” mirror the above description of “alkoxy” wherein the prefix “alk” is replaced with “alken” or “alkyn” respectively, and the parent “alkenyl” or “alkynyl” terms are as described herein.

Amide: The term “amide” or “amido” refers to a chemical moiety with formula —C(O)N(R′)₂, —C(O)N(R′)—, —NR′C(O)R′, or —NR′C(O)—, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, or heterocycloalkyl (bonded through a ring carbon), unless stated other-wise in the specification, each of which moiety can itself be optionally substituted as described herein, or two R′ can combine with the nitrogen atom to form a 3-, 4-, 5-, 6-, or 7-membered ring.

Ureido: The term “ureido” refers to a chemical moiety with formula —NR′C(O)NR′—, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, or heterocycloalkyl (bonded through a ring carbon), unless stated other-wise in the specification, each of which moiety can itself be optionally substituted as described herein, or two R′ can combine with the nitrogen atom to form a 3-, 4-, 5-, 6-, or 7-membered ring.

Amino: The term “amino” or “amine” refers to a —N(R′)₂ group, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, or heterocycloalkyl (bonded through a ring carbon), unless stated otherwise in the specification, each of which moiety can itself be optionally substituted as described herein, or two R′ can combine with the nitrogen atom to form a 3-, 4-, 5-, 6-, or 7-membered ring. In embodiments, an amino group is —NHR′, where R′ is aryl (“arylamino”), heteroaryl (“heteroarylamino”), or alkyl (“alkylamino”).

Aryl: The term “aryl” used alone or as part of a larger moiety as in “aralkyl,” refers to a monocyclic, bicyclic, or tricyclic carbocyclic ring system having a total of six to fourteen ring members, wherein said ring system has a single point of attachment to the rest of the molecule, wherein at least one ring in the system is aromatic, and wherein each ring in the system contains 4 to 7 ring members. In some embodiments, an aryl group has 6 ring carbon atoms (“C₆ aryl,” e.g., phenyl). In some embodiments, an aryl group has 10 ring carbon atoms (“C₁, aryl,” e.g., naphthyl such as 1-naphthyl and 2-naphthyl). In some embodiments, an aryl group has 14 ring carbon atoms (“C₁₄ aryl,” e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Exemplary aryls include phenyl, naphthyl, and anthracene.

Arylalkyl: The term “arylalkyl” refers to an -(alkylene)-aryl radical where aryl and alkylene are as disclosed herein and which are optionally substituted by one or more of the exemplary substituent groups described herein. The “arylalkyl” group is bonded to the parent molecular structure through the alkylene moiety. The term “arylalkoxy” refers to an —O-[arylalkyl] radical (—O-[(alkylene)-aryl]), which is attached to the parent molecular structure through the oxygen.

Arylene: The term “arylene” as used herein refers to an aryl group that is divalent (that is, having two points of attachment to the molecule). Exemplary arylenes include phenylene (e.g., unsubstituted phenylene or substituted phenylene).

Cyclic: The term “cyclic” as used herein, refers to any covalently closed structure. Cyclic moieties include, for example, carbocycles (e.g., aryls and cycloalkyls), heterocycles (e.g., heteroaryls and heterocycloalkyls), aromatics (e.g. aryls and heteroaryls), and non-aromatics (e.g., cycloalkyls and heterocycloalkyls). In some embodiments, cyclic moieties are optionally substituted. In some embodiments, cyclic moieties form part of a ring system.

Cycloaliphatic: The term “cycloaliphatic” refers to a monocyclic or polycyclic radical that contains only carbon and hydrogen, and can be saturated or partially unsaturated. Fully saturated cycloaliphatics can be termed “cycloalkyl”. Partially unsaturated cycloalkyl groups can be termed “cycloalkenyl” if the carbocycle contains at least one double bond, or “cycloalkynyl” if the carbocycle contains at least one triple bond. Cycloaliphatic groups include groups having from 3 to 13 ring atoms (e.g., C₃₋₁₃ cycloalkyl). Whenever it appears herein, a numerical range such as “3 to 10” refers to each integer in the given range; e.g., “3 to 10 carbon atoms” means that the cycloaliphatic group (e.g., cycloalkyl) can consist of 3 carbon atoms, 4 carbon atoms, 5 carbon atoms, etc., up to and including 10 carbon atoms. The term “cycloaliphatic” also includes bridged and spiro-fused cyclic structures containing no heteroatoms. The term also includes monocyclic or fused-ring polycyclic (i.e., rings which share adjacent pairs of ring atoms) groups. Polycyclic cycloaliphatic groups include bicycles, tricycles, tetracycles, and the like. In some embodiments, “cycloalkyl” can be a C₃₋₈ cycloalkyl group. In some embodiments, “cycloalkyl” can be a C₃₋₅ cycloalkyl group. Illustrative examples of cycloaliphatic groups include, but are not limited to the following moieties: C₃₋₆ cycloaliphatic groups include, without limitation, cyclopropyl (C₃), cyclobutyl (C₄), cyclopentyl (C₅), cyclopentenyl (C₅), cyclohexyl (C₆), cyclohexenyl (C₆), cyclohexadienyl (C₆) and the like. Examples of C₃₋₇ cycloaliphatic groups include norbornyl (C₇). Examples of C₃₋₈ cycloaliphatic groups include the aforementioned C₃₋₇ carbocyclyl groups as well as cycloheptyl (C₇), cycloheptadienyl (C₇), cyclohept-atrienyl (C₇), cyclooctyl (C₈), bicyclo[2.2.1]heptanyl, bicyclo[2.2.2]octanyl, and the like. Examples of C₃₋₁₃ cycloaliphatic groups include the aforementioned C₃₋₈ carbocyclyl groups as well as octahydro-1H indenyl, decahydronaphthalenyl, spiro[4.5]decanyl, and the like.

Cyano: The term “cyano” refers to a —CN group.

Deuterium: The term “deuterium” is also called heavy hydrogen. Deuterium is isotope of hydrogen with a nucleus consisting of one proton and one neutron, which is double the mass of the nucleus of ordinary hydrogen (one proton). In embodiments, deuterium can also be identified as ²H.

Ester: The term “ester” refers to a group of formula —C(O)OR′ or R′OC(O)—, where R′ is selected from alkyl, alkenyl, alkynyl, heteroalkyl (bonded through a chain carbon), cycloalkyl, aryl, arylalkyl, heteroaryl, heteroarylalkyl, or heterocycloalkyl as described herein.

Halogen or Halo: As used herein, the term “halogen” or “halo” means fluorine, chlorine, bromine, or iodine.

Heteroalkyl: The term “heteroalkyl” is meant a branched or unbranched alkyl, alkenyl, or alkynyl group having from 1 to 14 carbon atoms in addition to 1, 2, 3 or 4 heteroatoms independently selected from the group consisting of N, O, S, and P. Heteroalkyls include tertiary amines, secondary amines, ethers, thioethers, amides, thioamides, carbamates, thiocarbamates, hydrazones, imines, phosphodiesters, phosphoramidates, sulfonamides, and disulfides. A heteroalkyl group may optionally include monocyclic, bicyclic, or tricyclic rings, in which each ring desirably has three to six members. Examples of heteroalkyls include polyethers, such as methoxymethyl and ethoxyethyl. Accordingly, the term “heteroalkoxy” refers to the group —O-heteroalkyl, where the group is attached to the parent molecular structure via the oxygen.

Heteroalkylene: The term “heteroalkylene,” as used herein, represents a divalent form of a heteroalkyl group as described herein.

Heteroaryl: The term “heteroaryl,” as used herein, refers to a monocyclic, bicyclic, or tricyclic carbocyclic ring system having a total of six to fourteen ring members, wherein said ring system has a single point of attachment to the rest of the molecule, wherein at least one ring in the system is aromatic, wherein each ring in the system contains 4 to 7 ring members, and wherein at least one ring atom is a heteroatom such as, but not limited to, nitrogen, oxygen, or sulfur. Examples of heteroaryl groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The term “N-containing heteroaryl” refers to heteroaryl groups which comprise at least one nitrogen in the ring system (e.g., a heteroaryl comprising 1, 2, or 3 nitrogen atoms). Further, the term “heteroaryloxy” refers to the group —O-heteroaryl, where the group is attached to the parent molecular structure via the oxygen.

Heteroarylene: The term “heteroalkylene,” as used herein, represents a divalent form of a heteroaryl group as described herein.

Heteroarylalkyl: The term “heteroarylalkyl” refers to an -(alkylene)-heteroaryl radical where heteroaryl and alkylene are as disclosed herein and which are optionally substituted by one or more of the exemplary substituent groups described herein. The “heteroarylalkyl” group is bonded to the parent molecular structure through the alkylene moiety. The term “heteroarylalkoxy” refers to an —O-[heteroarylalkyl] radical (—O-[(alkylene)-heteroaryl]), which is attached to the parent molecular structure through the oxygen.

Heterocycloalkyl: The term “heterocycloalkyl,” as used herein, is a non-aromatic ring wherein at least one atom is a heteroatom such as, but not limited to, nitrogen, oxygen, sulfur, or phosphorus, and the remaining atoms are carbon. Examples of heterocycloalkyl groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. The heterocycloalkyl group can be substituted or unsubstituted.

Heterocycle: The term “heterocycle” or “heterocyclyl” refers to heteroaryl and heterocycloalkyl as used herein, refers to groups containing one to four heteroatoms each selected from O, S and N, wherein each heterocycle group has from 4 to 10 atoms in its ring system, and with the proviso that the ring of said group does not contain two adjacent O or S atoms. Herein, whenever the number of carbon atoms in a heterocycle is indicated (e.g., C₁-C₆-heterocycle), at least one other atom (the heteroatom) must be present in the ring. Designations such as “C₁-C₆-heterocycle” refer only to the number of carbon atoms in the ring and do not refer to the total number of atoms in the ring. In some embodiments, it is understood that the heterocycle ring has additional heteroatoms in the ring. Designations such as “4-6-membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In some embodiments, in heterocycles that have two or more heteroatoms, those two or more heteroatoms are the same or different from one another. In some embodiments, heterocycles are optionally substituted. In some embodiments, binding to a heterocycle is at a heteroatom or via a carbon atom. Heterocycloalkyl groups include groups having only 4 atoms in their ring system, but heteroaryl groups must have at least 5 atoms in their ring system. The heterocycle groups include benzo-fused ring systems. An example of a 4-membered heterocycle group is azetidinyl (derived from azetidine). An example of a 5-membered heterocycle group is thiazolyl. An example of a 6-membered heterocycle group is pyridyl, and an example of a 10-membered heterocycle group is quinolinyl. In some embodiments, the foregoing groups, as derived from the groups listed above, are C-attached or N-attached where such is possible. For instance, in some embodiments, a group derived from pyrrole is pyrrol-1-yl (N-attached) or pyrrol-3-yl (C-attached). Further, in some embodiments, a group derived from imidazole is imidazol-1-yl or imidazol-3-yl (both N-attached) or imidazol-2-yl, imidazol-4-yl or imidazol-5-yl (all C-attached). The heterocycle groups include benzo-fused ring systems and ring systems substituted with one or two oxo (═O) moieties such as pyrrolidin-2-one. In some embodiments, depending on the structure, a heterocycle group is a monoradical or a diradical (i.e., a heterocyclene group). The heterocycles described herein are substituted with 0, 1, 2, 3, or 4 substituents independently selected from alkenyl, alkoxy, alkoxyalkyl, alkoxycarbonyl, alkyl, alkylcarbonyl, alkylcarbonyloxy, alkylthio, alkylthioalkyl, alynyl, carboxy, cyano, formyl, haloalkoxy, haloalkyl, halogen, hydroxyl, hydroxyalkylene, mercapto, nitro, amino, and amido moities.

Isotope: The term “isotope” refers to a variant of a particular chemical element which differs in neutron number, and consequently in nucleon number. All isotopes of a given element have the same number of protons but different numbers of neutrons in each atom.

Nitro: The term “nitro” refers to a —NO₂ group.

Sulfonamide: The term “sulfonamide” or sulfonamido” refers to the following groups: —S(═O)₂—(R′)₂, —N(R′)—S(═O)₂—R′, —S(═O)₂—N(R′)—, or —N(R′)—S(═O)₂—, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, or heterocycloalkyl (bonded through a ring carbon), unless stated other-wise in the specification, each of which moiety can itself be optionally substituted as described herein, or two R′ can combine with the nitrogen atom to form a 3-, 4-, 5-, 6-, or 7-membered ring.

Nitrogen protecting group: In certain embodiments, the substituent present on a nitrogen atom is a nitrogen protecting group (also referred to as an amino protecting group or an N-protecting group). Nitrogen protecting groups are well known in the art and include those described in detail in Protecting Groups in Organic Synthesis, T. W. Greene and P. G. M. Wuts, 3rd edition, John Wiley & Sons, 1999, incorporated herein by reference.

For example, nitrogen protecting groups such as amide groups (e.g., —C(═O)R^{aa} include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoe), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (lpaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthryl methyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-triethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl 4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pme), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetrammethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fecm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenanide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

Moiety: The term “moiety” refers to a specific segment or functional group of a molecule. Chemical moieties are often recognized chemical entities embedded in or appended to a molecule.

Molecular groups herein may be substituted or unsubstituted (e.g., as described herein). The term “substituted” means that the specified group or moiety bears one or more substituents: at least one hydrogen present on a group atom (e.g., a carbon or nitrogen atom) is replaced with a permissible substituent, e.g., a substituent which upon substitution for the hydrogen results in a stable compound, e.g., a compound which does not spontaneously undergo transformation such as by rearrangement, cyclization, elimination, or other reaction. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In embodiments, a group described herein is substituted. In embodiments, a group described herein is unsubstituted. In cases where a specified moiety or group is not expressly noted as being optionally substituted or substituted with any specified substituent, it is understood that such a moiety or group is intended to be unsubstituted.

A wide variety of substituents are well known, and methods for their formation and introduction into a variety of parent groups are also well known. Representative substituents include but are not limited to alkyl, cycloalkyl, alkenyl, cycloalkenyl, alkynyl, arylalkyl, alkylaryl, aryl, heteroaryl, heterocycloalkyl, hydroxyalkyl, arylalkyl, aminoalkyl, haloalkyl, thioalkyl, alkylthioalkyl, carboxyalkyl, imidazolylalkyl, indolylalkyl, mono-, di- and trihaloalkyl, mono-, di- and trihaloalkoxy, amino, alkylamino, dialkylamino, alkoxy, hydroxy, halo (e.g., —C₁ and —Br), nitro, oximino, —COOR⁵⁰, —COR⁵⁰, —SO₀₋₂R⁵⁰, —SO₂NR⁵⁰R⁵¹, —NR⁵²SO₂R⁵⁰, —C(R⁵⁰R⁵¹), ═N—OR⁵⁰, ═N—CN, ═C(halo)₂, ═S, ═O, —CON(R⁵⁰R⁵¹), —OCOR¹⁰, —OCON(R⁵⁰R⁵¹), —N(R⁵²)CO(R⁵⁰), —N(R⁵²)COOR⁵⁰, —N(R⁵²)CON(R⁵⁰(R⁵¹), —P(OR⁵⁰)₂, —P(O)R⁵⁰R⁵¹, and —P(O)OR⁵⁰OR⁵¹, wherein R⁵⁰, R⁵¹ and R⁵² may be independently selected from the following: a hydrogen atom and a branched or straight-chain, C₁₋₆ alkyl, C₃₋₆-cycloalkyl, C₄₋₆-heterocycloalkyl, heteroaryl and aryl group, with or without substituents. When permissible, R⁵⁰ and R⁵¹ can be joined together to form a carbocyclic or heterocyclic ring system.

In preferred embodiments, the substituent is selected from halogen, —COR′, —CO₂H, —CO₂R′, —CN, —OH, —OR′, —OCOR′, —OCO₂R′, —NH₂, —NHR′, —N(R′)₂, —SR′, and —SO₂R′, wherein each instance of R′ independently is C₁-C₂₀ aliphatic (e.g., C₁-C₂₀ alkyl, C₁-C₁₅ alkyl, C₁-C₁₀alkyl, or C₁-C₃ alkyl). In certain embodiments thereof, R′ independently is an unsubstituted alkyl (e.g., unsubstituted C₁-C₂₀ alkyl, C₁-C₁₅ alkyl, C₁-C₁₀ alkyl, or C₁-C₃ alkyl). Preferably, R′ independently is unsubstituted C₁-C₃ alkyl.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to embrace hydrates, solvates, and polymorphs of such compounds, and mixtures thereof.

### Compounds of the Invention

Described herein are new compounds that can be effective inhibitors of EGFR. Such compounds can be useful for treating various diseases and disorders, including EGFR-driven cancers such as non-small cell lung cancer (NSCLC) characterized by mutant EGFR.

Exemplary compounds and exemplary structural features are described herein.

Compounds of Formulas (I)-XIII)

In one aspect, provided herein are compounds having a structure according to Formula (I):

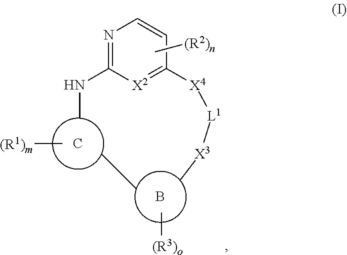

or a pharmaceutically acceptable salt thereof, wherein
X² is independently N or CR⁵;
each of X³ and X⁴ is independently a covalent bond, O, S, NR⁶, C(O)NR⁶, NR⁶C(O), NR⁶C(O)NR⁶, or (C(R⁷)₂)_{q};
L¹ is independently a covalent bond, C₁₋₆ heteroalkylene, C₁₋₆ alkylene, C₂₋₆ alkenylene, C₂₋₆ alkynylene, C₃₋₆ cycloalkylene, 3- to 10-membered heterocyclylene, phenylene, naphthylene, or 5- to 10-membered heteroarylene;
each R¹ and R² is independently

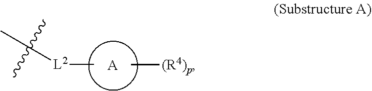

OH, CN, halogen, C₁₋₆ aliphatic, C₁₋₆ alkoxy, NR⁸R⁹, C(O)R¹⁰, CO₂R¹⁰, ((O)NR⁸R⁹, NR¹¹C(O)R¹⁰, NR¹¹CO₂R¹⁰, NR¹¹C(O)NR⁸R⁹, or (CH₂),R²; or two R¹ or two R², together to which the atoms they are attached form a 5- to 10-membered ring;
L² is independently a covalent bond, O, NR^{L}, C(O), C(O)NR^{L}, NR^{L}C(O), CR^{L}₂;
R^{L} is independently H or C₁₋₆ alkyl;
A is independently phenyl, naphthyl, 5- to 13-membered heteroaryl, C₃-C₁₀ cycloaliphatic, or 3- to 10-membered heterocyclyl;
B is independently phenyl, naphthyl, 5- to 13-membered heteroaryl, C₃-C₁₀ cycloaliphatic, or 3- to 10-membered heterocyclyl;
C is independently 5- or 6-membered heteroaryl;
each R³ is independently OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, or (CH₂)ᵣR¹²; each R⁴ is independently H, OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, NR¹¹(CH₂)ₛNR⁸R⁹, (CH₂)ₜNR⁸R⁹, (CH₂)ₜOH, (CH₂)ₜOCH₃, O(CH₂)OH, O(CH₂)ₜOCH₃, O(CH₂)ᵣR, or (CH₂)ᵣR²; or R⁴ and R⁶, or R⁴ and R⁷, together with the atoms to which they are attached, form a 5- to 6-membered ring;
each R⁵ is independently H, OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, or (CH₂),R²;
each R⁶ is independently H, a N-protecting group, or C₁₋₆ alkyl; or R⁶ and R⁴, together with the atoms to which they are attached, form a 5- to 6-membered ring;
each R⁷ is independently H or C₁₋₆ alkyl; or two R⁷ on the same carbon combine to from an oxo (═O) group; or R⁷ and R⁴, together with the atoms to which they are attached, form a 5- to 6-membered ring;
each R⁸, R⁹, and R¹¹ is independently H or C₁₋₆ alkyl; or R⁸ and R⁹, together with the nitrogen atom to which they are attached, form a 3- to 10-membered heterocyclyl, or R and R¹¹, together with the atoms to which they are attached, form a 3- to 10-membered heterocyclyl;
each R¹⁰ is independently C₁₋₆ aliphatic, C₃-C₁₀ cycloaliphatic, 3- to 10-membered heterocyclyl, phenyl, naphthyl, or a 5- to 12-membered heteroaryl; or R¹⁰ and R¹¹, together with the atoms to which they are attached, form a 3- to 10-membered heterocyclyl;
each R¹² is independently C₃-C₁₀ cycloaliphatic, 3- to 10-membered heterocyclyl, phenyl, naphthyl, or a 5- to 12-membered heteroaryl;
each m, n, and o is independently 0, 1, or 2;
each p is independently 0, 1, 2; 3, or 4;
each q is independently 1 or 2;
each r is independently an integer of 0-4;
each s is independently an integer of 2-6; and
each t is independently an integer of 1-6.

In embodiments, each R⁴ is independently H, OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, NR¹¹(CH₂)ₛNR⁸R⁹, (CH₂)ₜNR⁸R⁹, or (CH₂)ᵣR¹²; or R⁴ and R⁶, or R⁴ and R⁷, together with the atoms to which they are attached, form a 5- to 6-membered ring.

In embodiments, n is 0. In embodiments, n is 1. In embodiments, m is 2. In embodiments, n is 1 or 2.

In embodiments, n is 0. In embodiments, n is 1. In embodiments, n is 2. In embodiments, n is 1 or 2.

In embodiments, m is not 0. In embodiments, n is not 0. In embodiments, at least one m or n is not 0. In embodiments, n is 1, and n is 0. In embodiments, n is 1, and m is 0.

In embodiments, p is 0. In embodiments, p is 1. In embodiments, p is 2. In embodiments, p is 3. In embodiments, p is 4.

In embodiments, R¹ is present. In embodiments, R² is present. In embodiments, at least one of R¹ and R² is present. In embodiments, one of R and R¹ is present. In embodiments, no more than one of R¹ and R² is present. In embodiments, one of R¹ and R² is present and is

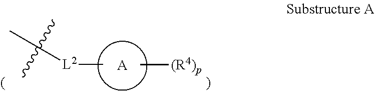

or halogen (e.g., F, Cl, Br, or I). In embodiments, one of R¹ and R² is present and is

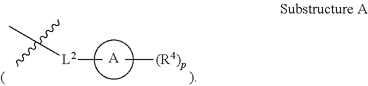

In embodiments,

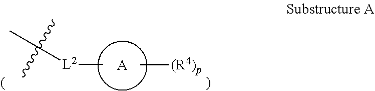

is not present. In embodiments, one Substructure A group is present. In embodiments, no more than one Substructure A is present. In embodiments, two Substructure A groups are present (e.g., two Substructure A groups with identical or different structures). In embodiments, more than two Substructure A groups are present (e.g., more than two Substructure A groups with identical or different structures). In embodiments, no more than one Substructure A group is present.

In embodiments, C is 5- or 6-membered N-containing heteroaryl. In embodiments, C is pyridyl, pyrimidyl, pyrazolyl, pyrrolyl, thiazolyl, oxazolyl, or imidazolyl.

In embodiments, A is pyridyl, pyrimidyl, pyrazolyl, pyrrolyl, thiazolyl, oxazolyl, or imidazolyl.

In embodiments, B is pyridyl, pyrimidyl, pyrazolyl, pyrrolyl, thiazolyl, oxazolyl, or imidazolyl.

In embodiments, each of A and B is pyrazolyl. In embodiments, In embodiments, A is pyridyl or pyrimidyl.

In embodiments, a compound of Formula (I) has a structure according to Formula (I-A),

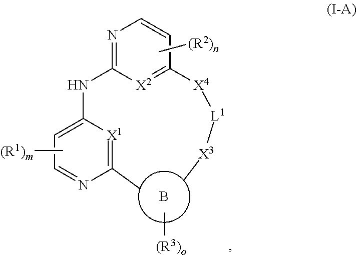

or a pharmaceutically acceptable salt thereof wherein X¹ is N or CR⁵.

In embodiments, B, R¹, R², R³, L¹, X², X³, X⁴, m, o, and p are according to any embodiment described herein.

In embodiments, C is pyrazolyl.

In embodiments, a compound of Formula (I) has a structure according to Formula (I-B),

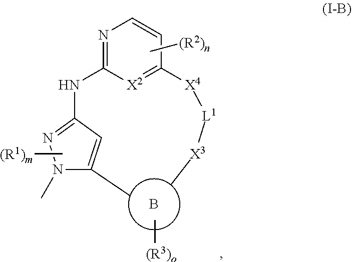

or a pharmaceutically acceptable salt thereof, wherein m is 0 or 1.

In embodiments, B, R¹, R², R³, L¹, X², X³, X⁴, o, and p are according to any embodiment described herein.

In embodiments, C is thiazolyl.

In embodiments, a compound of Formula (I) has a structure according to Formula (I-C).

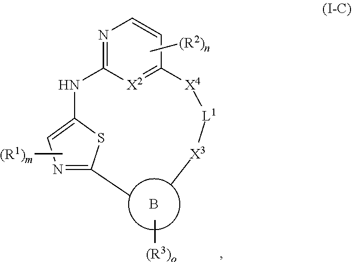

or a pharmaceutically acceptable salt thereof, wherein m is 0 or 1.

In embodiments, B, R¹, R², R³, L¹, X², X³, X⁴, o, and p are according to any embodiment described herein.

In embodiments, a compound of Formula (I) has a structure according to Formula (II),

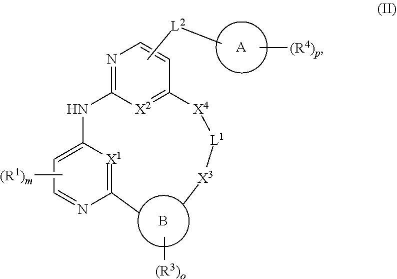

or a pharmaceutically acceptable salt thereof, wherein
each R¹ is independently OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, or R¹².

In embodiments, A, B, R¹, R³, R⁴, L¹, X¹, X², X³, X⁴, m, o, and p are according to any embodiment described herein.

In embodiments, R¹ is independently OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, or R¹².

In embodiments, a compound of Formula (I) or Formula (II) has a structure according to Formula (II-A),

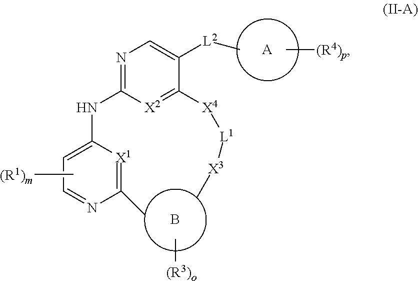

or a pharmaceutically acceptable salt thereof.

In embodiments, A, B, R¹, R³, R⁴, L¹, X¹, X², X³, X⁴, m, o, and p are according to any embodiment described herein.

In embodiments, X³ is a covalent bond, O, S, NR⁶, C(O)NR⁶, NR⁶C(O), NR⁶C(O)NR¹⁰, or (C(R⁷)₂)_{q}. In embodiments, X³ is O. In embodiments, X⁴ is a covalent bond, O, S, NR⁶, C(O)NR⁶, NR⁶C(O), NR⁶C(O)NR⁶, or (C(R⁷)₂)_{q}. In embodiments, X⁴ is O. In embodiments, X³ and X⁴ are the same. In embodiments, X³ and X⁴ are different. In embodiments, V and V are both O.

In embodiments, a compound of Formula (I) has a structure according to Formula (III),

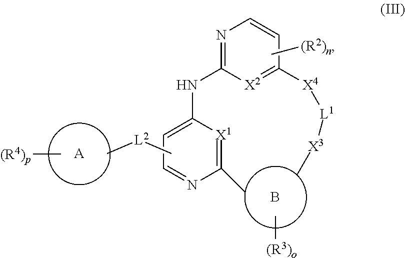

or a pharmaceutically acceptable salt thereof, wherein
each R² is independently OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, or R².

In embodiments, A, B, R², R³, R⁴, L¹, X¹, X², X³, X⁴, X⁴, o, and p are according to any embodiment described herein.

In embodiments, each R² is independently OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, or R¹².

In embodiments, a compound of Formula (I) or Formula (III) has a structure according to Formula (III-A),

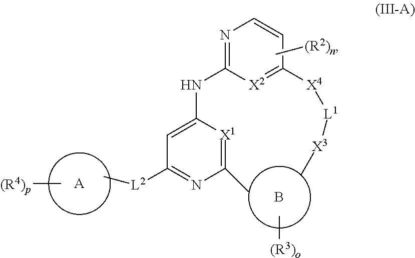

or a pharmaceutically acceptable salt thereof.

In embodiments, A, B, R², R³, R⁴, L¹, X¹, X², X³, X⁴, n, o, and p are according to any embodiment described herein.

In embodiments, X³ is a covalent bond, O, S, NR⁶, C(O)NR, NR⁶C(O), NR⁶C(O)NR⁶, or (C(R⁷)₂)_{q}. In embodiments, X³ is O. In embodiments, 4 is a covalent bond, O, S, NR⁶, C(O)NR⁶, NR⁶C(O), NR⁶C(O)NR⁶, or (C(R⁷)₂)_{q}. In embodiments, 4 is O. In embodiments, X¹ and X⁴ are the same. In embodiments, X¹ and X⁴ are different. In embodiments, X³ and X⁴ are both O.

In embodiments, a compound of Formula (I) has a structure according to Formula (IV),

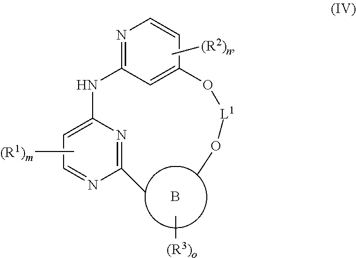

or a pharmaceutically acceptable salt thereof, wherein
L is unsubstituted linear or branched C₂₋₆ alkylene;
B is phenyl or 5- to 6-membered heteroaryl;
R³ is methyl, halogen, or CN;
o is 0 or 1; and
one of R¹ and R² is present as Substructure A.

In embodiments, B, R¹, R², R³, L¹, m, n, and o are according to any embodiment described herein.

In embodiments, L¹ is unsubstituted linear or branched C₂₋₆ alkylene (e.g., —(CH₂)₃— or —CH(CH₃)CH₂CH₂—).

In embodiments, B is phenyl. In embodiments, B is 5- to 6-membered heteroaryl (e.g., pyrazolyl).

In embodiments, R³ is methyl, halogen, or CN. In embodiments, R³ is methyl.

In embodiments, o is 0. In embodiments, o is 1.

In embodiments, one of R¹ and R² is present as Substructure A.

In embodiments, a compound of Formula (I) or Formula (IV) has a structure according to Formula (V),

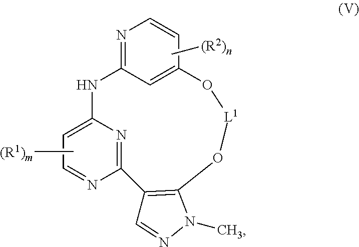

or a pharmaceutically acceptable salt thereof, wherein
L¹ is —(CH₂)₃— or —CH(CH₃)CH₂CH₂—.

In embodiments, R¹, R², L¹, m, and n are according to any embodiment described herein.

In embodiments, L is —(CH₂)₃—. In embodiments, L¹ is —CH(CH₃)CH₂CH₂—.

In embodiments, one of R¹ and R² is present as Substructure A.

In embodiments, a compound of Formula (I) or Formula (V) has a structure according to Formula (VI-1) or Formula (VI-2),

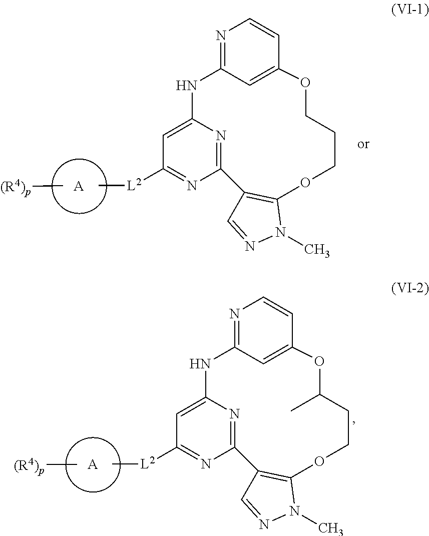

or a pharmaceutically acceptable salt thereof.

In embodiments, R⁴ and p are according to any embodiment described herein.

In embodiments, the sp³ carbon substituted by CH₃ has the (R)-configuration.

In embodiments, the sp³ carbon substituted by CH₃ has the (S)-configuration.

In embodiments, a compound of Formula (I), Formula (V), or Formula (VI-2) has a structure according to Formula (VI-3) or Formula (VI-4),

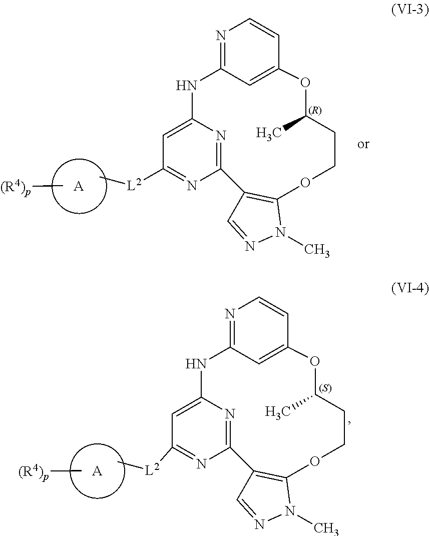

or a pharmaceutically acceptable salt thereof.

In embodiments, R⁴ and p are according to any embodiment described herein.

In embodiments, a compound of Formula (I), Formula (V), has a structure according to Formula (VII-1) or Formula (VII-2),

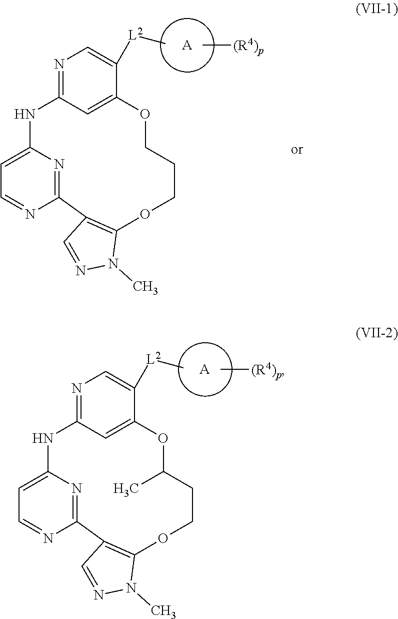

or a pharmaceutically acceptable salt thereof.

In embodiments, R⁴ and p are according to any embodiment described herein.

In embodiments, the sp³ carbon substituted by CH₃ has the (R)-configuration.

In embodiments, the sp³ carbon substituted by CH₃ has the (S)-configuration.

In embodiments, a compound of Formula (I), Formula (V), or Formula (VII-2) has a structure according to Formula (VII-3) or Formula (VII-4),

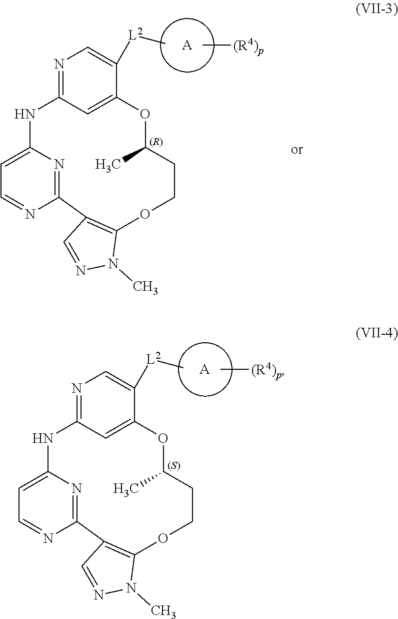

or a pharmaceutically acceptable salt thereof.

In embodiments, R⁴ and p are according to any embodiment described herein.

In embodiments, a compound of Formula (I), has a structure according to Formula (VIII),

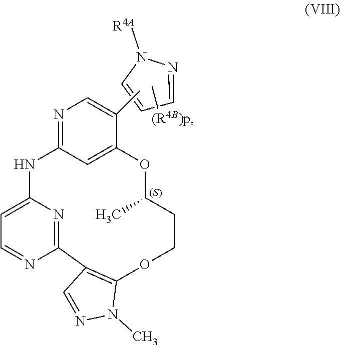

or a pharmaceutically acceptable salt thereof, wherein R^{4A} is a first R⁴ group, R⁴¹ is a second R⁴ group, and p is 0 or 1.

In embodiments, any occurrence of R⁴ is independently according to any embodiment described herein. In embodiments, R^{4A} and R^{4B} are independently according to any embodiment described herein.

In embodiments, a compound of Formula (VIII), has a structure according to Formula (VIII-1),

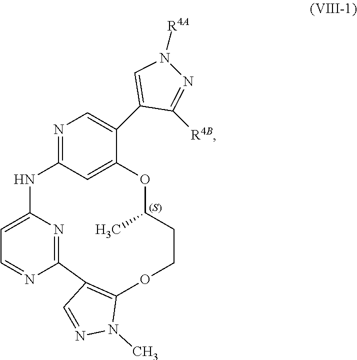

or a pharmaceutically acceptable salt thereof. In embodiments, R^{4A} and R^{4B} are independently according to any embodiment described herein.

In embodiments, a compound of Formula (VIII), has a structure according to Formula (VIII-2),

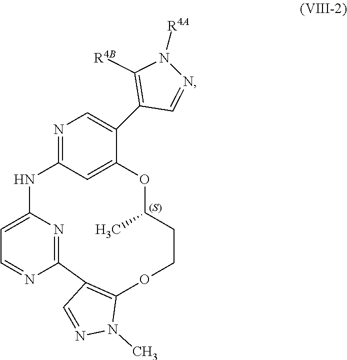

or a pharmaceutically acceptable salt thereof. In embodiments, R^{4A} and R^{4B} are independently according to any embodiment described herein.

In embodiments, a compound of Formula (VIII), has a structure according to Formula (VIII-3),

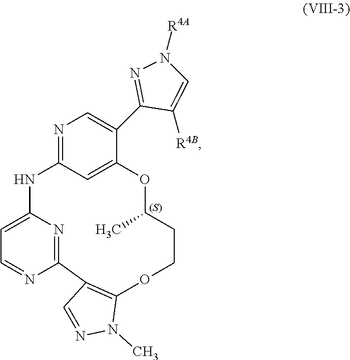

or a pharmaceutically acceptable salt thereof. In embodiments, R^{4A} and R^{4B} are independently according to any embodiment described herein.

In embodiments, a compound of Formula (VIII), has a structure according to Formula (VIII-4),

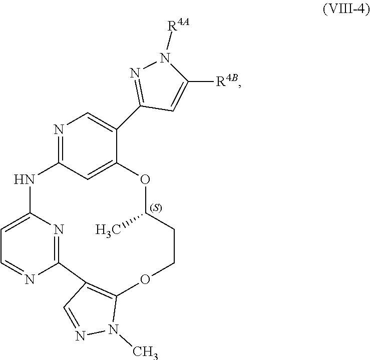

or a pharmaceutically acceptable salt thereof. In embodiments, R^{4A} and R^{4B} are independently according to any embodiment described herein.

In embodiments, a compound of Formula (VIII), has a structure according to Formula (VIII-5),

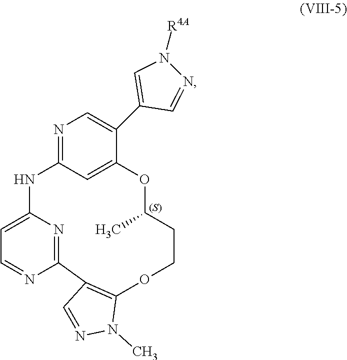

or a pharmaceutically acceptable salt thereof. In embodiments, R^{4A} is according to any embodiment described herein.

In embodiments, a compound of Formula (VIII), has a structure according to Formula (VIII-6),

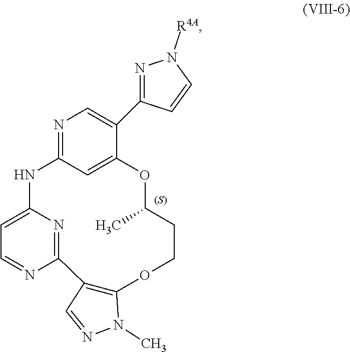

or a pharmaceutically acceptable salt thereof. In embodiments, R^{4A} is according to any embodiment described herein.

In embodiments, a compound of Formula (I), has a structure according to Formula (IX),

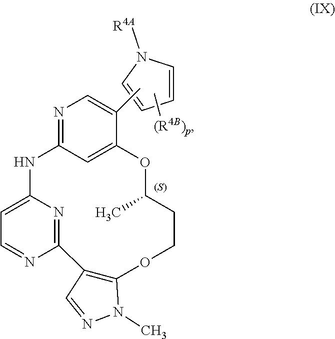

or a pharmaceutically acceptable salt thereof, wherein R^{4A} is a first R⁴ group, R^{4B} is a second R⁴ group, and p is 0 or 1.

In embodiments, any occurrence of R⁴ is independently according to any embodiment described herein. In embodiments, R^{4A} and R^{4B} are independently according to any embodiment described herein.

In embodiments, a compound of Formula (IX), has a structure according to Formula (IX-1),

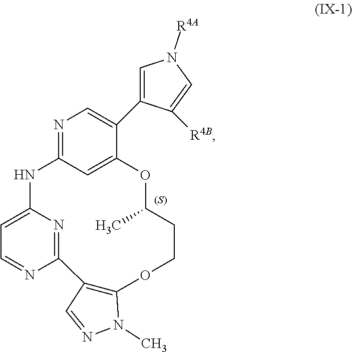

or a pharmaceutically acceptable salt thereof. In embodiments, R^{4A} and R^{4B} are independently according to any embodiment described herein.

In embodiments, a compound of Formula (IX), has a structure according to Formula (IX-2),

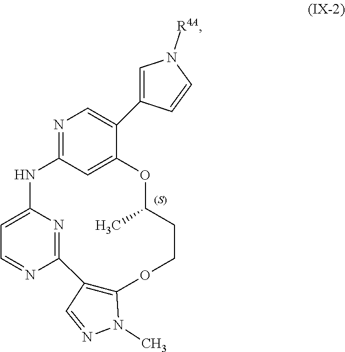

or a pharmaceutically acceptable salt thereof. In embodiments, R^{4A} is independently according to any embodiment described herein.

In embodiments, a compound of Formula (I), has a structure according to Formula (X),

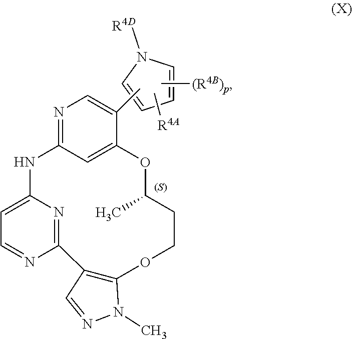

or a pharmaceutically acceptable salt thereof, wherein R^{4A} is a first R⁴ group, R^{4B} is a second R⁴ group, p is 0 or 1; and R^{4D} is third R⁴ group.

In embodiments, any occurrence of R⁴ is independently according to any embodiment described herein. In embodiments, R^{4A} and R^{4B} are independently according to any embodiment described herein. In embodiments, R^{4D} is a R⁴ group that is unsubstituted C₁₋₆ alkyl.

In embodiments, a compound of Formula (X), has a structure according to Formula (X-1),

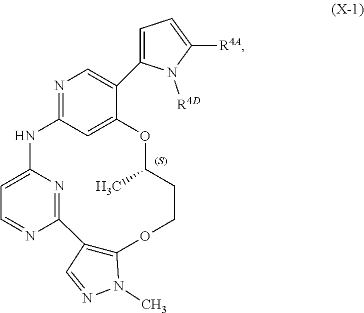

or a pharmaceutically acceptable salt thereof. In embodiments, R^{4A} and R^{4D} are independently according to any embodiment described herein.

In embodiments, a compound of Formula (X), has a structure according to Formula (X-2),

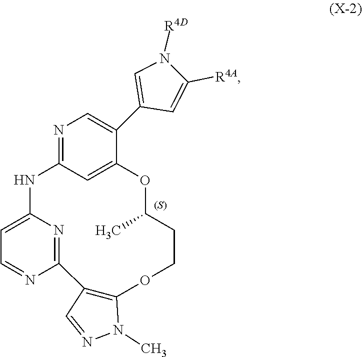

or a pharmaceutically acceptable salt thereof. In embodiments, R^{4A} and R^{4D} are independently according to any embodiment described herein.

In embodiments, a compound of Formula (X), has a structure according to Formula (X-3),

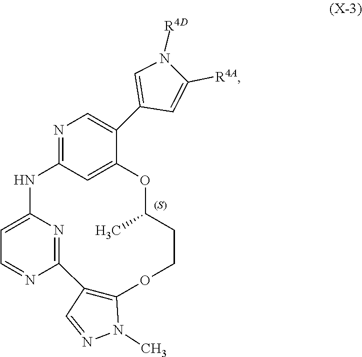

or a pharmaceutically acceptable salt thereof. In embodiments, R^{4A} and R^{4D} are independently according to any embodiment described herein.

In embodiments, a compound of Formula (X), has a structure according to Formula (X-4),

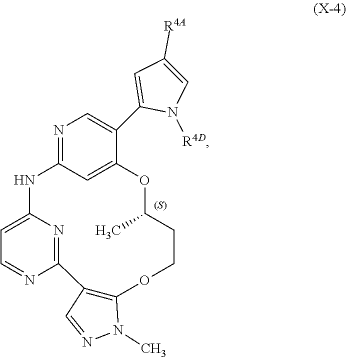

or a pharmaceutically acceptable salt thereof. In embodiments, R^{4A} and R^{4D} are independently according to any embodiment described herein.

In embodiments, a compound of Formula (I), has a structure according to Formula (XI),

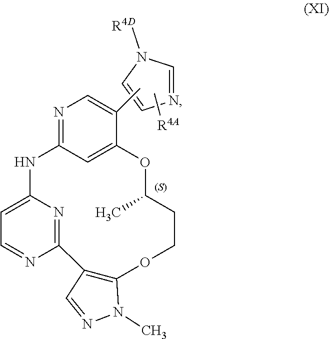

or a pharmaceutically acceptable salt thereof, wherein R^{4A} is a first R⁴ group, and R^{4D} is a second R⁴ group.

In embodiments, any occurrence of R⁴ is independently according to any embodiment described herein. In embodiments, R^{4A} and R^{4D} are independently according to any embodiment described herein. In embodiments, R^{4D} is a R⁴ group that is unsubstituted C₁₋₆ alkyl.

In embodiments, a compound of Formula (XI), has a structure according to Formula (XI-1),

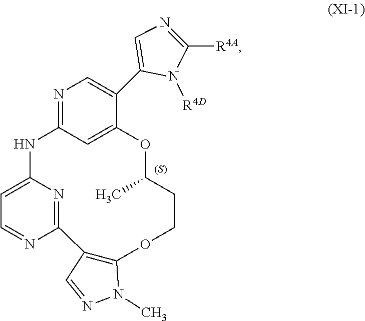

or a pharmaceutically acceptable salt thereof. In embodiments, R^{4A} and R^{4D} are independently according to any embodiment described herein.

In embodiments, a compound of Formula (I), has a structure according to Formula (XII),

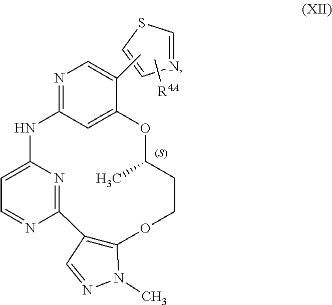

or a pharmaceutically acceptable salt thereof, wherein R^{4A} is a first R⁴ group.

In embodiments, R^{4A} is independently according to any embodiment described herein.

In embodiments, a compound of Formula (XII), has a structure according to Formula (XII-1),

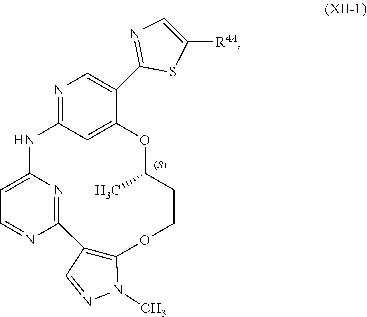

or a pharmaceutically acceptable salt thereof. In embodiments, R^{4A} is independently according to any embodiment described herein.

In embodiments, a compound of Formula (I), has a structure according to Formula (XIII),

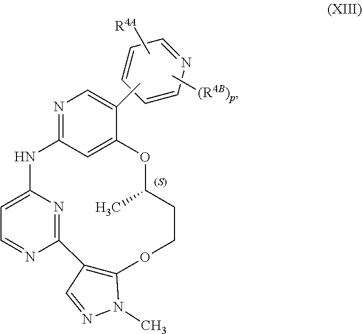

or a pharmaceutically acceptable salt thereof, wherein R^{4A} is a first R⁴ group, R^{4B} is a second R⁴ group, and p is 0 or 1.

In embodiments, R^{4A} and R^{4B} are independently according to any embodiment described herein.

In embodiments, a compound of Formula (XIII), has a structure according to Formula (XIII-1),

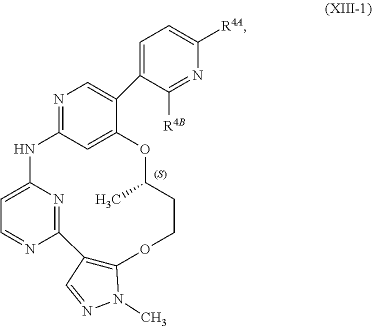

or a pharmaceutically acceptable salt thereof. In embodiments, R^{4A} and R^{4B} are independently according to any embodiment described herein.

In embodiments, a compound of Formula (XIII), has a structure according to Formula (XIII-2),

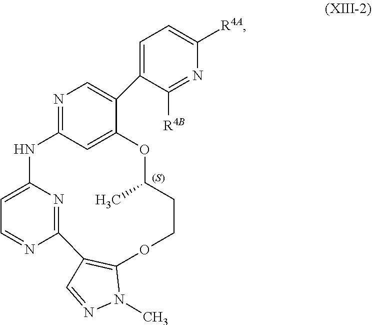

or a pharmaceutically acceptable salt thereof. In embodiments, R^{4A} and R^{4B} are independently according to any embodiment described herein.

In embodiments, a compound of Formula (XIII), has a structure according to Formula (XIII-3),

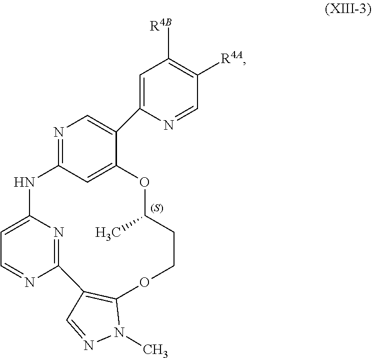

or a pharmaceutically acceptable salt thereof. In embodiments, R^{4A} and R^{4B} are independently according to any embodiment described herein.

In embodiments, a compound of Formula (XIII), has a structure according to Formula (XIII-4),

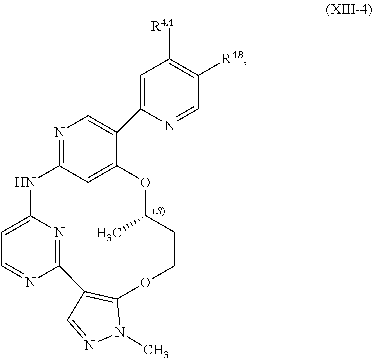

or a pharmaceutically acceptable salt thereof. In embodiments, R^{4A} and R^{4B} are independently according to any embodiment described herein.

In embodiments, a compound of Formula (XIII), has a structure according to Formula (XIII-5),

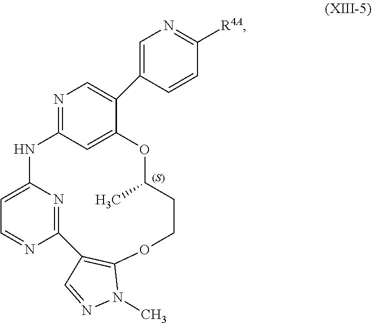

or a pharmaceutically acceptable salt thereof. In embodiments, R^{4A} and R^{4B} are independently according to any embodiment described herein.

In embodiments, a compound of Formula (I), has a structure according to Formula (XIV),

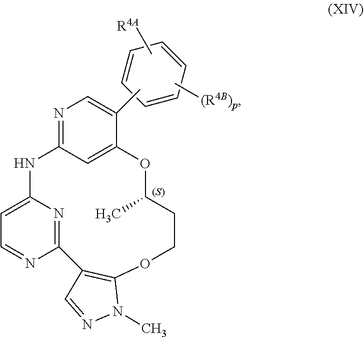

or a pharmaceutically acceptable salt thereof, wherein R^{4A} is a first R⁴ group, R^{4B} is a second R⁴ group, and p is 0 or 1.

In embodiments, any occurrence of R⁴ is independently according to any embodiment described herein. In embodiments, R^{4A} and R^{4B} are independently according to any embodiment described herein.

In embodiments, a compound of Formula (XIV), has a structure according to Formula (XIV-1),

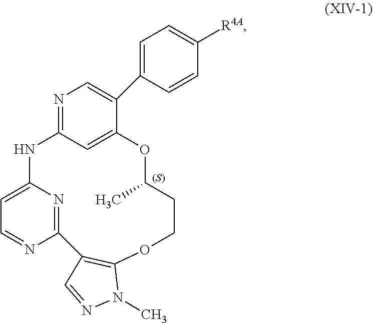

or a pharmaceutically acceptable salt thereof: In embodiments, R^{4A} is independently according to any embodiment described herein.

In embodiments, a compound of Formula (XIV), has a structure according to Formula (XIV-2),

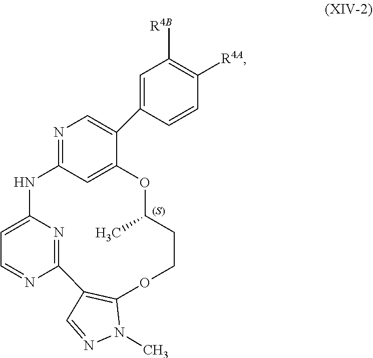

or a pharmaceutically acceptable salt thereof. In embodiments, R^{4A} and R^{4B} are independently according to any embodiment described herein.

In embodiments, a compound of Formula (XIV), has a structure according to Formula (XIV-3),

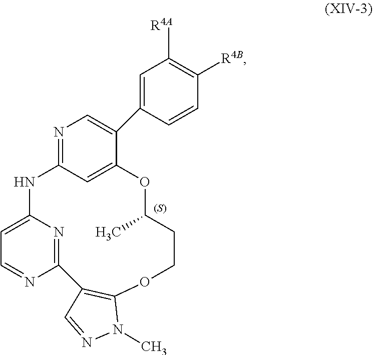

or a pharmaceutically acceptable salt thereof. In embodiments, R^{4A} and R^{4B} are independently according to any embodiment described herein.

In embodiments, a compound of Formula (XIV), has a structure according to Formula (XIV-4),

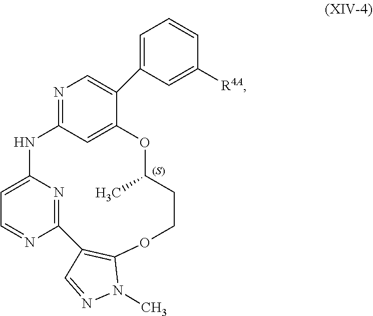

or a pharmaceutically acceptable salt thereof. In embodiments, R^{4A} is independently according to any embodiment described herein.

In embodiments, a compound of Formula (I), has a structure according to Formula (XV),

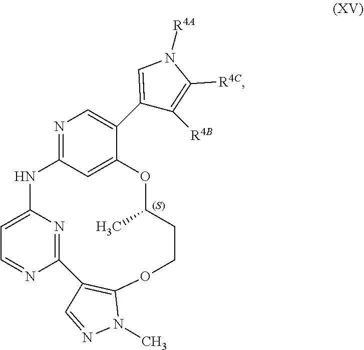

or a pharmaceutically acceptable salt thereof, wherein R^{4A} is a first R⁴ group, R^{4B} is a second R⁴ group, and R^{4C} is a third R⁴ group.

In embodiments, any occurrence of R⁴ is independently according to any embodiment described herein. In embodiments, R^{4A}, R^{4B}, and R^{4C} are independently according to any embodiment described herein.

In embodiments, a compound of Formula (I), has a structure according to Formula (XVI),

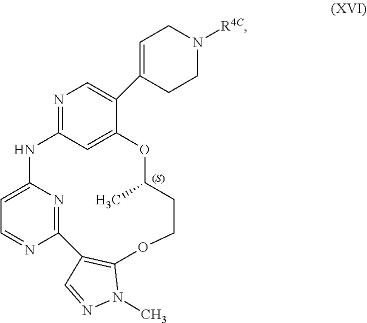

or a pharmaceutically acceptable salt thereof, wherein R^{4C} is a first R⁴ group.

In embodiments, R^{4C} is independently according to any embodiment described herein.

In embodiments, a compound of Formula (I), has a structure according to Formula (XVII),

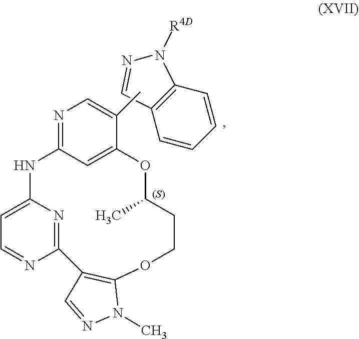

or a pharmaceutically acceptable salt thereof, wherein R^{4D} is a R⁴ group. In embodiments, R^{4D} is unsubstituted C₁₋₆ alkyl.

In embodiments, any occurrence of R^{4D} is independently according to any embodiment described herein.

In embodiments, a compound of Formula (XVII), has a structure according to Formula (XVII-1),

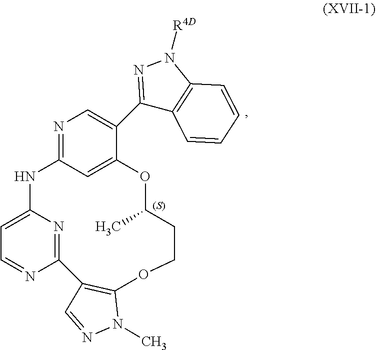

or a pharmaceutically acceptable salt thereof. In embodiments, any occurrence of R^{4D} is independently according to any embodiment described herein.

In embodiments, a compound of Formula (I), has a structure according to Formula (XVIII),

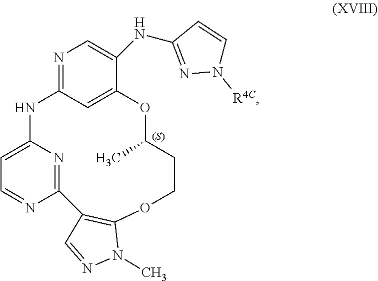

or a pharmaceutically acceptable salt thereof, wherein R^{4C} is a first R⁴ group.

In embodiments, any occurrence of R^{4C} is independently according to any embodiment described herein.

In embodiments, a compound of Formula (I), has a structure according to Formula (XIX),

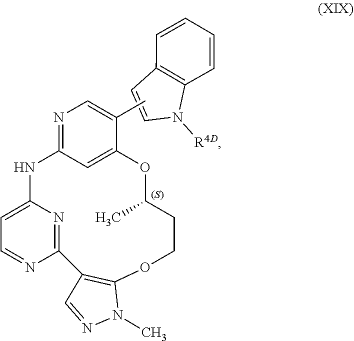

or a pharmaceutically acceptable salt thereof, wherein R^{4D} is a R⁴ group. In embodiments, R^{4D} is unsubstituted C₁₋₆ alkyl.

In embodiments, any occurrence of R^{4D} is independently according to any embodiment described herein.

In embodiments, a compound of Formula (XIX), has a structure according to Formula (XIX-1),

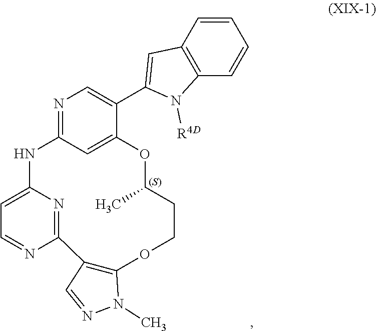

or a pharmaceutically acceptable salt thereof. In embodiments, any occurrence of R^{4D} is independently according to any embodiment described herein.

In embodiments, a compound of Formula (XIX), has a structure according to Formula (XIX-2),

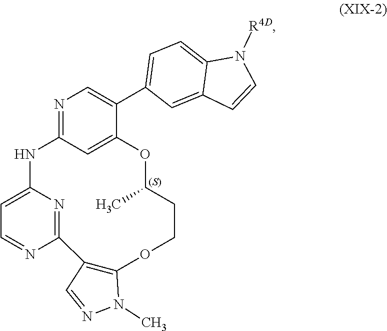

or a pharmaceutically acceptable salt thereof. In embodiments, any occurrence of R^{4D} is independently according to any embodiment described herein.

In embodiments, a compound of Formula (I), has a structure according to Formula (XX),

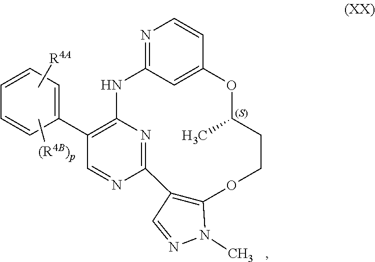

or a pharmaceutically acceptable salt thereof, wherein R^{4A} is a first R⁴ group, R^{4B} is a second R⁴ group, and p is 0 or 1.

In embodiments, any occurrence of R^{4A} and R^{4B} is independently according to any embodiment described herein.

In embodiments, a compound of Formula (XX), has a structure according to Formula (XX-1),

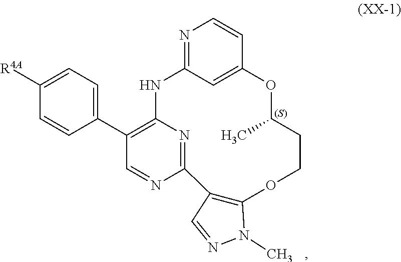

or a pharmaceutically acceptable salt thereof. In embodiments, any occurrence of R^{4A} is independently according to any embodiment described herein.

In embodiments, a compound of Formula (XX), has a structure according to Formula (XX-2),

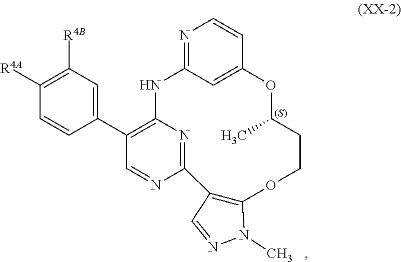

or a pharmaceutically acceptable salt thereof. In embodiments, each of R^{4A} and R^{4B} is independently according to any embodiment described herein.

In embodiments, a compound of Formula (XX), has a structure according to Formula (XX-3),

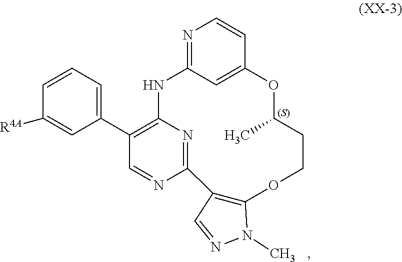

or a pharmaceutically acceptable salt thereof. In embodiments, R^{4A} is independently according to any embodiment described herein.

In embodiments, a compound of Formula (XX), has a structure according to Formula (XX-4),

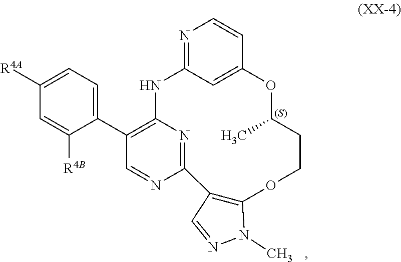

or a pharmaceutically acceptable salt thereof. In embodiments, any occurrence of R^{4A} and R^{4B} is independently according to any embodiment described herein.

In embodiments, a compound of Formula (I), has a structure according to Formula (XXI),

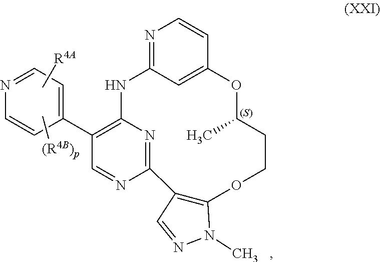

or a pharmaceutically acceptable salt thereof, wherein R^{4A} is a first R⁴ group, R^{4B} is a second R⁴ group, and p is 0 or 1.

In embodiments, any occurrence of R⁴ is independently according to any embodiment described herein. In embodiments, any occurrence of R^{4A} and R^{4B} is independently according to any embodiment described herein.

In embodiments, a compound of Formula (XXI), has a structure according to Formula (XXI-1),

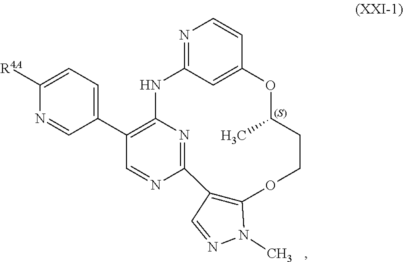

or a pharmaceutically acceptable salt thereof. In embodiments, R^{4A} is independently according to any embodiment described herein.

In embodiments, a compound of Formula (I), has a structure according to Formula (XXII),

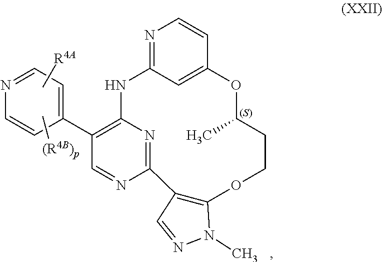

or a pharmaceutically acceptable salt thereof, wherein R^{4A} is a first R⁴ group, R^{4B} is a second R⁴ group, p is 0 or 1, and R^{4D} is a R⁴ group that is unsubstituted C₁₋₆ alkyl.

In embodiments, any occurrence of R⁴ is independently according to any embodiment described herein. In embodiments, any occurrence of R^{4A}, R^{4B}, and R^{4D} is independently according to any embodiment described herein.

In embodiments, a compound of Formula (XXII), has a structure according to Formula (XXII-1),

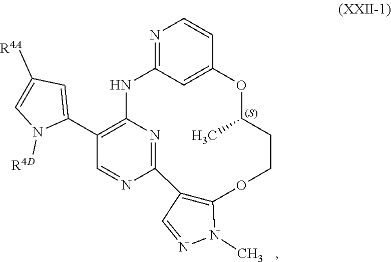

or a pharmaceutically acceptable salt thereof. In embodiments, any occurrence of R^{4A} and R^{4D} is independently according to any embodiment described herein.

In embodiments, a compound of Formula (I), has a structure according to Formula (XXIII),

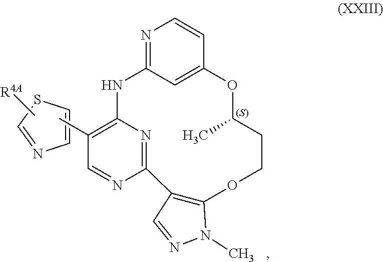

or a pharmaceutically acceptable salt thereof, wherein R^{4A} is a first R⁴ group.

In embodiments, any occurrence of R^{4A} is independently according to any embodiment described herein.

In embodiments, a compound of Formula (XXIII), has a structure according to Formula (XXIII-1),

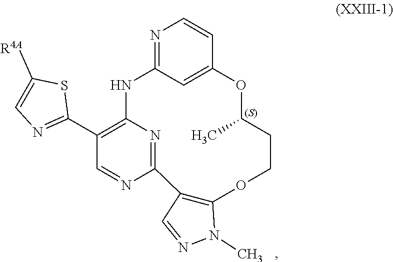

or a pharmaceutically acceptable salt thereof. In embodiments, R^{4A} is independently according to any embodiment described herein.

### Exemplary Embodiments of Structural Features

Provided herein are still further exemplary embodiments of structural features which may be present in any formula described herein (e.g., any of Formulas (I)-(XXIII) or any other formula described herein). An exemplary embodiment of a structural feature may occur in combination with any other exemplary structural feature described herein.

In embodiments, X¹ is N. In embodiments, X² is CR⁵ (e.g., CH).

In embodiments, X² is N. In embodiments, X² is CR⁵ (e.g., CH).

In embodiments, X³ is a covalent bond.

In embodiments, X³ is O.

In embodiments, X³ is S.

In embodiments, X³ is NR⁶, C(O)NR, NR⁶C(O), or NR⁶C(O)NR⁶. In embodiments, R⁶ is H. In embodiments, R⁶ is an N-protecting group (e.g., an amide group, a carbamate group, or a sulfonamide group). In embodiments, R⁶ is C₁₋₆ alkyl. In embodiments, a C₁₋₆ alkyl is unsubstituted. In embodiments, a C₁₋₆ alkyl is substituted (e.g., comprising 1, 2, or 3 substituent groups).

In embodiments, X³ is (C(R⁷)₂)_{q}. In embodiments, q is 1. In embodiments, q is 2. In embodiments, R⁷ is H. In embodiments, R⁷ is C₁₋₆ alkyl. In embodiments, a C₁₋₆ alkyl is unsubstituted. In embodiments, a C₁₋₆ alkyl is substituted (e.g., comprising 1, 2, or 3 substituent groups). In embodiments, two R⁷ on the same carbon combine to from an oxo (═O) group. In embodiments, X³ is C(O), CH₂, CHCH₃, or C(CH₃)₂.

In embodiments, X⁴ is a covalent bond.

In embodiments, X⁴ is O.

In embodiments, X⁴ is S.

In embodiments, 4 is NR⁶, C(O)NR⁶, NR⁶C(O), or NR⁶C(O)NR⁶. In embodiments, R⁶ is H. In embodiments, R⁶ is an N-protecting group (e.g., an amide group, a carbamate group, or a sulfonamide group). In embodiments, R⁶ is C₁₋₆ alkyl. In embodiments, a C₁₋₆ alkyl is unsubstituted. In embodiments, a C₁₋₆ alkyl is substituted (e.g., comprising 1, 2, or 3 substituent groups).

In embodiments, 4 is (C(R⁷)₂)_{q}. In embodiments, q is 1. In embodiments, q is 2. In embodiments, R⁷ is H. In embodiments, R⁷ is C₁₋₆ alkyl. In embodiments, a C₁₋₆ alkyl is unsubstituted. In embodiments, a C₁₋₆ alkyl is substituted (e.g., comprising 1, 2, or 3 substituent groups). In embodiments, two R⁷ on the same carbon combine to from an oxo (═O) group. In embodiments, 4 is C(O), CH₂, CHCH₃, or C(CH₃)₂.

In embodiments, X³ is 0 and X¹ is O.

In embodiments, X² is CH, X³ is O, and X⁴ is O. In embodiments, X¹ is N.

In embodiments, R⁶ and R⁴, together with the atoms to which they are attached, form a 5- to 6-membered ring. In embodiments, a 5- to 6-membered ring has a structure

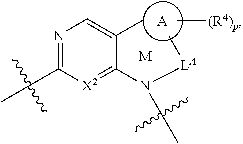

where the M ring is the newly-formed ring. In embodiments, L^{A} is a covalent bond. In embodiments, L^{A} is an alkylene (e.g., —CH₂—). In embodiments, an alkylene is unsubstituted. In embodiments, an alkylene is substituted (e.g., comprising 1 or 2 substituent groups).

In embodiments, R⁷ and R⁴, together with the atoms to which they are attached, form a 5- to 6-membered ring. In embodiments, a 5- to 6-membered ring has a structure

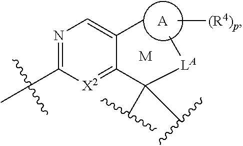

where the M ring is the newly-formed ring. In embodiments, L^{A} is a covalent bond. In embodiments, L^{A} is an alkylene (e.g., —CH₂—). In embodiments, an alkylene is unsubstituted. In embodiments, an alkylene is substituted (e.g., comprising 1 or 2 substituent groups).

In embodiments, X⁷ and X⁴ are the same. In embodiments, X³ and X⁴ are different. In embodiments, X³ and X⁴ are both O.

In embodiments, L¹ is a covalent bond.

In embodiments, L¹ is a C₁₋₆ heteroalkylene (e.g., comprises 1, 2, or 3 heteroatoms that are independently oxygen or nitrogen). In embodiments, LV is a branched C₁₋₆ heteroalkylene. In embodiments, L¹ is a linear C₁₋₆ heteroalkylene. In embodiments, L¹ is unsubstituted C_{J}-heteroalkylene. In embodiments, L¹ is unsubstituted branched C₁₋₆ heteroalkylene. In embodiments, L¹ is unsubstituted linear C₁₋₆ heteroalkylene. In embodiments, L¹ is substituted C₁₋₆ heteroalkylene (e.g., comprising 1, 2, or 3 substituent groups such as OH, oxo (═O), or unsubstituted C₁₋₆ alkyl). In embodiments, L¹ is substituted branched C₁₋₆ heteroalkylene (e.g., comprising 1, 2, or 3 substituent groups such as OH, oxo (═O), or unsubstituted C₁₋₃ alkyl). In embodiments, L¹ is substituted linear C₁₋₆ heteroalkylene (e.g., comprising 1, 2, or 3 substituent groups such as OH, oxo (═O), or unsubstituted C₁₋₃ alkyl). In embodiments, a C₁₋₆ heteroalkylene is —O(CH₂)ᵤ—, —(CH₂)ᵤO—, —O(CH₂)ᵤO—, —OCH₂OCH₂CH₂OCH₂—, —CH₂OCH₂CH₂O—, —OCH₂CH₂OCH₂—, —NH(CH₂)ᵤ—, —(CH₂)ᵤNH—, or —NH(CH₂)ᵤNH—, and wherein u is an integer of 1-4. In embodiments, u is 1. In embodiments, u is 2. In embodiments, u is 3. In embodiments, u is 4.

In embodiments, L is a C₁₋₆ alkylene (e.g., CH₂, (CH₂)₂, (CH₂)₃, (CH₂)₄, (CH₂)₅, or (CH₂)₆). In embodiments, L¹ is a branched C₁₋₆ alkylene. In embodiments, L¹ is a linear C₁₋₆ alkylene. In embodiments, L¹ is unsubstituted C₁₋₆ alkylene. In embodiments, L¹ is unsubstituted branched C₁₋₆ alkylene. In embodiments, L¹ is unsubstituted linear C₁-6 alkylene. In embodiments, L¹ is substituted C₁₋₆ alkylene (e.g., comprising 1, 2, or 3 substituent groups such as OH, oxo (═O), or unsubstituted C₁₋₃ alkyl). In embodiments, L¹ is substituted branched C₁₋₆ alkylene (e.g., comprising 1, 2, or 3 substituent groups such as OH, oxo (═O), or unsubstituted C₁₋₃ alkyl). In embodiments, L is substituted linear C₁₋₆ alkylene (e.g., comprising 1, 2, or 3 substituent groups such as OH, oxo (═O), or unsubstituted C₁₋₃ alkyl). In embodiments, L¹ is unsubstituted C₁₋₆ alkylene. In embodiments, L¹ is unsubstituted branched C₂₋₆ alkylene. In embodiments, L¹ is unsubstituted linear C₂₋₆ alkylene.

In embodiments, L¹ is a C₂₋₆ alkenylene (e.g., C₂H₄, C₃H₆, C₄H₈, C₅H₁₀, or C₆H₁₂). In embodiments, L¹ is unsubstituted C₂₋₆ alkenylene. In embodiments, L¹ is substituted C₂₋₆ alkenylene (e.g., comprising 1, 2, or 3 substituent groups).

In embodiments, L¹ is a C₂₋₆ alkynylene (e.g., C₂H₂, C₃H₄, C₄H₆, C₅H₈, or C₆H₁₀). In embodiments, L¹ is unsubstituted C₂₋₆ alkynylene. In embodiments, L¹ is substituted C₂₋₆ alkynylene (e.g., comprising 1, 2, or 3 substituent groups).

In embodiments, L¹ is a C₃₋₆ cycloalkylene (e.g., cyclopropylene, cyclobutylene, cyclopentylene, or cyclohexylene). In embodiments, L¹ is unsubstituted C₃₋₆ cycloalkylene. In embodiments, L¹ is substituted C₃₋₆ cycloalkylene (e.g., comprising 1, 2, or 3 substituent groups).

In embodiments, the sp³ carbon of a L¹ group has the (R)-configuration.

In embodiments, the sp³ carbon of a L¹ group has the (S)-configuration.

In embodiments, L is a 3- to 10-membered heterocyclylene (e.g., monocyclic or bicyclic heterocyclylene). In embodiments, L¹ is unsubstituted 3- to 10-membered heterocyclylene. In embodiments, L¹ is substituted 3- to 10-membered heterocyclylene (e.g., comprising 1, 2, or 3 substituent groups).

In embodiments, L¹ is a phenylene or naphthylene. In embodiments, L¹ is unsubstituted phenylene or unsubstituted naphthylene. In embodiments, L¹ is substituted phenylene or substituted naphthylene (e.g., comprising 1, 2, or 3 substituent groups).

In embodiments, L¹ is a 5- to 10-membered heteroarylene. In embodiments, L¹ is unsubstituted 5- to 10-membered heteroarylene. In embodiments, L¹ is substituted 5- to 10-membered heteroarylene (e.g., comprising 1, 2, or 3 substituent groups).

In embodiments, L¹ is an unsubstituted linear C₄₋₆ alkylene or an unsubstituted branched C₄₋₆ alkylene.

In embodiments, L¹ is —(CH₂)₃—. In embodiments, L¹ is —CH(CH³)CH₂CH₂—.

In embodiments, L¹ is

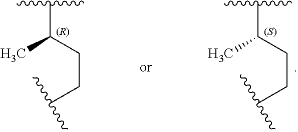

In embodiments, L¹ is

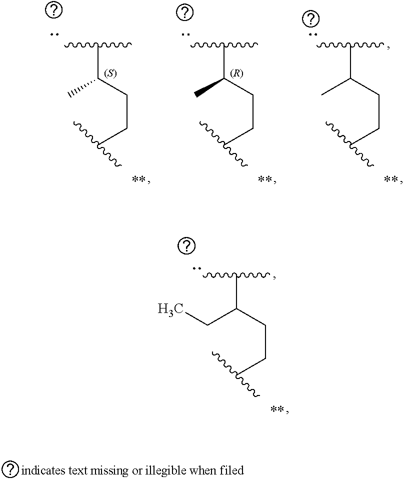

where * denotes the point of covalent attachment to X⁴, and ** denotes the point of covalent attachment to X³.

In embodiments, —X⁴-L¹-X³— is —O-L¹-O—.

In embodiments, —X⁴-L¹-X³— is —O(CH₂)₃O—.

In embodiments, —X⁴-L¹-X³— is —OCH(CH₃)CH₂CH₂O—.

In embodiments, —X⁴-L¹-X³— is

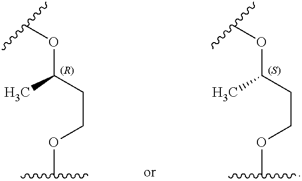

In embodiments, X⁴-L¹-X³ forms or comprises a urea group (e.g., NHC(O)NH). In embodiments, X³ and/or X⁴ is NR⁶C(O)NR⁶. In embodiments, one of X³ and X⁴ is NR⁶C(O)NR⁶.

In embodiments, X⁴-L¹-X³ forms or comprises a carboxamide group (e.g., C(O)NH or NH(CO)). In embodiments, X³ and/or X⁴ is C(O)NR⁶ or NR⁶C(O). In embodiments, one of X³ and X⁴ is C(O)NR⁶ or NR⁶C(O).

In embodiments, X⁴-L¹-X³ is —CHR⁷—O(C₁₋₂ alkylene)—OCHR⁷— or —CHR⁷—O(C₁₋₂ alkylene)-O—.

In embodiments, B is phenyl. In embodiments, B is naphthyl. In embodiments, B is 5- to 13-membered heteroaryl (e.g., monocyclic or bicyclic heteroaryl). In embodiments, B is a bicyclic 8- to 12-membered heteroaryl (e.g., nitrogen-containing, bicyclic 8- to 12-membered heteroaryl). In embodiments, B is a monocyclic 5- to 6-membered heteroaryl. Exemplary monocyclic 5- to 6-membered heteroaryls include but are not limited to pyridyl, pyrimidyl, pyrazolyl, pyrrolyl, thiazolyl, oxazolyl, and imidazolyl. In embodiments, B is phenyl or 5- to 6-membered heteroaryl. In embodiments, B is phenyl, pyridyl, pyrimidyl, pyrazolyl, pyrrolyl, thiazolyl, oxazolyl, or imidazolyl. In embodiments, B is pyrazolyl.

In embodiments, B is unsubstituted phenyl. In embodiments, B is unsubstituted naphthyl. In embodiments, B is unsubstituted 5- to 13-membered heteroaryl (e.g., unsubstituted monocyclic or bicyclic heteroaryl). In embodiments, B is unsubstituted bicyclic 8- to 12-membered heteroaryl (e.g., unsubstituted nitrogen-containing, bicyclic 8- to 12-membered heteroaryl). In embodiments, B is unsubstituted monocyclic 5- to 6-membered heteroaryls. In embodiments, B is unsubstituted pyridyl, unsubstituted pyrimidyl, unsubstituted pyrazolyl, unsubstituted pyrrolyl, unsubstituted thiazolyl, unsubstituted oxazolyl, or unsubstituted imidazolyl.

In embodiments, B is substituted phenyl (e.g., comprising 1 or 2 substituents as described herein). In embodiments, B is substituted naphthyl (e.g., comprising 1 or 2 substituents as described herein). In embodiments, B is substituted 5- to 13-membered heteroaryl (e.g., substituted monocyclic or bicyclic heteroaryl comprising 1 or 2 substituents as described herein). B is substituted bicyclic 8- to 12-membered heteroaryl (e.g., substituted nitrogen-containing, bicyclic 8- to 12-membered heteroaryl). In embodiments, B is substituted monocyclic 5- to 6-membered heteroaryls. In embodiments, B is substituted pyridyl, substituted pyrimidyl, substituted pyrazolyl, substituted pyrrolyl, substituted thiazolyl, substituted oxazolyl, or substituted imidazolyl. In embodiments, B is substituted pyrazolyl (e.g., N-substituted pyrazolyl such as N-methyl pyrazolyl). In embodiments, B is substituted with one or more R³ groups as described herein (e.g., methyl, halogen, or CN).

In embodiments, B is

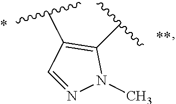 [SMILES: *c1cnn(C)c1*]

where * denotes the point of covalent attachment to C, and ** denotes the point of covalent attachment to X³.

In embodiments, C is 5- or 6-membered heteroaryl. In embodiments, C is 5- or 6-membered N-containing heteroaryl. Exemplary 5- or 6-membered heteroaryls include but are not limited to pyridyl, pyrimidyl, pyrazolyl, pyrrolyl, thiazolyl, oxazolyl, and imidazolyl. In embodiments, C is pyridyl or pyrimidyl. In embodiments, C is pyrazolyl or thiazolyl.

In embodiments, C is unsubstituted 5- or 6-membered heteroaryl. In embodiments, C is unsubstituted 5- or 6-membered N-containing heteroaryl. Examplary unsubstituted 5- or 6-membered heteroaryls include but are not limited to unsubstituted pyridyl, unsubstituted pyrimidyl, unsubstituted pyrazolyl, unsubstituted pyrrolyl, unsubstituted thiazolyl, unsubstituted oxazolyl, and unsubstituted imidazolyl. In embodiments, C is unsubstituted pyridyl or pyrimidyl. In embodiments, C is unsubstituted pyrazolyl or thiazolyl.

In embodiments, C is substituted 5- or 6-membered heteroaryl (e.g., comprising 1 or 2 substituents as described herein). In embodiments, C is substituted 5- or 6-membered N-containing heteroaryl (e.g., comprising 1 or 2 substituents as described herein). In embodiments, C is substituted pyridyl, substituted pyrimidyl, substituted pyrazolyl, substituted pyrrolyl, substituted thiazolyl, substituted oxazolyl, or substituted imidazolyl. In embodiments, C is substituted pyridyl (e.g., substituted with Substructure A). In embodiments, C is substituted pyrimidyl (e.g., substituted with Substructure A). In embodiments, C is substituted pyrazolyl (e.g., N-substituted pyrazolyl such as N-methyl pyrazolyl). In embodiments, C is substituted thiazolyl (e.g., methyl substituted thiazolyl). In embodiments, C is substituted with one or more R¹ groups as described herein (e.g., Substructure A or methyl).

In embodiments, A is pyrazolyl, B is pyrazolyl, and C is pyridyl or pyrimidyl. In embodiments, A and B are substituted.

In embodiments, m is 0. In embodiments, m is 1. In embodiments, in is 2. In embodiments, m is 1 or 2.

In embodiments, m is not 0. In embodiments, R¹ is present.

In embodiments, R¹ is

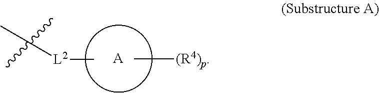

In embodiments, L² is independently a covalent bond, O, NR^{L}, C(O) C(O)NR^{L}, NR^{L}C(O), CR^{L}₂, wherein R^{L} is independently H or C₁₋₆ alkyl. In embodiments, R^{L} is unsubstituted C₁₋₆ alkyl. In embodiments, R^{L} is substituted C₁₋₆ alkyl (e.g., comprising 1, 2, or 3 substituent groups). In embodiments, L² is covalent bond.

In embodiments, each R⁴ is independently H, OH, CN, halogen, C₁₋₆ aliphatic, C₁₋₆ alkoxy, NR⁸R⁹, C(O)R¹⁰, CO₂R¹⁰, C(O)NR¹¹R¹⁰, NR¹¹C(O)R¹⁰, NR¹¹CO₂R¹⁰, NR¹¹C(O)NR⁸R⁹, NR¹¹(CH₂)ₛNR⁸R⁹, (CH₂)ₜNR⁸R⁹, or (CH₂)ᵣR¹². In embodiments, p is 0. In embodiments, p is 1. In embodiments, p is 2.

In embodiments, R¹ is OH. In embodiments, R¹ is CN. In embodiments, R¹ is halogen (e.g., F, Cl, Br, or I). In embodiments, R¹ is C₁₋₆ aliphatic. In embodiments, R¹ is unsubstituted C₁₋₆ aliphatic. In embodiments, R¹ is substituted C₁₋₆ aliphatic (e.g., comprising 1, 2, or 3 substituent groups). In embodiments, R¹ is C₁₋₆ alkoxy. In embodiments, R¹ is unsubstituted C₁₋₆ alkoxy. In embodiments, R is substituted C₁₋₆ alkoxy (e.g., comprising 1, 2, or 3 substituent groups). In embodiments, R¹ is NR⁸R⁹. In embodiments, R¹ is C(O)R¹⁰. In embodiments, R¹ is CO₂R¹. In embodiments, R¹ is C(O)NR⁸R⁹. In embodiments, R¹ is N¹¹C(O)R¹⁰. In embodiments, R¹ is NR¹¹CO₂R¹⁰. In embodiments, R¹ is NR¹¹C(O)NR⁸R⁹. In embodiments, R¹ is (CH₂)ᵣR¹². In embodiments, r is 0. In embodiments, r is 1. In embodiments, r is 2. In embodiments, r is 3. In embodiments, r is 4. In embodiments, r is 0 or 1. In embodiments, R¹ is halogen (e.g., F, Cl, Br, or I).

In embodiments, n is 0. In embodiments, n is 1. In embodiments, n is 2. In embodiments, n is 1 or 2.

In embodiments, n is not 0. In embodiments, R² is present.

In embodiments, R² is

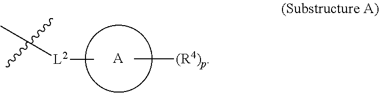

In embodiments, L² is independently a covalent bond, O, NR^{L}, C(O), C(O)NR^{L}, NR^{L}C(O), CR^{L}₂, wherein R^{L} is independently H or C₁₋₆ alkyl. In embodiments, R^{L} is unsubstituted C₁₋₆ alkyl. In embodiments, R^{L} is substituted C₁₋₆ alkyl (e.g., comprising 1, 2, or 3 substituent groups). In embodiments, L² is covalent bond.

In embodiments, each R⁴ is independently H, OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, NR¹¹(CH₂)ₛNR⁸R⁹, (CH₂)ₜNR⁸R⁹, or (CH₂)ᵣR¹². In embodiments, p is 0. In embodiments, p is 1. In embodiments, p is 2.

In embodiments, R² is OH. In embodiments, R² is CN. In embodiments, R² is halogen (e.g., F, Cl, Br, or I). In embodiments, R² is C₁₋₆ aliphatic. In embodiments, R² is unsubstituted C₁₋₆ aliphatic. In embodiments, R² is substituted C₁₋₆ aliphatic (e.g., comprising 1, 2, or 3 substituent groups). In embodiments, R² is C₁₋₆ alkoxy. In embodiments, R² is unsubstituted C₁₋₆ alkoxy. In embodiments, R² is substituted C₁₋₆ alkoxy (e.g., comprising 1, 2, or 3 substituent groups). In embodiments, R² is NR⁸R⁹. In embodiments, R² is C(O)R¹⁰. In embodiments, R² is CO₂R¹⁰. In embodiments, R² is C(O)NR⁸R⁹. In embodiments, R² is NR¹¹C(O)R¹⁰. In embodiments, R² is NR¹¹CO₂R¹⁰. In embodiments, R² is NR¹¹C(O)NR⁸R⁹. In embodiments, R² is (CH₂)ᵣR¹². In embodiments, r is 0. In embodiments, r is 1. In embodiments, r is 2. In embodiments, r is 3. In embodiments, r is 4. In embodiments, r is 0 or 1. In embodiments, R² is halogen (e.g., F, Cl, Br, or I).

In embodiments, R¹ is present. In embodiments, R² is present. In embodiments, one of R¹ and R² is present. In embodiments, one of R¹ and R² is present and is

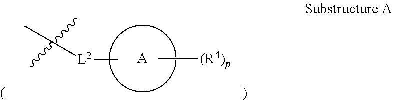

or halogen (e.g., F, Cl, Br, or I). In embodiments, one of R¹ and R² is present and is

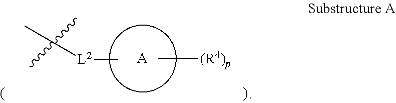

In embodiments, two R¹ or two R², together to which the atoms they are attached form a 5- to 10-membered ring (e.g., 5- to 10-membered carbocyclic, heterocyclic, aryl, or heteroaryl ring).

In embodiments, o is 0. In embodiments, o is 1. In embodiments, o is 2. In embodiments, o is 1 or 2. In embodiments, o is 0 or 1.

In embodiments, o is not 0. In embodiments, R³ is present.

In embodiments, R³ is OH. In embodiments, R³ is CN. In embodiments, R³ is halogen (e.g., F, Cl, Br, or I). In embodiments, R³ is C₁₋₆ aliphatic. In embodiments, R³ is unsubstituted C₁₋₆ aliphatic. In embodiments, R³ is substituted C₁₋₆ aliphatic (e.g., comprising 1, 2, or 3 substituent groups). In embodiments, R³ is C_{A-6} alkoxy. In embodiments, R³ is unsubstituted C₁₋₆ alkoxy. In embodiments, R³ is substituted C₅₋₆ alkoxy (e.g., comprising 1, 2, or 3 substituent groups). In embodiments, R³ is NR⁸R⁹. In embodiments, R³ is C(O)R¹⁰. In embodiments, R³ is CO₂R¹⁰. In embodiments, R³ is C(O)NR⁸R⁹. In embodiments, R³ is NR¹C(O)R¹⁰. In embodiments, R³ is NR¹¹CO₂R¹⁰. In embodiments, R³ is NR¹¹C(O)NR⁸R⁹. In embodiments, R⁵ is (CH₂)ᵣR¹². In embodiments, r is 0. In embodiments, r is 1. In embodiments, r is 2. In embodiments, r is 3. In embodiments, r is 4. In embodiments, r is 0 or 1. In embodiments, R³ is methyl, halogen, or CN. In embodiments, R³ is methyl.

In embodiments, R⁵ is H.

In embodiments, R⁵ is OH. In embodiments, R⁵ is CN. In embodiments, R⁵ is halogen (e.g., F, Cl, Br, or I). In embodiments, R⁵ is C₁₋₆ aliphatic. In embodiments, R⁵ is unsubstituted C₁₋₆ aliphatic. In embodiments, R⁵ is substituted C₁₋₆ aliphatic (e.g., comprising 1, 2, or 3 substituent groups). In embodiments, R⁵ is C₁₋₆ alkoxy. In embodiments, R⁵ is unsubstituted C₁₋₆ alkoxy. In embodiments, R⁵ is substituted C₁₋₆ alkoxy (e.g., comprising 1, 2, or 3 substituent groups). In embodiments, R⁵ is NR⁸R⁹. In embodiments, R⁵ is C(O)R¹⁰. In embodiments, R¹ is CO₂R¹⁰. In embodiments, R⁵ is C(O)NR⁸R⁹. In embodiments, R⁵ is NR¹¹C(O)R¹⁰. In embodiments, R⁵ is NR¹¹CO₂R¹⁰. In embodiments, R⁵ is NR¹¹C(O)NR⁸R⁹. In embodiments, R⁵ is (CH₂)ᵣR¹². In embodiments, r is 0. In embodiments, r is 1. In embodiments, r is 2. In embodiments, r is 3. In embodiments, r is 4. In embodiments, r is 0 or 1.

In embodiments, R⁸ is H. In embodiments, R⁸ is C₁₋₆ alkyl. In embodiments, a C₁₋₆ alkyl is unsubstituted. In embodiments, a C₁₋₆ alkyl is substituted (e.g., comprising 1, 2, or 3 substituent groups).

In embodiments, R⁹ is H. In embodiments, R⁹ is C₁₋₆ alkyl. In embodiments, a C₁₋₆ alkyl is unsubstituted. In embodiments, a C₁₋₆ alkyl is substituted (e.g., comprising 1, 2, or 3 substituent groups).

In embodiments, R¹¹ is H. In embodiments, R¹¹ is C₁₋₆ alkyl. In embodiments, a C₁₋₆ alkyl is unsubstituted. In embodiments, a C₁₋₆ alkyl is substituted (e.g., comprising 1, 2, or 3 substituent groups).

In embodiments, R⁸ and R⁹, together with the nitrogen atom to which they are attached, form a 3- to 10-membered heterocyclyl. In embodiments, a 3- to 10-membered heterocyclyl is unsubstituted. In embodiments, a 3- to 10-membered heterocyclyl is unsubstituted (e.g., comprising 1, 2, or 3 substituent groups).

In embodiments, R⁸ and R¹¹, together with the atoms to which they are attached, form a 3- to 10-membered heterocyclyl. In embodiments, a 3- to 10-membered heterocyclyl is unsubstituted. In embodiments, a 3- to 10-membered heterocyclyl is unsubstituted (e.g., comprising 1, 2, or 3 substituent groups).

In embodiments, R¹⁰ is C₁₋₆ aliphatic. In embodiments, R¹⁰ is C₃-C₁₀ cycloaliphatic (e.g., monocyclic or bicyclic cycloaliphatic). In embodiments, R¹⁰ is 3- to 10-membered heterocyclyl (e.g., monocyclic or bicyclic heterocyclyl). In embodiments, R¹⁰ is phenyl. In embodiments, R¹⁰ is naphthyl. In embodiments, R¹⁰ is 5- to 12-membered heteroaryl (e.g., monocyclic or bicyclic heteroaryl).

In embodiments, R¹⁰ is unsubstituted C₁₋₆ aliphatic. In embodiments, R¹⁰ is unsubstituted C₃-C₁f cycloaliphatic. In embodiments, R¹⁰ is unsubstituted 3- to 10-membered heterocyclyl. In embodiments, R¹⁰ is unsubstituted phenyl. In embodiments, R¹⁰ is unsubstituted naphthyl. In embodiments, R¹⁰ is unsubstituted 5- to 12-membered heteroaryl.

In embodiments, R¹⁰ is substituted C₁₋₆ aliphatic. In embodiments, R¹⁰ is substituted C₃-C₁₀ cycloaliphatic. In embodiments, R¹⁰ is substituted 3- to 10-membered heterocyclyl. In embodiments, R¹⁰ is substituted phenyl. In embodiments, R¹⁰ is substituted naphthyl. In embodiments, R¹⁰ is substituted 5- to 12-membered heteroaryl. In embodiments, a substituted group comprises 1, 2, or 3 substituent groups as described herein.

In embodiments, R¹⁰ and R¹, together with the atoms to which they are attached, form a 3- to 10-membered heterocyclyl. In embodiments, a 3- to 10-membered heterocyclyl is unsubstituted. In embodiments, a 3- to 10-membered heterocyclyl is substituted (e.g., comprising 1, 2, or 3 substituent groups).

In embodiments, R¹² is C₃-C₁₀ cycloaliphatic (e.g., monocyclic or bicyclic cycloaliphatic). In embodiments, R¹² is 3- to 10-membered heterocyclyl (e.g., monocyclic or bicyclic heterocyclyl). In embodiments, R¹² is phenyl. In embodiments, R¹² is naphthyl. In embodiments, R¹² is 5- to 12-membered heteroaryl (e.g., monocyclic or bicyclic heteroaryl).

In embodiments, R¹² is unsubstituted C₃-C₁w cycloaliphatic. In embodiments, R¹² is unsubstituted 3- to 10-membered heterocyclyl. In embodiments, R¹² is unsubstituted phenyl. In embodiments, R¹² is unsubstituted naphthyl. In embodiments, R¹² is unsubstituted 5- to 12-membered heteroaryl.

In embodiments, R¹² is substituted C₃-C₁₃ cycloaliphatic. In embodiments, R¹² is substituted 3- to 10-membered heterocyclyl. In embodiments, R¹² is substituted phenyl. In embodiments, R¹² is substituted naphthyl. In embodiments, R¹² is substituted 5- to 12-membered heteroaryl. In embodiments, a substituted group comprises 1, 2, or 3 substituent groups as described herein.

### Substructure A

Still further exemplary Substructure A groups are described herein. That is, embodiments of compounds of Formula (I) (e.g., any compound according to Formula (I)-(XXIII) and subformulas thereof) can feature any Substructure A group described herein.

In embodiments,

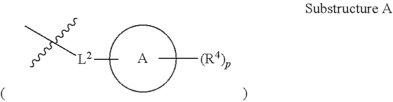

is not present. In embodiments, one Substructure A group is present. In embodiments, two Substructure A groups are present (e.g., two Substructure A groups with identical or different structures). In embodiments, more than two Substructure A groups are present (e.g., more than two Substructure A groups with identical or different structures). In embodiments, no more than one Substructure A group is present.

In embodiments, L² is a covalent bond. In embodiments, Substructure A has a structure of

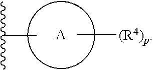

In embodiments, L² is O. In embodiments, Substructure A has a structure of

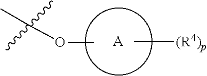

In embodiments, L² is NR^{L}. In embodiments, R^{L} is H. In embodiments, R^{L} is C₁₋₆ alkyl. In embodiments, R^{L} is unsubstituted C₁₋₆ alkyl. In embodiments, R^{L} is substituted C₁₋₆ alkyl (e.g., comprising 1, 2, or 3 substituent groups). In embodiments, Substructure A has a structure of

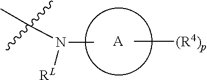

In embodiments, L² is C(O). In embodiments, Substructure A has a structure of

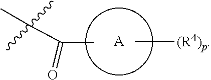

In embodiments, L² is C(O)NR^{L}. In embodiments, R^{L} is H. In embodiments, R^{L} is C₁₋₆ alkyl. In embodiments, R^{L} is unsubstituted C₁₋₆ alkyl. In embodiments, R^{L} is substituted C₁₋₆ alkyl (e.g., comprising 1, 2, or 3 substituent groups). In embodiments, Substructure A has a structure of

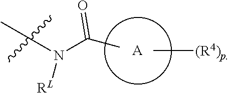

In embodiments, L² is NR^{L}C(O). In embodiments, R^{L} is H. In embodiments, R^{L} is C₁₋₆ alkyl. In embodiments, R^{L} is unsubstituted C₁₋₆ alkyl. In embodiments, R^{L} is substituted C₁₋₆ alkyl (e.g., comprising 1, 2, or 3 substituent groups). In embodiments, Substructure A has a structure of

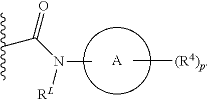

In embodiments, L² is CR^{L}₂. In embodiments, R^{L} is H. In embodiments, R^{L} is C₁₋₆ alkyl. In embodiments, R^{L} is unsubstituted C₁₋₆ alkyl. In embodiments, R^{L} is substituted C₁₋₆ alkyl (e.g., comprising 1, 2, or 3 substituent groups). In embodiments, Substructure A has a structure of

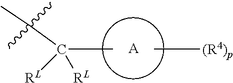

In embodiments, A is phenyl. In embodiments, A is naphthyl. In embodiments, A is 5- to 13-membered heteroaryl (e.g., monocyclic or bicyclic heteroaryl). In embodiments, A is a bicyclic 8- to 12-membered heteroaryl (e.g., nitrogen-containing, bicyclic 8- to 12-membered heteroaryl). In embodiments, A is a monocyclic 5- to 6-membered heteroaryl. Examplary monocyclic 5- to 6-membered heteroaryls include but are not limited to pyridyl, pyrimidyl, pyrazolyl, pyrrolyl, thiazolyl, oxazolyl, and imidazolyl. In embodiments, A is phenyl or 5- to 6-membered heteroaryl. In embodiments, A is phenyl, pyridyl, pyrimidyl, pyrazolyl, pyrrolyl, thiazolyl, oxazolyl, or imidazolyl.

In embodiments, A is unsubstituted phenyl. In embodiments, A is unsubstituted naphthyl. In embodiments, A is unsubstituted 5- to 13-membered heteroaryl (e.g., unsubstituted nonocyclic or bicyclic heteroaryl). In embodiments, A is unsubstituted bicyclic 8- to 12-membered heteroaryl (e.g., unsubstituted nitrogen-containing, bicyclic 8- to 12-membered heteroaryl). In embodiments, A is unsubstituted monocyclic 5- to 6-membered heteroaryls. In embodiments, A is unsubstituted pyridyl, unsubstituted pyrimidyl, unsubstituted pyrazolyl, unsubstituted pyrrolyl, unsubstituted thiazolyl, unsubstituted oxazolyl, or unsubstituted imidazolyl.

In embodiments, A is substituted phenyl (e.g., comprising 1 or 2 substituents as described herein). In embodiments, A is substituted naphthyl (e.g., comprising 1 or 2 substituents as described herein). In embodiments, A is substituted 5- to 13-membered heteroaryl (e.g., substituted monocyclic or bicyclic heteroaryl comprising 1 or 2 substituents as described herein). A is substituted bicyclic 8- to 12-membered heteroaryl (e.g., substituted nitrogen-containing, bicyclic 8- to 12-membered heteroaryl). In embodiments, A is substituted monocyclic 5- to 6-membered heteroaryls. In embodiments, A is substituted pyridyl, substituted pyrimidyl, substituted pyrazolyl, substituted pyrrolyl, substituted thiazolyl, substituted oxazolyl, or substituted imidazolyl.

In embodiments, A is substituted pyrazolyl.

In embodiments, A is substituted pyridyl.

In embodiments, A is substituted pyrimidyl.

In embodiments, A is substituted pyrrolyl.

In embodiments, A is substituted thiazolyl.

In embodiments, A is substituted oxazolyl.

In embodiments, A is substituted imidazolyl.

In embodiments, A is substituted with one or more R⁴ groups as described herein. In embodiments, A is substituted with 1-3 R⁴ groups as described herein.

In embodiments, p is 0. In embodiments, p is 1. In embodiments, p is 2. In embodiments, p is 1 or 2.

In embodiments, p is not 0. In embodiments, R⁴ is present.

In embodiments, R⁴ is H. In embodiments, when R⁴ is present and is a non-hydrogen moiety, R⁴ represents a substituent group. Accordingly, it is also understood that for any value of p described herein, hydrogens are present as appropriate in order to complete valency requirements at constituent atoms of A such that the molecule is a stable compound (e.g., the molecule is a compound which does not spontaneously undergo transformation such as by rearrangement, cyclization, elimination, or other reaction). Exemplary embodiments of A, R⁴, and p are described herein.

In embodiments, R⁴ is OH. In embodiments, R⁴ is CN. In embodiments, R⁴ is halogen (e.g., F, Cl, Br, or I).

In embodiments, R⁴ is C₁₋₆ aliphatic (e.g., C₁₋₆ alkyl). In embodiments, R⁴ is unsubstituted C₁₋₆ aliphatic (e.g., unsubstituted C₁₋₆ alkyl). In embodiments, R⁴ is substituted C₁₋₆ aliphatic (e.g., comprising 1, 2, or 3 substituent groups).

In embodiments, R⁴ is C₁₋₆ alkoxy. In embodiments, R⁴ is unsubstituted C₁₋₆ alkoxy (e.g., O-(unsubstituted C₁₋₆ alkyl)). In embodiments, R⁴ is substituted C₁₋₆ alkoxy (e.g., comprising 1, 2, or 3 substituent groups, or O—(C₁₋₆ haloalkyl)).

In embodiments, R⁴ is NR⁸R⁹.

In embodiments, R⁴ is C(O)R¹⁰.

In embodiments, R⁴ is CO₂R¹⁰ (e.g., CO₂(unsubstituted C₁₋₆ alkyl)).

In embodiments, R⁴ is C(O)NR⁸R⁹.

In embodiments, R⁴ is NR¹¹C(O)R¹⁰.

In embodiments, R⁴ is NR¹¹CO₂R¹⁰.

In embodiments, R⁴ is NR¹¹C(O)NR⁸R⁹.

In embodiments, R⁴ is NR¹¹(CH₂)ₛNR⁸R⁹ (e.g., NH(CH₂)ₛNMe₂). In embodiments, s is an integer of 2-6. In embodiments, s is 2. In embodiments, s is 3. In embodiments, s is 4. In embodiments, s is 5. In embodiments, s is 6. In embodiments, s is an integer of 2-4.

In embodiments, R⁴ is (CH₂)ₜNR⁸R⁹ (e.g., (CH₂)ₜNMe₂). In embodiments, t is an integer of 1-6. In embodiments, t is 1. In embodiments, t is 2. In embodiments, t is 3. In embodiments, t is 4. In embodiments, t is 5. In embodiments, t is 6.

In embodiments, R⁴ is (CH₂)ᵣR¹². In embodiments, r is an integer of 0-4. In embodiments, r is 0. In embodiments, r is 1. In embodiments, r is 2. In embodiments, r is 3. In embodiments, r is 4. In embodiments, r is 0 or 1.

In embodiments, R⁴ and R⁶, or R⁴ and R⁷, together with the atoms to which they are attached, form a 5- to 6-membered ring.

In embodiments, R⁴ is

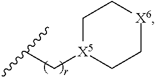

wherein
X⁵ is independently CH or N;
X⁶ is independently O, CHR¹³ or NR¹³;
R¹³ is independently H, C₁₋₆ alkyl, or C₃₋₆ cycloalkyl;
r is 0 or 1;

In embodiments, X⁵ is CH. In embodiments, X⁵ is N;

In embodiments, X⁶ is O. In embodiments, X⁶ is CHR¹³. In embodiments, X⁶ is NR¹³ (e.g., NMe or N(cyclopropyl)).

In embodiments, R¹³ is H. In embodiments, R¹³ is C₁₋₆ alkyl (e.g., Me). In embodiments, R¹³ is C₃₋₆ cycloalkyl (e.g., cyclopropyl).

In embodiments, r is 0. In embodiments, r is 1.

In embodiments, a compound described herein comprises one R⁴ group. In embodiments, a compound described herein comprises multiple R⁴ groups. In embodiments, a compound described herein comprises two R⁴ groups. In embodiments, a compound described herein comprises three R⁴ groups. In embodiments, a compound described herein comprises four R⁴ groups. R⁴ groups are independently selected, including any combination of any of the exemplary embodiments described herein.

In embodiments, R⁴ is selected from the group consisting of —CO₂CH₃, —OCH₂CF₃, —CH₃, —CH₂CH₃, —OCH₃, —OCH₂CH₃, —NHCH₂CH₂N(CH₃)₂, —CH₂N(CH₃)₂,

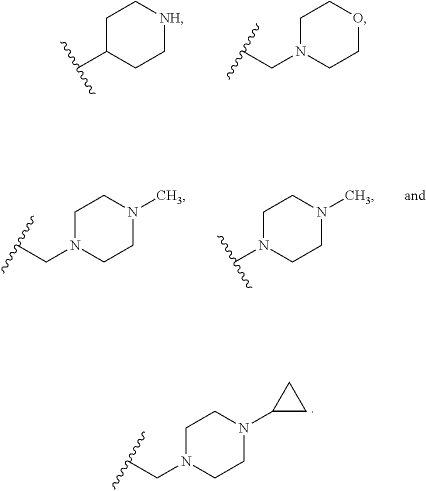 [SMILES: *C1CCNCC1 ; *CN1CCOCC1 ; *CN1CCN(C)CC1 ; *N1CCN(C)CC1 ; "and" ; *CN1CCN(C2CC2)CC1]

In embodiments, R⁴ is selected from: —C≡N; —C≡CH; a saturated linear or branched C₁₋₆ aliphatic or C₁₋₆ alkoxy comprising 0-4 fluoro substituents; NR¹¹(CH₂)ₛNR⁸R⁹; (CH₂)ₜNR⁸R⁹; O(CH₂)ₜOCH₃; O(CH₂)ᵣR¹²; and (CH₂)ᵣR¹². In embodiments, R² is selected from the group consisting of: a C₃₋₆ cycloalkyl; a 3-9 membered heterocyclyl comprising 1-3 heteroatoms selected from O, N, and S; and 5- to 6-membered heteroaryl. In embodiments, R¹² is selected from cyclopropyl, cyclobutyl, cyclopentyl, cyclohexyl, oxetanyl, tetrahydrofuryl, tetrahydropyanyl, azetidine, pyrroldinyl, piperidinyl, piperazinyl, morpholino. In embodiments, R¹² is substituted with 0-4 R¹⁴, wherein each R¹⁴ is independently selected from —CN, oxo (═O), halogen, —OH, —NH₂, monoalkylamino, dialkylamino, unsubstituted C₃₋₆ cycloalkyl, or unsubstituted 3- to 4-membered heterocyclyl. In embodiments, each R¹⁴ is independently selected from —CN, —F, —OH, —NH₂, —NHCH₃, —N(CH₃)₂, —NHCH₂CH₃, —N(CH₂CH₃)₂, —CH₃, —CH₂F, —CHF₂, —C F₃, —CH₂CH₃, —CH₂CH₂F, —CH₂CHF₂, —CH₂CF₃, —CH₂CH₂CH₃, —CH₂CH₂CH₂F, —CH₂CH₂CHF₂, CH₂CH₂CF₃, —CH₂CH₂OCH₃, —COCH₃, —COCH₂CH₃, —CH₂COCH₃, —CH₂COCH₂CH₃, cyclopropyl, cyclobutyl, oxetanyl, and azetidinyl.

In embodiments, one or more R⁴ groups is independently selected from a first group of permitted R⁴ moieties, wherein said group is: —CN, —CH₃, —CH₂F, —CHF₂, —CF₃, —CH₂CH₃, —CH₂CFH₂, —CH₂CHF₂, —CH₂CF₃, —CH(CH₃)₂, —C(CH₃)₃, —C≡CH,

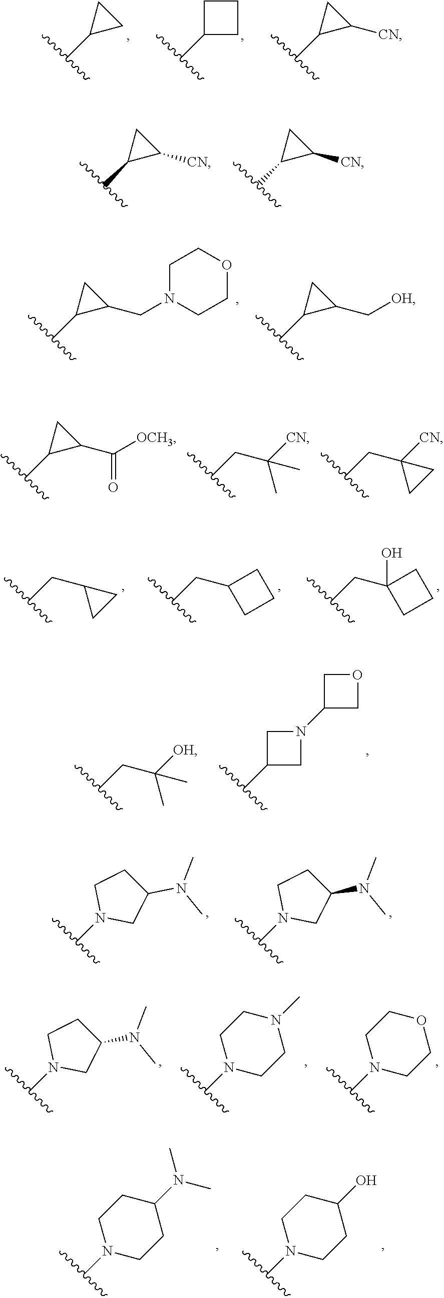

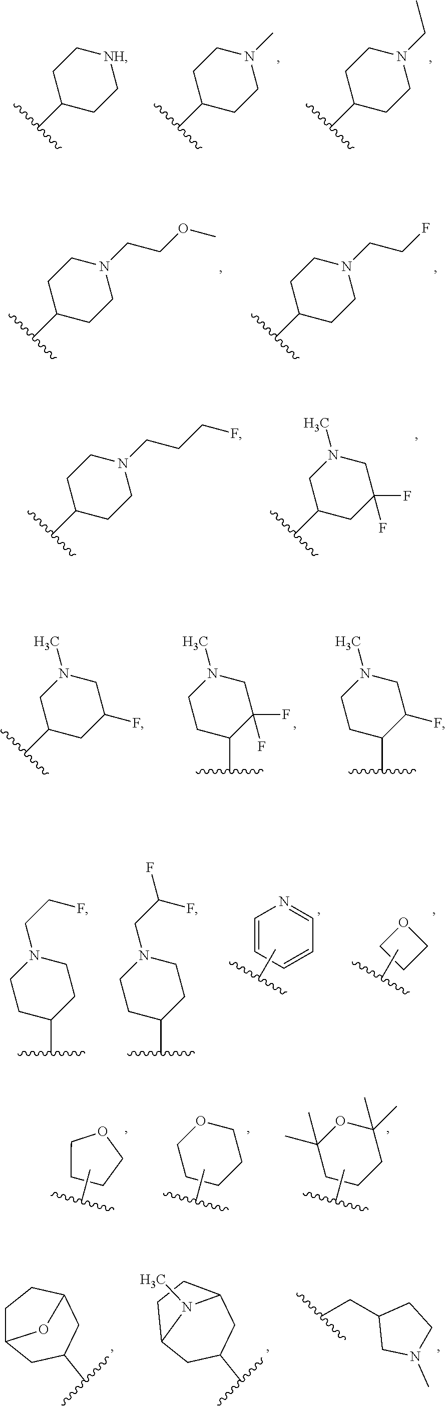

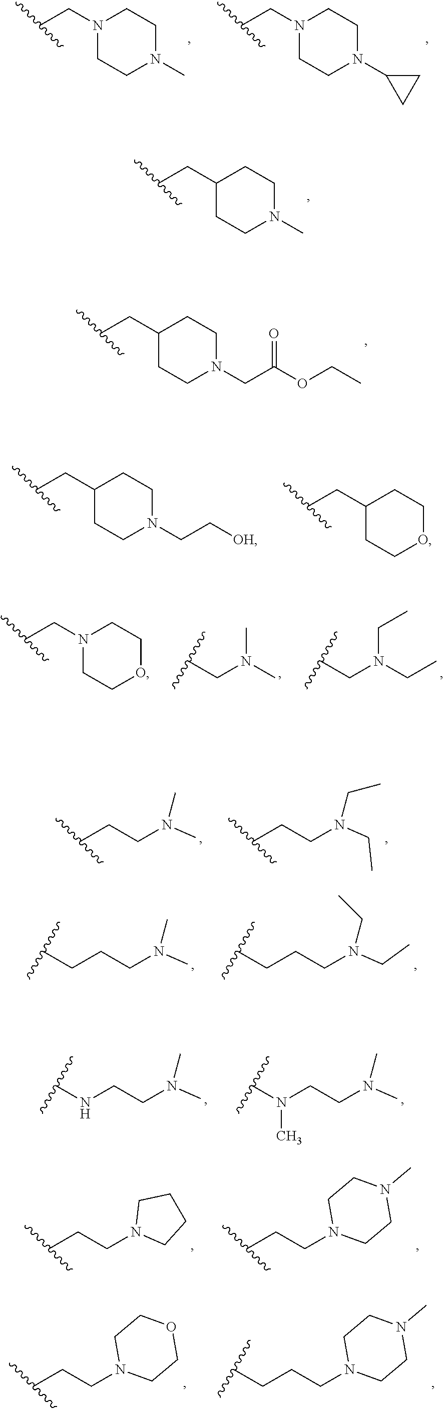

In embodiments, one or more R⁴ groups is independently selected from a second group of permitted R⁴ moieties, wherein said group is: —CH₂OCH₃, —OCH₃, —OCH₂F, —OCHF₂, —OCF₃, —OCH₂CH₃, —OCH₂CH₂F, —OCH₂CHF₂, —OCH₂CF₃, —OCH₂CH₂CH₃, —O CH₂CH(CH₃)₂, —OCH₂CH₂OCH₃,

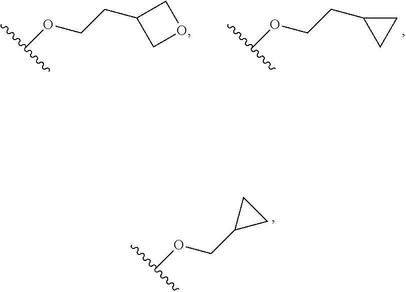 [SMILES: *OCCC1COC1 ; *OCCC1CC1 ; *OCC1CC1]

—CO₂CH₃, and CH₃.

In embodiments, a compound comprises (1) one or more R⁴ groups independently selected from the first group of permitted R⁴ moieties described herein and (2) one or more R⁴ groups independently selected from a second group of permitted R⁴ moieties described herein.

In embodiments, a R⁴ group is R^{4A} as described herein. In embodiments, a R^{4A} is according to any of embodiment of R⁴ described herein. In embodiments, a R^{4A} is selected from the first group of permitted r⁴ moieties described herein.

In embodiments, a R⁴ group is R^{4B} as described herein. In embodiments, a R^{4B} is according to any of embodiment of R⁴ described herein. In embodiments, a R^{4B} is selected from the first group of permitted R⁴ moieties described herein.

In embodiments, a R⁴ group is R^{4C} as described herein. In embodiments, a R⁴ is according to any of embodiment of R⁴ described herein. In embodiments, a R^{4C} is selected from the first group of permitted R⁴ moieties described herein.

In embodiments, a R⁴ group is R^{4D} as described herein. In embodiments, a R^{4D} is according to any of embodiment of R⁴ described herein. In embodiments, R^{4D} is C₁₋₆ alkyl. In embodiments, R^{4D} is unsubstituted C₁₋₆ alkyl.

In embodiments, each R^{4A}, R^{4B}, and R^{4C}, when present, is independently selected from: —C≡N; —C≡CH; a saturated linear or branched C₁₋₆ aliphatic or C₁₋₆ alkoxy comprising 0-4 fluoro substituents; NR¹¹(CH₂)ₛNR⁸R⁹; (CH₂)ₜNR⁸R⁹; O(CH₂),OCH₃; O(CH₂)ᵣR¹²; and (CH₂)ᵣR¹².

In embodiments, an R^{4A} and/or a R^{4C} group, when present, is selected from: —CN, —CH₃, —CH₂F, —CHF₂, —CF₃, —CH₂CH₃, —CH₂CFH₂, —CH₂CHF₂, —CH₂CF₃, —CH(CH₃)₂, —C(CH₃)₃, —C≡CH,

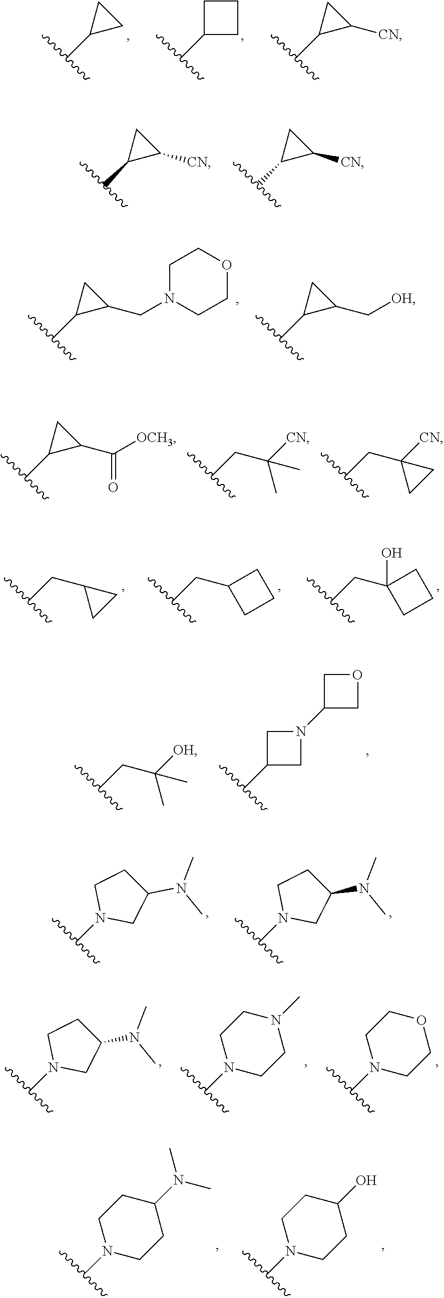

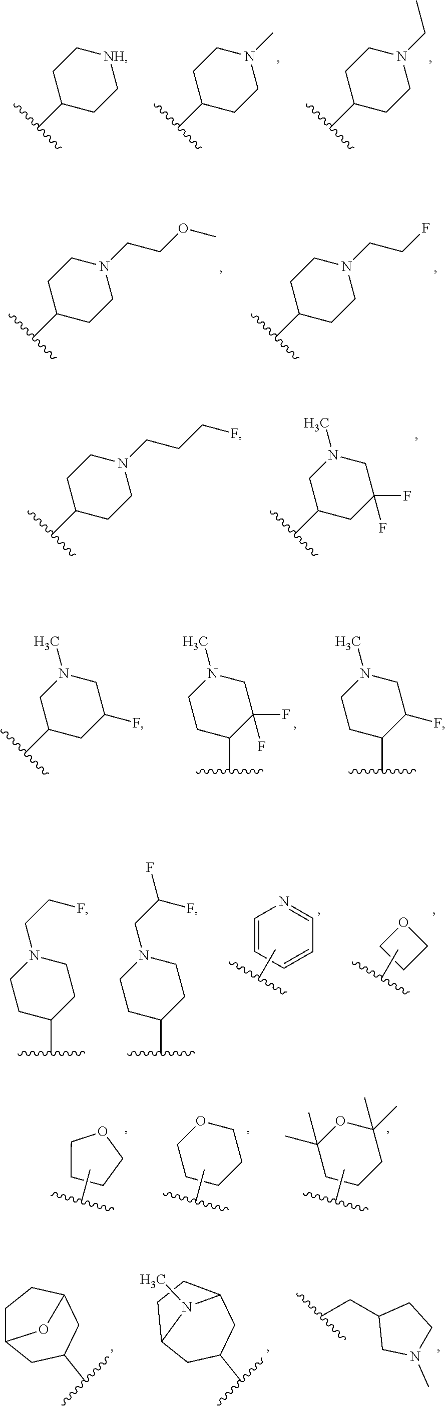

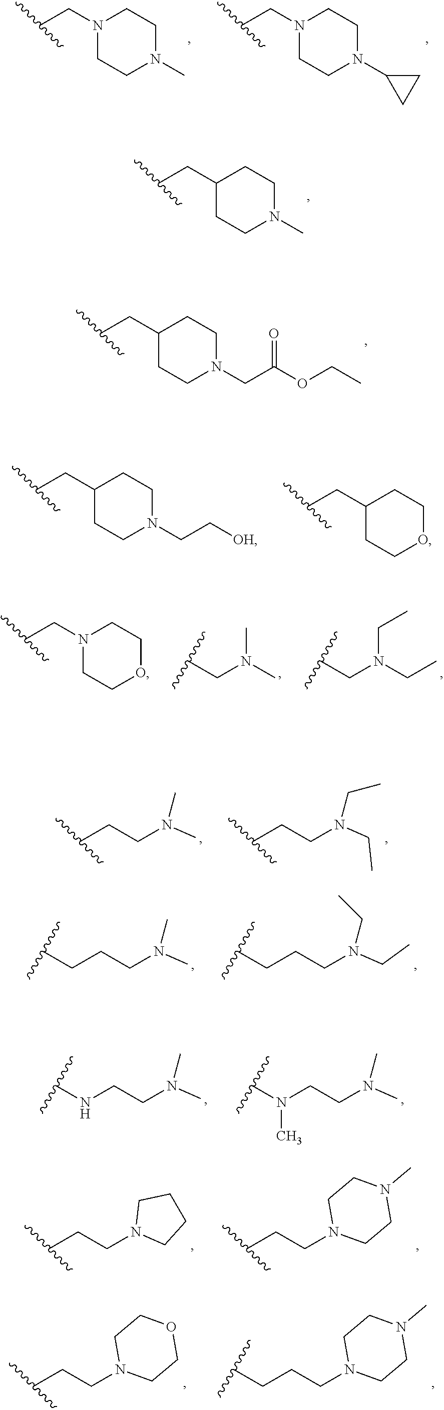

In embodiments, an R^{4B} group, when present, is selected from —CH₂OCH₃, —OCH₃, —OCH₂F, —OCHF₂, —OCF₃, —OCH₂CH₃, —OCH₂CH₂F, —OCH₂C HF₂, —OCH₂CF₃, —OCH₂CH₂CH₃, —OCH₂CH(CH₃)₂, —OCH₂CH₂OCH₃,

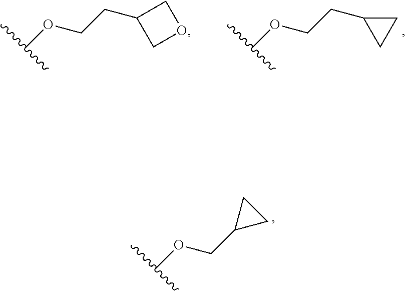 [SMILES: *OCCC1COC1 ; *OCCC1CC1 ; *OCC1CC1]

—CO₂CH₃, and CH₃.

In embodiments,

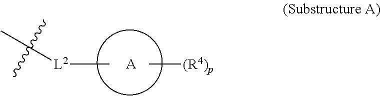

comprises a R⁴ that is

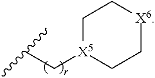

In embodiments,

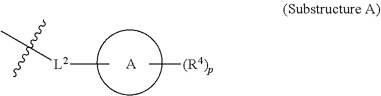

comprises a R⁴ that is

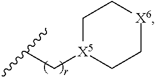

and a second R⁴ group. In embodiments, a second R⁴ group is selected from the group consisting of unsubstituted C₁₋₆ alkyl (e.g., —CH₃ or —CH₂CH₃), CO₂(unsubstituted C₁₋₆ alkyl) (e.g., —CO₂CH₃), O-(unsubstituted C₁₋₆ alkyl) (e.g., —OCH₃ or —OCH₂CH₃), O—(C₁₋₆ haloalkyl) (e.g., —OCH₂CF₃), NH(CH₂)ₛNMe₂ (e.g., —NHCH₂CH₂N(CH₃)₂), and (CH₂)ₜNMe₂ (e.g., —CH₂N(CH₃)₂).

In embodiments,

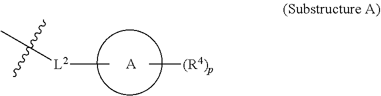

is

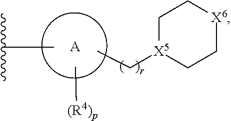

wherein
A is phenyl or 5- to 6-membered heteroaryl.
X⁵ is independently CH or N;
X⁶ is independently O, CHR³, or NR³;
R¹³ is independently H, unsubstituted C₁₋₆ alkyl, or unsubstituted C₃₋₆ cycloalkyl;
r is 0 or 1;
R⁴ is selected from unsubstituted C₁₋₆ alkyl, CO₂(unsubstituted C₁₋₆ alkyl), O-(unsubstituted C₁₋₆ alkyl), O—(C₁₋₆ haloalkyl), or NH(CH₂)ₛNMe₂;
p is 0 or 1; and
s is an integer of 2-6.

In embodiments,

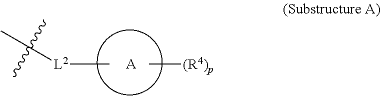

is

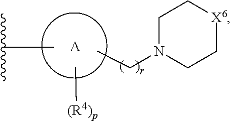

wherein X⁶ is O, NCH₃, or N(cyclopropyl).

In embodiments,

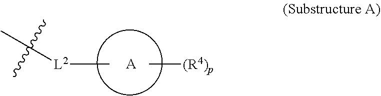

is selected from the group consisting of:

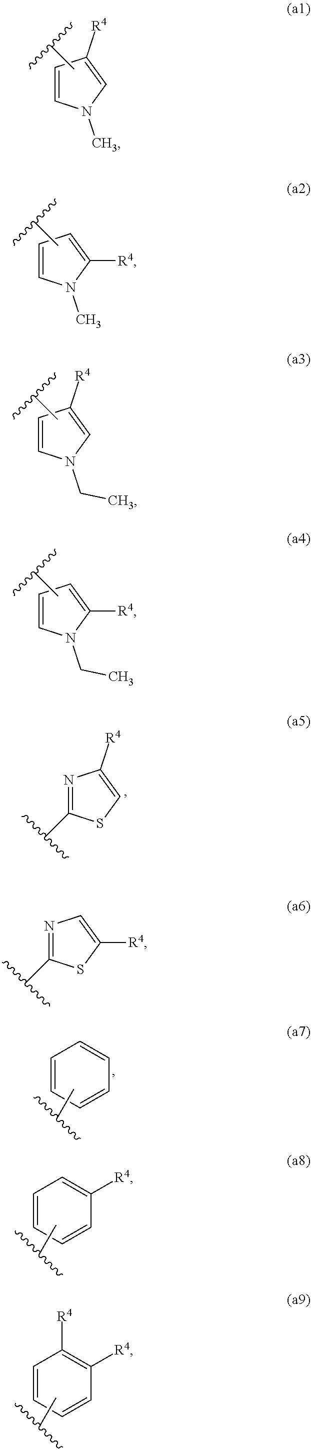

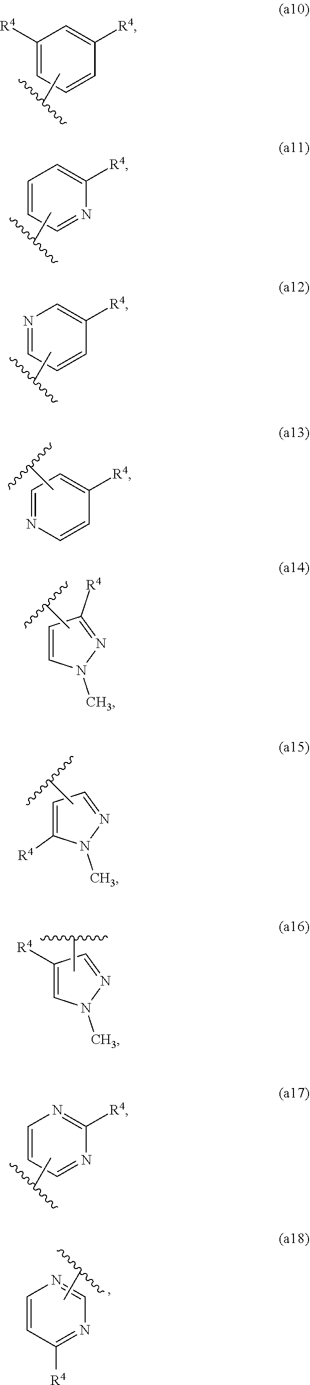

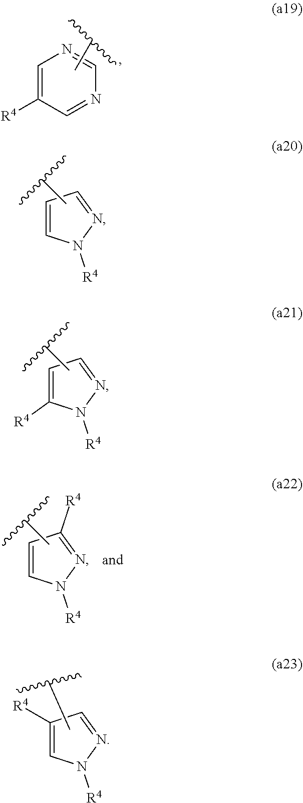

In embodiments. Substructure A is selected from the group consisting of (a1)-(a20).

In embodiments, Substructure A is selected from the group consisting of (a20)-(a23).

In embodiments,

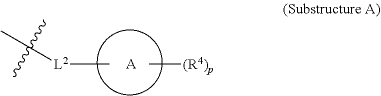

is selected from the group consisting of:

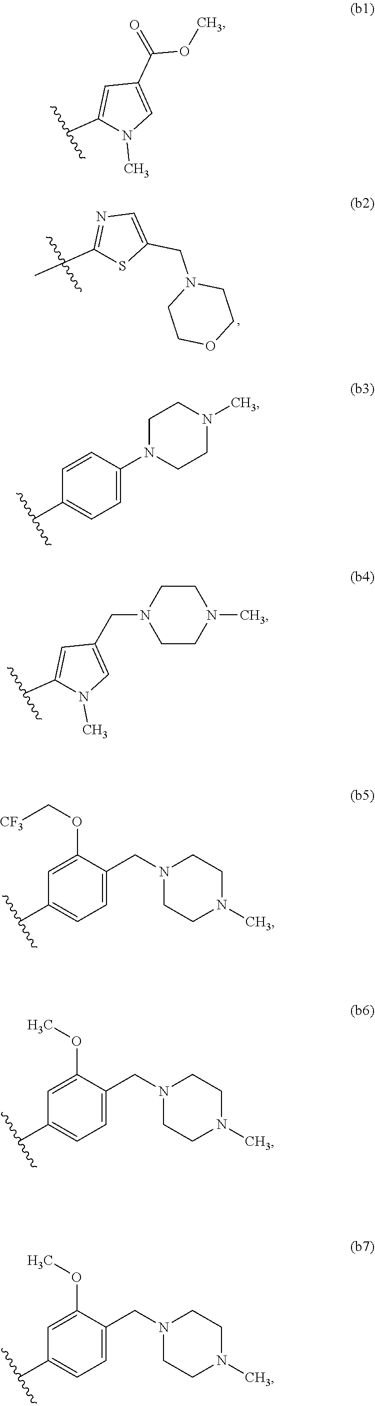

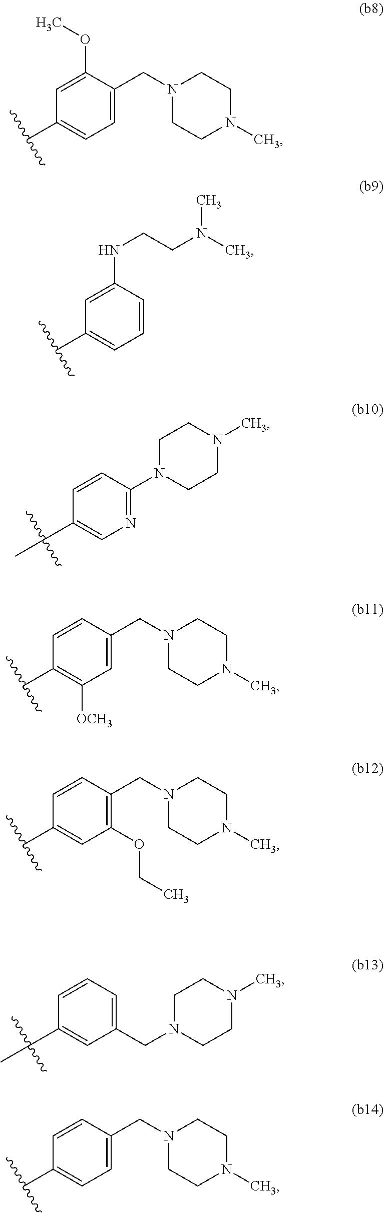

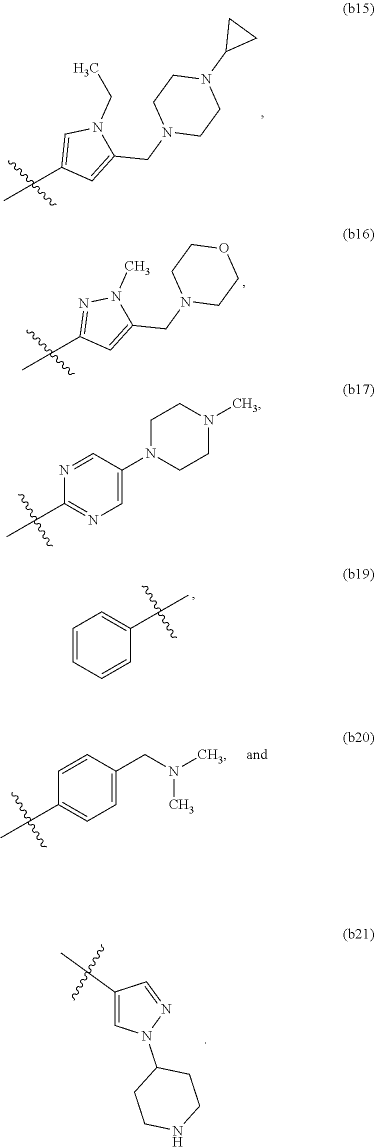 [SMILES: *c1cc(CN2CCN(C3CC3)CC2)n(CC)c1 ; *c1cc(CN2CCOCC2)n(C)n1 ; *c1ncc(N2CCN(C)CC2)cn1 ; *c1ccccc1 ; *c1ccc(CN(C)C)cc1 ; *c1cnn(C2CCNCC2)c1]

In embodiments,

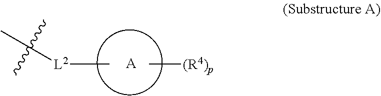

is

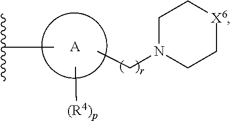

and p is 1. In embodiments, Substructure A is selected from the group consisting of:

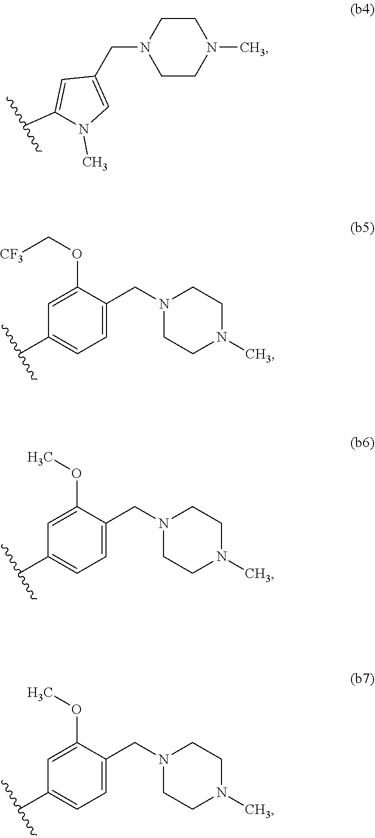 [SMILES: *c1cc(CN2CCN(C)CC2)cn1C ; *c1ccc(CN2CCN(C)CC2)c(OCC(F)(F)F)c1 ; *c1ccc(CN2CCN(C)CC2)c(OC)c1 ; *c1ccc(CN2CCN(C)CC2)c(OC)c1]

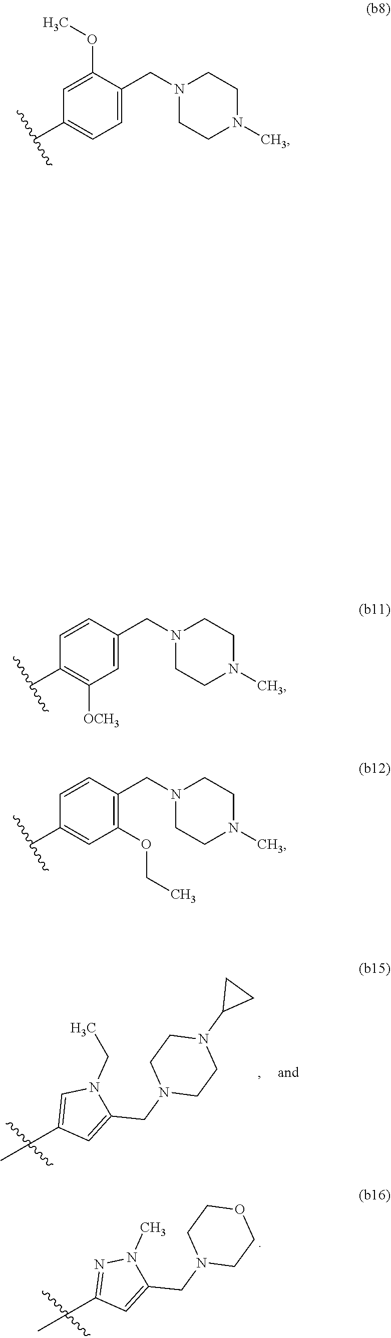 [SMILES: *c1ccc(CN2CCN(C)CC2)c(OC)c1 ; *c1ccc(CN2CCN(C)CC2)cc1OC ; *c1ccc(CN2CCN(C)CC2)c(OCC)c1 ; *c1cc(CN2CCN(C3CC3)CC2)n(CC)c1 ; *c1cc(CN2CCOCC2)n(C)n1]

### Exemplary Compounds

Exemplary compounds (e.g., according to Formulas (I)-(XXIII) or any other formula described herein) include any one of the following compounds in Table A. Accordingly, exemplary compounds include any of Compounds (1)-(169), or a pharmaceutically acceptable salt thereof.

TABLE A
Exemplary Compounds

| | | ESI-MS |
|---|---|---|
| | | m/z |
| No. | Structure | [M + H]⁺ |
| (1) | 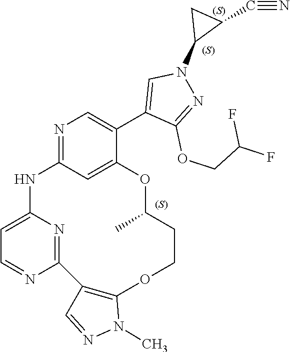 | 550.2 |
| (2) | 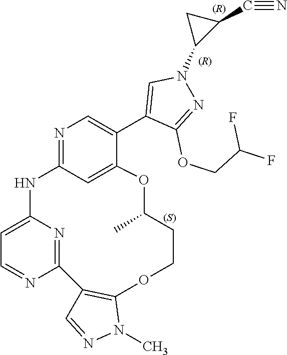 | 550.2 |
| (3) | 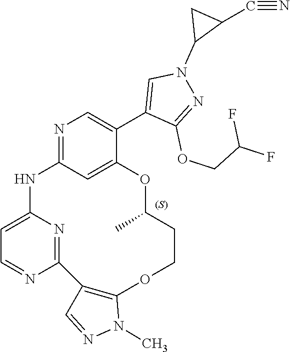 | 550.1 |
| (4) | 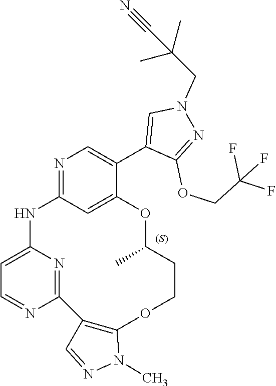 | 584.1 |
| (5) | 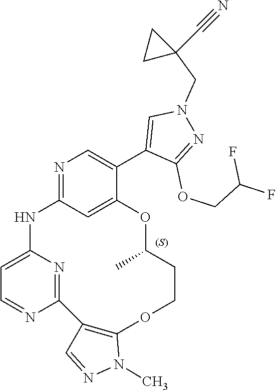 | 564.1 |
| (6) | 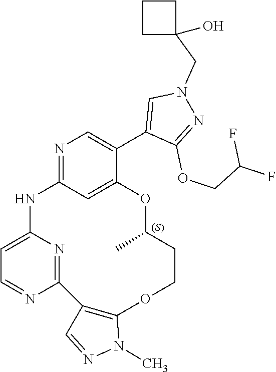 | 569.1 |
| (7) | 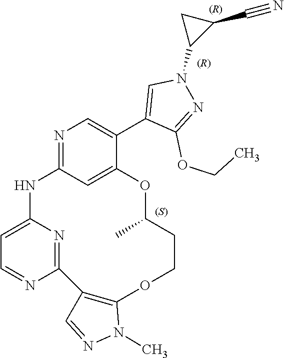 | 514.2 |
| (8) | 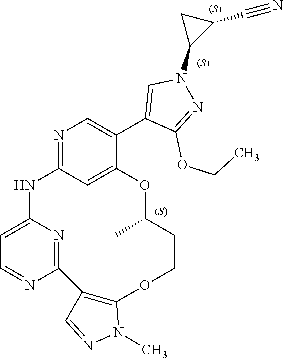 | 514.2 |
| (9) | 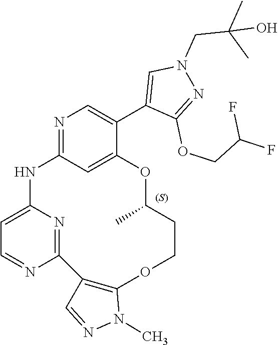 | 557.1 |
| (10) | 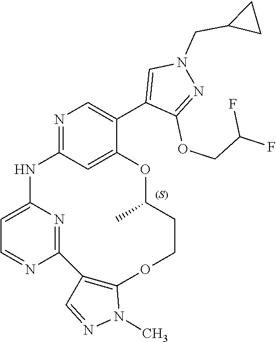 | 539.1 |
| (11) | 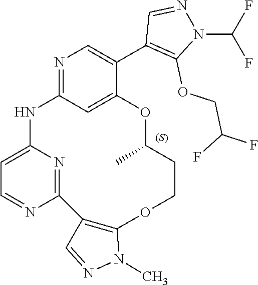 | 535.1 |
| (12) | 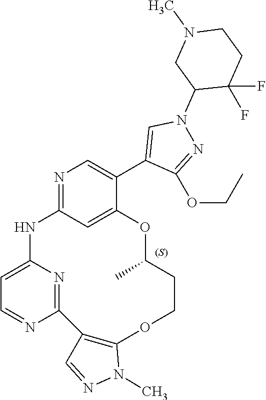 | 582.2 |
| (13) | 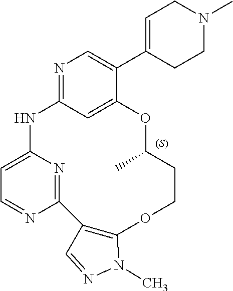 | 434.1 |
| (14) | 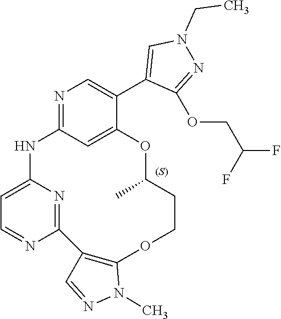 | — |
| (15) | 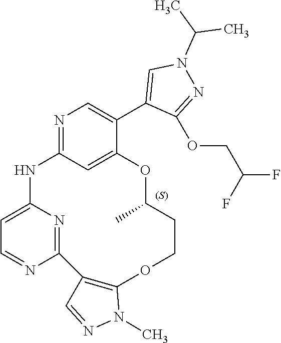 | 527.1 |
| (16) | 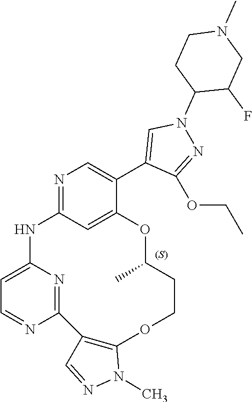 | 564.2 |
| (17) | 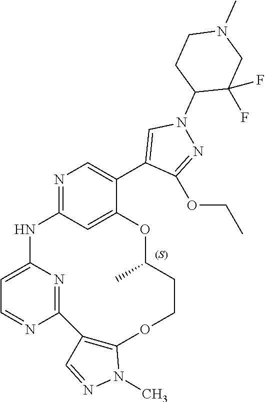 | 582.2 |
| (18) | 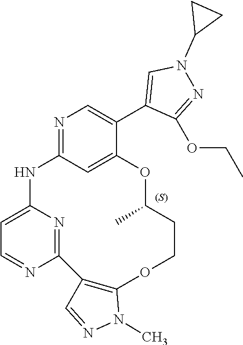 | — |
| (19) | 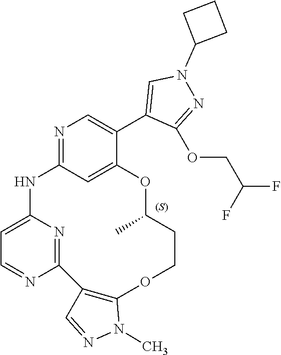 | 539.3 |
| (20) | 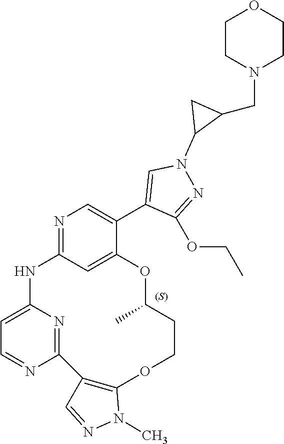 | 588.3 |
| (21) | 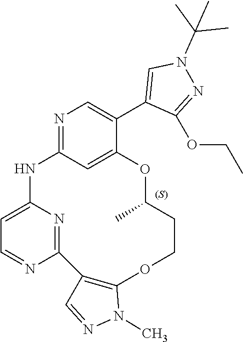 | 505.2 |
| (22) | 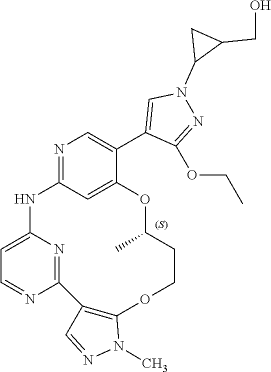 | 519.1 |
| (23) | 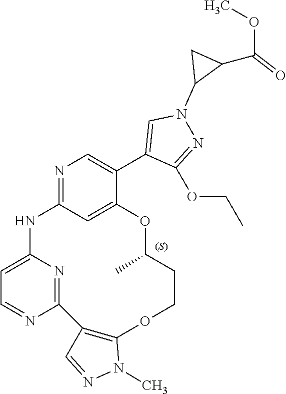 | 547.1 |
| (24) | 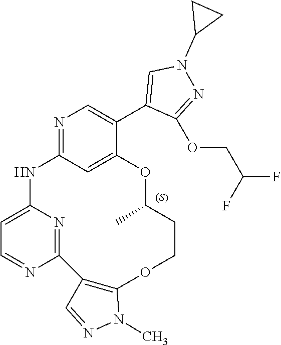 | 525 |
| (25) | 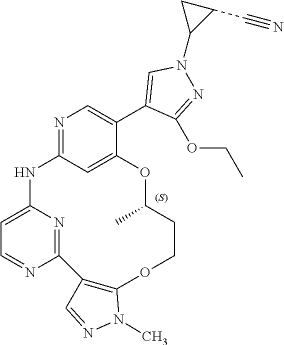 | 514.1 |
| (26) | 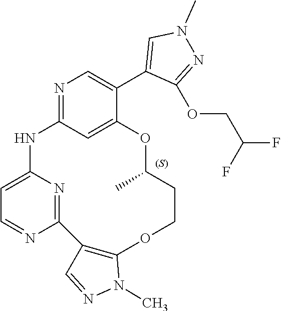 | 499.1 |
| (27) | 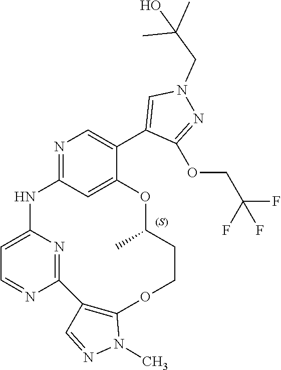 | 575.2 |
| (28) | 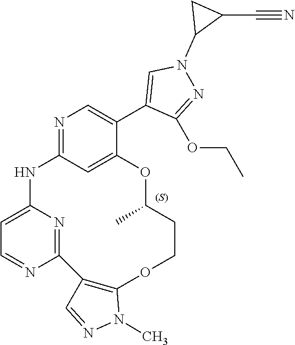 | 514.1 |
| (29) | 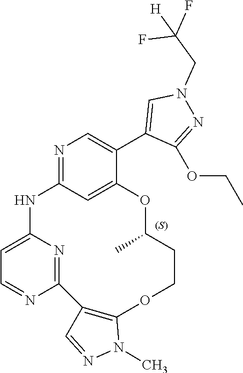 | 513.1 |
| (30) | 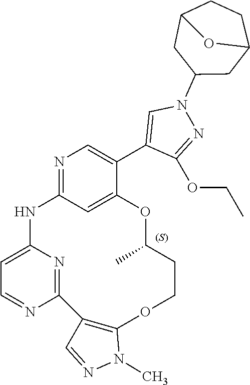 | 559.1 |
| (31) | 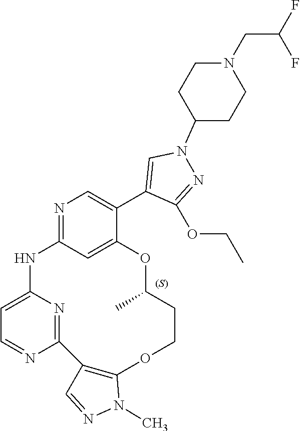 | 596.3 |
| (32) | 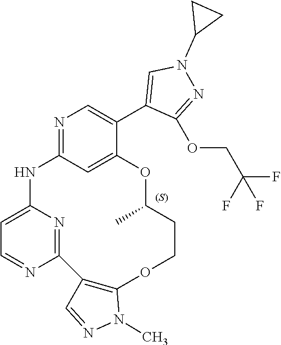 | 542.1 |
| (33) | 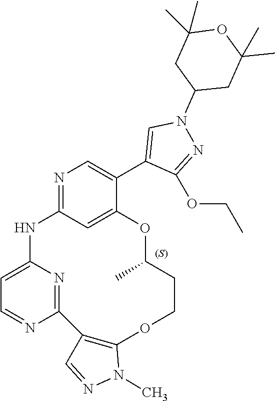 | 589.3 |
| (34) | 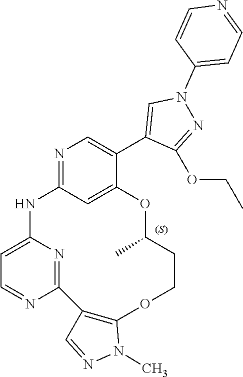 | 526.1 |
| (35) | 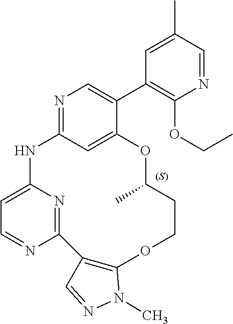 | 474.5 |
| (36) | 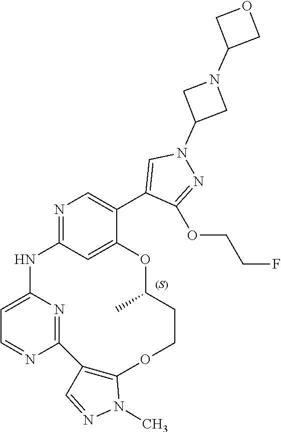 | 578.2 |
| (37) | 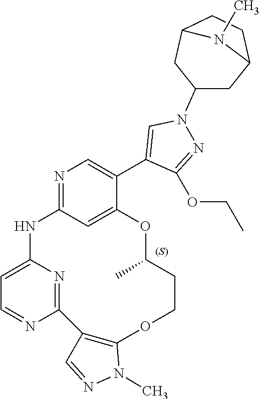 | 572.2 |
| (38) | 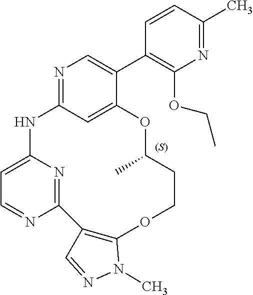 | 474.1 |
| (39) | 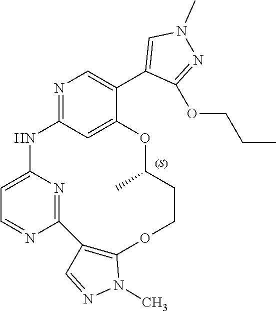 | 477.1 |
| (40) | 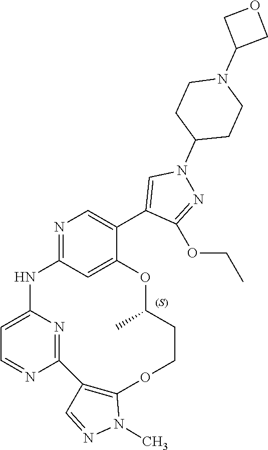 | 588.3 |
| (41) | 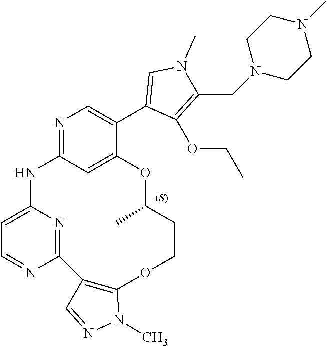 | 574.6 |
| (42) | 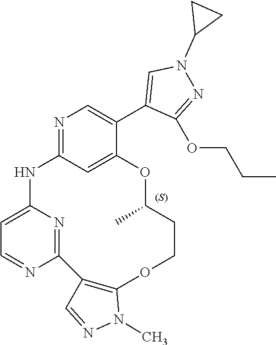 | 503.2 |
| (43) | 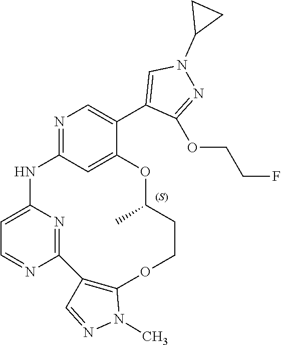 | 507.1 |
| (44) | 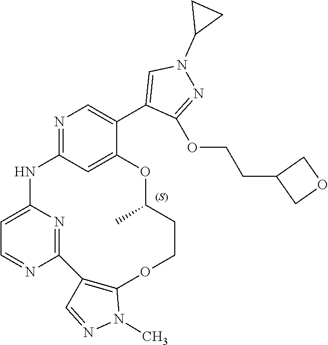 | 545.1 |
| (45) | 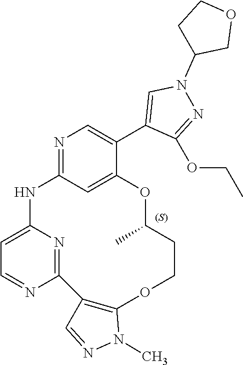 | 519.3 |
| (46) | 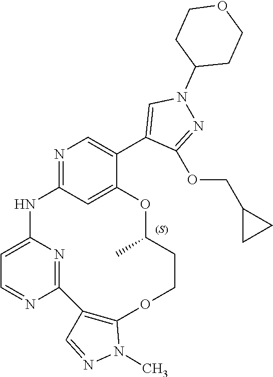 | 559.1 |
| (47) | 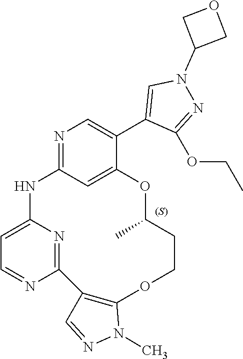 | 505.2 |
| (48) | 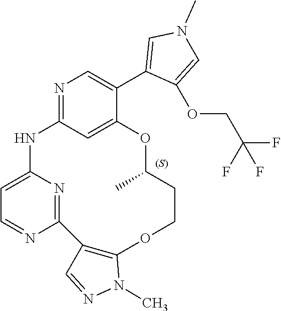 | 516.1 |
| (49) | 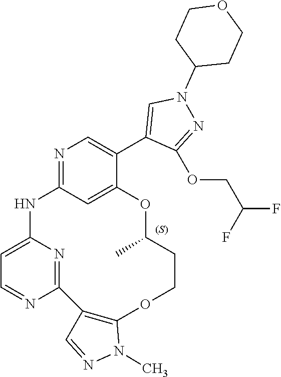 | 569.2 |
| (50) | 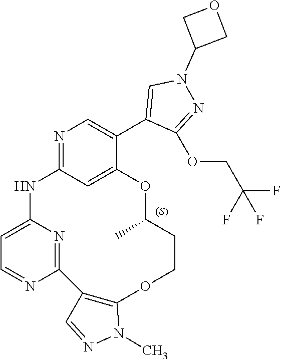 | 559.1 |
| (51) | 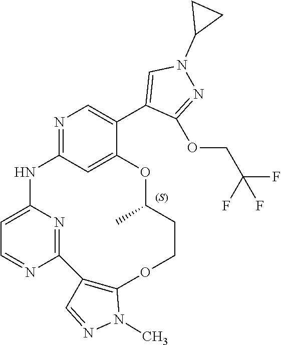 | 543.2 |
| (52) | 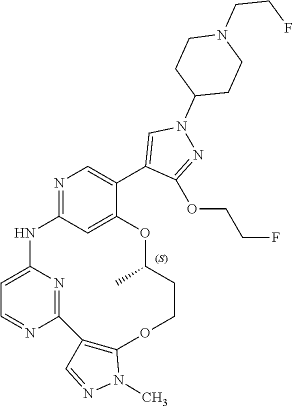 | 618.2 (+Na) |
| (53) | 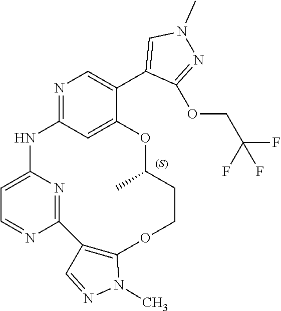 | 517.1 |
| (54) | 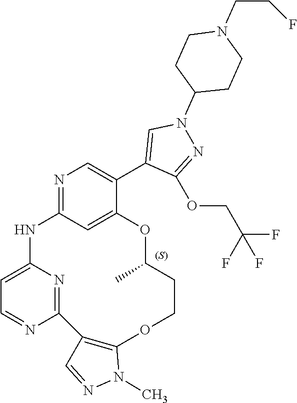 | 632.3 |
| (55) | 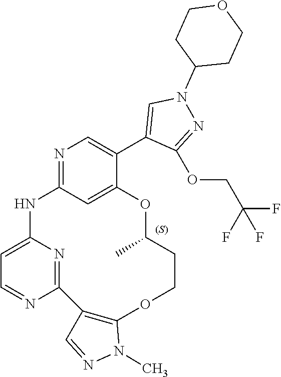 | 587.3 |
| (56) | 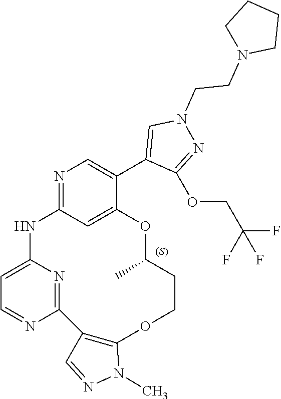 | — |
| (57) | 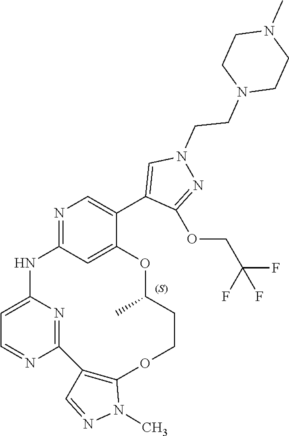 | 629.3 |
| (58) | 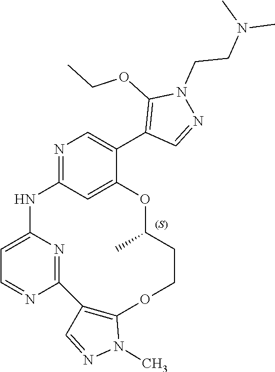 | 520.2 |
| (59) | 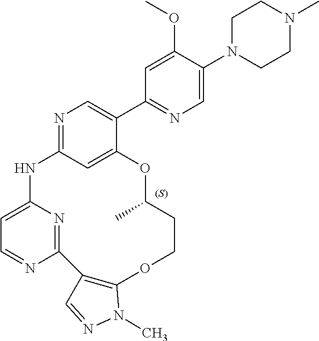 | 544.3 |
| (60) | 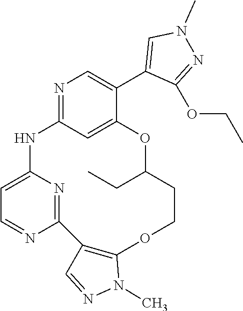 [SMILES: CCOc1nn(C)cc1-c1cnc2cc1OC(CC)CCOc1c(cnn1C)-c1nccc(n1)N2] | 477.2 |
| (61) | 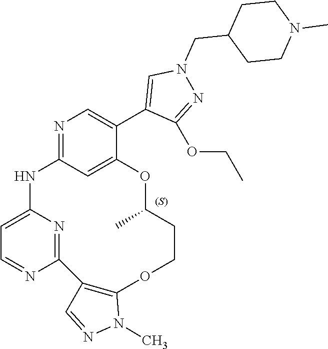 | 560.3 |
| (62) | 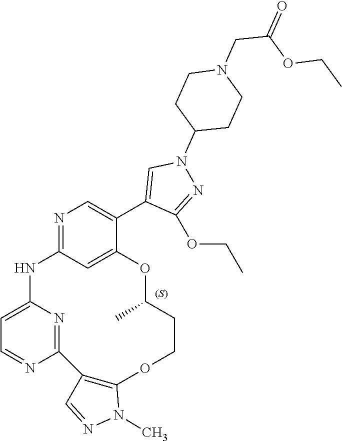 | 618.3 |
| (63) | 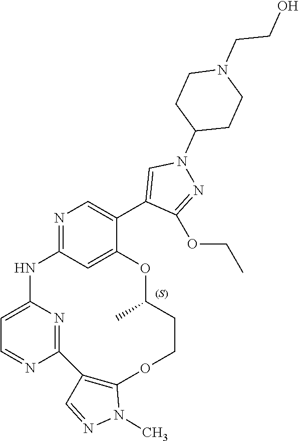 | 576.3 |
| (64) | 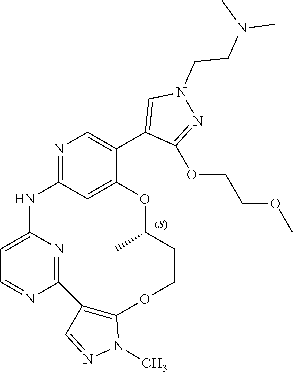 | 550.4 |
| (65) | 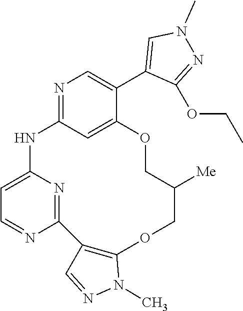 [SMILES: CCOc1nn(C)cc1-c1cnc2cc1OCC(C)COc1c(cnn1C)-c1nccc(n1)N2] | 463.2 |
| (66) | 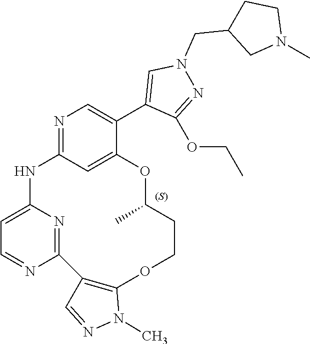 | 546.4 |
| (67) | 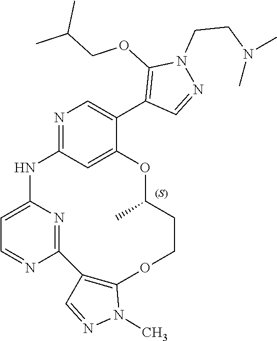 | 548.3 |
| (68) | 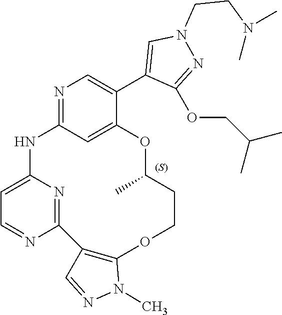 | 548.3 |
| (69) | 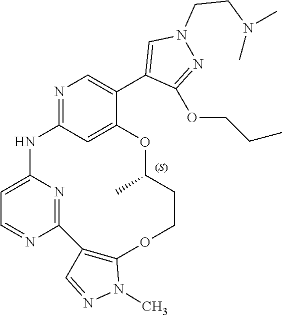 | 534.3 |
| (70) | 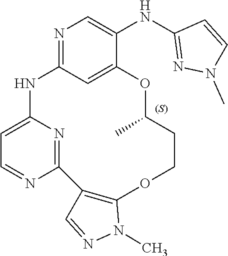 | 434.1 |
| (71) | 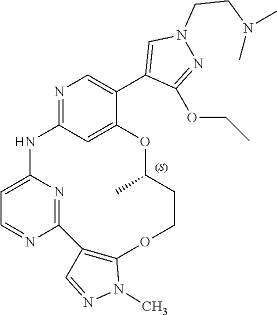 | 520.2 |
| (72) | 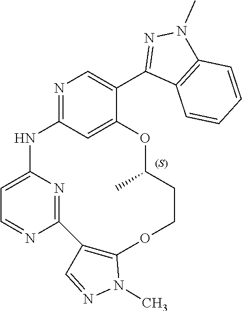 | 469.2 |
| (73) | 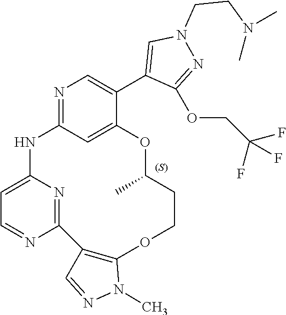 | 574.3 |
| (74) | 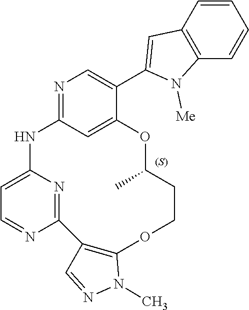 | 468.2 |
| (75) | 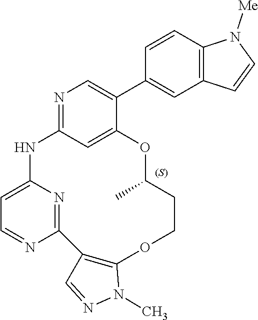 | 468.2 |
| (76) | 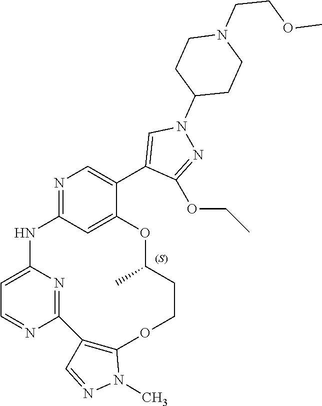 | 590.3 |
| (77) | 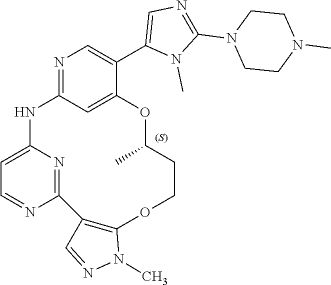 | 517.2 |
| (78) | 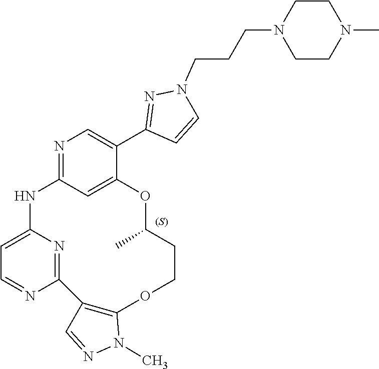 | 567.2 (+Na) |
| (79) | 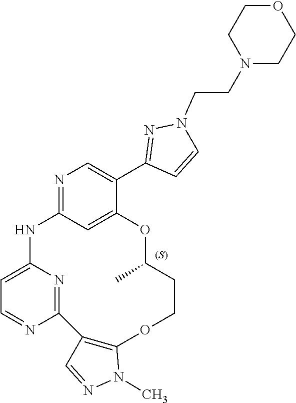 | 518.2 |
| (80) | 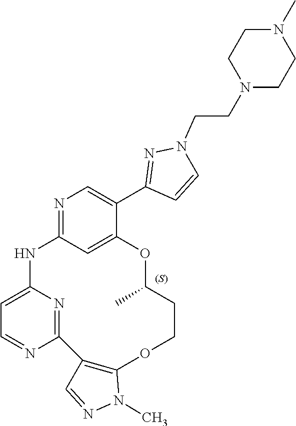 | 553.2 (+Na) |
| (81) | 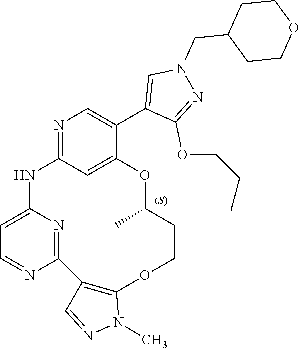 | 547.2 |
| (82) | 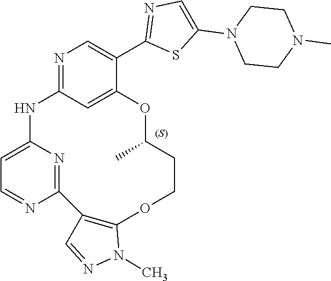 | 520.1 |
| (83) | 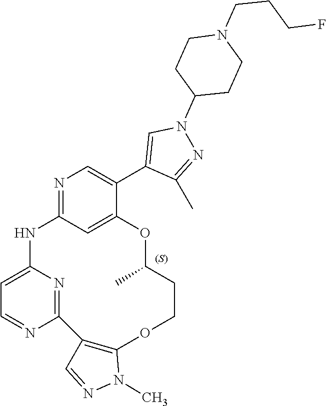 | 562.3 |
| (84) | 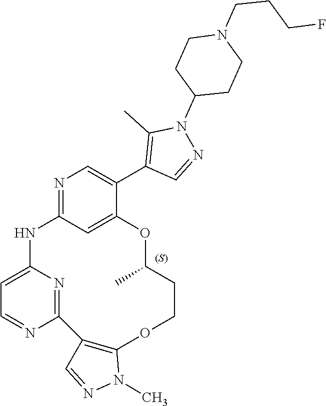 | 562.2 |
| (85) | 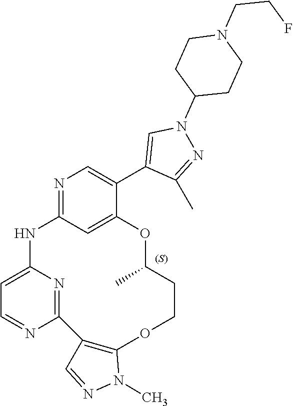 | 548.2 |
| (86) | 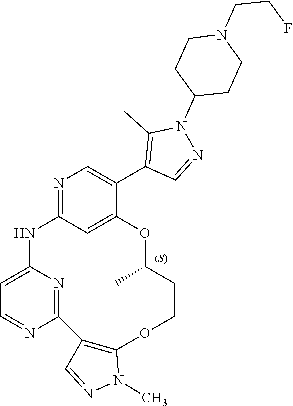 | 548.2 |
| (87) | 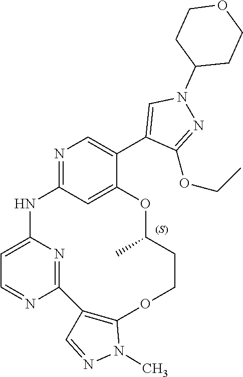 | 533.1 |
| (88) | 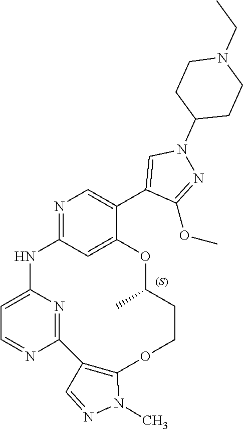 | 546.2 |
| (89) | 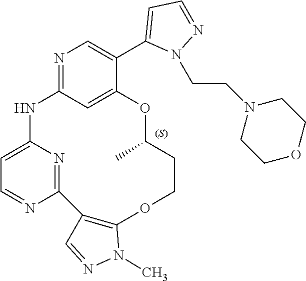 | 518.2 |
| (90) | 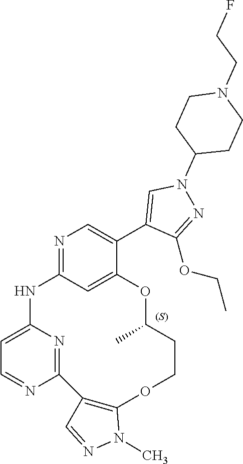 | 578.3 |
| (91) | 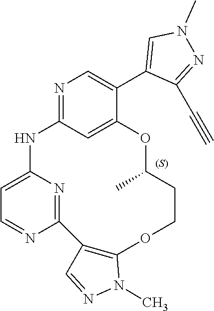 | 443.1 |
| (92) | 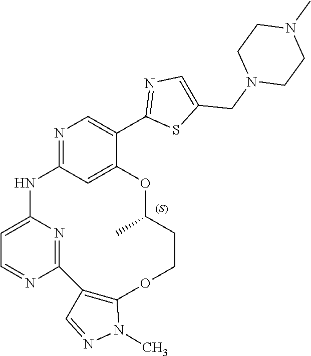 | 534.1 |
| (93) | 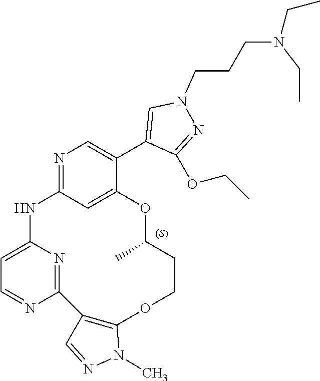 | 562.3 |
| (94) | 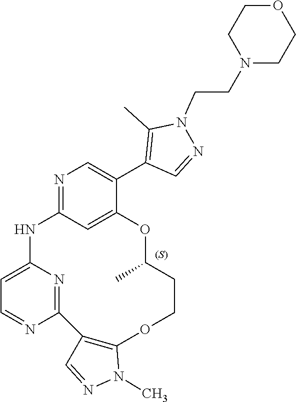 | 532.1 |
| (95) | 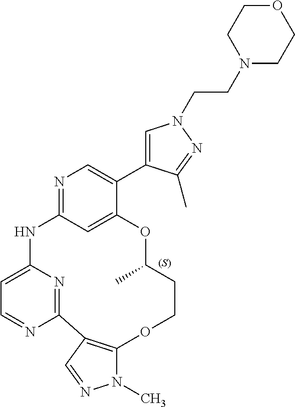 | — |
| (96) | 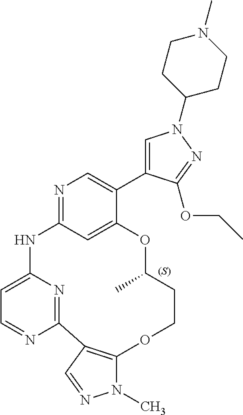 | 545.2 |
| (97) | 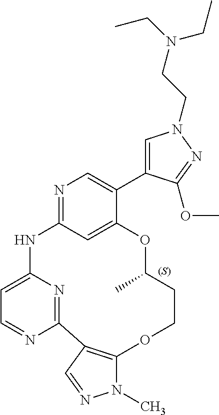 | 534.2 |
| (98) | 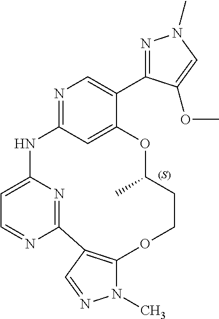 | 449 |
| (99) | 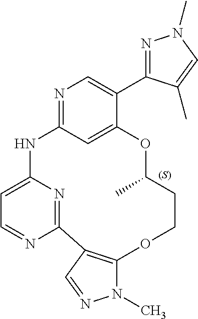 | 433.1 |
| (100) | 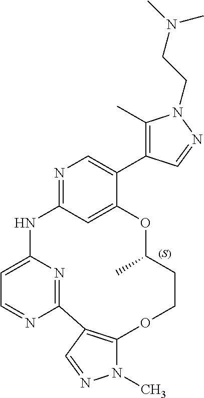 | 490.1 |
| (101) | 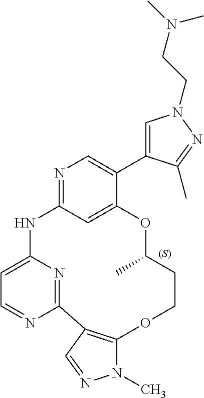 | 490.1 |
| (102) | 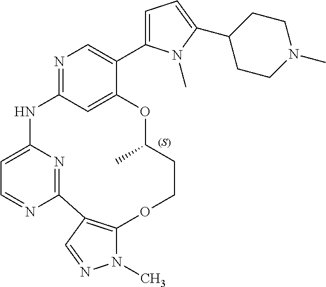 | 515.2 |
| (103) | 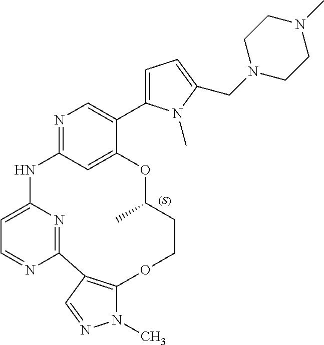 | 530.4 |
| (104) | 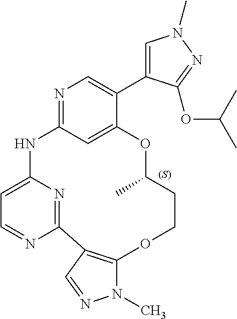 | 477.2 |
| (105) | 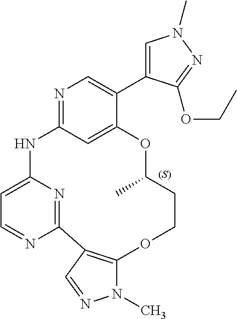 | 463.1 |
| (106) | 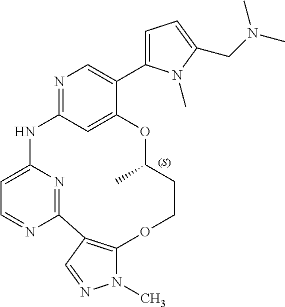 | 475.1 |
| (107) | 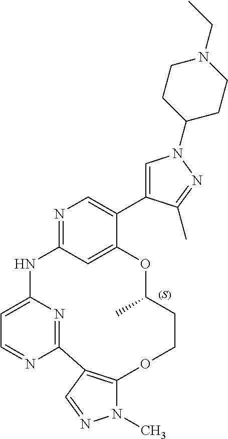 | 530.2 |
| (108) | 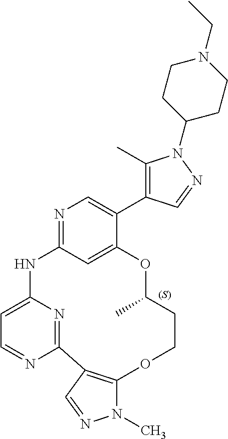 | 530.2 |
| (109) | 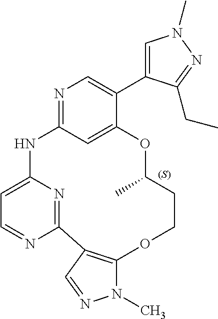 | 447.2 |
| (110) | 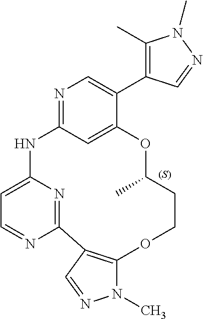 | 433.1 |
| (111) | 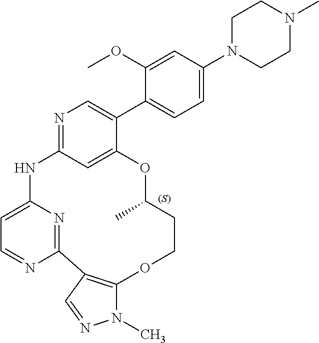 | 543.4 |
| (112) | 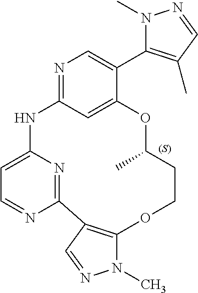 | 433.2 |
| (113) | 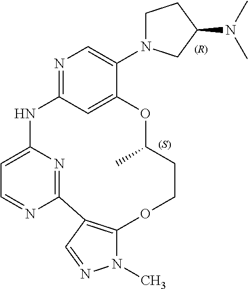 | 451.1 |
| (114) | 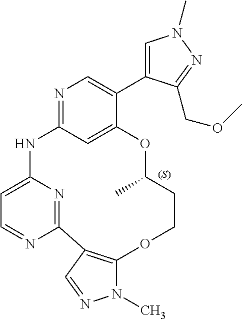 | 463.1 |
| (115) | 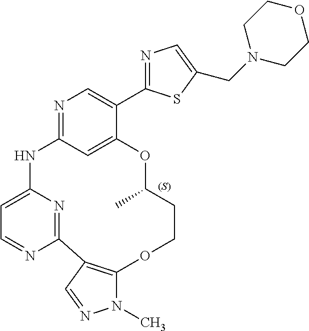 | 521.1 |
| (116) | 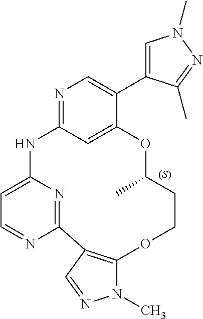 | 433.1 |
| (117) | 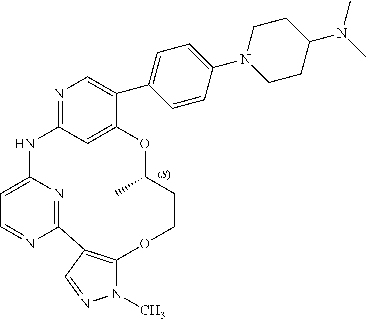 | 541.1 |
| (118) | 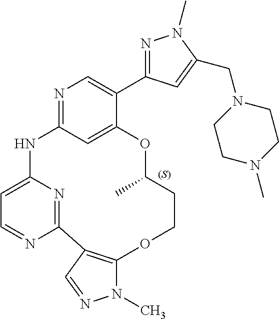 | 531.1 |
| (119) | 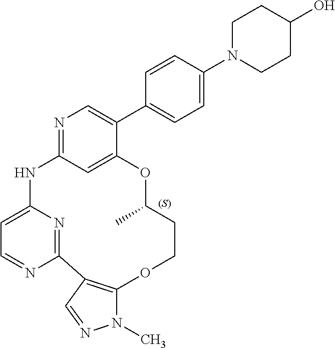 | 514.1 |
| (120) | 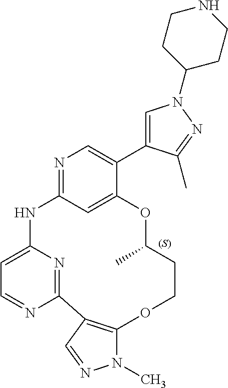 | — |
| (121) | 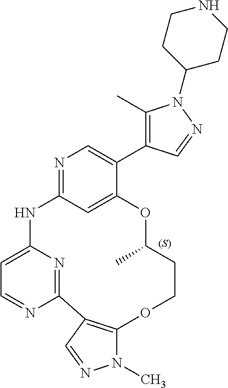 | — |
| (122) | 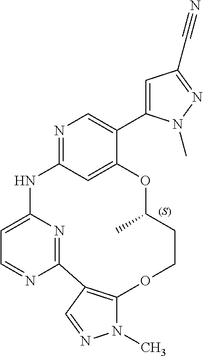 | 444 |
| (123) | 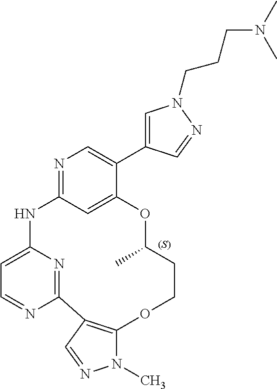 | 490.1 |
| (124) | 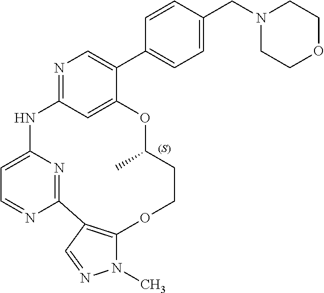 | 514.3 |
| (125) | 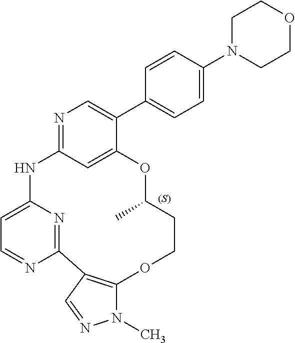 | 500 |
| (126) | 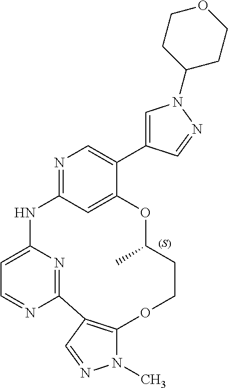 | 489.1 |
| (127) | 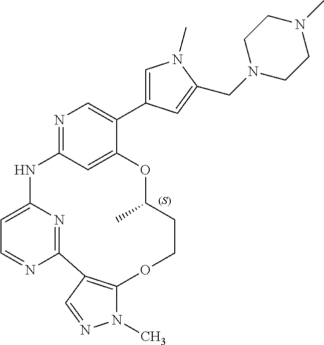 | 530.1 |
| (128) | 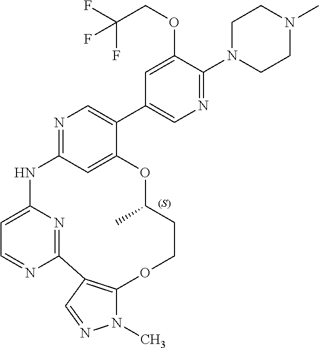 | 612.1 |
| (129) | 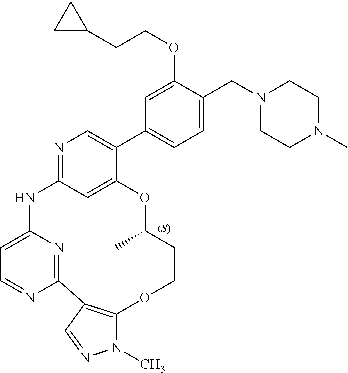 | 611.2 |
| (130) | 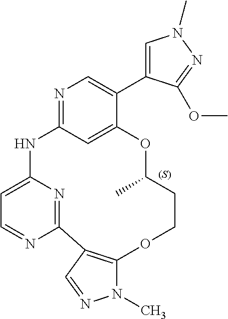 | 449.2 |
| (131) | 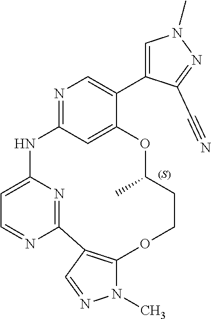 | 444 |
| (132) | 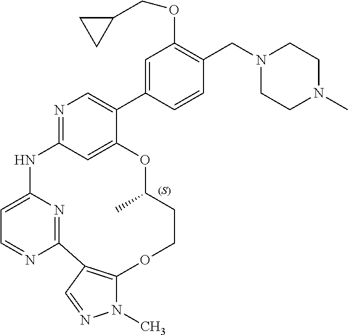 | 597.1 |
| (133) | 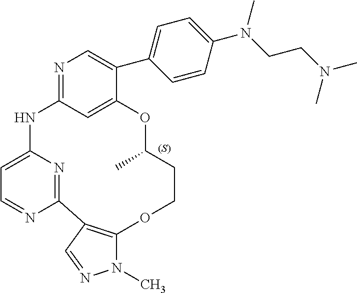 | 515.1 |
| (134) | 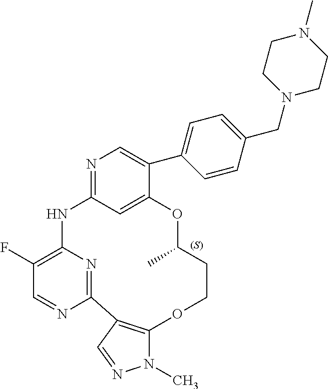 | 545.1 |
| (135) | 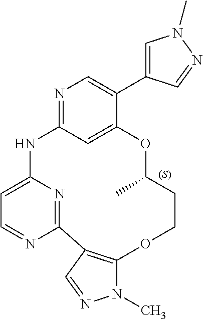 | 419 |
| (136) | 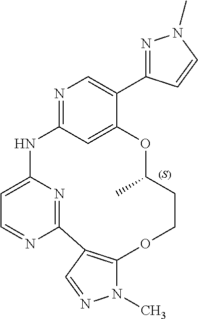 | 419 |
| (137) | 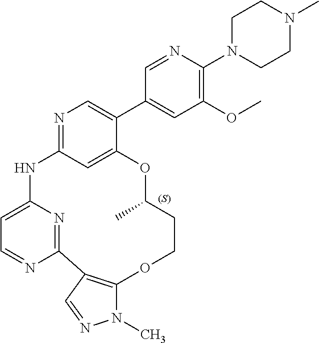 | 544.1 |
| (138) | 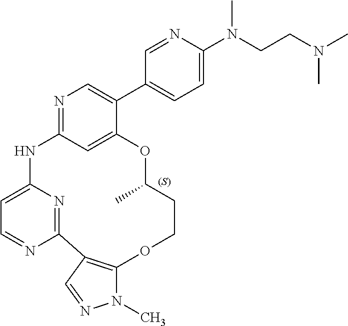 | 516.1 |
| (139) | 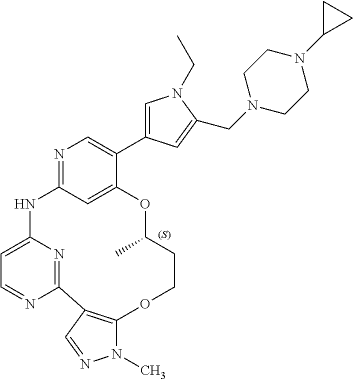 | 570.2 |
| (140) | 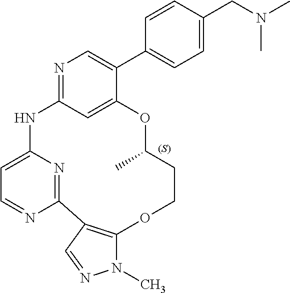 | 472.2 |
| (141) | 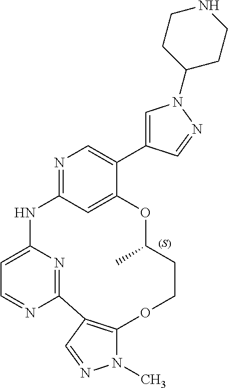 | 488.1 |
| (142) | 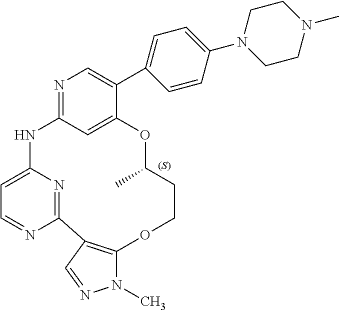 | 513.1 |
| (143) | 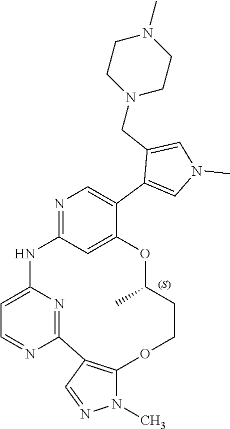 | 530 |
| (144) | 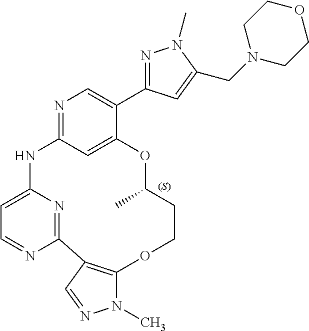 | 518.1 |
| (145) | 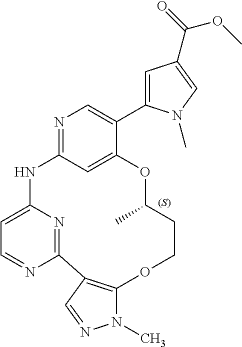 | 476 |
| (146) | 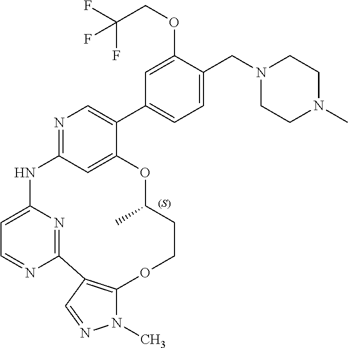 | 625.1 |
| (147) | 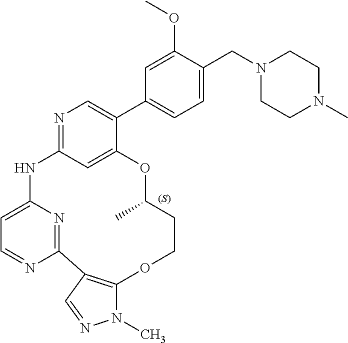 | 557.1 |
| (148) | 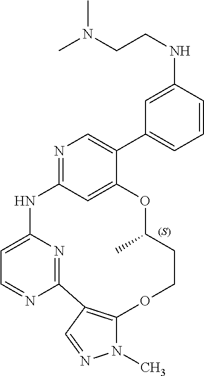 | 501.1 |
| (149) | 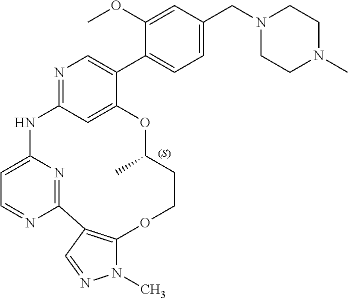 | 557.3 |
| (150) | 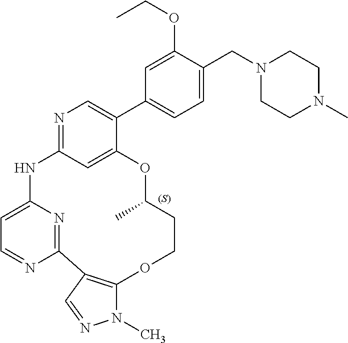 | 571.3 |
| (151) | 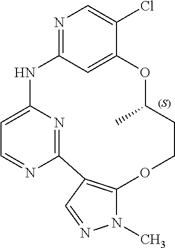 | 373.1 |
| (152) | 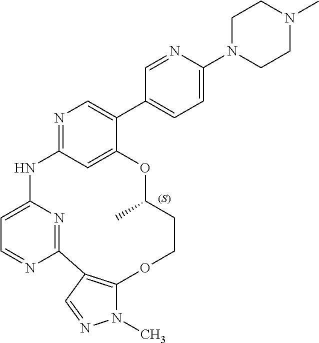 | 514.1 |
| (153) | 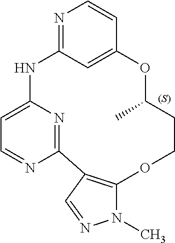 | 338.9 |
| (154) | 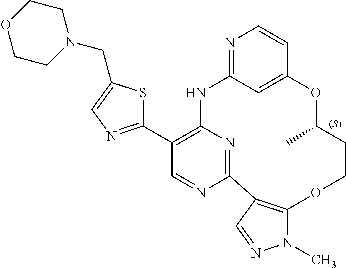 | 521.1 |
| (155) | 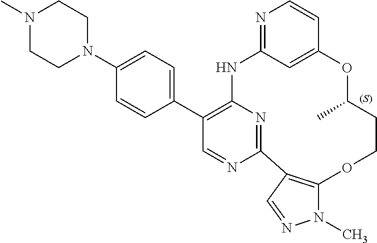 | 513.1 |
| (156) | 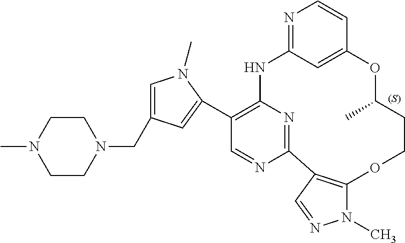 | 530.1 |
| (157) | 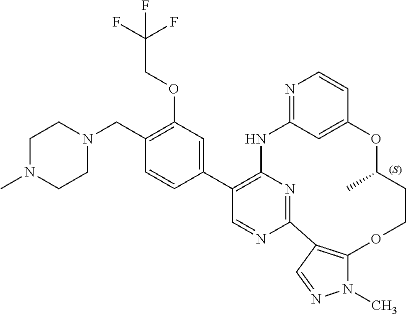 | 625.3 |
| (158) | 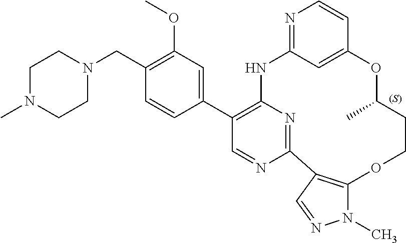 | 557.1 |
| (159) | 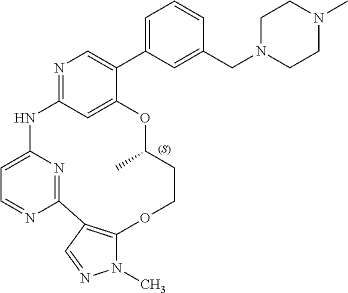 | 527.3 |
| (160) | 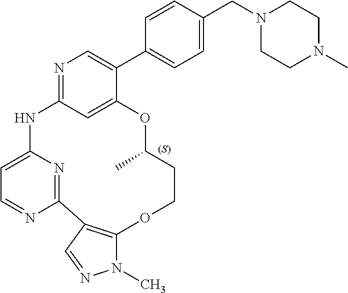 | 527.5 |
| (161) | 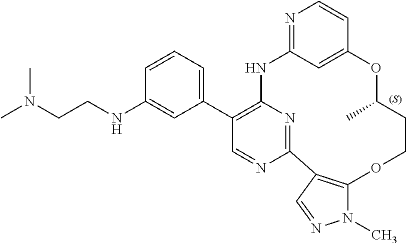 | 501.1 |
| (162) | 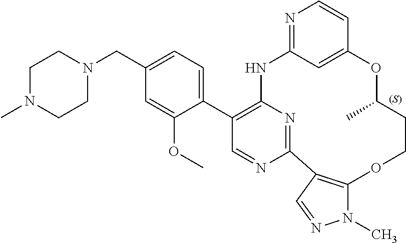 | 557.1 |
| (163) | 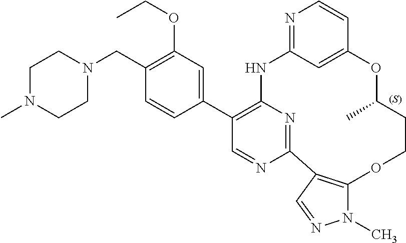 | 571.2 |
| (164) | 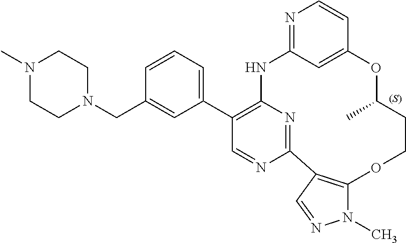 | 527.4 |
| (165) | 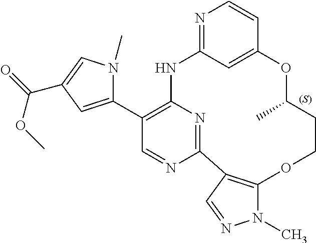 | 476.2 |
| (166) | 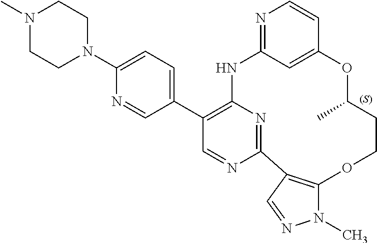 | 514.3 |
| (167) | 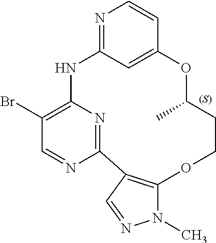 | 417.1 |
| (168) | 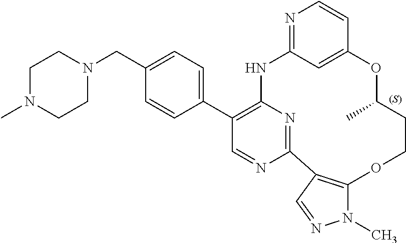 | 527.3 |
| (169) | 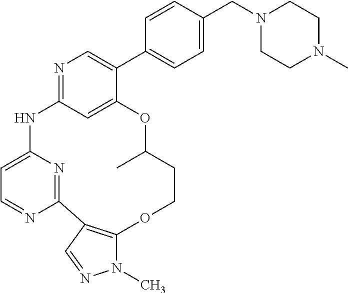 [SMILES: CC1CCOc2c(cnn2C)-c2nccc(n2)Nc2cc(c(-c3ccc(CN4CCN(C)CC4)cc3)cn2)O1] | 527.1 |

In embodiments, compounds described herein can be potent, reversible inhibitors of kinases such as EGFR. Accordingly, in embodiments, compounds described herein (e.g., any compound of Formulas (I)-(XXIII), including as exemplified by any of Compounds (1)-(169)) do not comprise functional groups selected from acrylamides, vinyl sulfonates, quinones, alkynyl amides, propargylic acid derivatives, α-halo ketones, thiocyanates, nitriles, epoxides, sulfonyl fluorides, and cyclic 1,3-diketones as permitted groups for any variable in that structure.

### Deuterated Compounds

Compounds described herein can comprise atoms that exhibit their natural isotopic abundances, or one or more of the atoms may be artificially enriched in a particular isotope having the same atomic number, but an atomic mass or mass number different from the atomic mass or mass number predominately found in nature. The term “isotopologue” refers to a species that has the same chemical structure and formula as a specific compound provided herein, with the exception of the positions of isotopic substitution and/or level of isotopic enrichment at one or more positions, e.g., hydrogen vs. deuterium. The present invention is meant to include all suitable isotopic variations of the compounds of the compounds described herein. For example, different isotopic forms of hydrogen (H) include protium (¹H), deuterium (²H), and tritium (³H), as well as compositions enriched in isotopologues of any compound described herein.

In embodiments, one or more of the hydrogens of the compounds described herein is replaced by a deuterium. When a position is designated as “H” or “hydrogen”, the position is understood to have hydrogen at its natural abundance isotopic composition. When a position is designated as “²H” or “deuterium”, the position is understood to have deuterium at an abundance that is at least 3340 times greater than the natural abundance of deuterium, which is 0.015% (i.e., the term “²H” or “deuterium” indicates at least 50.1% incorporation of deuterium). Accordingly, the invention also features compositions enriched in deuterated compounds.

In embodiments, compositions of any compound provided herein may have an isotopic enrichment factor for each deuterium present at a site designated as a potential site of deuteration on the compound of at least 3500 (52.5% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Synthetic Methods

Compounds described herein can be prepared according to methods known in the art. For example, the exemplary synthetic methods described in the instant Examples can be used to prepare still other compounds of the invention.

Accordingly, disclosed compounds can generally be synthesized by an appropriate combination of generally well known synthetic methods. Techniques useful in synthesizing these chemical entities are both readily apparent and accessible to those of skill in the relevant art, based on the instant disclosure. Many of the optionally substituted starting compounds and other reactants are commercially available, e.g., from Aldrich Chemical Company (Milwaukee, Wis.) or can be readily prepared by those skilled in the art using commonly employed synthetic methodology.

Exemplary synthetic schemes for preparing certain compounds according to the invention are provided in Schemes 1-3.

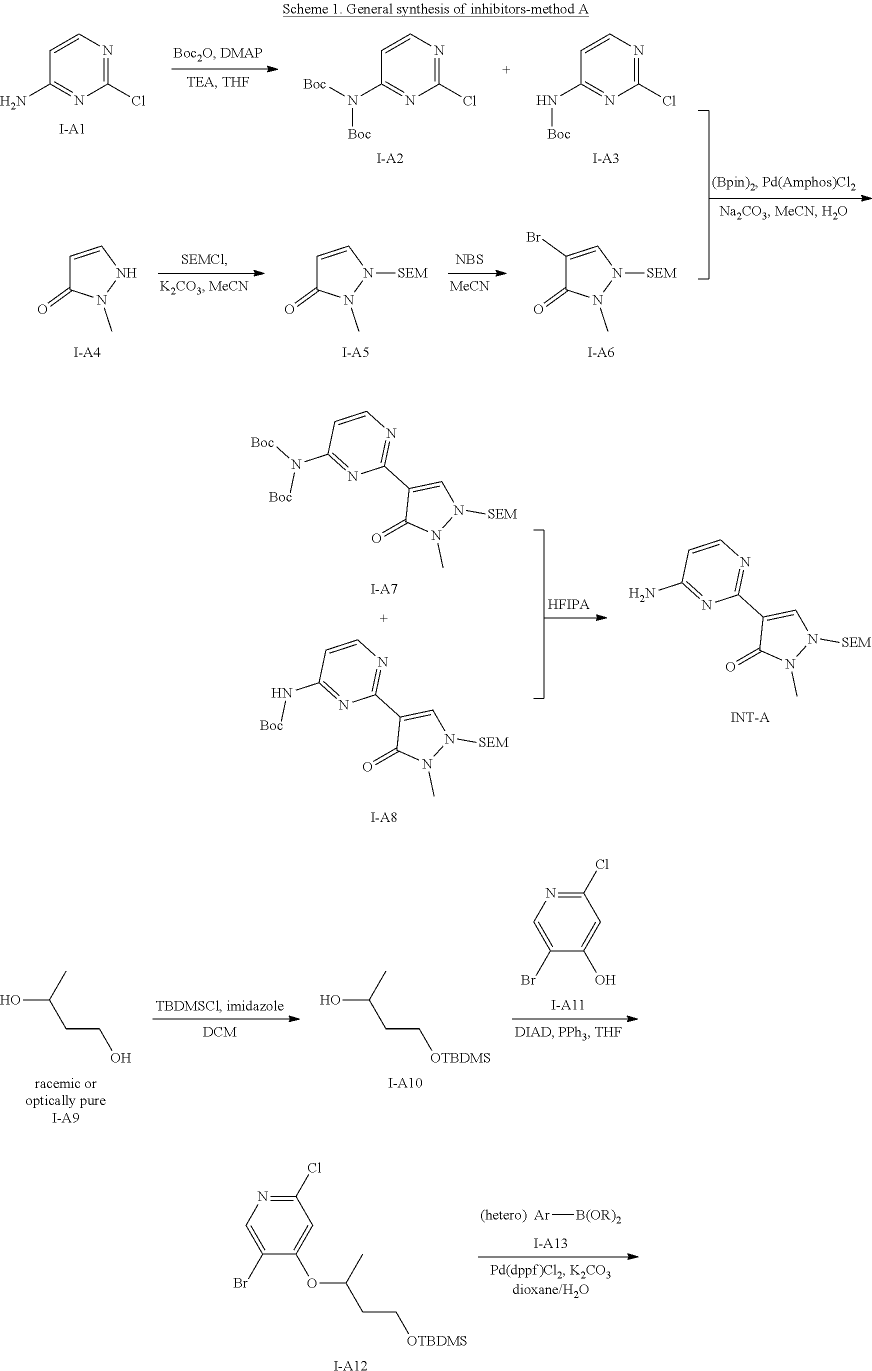

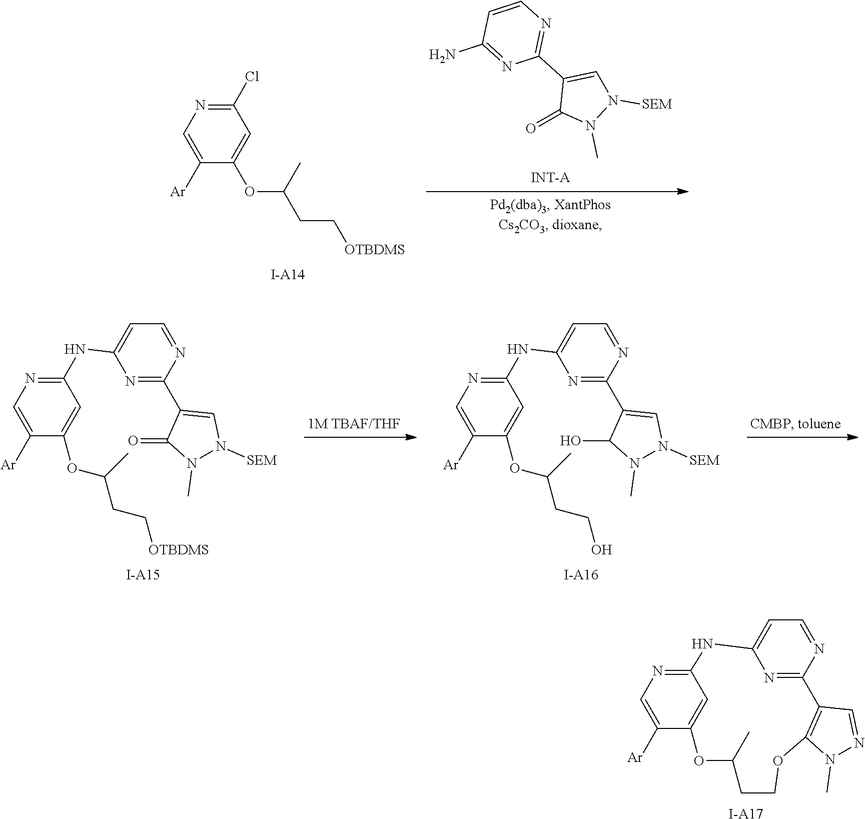

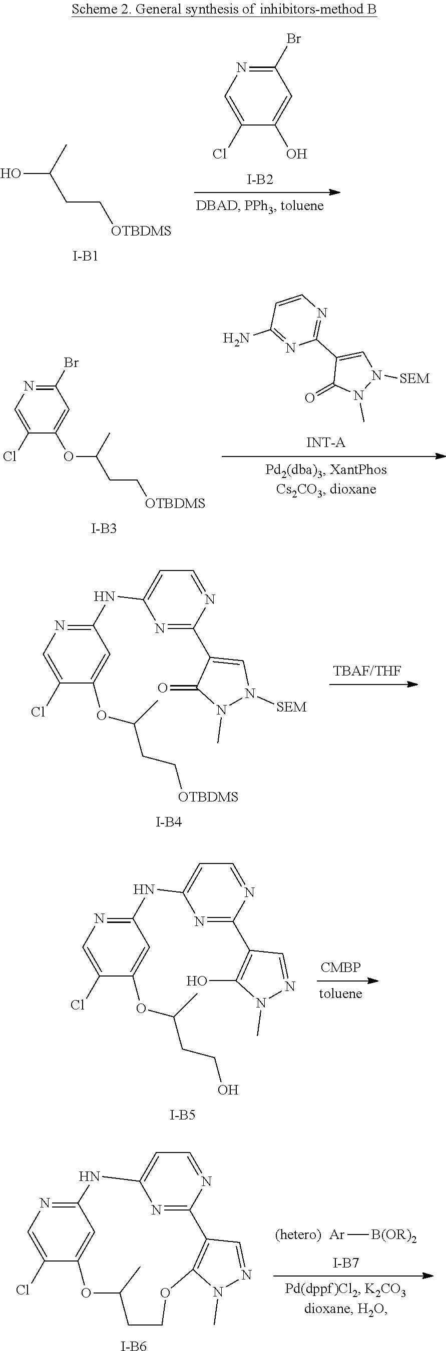

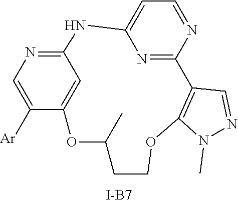

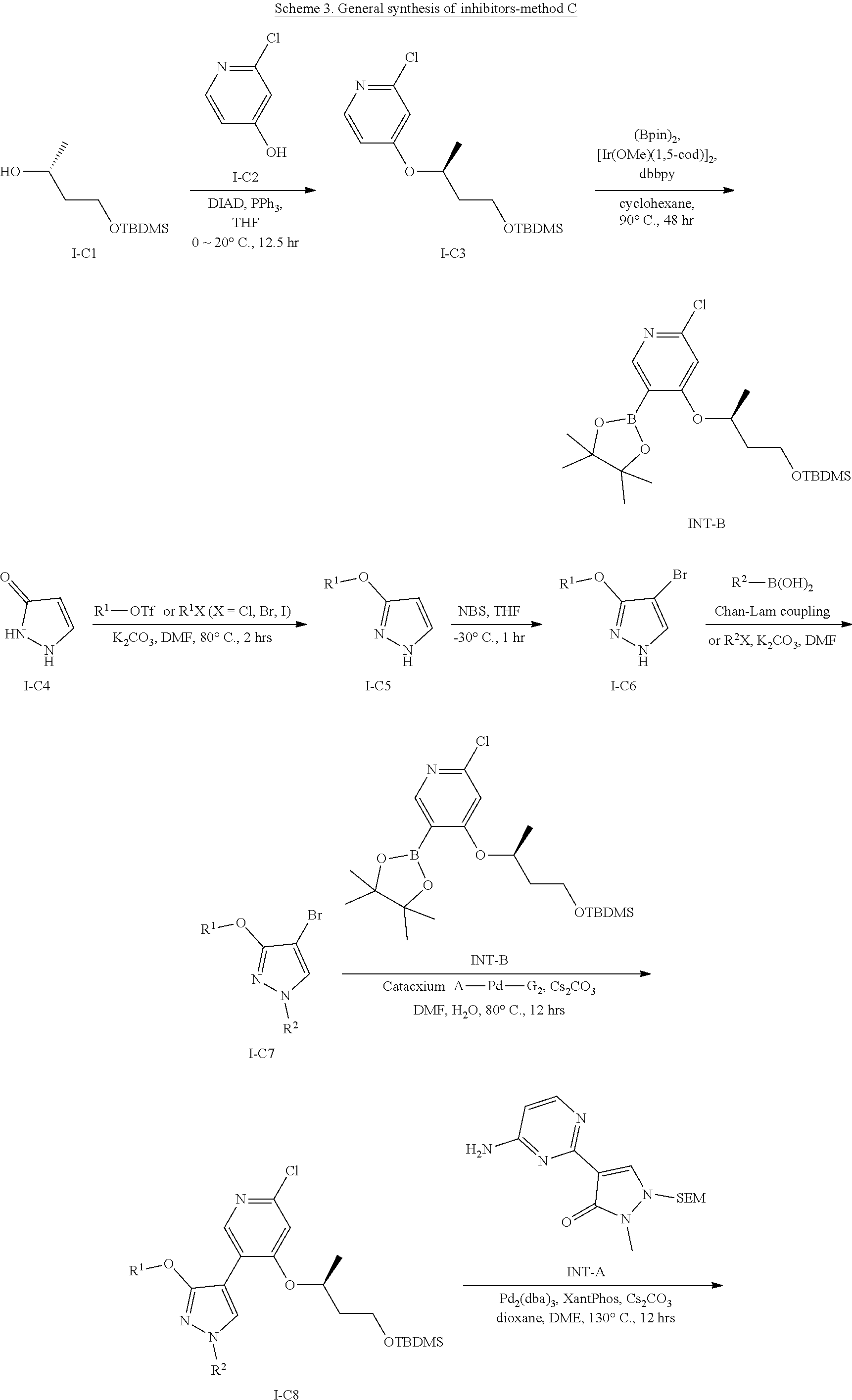

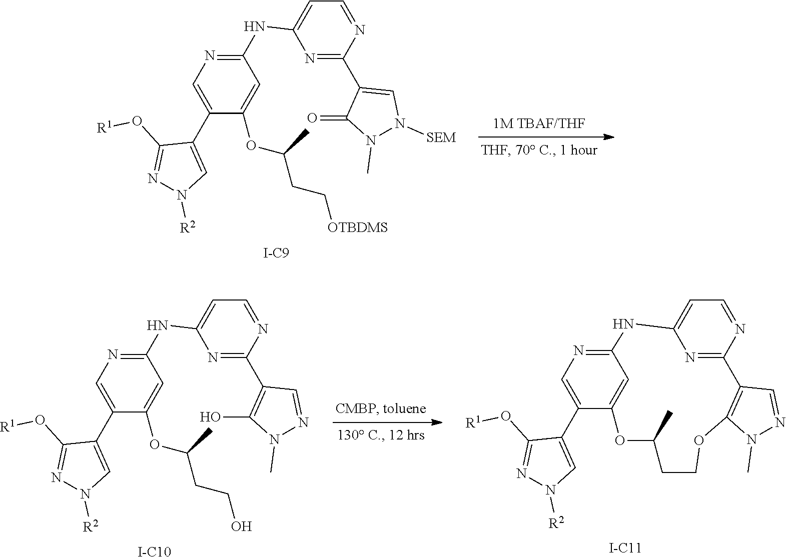

Table A herein summarizes MS characterization of exemplary compounds of Formula (I).

### Pharmaceutical Compositions

In another exemplary aspect, the invention features pharmaceutical compositions comprising any compound herein, or a pharmaceutically acceptable form thereof (e.g., any compound of Formulas (I)-(XXIII), such as any of Compounds (1)-(169), or a pharmaceutically acceptable salt thereof).

In embodiments, a pharmaceutical composition comprises a therapeutically effective amount of any compound described herein, or any pharmaceutically acceptable form thereof.

In embodiments, a pharmaceutically acceptable form of a compound includes any pharmaceutically acceptable salts, hydrates, solvates, isomers, prodrugs, and isotopically labeled derivatives thereof.

In embodiments, a pharmaceutical composition comprises any compound described herein, or a pharmaceutically acceptable salt thereof.

In embodiments, a pharmaceutical composition comprises a pharmaceutically acceptable excipient.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present invention have improved cellular potency, pharmacokinetic properties, as well as improved oral bioavailability.

Accordingly, in some embodiments, provided herein are pharmaceutical compositions comprising one or more compounds as disclosed herein, or a pharmaceutically acceptable form thereof (e.g., pharmaceutically acceptable salts, hydrates, solvates, isomers, prodrugs, and isotopically labeled derivatives), and one or more pharmaceutically acceptable excipients, carriers, including inert solid diluents and fillers, diluents, including sterile aqueous solution and various organic solvents, permeation enhancers, solubilizers and adjuvants. In some embodiments, a pharmaceutical composition described herein includes a second active agent such as an additional therapeutic agent, (e.g., a chemotherapeutic).

Accordingly, the present teachings also provide pharmaceutical compositions that include at least one compound described herein, or any pharmaceutically salt thereof thereof, and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, PA (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the composition and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known 5-hydroxytryptamine receptor 7 activity modulators. Pharmaceutical compositions in the form of oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations described herein herein can utilize standard delay or time-release formulations to alter the absorption of the compound(s). An oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

In embodiments, a pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.]

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

### Kits

In some embodiments, provided herein are kits. The kits can include a compound or pharmaceutically acceptable form thereof, or pharmaceutical composition as described herein, in suitable packaging, and written material that can include instructions for use, discussion of clinical studies, listing of side effects, and the like. Kits are well suited for the delivery of solid oral dosage forms such as tablets or capsules. Such kits can also include information, such as scientific literature references, package insert materials, clinical trial results, and/or summaries of these and the like, which indicate or establish the activities and/or advantages of the pharmaceutical composition, and/or which describe dosing, administration, side effects, drug interactions, or other information useful to the health care provider. Such information can be based on the results of various studies, for example, studies using experimental animals involving in vivo models and studies based on human clinical trials.

### Therapeutic Methods

Compounds of the present teachings (e.g., any compound of Formulas (I)-(XXIII), such as any of Compounds (1)-(169), or a pharmaceutically acceptable salt thereof) can be useful for the treatment or inhibition of a pathological condition or disorder in a mammal, for example, a human subject. The present teachings accordingly provide methods of treating or inhibiting a pathological condition or disorder by providing to a mammal a compound of the present teachings (including its pharmaceutically acceptable salt) or a pharmaceutical composition that includes one or more compounds of the present teachings in combination or association with pharmaceutically acceptable carriers. Compounds of the present teachings can be administered alone or in combination with other therapeutically effective compounds or therapies for the treatment or inhibition of the pathological condition or disorder.

Accordingly, compounds described herein can be particularly useful in treating diseases or disorders associated with defects in various components of signal transduction pathways and which are responsive to modulation (e.g., inhibition) of protein kinases. In embodiments, a compound described herein modulates (e.g., inhibitors) a protein kinase that is abl, Akt, ber-abl, Blk, Brk, c-kit, c-met, c-src, CDK1, CDK2, CDK3, CDK4, CDK5, CDK6, CDK7, CDK8, CDK9, CDK10, cRaf1, CSK, EGFR, ErbB2, ErbB3, ErbB4, Erk, Pak, fes, FGFR1, FGFR2, FGFR3, FGFR4, FGFR5, Fgr, flt-1, Fps, Frk, Fyn, Ick, IGF-1R, INS-R, Jak, KDR, Lck, Lyn, MEK, p38, PDGFR, PIK, PKC, PYK2, ros, tie, tie2, TRK or Zap70. In embodiments, a compound described herein modulates (e.g., inhibits) a wild-type form of a kinase (e.g., ECFR). In embodiments, a compound described herein modulates (e.g., inhibits) a mutant fonn of a kinase (e.g., EGFR).

In embodiments, a compound described herein, or any pharmaceutically acceptable form thereof such as a pharmaceutically acceptable salt thereof, modulates (e.g., inhibits) a kinase that is a tyrosine kinase (e.g., KIT, erb2, PDGFR, EGF R, VEGFR, src, or abl).

In embodiments, a compound described herein, or any pharmaceutically acceptable form thereof such as a pharmaceutically acceptable salt thereof, modulates (e.g., inhibits) a kinase that is a serine/threonine kinase (e.g., mTorC1, mTorC2, ATM, ATR, DNA-PK, or Akt).

In embodiments, a compound described herein, or any pharmaceutically acceptable form thereof such as a pharmaceutically acceptable salt thereof, can be used to treat or prevent a disease or disorder that is responsive to modulation (e.g., inhibition) of a protein kinase (e.g., abl, Akt, bcr-abl, Blk, Brk, c-kit, c-met, c-src, CDK1, CDK2, CDK3, CDK4, CDK5, CDK6, CDK7, CDK8, CDK9, CDK10, cRaf1, CSK, EGFR, ErbB2, ErbB3, Erb34, Erk, Pak, fes, FGFR1, FGFR2, FGFR3, FGFR4, FGFR5, Fgr, fit-1, Fps, Frk, Fyn, Hck, IGF-1R, INS-R, Jak, KDR, Lck, Lyn, MEK, p38, PDGFR, PIK, PKC, PYK2, ros, tie, tie2, TRK or Zap70).

In embodiments, a compound described herein, or any pharmaceutically acceptable form thereof such as a pharmaceutically acceptable salt thereof, can be used to treat or prevent a disease or disorder that is responsive to modulation (e.g., inhibition) of a tyrosine kinase (e.g., KIT, erb2, PDGFR, EGFR, VEGFR, src, or abl).

In embodiments, a compound described herein, or any pharmaceutically acceptable form thereof such as a pharmaceutically acceptable salt thereof, can be used to treat or prevent a disease or disorder that is responsive to modulation (e.g., inhibition) of a serine/threonine kinase (e.g., naTorC1, mTorC2, ATM, ATR, DNA-PK, or Akt).

In embodiments, a compound described herein modulates (e.g., inhibits) a wild-type form of a kinase (e.g., EGFR). In embodiments, a compound described herein modulates (e.g., inhibits) a mutant form of a kinase (e.g., EGFR).

### Selective Inhibition of Kinases

In some embodiments, a compound described herein, or any pharmaceutically acceptable salt thereof, inhibits a kinase or kinase form over other kinases or other kinase forms. Exemplary compounds include any compound of Formulas (I)-(XXIII), such as any of Compounds (1)-(169), or a pharmaceutically acceptable salt thereof.

The term “selective inhibition” or “selectively inhibit” as applied to a biologically active agent refers to the agent's ability to selectively reduce the target signaling activity as compared to off-target signaling activity, via direct or interact interaction with the target.

In some embodiments, a compound described herein, or any pharmaceutically acceptable salt thereof, selectively inhibits a kinase or kinase form over other kinases or other kinase forms. In embodiments, a compound selectively inhibits a mutant kinase form over the wild-type of the same kinase.

In embodiments, a compound described herein, or any pharmaceutically acceptable salt thereof, selectively inhibits a kinase (e.g., EGFR) over other kinases.

In embodiments, a compound described herein, or any pharmaceutically acceptable salt thereof, selectively inhibits a kinase form (e.g., mutant EGFR) over other kinase forms (e.g., wild-type EGFR).

By way of non-limiting example, the ratio of selectivity can be greater than a factor of about 10, greater than a factor of about 20, greater than a factor of about 30, greater than a factor of about 40, greater than a factor of about 50, greater than a factor of about 60, greater than a factor of about 70, greater than a factor of about 80, greater than a factor of about 100, greater than a factor of about 120, or greater than a factor of about 150, where selectivity can be measured by in vitro assays known in the art. Non-limiting examples of assays to measure selectivity include enzymatic assays, cellular proliferation assays, and EGFR phosphorylation assays. In one embodiment, selectivity can be determined by cellular proliferation assays. In another embodiment, selectivity can be determined by EGFR phosphorylation assays. In some embodiments, the mutant EGFR inhibitory activity of a compound as disclosed herein can be less than about 1000 nM, less than about 100 nM, less than about 50 nM, less than about 30 nM, or less than about 10 nM.

In embodiments, the IC₅₀ of a kinase inhibitor compound can be less than about 100 nM, less than about 50 nM, less than about 10 nM, less than about 1 nM, less than about 0.5 nM, or less than about 1 pM.

Determination of IC₅₀ values can be performed according to methods known in the art.

In embodiments, a compound described herein, or any pharmaceutically acceptable form thereof such as a pharmaceutically acceptable salt thereof, can be used to treat or prevent a disease or disorder that is cancer, an inflammatory disorder, a metabolic disorder, vascular disease, or neuronal disease.

Compounds described herein, or any pharmaceutically acceptable form thereof, or any pharmaceutical composition thereof, can be useful for treating diseases and disorders associated with abnormal cell proliferation.

In embodiments, a compound described herein, or a pharmaceutically acceptable form thereof (e.g., a pharmaceutically acceptable salt thereof), or a pharmaceutical composition thereof, can be used to treat cancer.

### Cancer

The compounds (e.g., any compound of Formulas (I)-(XXIII), such as any of Compounds (1)-(169), or a pharmaceutically acceptable salt thereof), as well as compositions thereof and methods provided herein can potentially be useful for the treatment of cancer including tumors such as astrocytic, breast, cervical, colorectal, endometrial, esophageal, gastric, head and neck, hepatocellular, laryngeal, lung, oral, ovarian, prostate and thyroid carcinomas and sarcomas.

In embodiments, a cancer is a cardiac cancer such as sarcoma (angiosarcoma, fibrosarcoma, rhabdomyosarcoma, liposarcoma), myxoma, rhabdomyoma, fibroma, lipoma or teratoma.

In embodiments, a cancer is a lung cancer such as bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, or mesothelioma.

In embodiments, a cancer is a gastrointestinal cancer such as: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma).

In embodiments, a cancer is a cancer of the genitourinary tract such as: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma).

In embodiments a cancer is a liver cancer such as hepatoma (hepatocellular carcinoma), cholangiocarcinoma, hepatoblastoma, angiosarcoma, hepatocellular adenoma, hemangioma.

In embodiments, a cancer is a bone cancer such as: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors.

In embodiments a cancer is a cancer of the central nervous system (CNS) such as: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma).

In embodiments, a cancer is a gynecological cancer such as: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma).

In embodiments, a cancer is a hematological cancer such as: blood (myeloid leukemia (acute and chronic), acute lymphoblastic leukemia, chronic lymphocytic leukemia, myeloproliferative diseases, multiple myeloma, myelodysplasia syndrome), Hodgkin's disease, non-Hodgkin's lymphoma (malignant lymphoma).

In embodiments, a cancer is a skin cancer such as: malignant melanoma, basal cell carcinoma, squamous cell carcinoma, Kaposi's sarcoma, moles dysplastic nevi, lipoma, angioma, dermatofibroma, keloids, psoriasis.

In embodiments, a cancer is a cancer of the adrenal glands such as neuroblastoma. Thus, the term “cancerous cell” as provided herein, includes a cell afflicted by any one of or related to the above identified conditions.

In embodiments, a cancer is an EGFR-driven cancer (e.g., as described herein). In embodiments, an EGF R-driven cancer is non-small cell lung cancer (NSCLC), squamous cell carcinoma, adenocarcinoma, adenocarcinoma, bronchioloalveolar carcinoma (BAC), BAC with focal invasion, adenocarcinoma with BAC features, and large cell carcinoma; neural tumors, such as glioblastomas; pancreatic cancer; head and neck cancers (e.g., squamous cell carcinoma); breast cancer; colorectal cancer; epithelial cancer, including squamous cell carcinoma; ovarian cancer; prostate cancer; or adenocarcinomas.

In embodiments, a cancer is an EGFR mutant cancer (e.g., as described herein). In embodiments, an EGFR mutant cancer is non-small cell lung cancer (NSCLC), squamous cell carcinoma, adenocarcinoma, adenocarcinoma, bronchioloalveolar carcinoma (BAC), BAC with focal invasion, adenocarcinoma with BAC features, and large cell carcinoma; neural tumors, such as glioblastomas; pancreatic cancer; head and neck cancers (e.g., squamous cell carcinoma); breast cancer; colorectal cancer; epithelial cancer, including squamous cell carcinoma; ovarian cancer; prostate cancer; or adenocarcinomas.

In one embodiment, the compositions and methods provided herein are useful for the treatment of lung cancer and pancreatic cancer, most specifically, non-small cell lung cancer (NSCLC).

In embodiments, a cancer is refractory to TKI therapies (e.g., erlotinib, gefitinib, dacomitinib, afatinib, osimertinib).

### Lung Cancer

In embodiments, a cancer is a lung cancer.

Lung cancer is the most common cause of cancer mortality globally and the second most common cancer in both men and women. About 14% of all new cancers are lung cancers. In the United States (US), there are projected to be 222,500 new cases of lung cancer (116,990 in men and 105,510 in women) and 155,870 deaths from lung cancer (84,590 in men and 71,280 in women) in 2017.

The two major forms of lung cancer are non-small cell lung cancer (NSCLC) and small cell lung cancer. NSCLC is a heterogeneous disease that consists of adenocarcinoma, large-cell carcinoma, and squamous cell carcinoma (sqNSCLC), and comprises approximately 80% to 85% of all lung cancers. Squamous cell carcinoma of the lung accounts for 20% to 30% of NSCLC. Despite advances in early detection and standard treatment, NSCLC is often diagnosed at an advanced stage, has poor prognosis, and is the leading cause of cancer deaths worldwide.

Platinum-based doublet therapy, maintenance chemotherapy, and anti-angiogenic agents in combination with chemotherapy have contributed to improved patient outcomes in advanced NSCLC.

In embodiments, an advanced lung cancer is stage III cancer or stage IV cancer. In embodiments, an advanced lung cancer is stage III cancer. In embodiments, an advanced lung cancer is stage IV cancer. In embodiments, an advanced lung cancer is locally advanced. In embodiments, an advanced lung cancer is metastatic.

In embodiments, a lung cancer is small cell lung cancer (SCLC).

In embodiments, a lung cancer is non-small cell lung cancer (NSCLC) such as adenocarcinoma, large-cell carcinoma, or squamous cell carcinoma (sqNSCLC). In embodiments, a NSCLC is lung adenocarcinoma. In embodiments, a NSCLC is large cell carcinoma of the lung. In embodiments, a NSCLC is squamous cell carcinoma of the lung (sqNSCLC).

In embodiments, a lung cancer (e.g., NSCLC) is an EGFR-mutant lung cancer (e.g., EGFR-mutant NSCLC). In embodiments, a cancer is NSCLC (e.g., advanced NSCLC) with an identified EGFR mutation.

### EGFR Driven and EGFR Mutant Cancers

The invention features compounds (e.g., any compound of Formulas (I)-(XXIII), such as any of Compounds (1)-(169), or a pharmaceutically acceptable salt thereof) which can be useful for treating patients who have an EGFR-driven cancer, including cancers which are, or have become, refractory to erlotinib, gefitinib, dacomitinib, afatinib, osimertinib, or cancers which bear an EGFR mutation identified herein, by administering a compound of formula (I) to a subject.

That is, compounds described herein can be effective inhibitors of mutant forms of EGFR, such as single, double, or mutant EGFR having L858R (“L”), T790M (“T”), C797S (“C”), and/or Exon19 (Del19 or “D”) mutations, or any combination thereof. Such inhibitors can be particularly beneficial in therapy of patients who have developed mutations after receiving certain other cancer therapies. For example, a patient may present with single mutants (D, L) but after certain treatments, a patient may develop secondary and even (e.g., after osimertinib treatment) tertiary mutations. Accordingly, new inhibitors that have activity against cancers characterized by single, double, and/or triple mutant EGFR can confer great benefit to cancer patients, including those who have developed resistance to previous therapies.

EGFR-driven cancers which can be treated using the compositions and method of the invention include, for example, EGFR mutants including one or more deletions, substitutions, or additions in the amino acid or nucleotide sequences of ECFR, or fragments thereof.

An EGFR-driven cancer may result from an EGFR fusion. For example, the N-terminal of EGFR can be linked to various fusion partners such as RAD51. Cancers (e.g., lung cancers) characterized by an EGFR-fusion (e.g., an EGFR-RAD51 fusion) may be particularly suitable for therapy using any compound described herein, or any pharmaceutically acceptable form (e.g., a pharmaceutically acceptable salt) thereof.

Mutations in EGFR can occur in any part of the EGFR sequence. Generally, EGFR mutants arise from mutations in the kinase domain (i.e., exons 18-24 in the EGFR sequence) or in the extracellular domain (i.e., exons 2-16 in the EGFR sequence).

A mutation in EGFR can be an activating mutation, which lead to a ligand-independent activation of TK activity. A mutation in EGFR can also be a resistance mutation, which can confer resistance to TKI therapies such as resistance to one or more of erlotinib, gefitinib, dacomitinib, afatinib, or osimertinib.

For example, mutations typically occur in the kinase domain, including one or more of a point mutation in exon 18 (e.g., L688P, V689M, P694L/S, N700D, L703V, E709K/Q/A/G/V, 1715S, L718P, G719C/A/S/R, or S720P/F), a deletion in exon 19 that may or may not include an insertion (e.g., delG719, delE746_E749, delE746_A750, delE746_A750insRP, delE746_A750insQP, de1E746_T751, delE746_T751insA/I/V, delE746_T751insVA, delE746_S752, delE746_S752insA/V/D, delE746_P53insLS, delL747_E749, delL747_A750, delL747_A750insP, delL747 T751, delL747_T751insP/S/Q, delL747_T751insPI, delL747_5752, delL747_S752insQ, delL747_P753, delL747_P753insS/Q, delL747_L754insSR, delE749_A750, delE749_A750insRP, delE749_T751, delT751_1759, delT751_I759insS/N, or delS752_1759), a duplication in exon 19 (e.g., K739_I44dupKIPVAI), a point mutation in exon 19 (e.g., L730F, W731Stop, P733L, G7355, V742A, E746V/K, A750P, T7511, S752Y, P753S, A754P, or D761Y), an in-frame insertion in exon 20 (e.g., 1)₇₆₁_E762ins EAFQ, A767_S768insTLA, V769_D770insY, V7691_D770insCV, V769 D770insASV, D770_N771insD/G, D770_N771insNPG, D770_N771insSVQ, P772_H773insN/V, P772_H773insYNP, or V774_C775insHV), a deletion in exon 20 that may or may not include an insertion (e.g., delM766_A767, delM766_A767insAI, delA767_V769, delD770, or delP772_H773insNP), a duplication in exon 20 (e.g., S768_D770dupSVD, A767_V769dupASV, or H773dupH), a point mutation in exon 20 (e.g., D761N, A763V, V765A/M, S7681, V769L/M, S768I, P772R, N771T, H773R/Y/L, V774M, R776G/H/C, G779S/F, T783A, T784F, L792P, L798H/F, T790M, R803W, K806E, or L814P), or a point mutation in exon 21 (e.g., G810S, N826S, L833V, H835L, L838V, A839T, 1K846R, T8471, H850N, V851W/A, 1853T, L858M/R, A859T, L861Q/R, G863D, A864T, E866K, or G873E).

In lung cancer, activation mutants are typical.

In embodiments, a mutation is a resistance mutation. In particular, drug resistance in 50% of lung cancers arises from the T790M point mutation. Other exemplary resistance mutation include point mutations such as: C797X (e.g., C797S, C797G, or C797N); G796X (e.g., G796R, G796S, or G3796D); L792X (e.g. L792H, L792F, L792R, or L792Y); G724S; L718X (e.g., L718P, L718Q, or L718V); S768I; or G719A.

In glioblastoma, mutations typically, but not exclusively, occur in the extracellular domain, including EGFR variant I (EGFRvI) lacking the extracellular domain and resembling the v-erbB oncoprotein; EGFRvII lacking 83 amino acids from domain IV; and EGFRvIII lacking amino acids 30-297 from domains I and II, which is the most common amplification and is reported in 30-50% of glioblastomas and 5% of squamous cell carcinoma. Other mutations for glioblastoma include one or more of point mutations in exon 2 (e.g., D46N or G63R), exon 3 (e.g., R108K in domain I), exon 7 (e.g., T263P or A289D/T/V in domain II), exon 8 (e.g., R324L or E330K), exon 15 (e.g., P596L or G598V in domain IV), or exon 21 (L861Q in the kinase domain).

EGFR mutants also include those with a combination of two or more mutations, as described herein. Exemplary combinations include S7681 and G719A; S7681 and V769L; H773R and W731Stop; R776G and L858R; R776H and L861Q; T790M and L858R; T790M and delE746_A750; R803W and delE746_T751insVA; delL747_E49 and A750P; delL747_S752 and E746V; delL747_S752 and P753S; P772_H773insYNP and H773Y; P772_H773insNP and H773Y; and D770_N771insG and N771T. Other exemplary combinations include any including T790M (e.g., T790M and L858R or T790M and delE746_A750.

EGFR mutants can be either activation mutants or resistant mutants. Activation mutants include those with substitutions that increase drug sensitivity (e.g., G719C/S/A, delE746 A750, or L858R). Resistant mutants include those with substitutions that increase drug resistance (e.g., T790M or any combination including T790M).

In embodiments, an EGFR mutation is a deletion in exon19 (del19). In embodiments, an EGFR mutation is a T790M mutation. In embodiments, an EGFR mutation is a L858R mutation. In embodiments, an EGFR mutation is a C797S mutation. In embodiments, an EGFR-driven cancer (e.g., non-small cell lung cancer) is characterized by at least one of these mutations. In embodiments, an EGFR-driven cancer (e.g., non-small cell lung cancer) is characterized by at least two of these mutations. In embodiments, an EGFR-driven cancer (e.g., non-small cell lung cancer) is characterized by at least three of these mutations.

EGFR-driven cancers include those having any mutant described herein. For example, EGFRvIII is commonly found in glioblastoma and has also been reported in breast, ovarian, prostate, and lung carcinomas. Exemplary EGFR-driven cancers: glioblastoma, lung cancer (e.g., squamous cell carcinoma, non-small cell lung cancer, adenocarcinoma, bronchioloalveolar carcinoma (BAC), BAC with focal invasion, adenocarcinoma with BAC features, and large cell carcinoma), pancreatic cancer, head and neck cancers (e.g., squamous cell carcinoma), breast cancer, colorectal cancer, epithelial cancer (e.g., squamous cell carcinoma), ovarian cancer, and prostate cancer.

In particular, the invention described herein would benefit patient populations having higher risk for TKI-resistant mutations. About 8,000 to 16,000 new cases per year can be estimated based on: incidence of non-small cell lung cancer (about 160,000 new cases in the U.S.), the response to erlotinib in the general population (about 10%, resulting in a sensitive population of 16,000), the presence of activation mutations (10-20% in white and 30-40% in Asian population, resulting in a sensitive population of 16,000-32,000), acquisition of secondary resistance (most if not all patients, resulting in a sensitive population of 16,000-32,000), and percentage of patients carrying the T790M point mutations (about 50%, resulting in a sensitive population of 8,000-16,000). Patients having TI-resistant mutations include those patients having cancers resistant to one or more of erlotinib, gefitinib, dacomitinib, afatinib, osimertinib, CL-387,785, 13113W 2992 (CAS Reg. No. 439081-18-2), CI-1033, neratinib (HKI-272), MP-412 (AV-412), PF-299804, AEE78, and XL64.

In particular, the inventions relate to treatment of EGFR-driven cancers having the T790M point mutation. Generally, irreversible inhibitors (e.g., CI-1033, neratinib (HKI-272), and PF-299804) are less potent in cell lines having the T790M mutation and do not inhibit T790M at clinically achievable concentrations. Since the ATP Km of T790M and WT are similar, concentrations that inhibit the mutant will inhibit the WT and result in gastrointestinal and cutaneous events.

An EGFR mutant also includes other amino acid and nucleotide sequences of EGFR with one or more deletions, substitutions, or additions, such as point mutations, that retain or increase tyrosine kinase or phosphorylation activity. Where the mutant is a protein or polypeptide, preferable substitutions are conservative substitutions, which are substitutions between amino acids similar in properties such as structural, electric, polar, or hydrophobic properties. For example, the substitution can be conducted between basic amino acids (e.g., Lys, Arg, and His), or between acidic amino acids (e.g., Asp and Glu), or between amino acids having non-charged polar side chains (e.g., Gly, Asn, Gin, Ser, Thr, Tyr, and Cys), or between amino acids having hydrophobic side chains (e.g., Ala, Val, Leu, Ile, Pro, Phe, and Met), or between amino acids having branched side chains (e.g., Thr, Val, Leu, and Ile), or between amino acids having aromatic side chains (e.g., Tyr, Trp, Phe, and His).

Where the mutant is a nucleic acid, the DNA encoding an EGFR mutant protein may comprise a nucleotide sequence capable of hybridizing to a complement sequence of the nucleotide sequence encoding an EGFR mutant, as defined herein, under stringent conditions. As used herein, the stringent conditions include low, medium or high stringent conditions. An example of the stringent conditions includes hybridization at approximately 42-55° C. in approximately 2-6×SSC, followed by wash at approximately 50-65° C. in approximately 0.1-1×SSC containing approximately 0.1-0.2% SDS, where 1×SSC is a solution containing 0.15 M NaCl and 0.015 M Na citrate, pH 7.0. Wash can be performed once or more. In general, stringent conditions may be set at a temperature approximately 5° C. lower than a melting temperature (Tm) of a specific nucleotide sequence at defined ionic strength and pH.

The amino acid and nucleotide sequences of EGFR and DNAs encoding them are available from known databases such as NCBI GenBank (USA), EMBL (Europe), etc. For example, GenBank accession numbers for EGFR [Homo sapiens] include MIM131550, AA128420, NM_005228, NP_005219.2, and GeneID: 1956.

### EGFR-Selective Inhibition

In some embodiments, a compound described herein (e.g., any compound of Formulas (I)-(XXIII), such as any of Compounds (1)-(169)), or any pharmaceutically acceptable salt thereof, selectively inhibits EGFR (including any mutant EGF R described herein) over other kinases.

In some embodiments, a compound described herein, or any pharmaceutically acceptable salt thereof, selectively inhibits mutant EGFR (e.g., any mutant EGFR described herein) over wild-type EGFR. In embodiments, a compound described herein selectively inhibits EGFR characterized by a mutation that is: a deletion in exon19 (del19), a T790M mutation, a L858R mutation, and/or a C797S mutation, or any combination thereof. Such inhibitors can be effective in ameliorating diseases and disorders associated with mutant EGFR activity.

By way of non-limiting example, the ratio of selectivity can be greater than a factor of about 10, greater than a factor of about 20, greater than a factor of about 30, greater than a factor of about 40, greater than a factor of about 50, greater than a factor of about 60, greater than a factor of about 70, greater than a factor of about 80, greater than a factor of about 100, greater than a factor of about 120, or greater than a factor of about 150, where selectivity can be measured by in vitro assays known in the art. Non-limiting examples of assays to measure selectivity include enzymatic assays, cellular proliferation assays, and EGFR phosphorylation assays. In one embodiment, selectivity can be determined by cellular proliferation assays. In another embodiment, selectivity can be determined by EGF R phosphorylation assays. In some embodiments, the mutant EGFR inhibitory activity of a compound as disclosed herein can be less than about 1000 nM, less than about 100 nM, less than about 50 nM, less than about 30 nM, or less than about 10 nM.

In embodiments, the IC₅₀ of a subject compound for mutant EGFR inhibition can be less than about 100 nM, less than about 50 nM, less than about 10 nM, less than about 1 nM, less than about 0.5 nM, or less than about 1 pM.

### Characterization of EGFR-Driven Cancers

The compositions and methods of the invention can be used to treat subjects having an EGFR-driven cancer (i.e., cancers characterized by EGFR mutant expression or overexpression). EGFR mutant expression or overexpression can be determined in a diagnostic or prognostic assay by evaluating levels of EGFR mutants in biological sample, or secreted by the cell (e.g., via an immunohistochemistry assay using anti-EGFR antibodies or anti-p-EGFR antibodies; FACS analysis, etc.). Alternatively, or additionally, one can measure levels of EGFR mutant-encoding nucleic acid or mRNA in the cell, e.g., via fluorescent in situ hybridization using a nucleic acid based probe corresponding to an EGFR mutant-encoding nucleic acid or the complement thereof; (FISH; see WO98/45479, published October, 1998), Southern blotting, Northern blotting, or polymerase chain reaction (PCR) techniques, such as real time quantitative PCR (RT-PCR). One can also study EGFR mutant expression by measuring shed antigen in a biological sample, such as serum, e.g., using antibody-based assays (see also, e.g., U.S. Pat. No. 4,933,294, issued Jun. 12, 1990; WO91/05264, published Apr. 18, 1991; U.S. Pat. No. 5,401,638,issued Mar. 28, 1995; and Sias et al., J. Immunol. Methods 132:73 (1990)). Aside from the above assays, various in vivo assays are available to the skilled practitioner. For example, one can expose cells within the body of the mammal to an antibody which is optionally labeled with a detectable label, e.g., a radioactive isotope, and binding of the antibody to cells in the mammal can be evaluated, e.g., by external scanning for radioactivity or by analyzing a biopsy taken from a mammal previously exposed to the antibody.

Examples of biological properties that can be measured in isolated cells include mRNA expression, protein expression, and DNA quantification. Additionally, the DNA of cells isolated by the methods of the invention can be sequenced, or certain sequence characteristics (e.g., polymorphisms and chromosomal abnormalities) can be identified using standard techniques, e.g., FISH or PCR. The chemical components of cells, and other analytes, may also be assayed after isolation. Cells may also be assayed without lysis, e.g., using extracellular or intracellular stains or by other observation, e.g., morphology or growth characteristics in various media.

While any hybridization technique can be used to detect the gene rearrangements, one preferred technique is fluorescent in situ hybridization (FISH). FISH is a cytogenetic technique which can be used to detect and localize the presence or absence of specific DNA or RNA sequences on chromosomes. FISH incorporates the use of fluorescently labeled nucleic acid probes which bind only to those parts of the chromosome with which they show a high degree of sequence similarity. Fluorescence microscopy can be used to find out where the fluorescent probe bound to the chromosome. The basic steps of FISH are outlined below. Exemplary FISH probes include Vysis EGFR SpectrumOrange/CEP SpectrumGreen Probe (Abbott, Downers Grove, IL), which hybridizes to band 7p12; and ZytoLight SPEC EGFR/CEN 7 Dual Color Probe (ZytoVision), which hybridizes to the alpha-satellite sequences of the centromere of chromosome 7.

For FISH, a probe is constructed that is long enough to hybridize specifically to its target (and not to similar sequences in the genome), but not too large to impede the hybridization process. Probes are generally labeled with fluorophores, with targets for antibodies, with biotin, or any combination thereof. This can be done in various ways, for example using random priming, nick translation, and PC R using tagged nucleotides.

Generally, a sample or aliquot of a population of cells is used for FISH analysis. For example, in one method of preparation, cells are trypsinized to disperse into single cells, cytospun onto glass slides, and then fixed with paraformaldehyde before storing in 70% ethanol. For preparation of the chromosomes for FISH, the chromosomes are firmly attached to a substrate, usually glass. After preparation, the probe is applied to the chromosome RNA and starts to hybridize. In several wash steps, all unhybridized or partially hybridized probes are washed away. If signal amplification is necessary to exceed the detection threshold of the microscope (which depends on many factors such as probe labeling efficiency, the kind of probe, and the fluorescent dye), fluorescent tagged antibodies or strepavidin are bound to the tag molecules, thus amplifying the fluorescence.

An epifluorescence microscope can be used for observation of the hybridized sequences. The white light of the source lamp is filtered so that only the relevant wavelengths for excitation of the fluorescent molecules arrive onto the sample. Emission of the fluorochromes happens, in general, at larger wavelengths, which allows one to distinguish between excitation and emission light by mean of another optical filter. With a more sophisticated filter set, it is possible to distinguish between several excitation and emission bands, and thus between several fluorochromes, which allows observation of many different probes on the same strand.

Depending on the probes used, FISH can have resolution ranging from huge chromosomes or tiny (˜100 kilobase) sequences. The probes can be quantified simply by counting dots or comparing color.

Allele-specific quantitative real time-PCR may also be used to identify a nucleic acid encoding a mutant EGFR protein (see, for e.g., Diagnostic Innovations DxS BCR-ABL T3151 Mutation Test Kit, and Singer et al., Methods in Molec. Biol. 181:145 (2001)). This technique utilizes Taq DNA polymerase, which is extremely effective at distinguishing between a match and a mismatch at the 3′-end of the primer (when the 3′-base is mismatched, no efficient amplification occurs). Using this technique, the 3′-end of the primer may be designed to specifically hybridize to a nucleic acid sequence that corresponds to a codon that encodes a mutant amino acid in an EGFR mutant, as described herein. In this way, the specific mutated sequences can be selectively amplified in a patient sample. This technique further utilizes a Scorpion probe molecule, which is a bifunctional molecule containing a PCR primer, a fluorophore, and a quencher. The fluorophore in the probe interacts with a quencher, which reduces fluorescence. During a PCR reaction, when the Scorpion probe binds to the amplicon, the fluorophore and quencher in the Scorpion probe become separated, which leads to an increase in fluorescence from the reaction tube. Any of the primers described herein may be used in allele-specific quantitative real time PCR.

A biological sample can be analyzed to detect a mutation in an EGFR gene, or expression levels of an EGFR gene, by methods that are known in the art. For example, methods such as direct nucleic acid sequencing, altered hybridization, aberrant electrophoretic gel migration, binding or cleavage mediated by mismatch binding proteins, single-strand conformational polymorphism (SSCP) analysis, or restriction fragment length polymorphism (RFLP) analysis of PCI products derived from a patient sample can be used to detect a mutation in an EGFR gene; ELISA can be used to measure levels of EGFR polypeptide; and PCR can be used to measure the level of an EGFR nucleic acid molecule.

Any of these techniques may be used to facilitate detection of a mutation in a candidate gene, and each is well known in the art; examples of particular techniques are described, without limitation, in Orita et al. (Proc. Natl. Acad. Sci. USA 86:2766 (1989)) and Sheffield et al. (Proc. Natl. Acad. Sci. USA 86:232 (1989)). Furthermore, expression of the candidate gene in a biological sample (e.g., a biopsy) may be monitored by standard Northern blot analysis or may be aided by PCR (see, e.g., Ausubel et al., Current Protocols in Molecular Biology, John Wiley & Sons, New York, NY (1995); PCR Technology: Principles and Applications for DNA Amplification, H. A. Ehrlich, Ed., Stockton Press, NY; Yap et al., Nucl. Acids. Res. 19:4294 (1991)).

One skilled in the art may identify in a nucleic acid or protein sequence a residue (e.g., amino acid or nucleotide) or codon that corresponds to a residue or codon in wild-type EGFR or EGFR mutants using a number of sequence alignment software programs (e.g., NCBI BLAST website). Such software programs may allow for gaps in the alignment of the compared sequences. Using such software, one skilled in the art may identify a nucleotide, amino acid, or amino acid that corresponding to a specific nucleotide, amino acid, or codon in wild-type EGFR or EGFR mutants.

Levels of EGFR expression (e.g., DNA, mRNA, or protein) in a biological sample can be determined by using any of a number of standard techniques that are well known in the art or described herein. Exemplary biological samples include plasma, blood, sputum, pleural effusion, bronchoalveolar lavage, or biopsy, such as a lung biopsy and lymph node biopsy. For example, EGFR expression in a biological sample (e.g., a blood or tissue sample) from a patient can be monitored by standard northern blot analysis or by quantitative PCR (see, e.g., Ausubel et al., supra; PCR Technology: Principles and Applications for DNA Amplification, H. A. Ehrlich, Ed., Stockton Press, NY; Yap et al., Nucl. Acids. Res. 19:4294 (1991)).

### Combination Therapies

In some embodiments, provided herein are methods for combination therapies in which an agent known to modulate other pathways, or other components of the same pathway, or even overlapping sets of target enzymes are used in combination with a compound as provided herein (e.g., any compound of Formulas (I)-(XXIII), such as any of Compounds (1)-(169)), or a pharmaceutically acceptable form thereof (e.g., pharmaceutically acceptable salts, hydrates, solvates, isomers, prodrugs, and isotopically labeled derivatives) thereof. In one aspect, such therapy includes, but is not limited to, the combination of the subject compound with chemotherapeutic agents, therapeutic antibodies, and radiation treatment, to provide a synergistic or additive therapeutic effect.

When administered as a combination, the therapeutic agents can be formulated as separate compositions that are administered at the same time or sequentially at different times, or the therapeutic agents can be given as a single composition. The phrase “combination therapy” in referring to the use of a disclosed compound together with another pharmaceutical agent, means the coadministration of each agent in a substantially simultaneous manner as well as the administration of each agent in a sequential manner, in either case, in a regimen that will provide beneficial effects of the drug combination. Coadministration includes, inter alia, the simultaneous delivery, e.g., in a single tablet, capsule, injection or other dosage form having a fixed ratio of these active agents, as well as the simultaneous delivery in multiple, separate dosage forms for each agent respectively. Thus, the administration of disclosed compounds can be in conjunction with additional therapies known to those skilled in the art in the prevention or treatment of cancer, such as radiation therapy or cytostatic agents, cytotoxic agents, other anti-cancer agents and other drugs to ameliorate symptoms of the cancer or side effects of any of the drugs.

In some embodiments, treatment can be provided in combination with one or more other cancer therapies, include surgery, radiotherapy (e.g., gamma-radiation, neutron beam radiotherapy, electron beam radiotherapy, proton therapy, brachytherapy, and systemic radioactive isotopes, etc.), endocrine therapy, biologic response modifiers (e.g., interferons, interleukins, and tumor necrosis factor (TNF)), hyperthermia, cryotherapy, agents to attenuate any adverse effects (e.g., antiemetics), and other cancer chemotherapeutic drugs. The other agent(s) can be administered using a formulation, route of administration and dosing schedule the same or different from that used with the compounds provided herein.

In embodiments, combination therapy comprises administration of a compound described herein, or any pharmaceutically acceptable form thereof (e.g., any pharmaceutically acceptable salt thereof), or a pharmaceutical composition thereof, in combination with anti-cancer drugs (e.g., antiproliferative agents, anti-angiogenic agents and other chemotherapeutic agents).

In embodiments, combination therapy comprises administration of a compound described herein, or any pharmaceutically acceptable form thereof (e.g., any pharmaceutically acceptable salt thereof), or a pharmaceutical composition thereof, in combination with an amount of an anti-cancer agent (e.g., a chemotherapeutic agent).

### EXAMPLES

### Example 1: Preparation of Compound (169)

### Synthesis of inhibitors: 5,10-dimethyl-13-[4-[(4-methylpiperazin-1-yl)methyl]phenyl]-7,11-dioxa-4,5,15,17,21,22-hexazatetracyclo[16.3.1.112,16.02,6]tricosa-1(22),2(6),3,12(23),13,15,18,20-octaene (compound (169))

### (I) Synthesis of Intermediate A (INT-A)

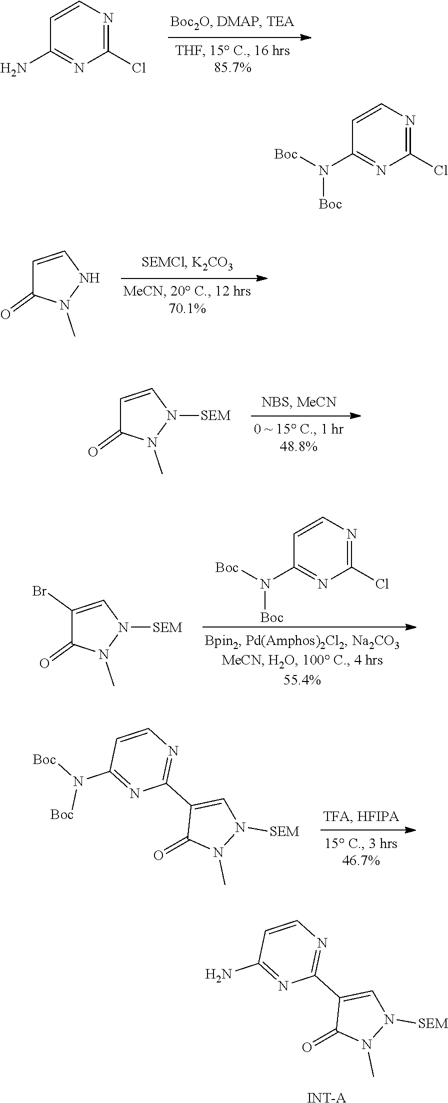

Step 1: a mixture of 2-chloropyrimidin-4-amine (2 g, 15.4 mmol, 1 eq) and DMAP (188.6 mg, 1.54 mmol, 0.1 eq) in THF (20 mL) was degassed and purged with nitrogen for 3 times, and TEA (6.25 g, 61.8 mmol, 4 eq) and Boc₂O (10.11 g, 46.3 mmol, 3 eq) were added. The mixture was stirred at 15° C. for 16 hours under nitrogen atmosphere. The reaction mixture was diluted with H₂O (50 mL) and extracted with EtOAc (50 mL*2). The combined organic layers were washed with brine (50 mL), dried over anhydrous Na₂SO₄, filtered and concentrated under reduced pressure. The residue was purified by flash silica gel chromatography (ISCO®; 12 g SepaFlash® Silica Flash Column, petroleum ether/EtOAc with EtOAc from 0˜20%, flow rate=20 mL/min) to afford tert-butyl N-tert-butoxycarbonyl-N-(2-chloropyrimidin-4-yl)carbamate (4.5 g, 85.7% yield, 97% purity) as an off white solid.

¹H NMR (400 MHz, DMSO) δ 8.72 (d, J=6.0 Hz, 1H), 7.73 (d, J=6.0 Hz, 1H), 1.52 (s, 18H).

Step 2: to a solution of 2-methylpyrazol-3-ol (3 g, 30.6 mmol, 1 eq) in MeCN (20 mL) were added SEMCl (II mL, 62.2 mmol, 2.03 eq) and K₂CO₃ (18.0 g, 0.130 mol, 4.26 eq). The mixture was stirred at 20° C. for 12 hours. The reaction mixture was filtered and concentrated under reduced pressure. The residue was purified by flash silica gel chromatography (ISCO®; 40 g SepaFlash® Silica Flash Column, DCM/MeOH with MeOH from 0˜8%, flow rate=40 mL/min) to afford 2-methyl-1-(2-trimethylsilylethoxymethyl)pyrazol-3-one (6.2 g, 70.1% yield, 79% purity) as a white solid.

¹H NMR (400 MHz, chloroform-d) δ 7.30 (d, J=3.6 Hz, 1H), 5.49 (d, J=3.6 Hz, 1H), 4.98 (s, 2H), 3.43-3.47 (m, 5H), 0.87 (t, J=8.0 Hz, 2H), −0.02 (s, 9H).

Step 3: to a mixture of 2-methyl-1-(2-trimethylsilylethoxymethyl)pyrazol-3-one (1.7 g, 7.44 mmol, 1 eq) in MeCN (20 mL) was added NBS (1.99 g, 11.2 mmol, 1.5 eq) at 0° C. tinder nitrogen and the mixture was stirred at 15° C. for 1 hour under nitrogen atmosphere. The reaction mixture was diluted with saturated Na₂S₂O₃ aqueous solution (50 mL) and extracted with EtOAc (50 mL*2). The combined organic layers were washed with brine (50 mL), dried over anhydrous Na₂SO₄, filtered and concentrated under reduced pressure. The residue was purified by flash silica gel chromatography (ISCO®; 12 g SepaFlash® Silica Flash Column, petroleum ether/EtOAc with EtOAc from 50˜100%, flow rate=30 mL/min) to afford 4-bromo-2-methyl-1-(2-trimethylsilylethoxymethyl)pyrazol-3-one (1.2 g, 48.8% yield, 93% purity) as a yellow solid.

¹H NMR (400 MHz, chloroform-d) δ 7.42 (s, 1H), 4.97 (s, 2H), 3.48-3.52 (m, 51H), 0.89 (t, J=8.0 Hz, 2H), −0.01 (s, 9H).

Step 4: a mixture of tert-butyl N-tert-butoxycarbonyl-N-(2-chloropyrimidin-4-yl)carbamate (2.68 g, 8.14 mmol, 5 eq), 4-bromo-2-methyl-1-(2-trimethylsilylethoxymethyl)pyrazol-3-one (500 mg, 1.63 mmol, 1 eq), 4,4,5,5-tetramethyl-2-(4,4,5,5-tetramethyl-1,3,2-dioxaborolan-2-yl)-1,3,2-dioxaborolane (2.07 g, 8.14 mmol, 5 eq), 4-ditert-butylphosphanyl-N,N-dimethyl-aniline; dichloropalladium (230.4 mg, 0.325 mmol, 0.2 eq) and Na₂CO₃ (862.4 mg, 8.14 mmol, 5 eq) in MeCN (25 mL) and 1-120 (2.5 mL) was degassed and purged with nitrogen for 3 times, and then the mixture was stirred at 100° C. for 4 hours under nitrogen atmosphere. The reaction mixture was diluted with 1120 (100 mL) and extracted with EtOAc (150 mL*2). The combined organic layers were washed with brine (200 mL), dried over anhydrous Na₂SO₄, filtered and concentrated under reduced pressure. The residue was purified by flash silica gel chromatography (ISCO®; 40 g SepaFlash® Silica Flash Column, DCM/MeOH with MeOH from 0˜10%, flow rate=30 mL/min) to afford tert-butyl N-tert-butoxycarbonyl-N-[2-[2-methyl-3-oxo-1-(2-trimethylsilylethoxymethyl)pyrazol-4-yl]pyrimidin-4-yl]carbamate (2 g, 55.4% yield, 47% purity) as a brown oil.

LCMS [M+H]⁺ m/z: calcd 522.3, found 522.4.

Step 5: to a solution of tert-butyl N-tert-butoxycarbonyl-N-[2-[2-methyl-3-oxo-1-(2-trimethylsilylethoxymethyl)pyrazol-4-yl]pyrimidin-4-yl]carbamate (500 mg, 0.958 mmol, 1 eq) in 1,1,1,3,3,3-hexafluoropropan-2-ol (10 mL) was added TFA (1 mL, 13.5 mmol, 14.09 eq). The mixture was stirred at 15° C. for 3 hours. The reaction mixture was diluted with saturated NaHCO₃ aqueous solution (50 mL) and extracted with EtOAc (50 mL*2). The combined organic layers were washed with brine (50 mL), dried over anhydrous Na₂SO₄, filtered and concentrated under reduced pressure. The residue was purified by preparative TLC (silica, DCM/MeOH=10/1, 254 nm) to afford 4-(4-aminopyrimidin-2-yl)-2-methyl-1-(2-trimethylsilylethoxymethyl)pyrazol-3-one (150 mg 46.7% yield, 96% purity) as a red solid.

¹H NMR (400 MHz, methanol-d₄) δ 8.38 (br s, 1H), 8.02 (br s, 1H), 6.34 (br s, 1H), 5.40 (s, 2H), 3.52-3.62 (m, 5H), 0.92 (t, J=8.0 Hz, 2H), −0.00 (s, 9H).

### (II) Synthesis of Compound (169)

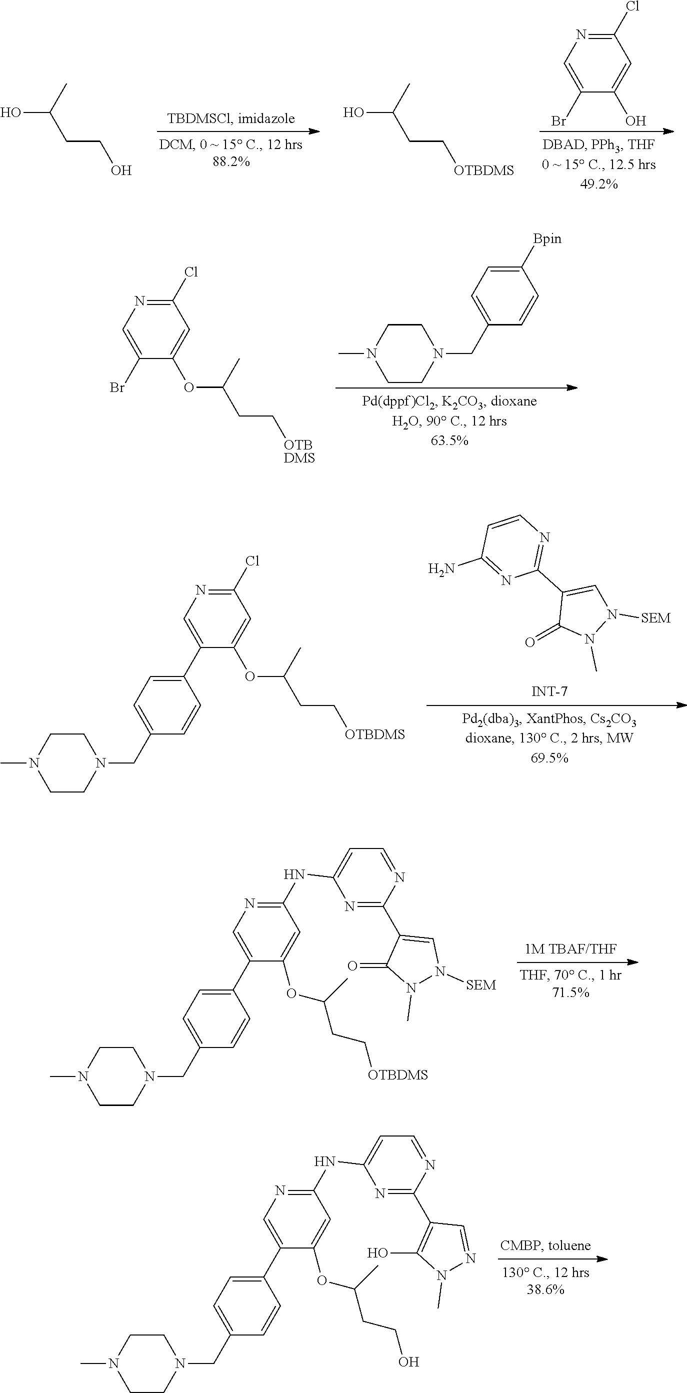

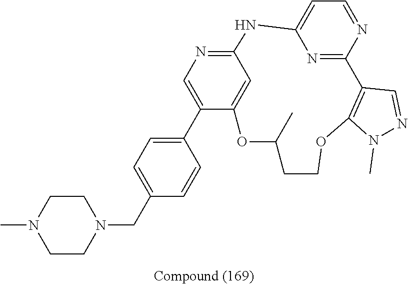 [SMILES: CC1CCOc2c(cnn2C)-c2nccc(n2)Nc2cc(c(-c3ccc(CN4CCN(C)CC4)cc3)cn2)O1]

Step 1: to a solution of butane-1,3-diol (5 g, 55.5 mmol, 1.0 eq) and IMIDAZOLE (4.15 g, 61.0 mmol, 1.1 eq) in DCM (80.0 mL) was added TBDMSCI (8.36 g, 55.5 mmol, 1.0 eq) at 0° C. The mixture was stirred at 15° C. for 12 hours. The reaction mixture was diluted with H₂O (100 mL) and extracted with DCM (100 mL*2). The combined organic layers were washed with brine (100 mL*1), dried over anhydrous Na₂SO₄, filtered and concentrated under reduced pressure. The residue was purified by flash/column chromatography (ISCO®; 40 g SepaFlash® Silica Flash Column, petroleum ether/EtOAc with EtOAc from 0˜30%, 100 mL/min, PMA) to afford 4-[tert-butyl(dimethyl)silyl]oxybutan-2-ol (10 g, 88.2% yield) as colourless oil.

¹H NMR (400 MHz, CDCl₃) δ ppm 4.07-3.98 (m, 1H), 3.92-3.86 (m, 1H), 3.84-3.78 (m, 1H), 1.70-1.60 (m, 2H), 1.19 (d, J=6.4 Hz, 31), 0.90 (s, 9H), 0.10-0.06 (m, 611).

Step 2: a mixture of 5-bromo-2-chloro-pyridin-4-ol (1 g, 4.80 mmol, 1.0 eq), PPh₃ (3.77 g, 14.4 mmol, 3.0 eq) and tert-butyl (NE)-N-tert-butoxycarbonyliminocarbamate (3.31 g, 14.4 mmol, 3.0 eq) in THE (15.0 mL) was stirred at 15° C. for 30 minutes under N₂, then cooled to 0° C., 4-[tert-butyl(dimethyl)silyl]oxybutan-2-ol (1.18 g, 5.76 mmol, 1.2 eq) in THF (5.0 mL) was added dropwise at 0° C., the mixture was stirred at 15° C. for 12 hours under N₂ atmosphere. The reaction mixture was diluted with H₂O (30 mL) and extracted with EtOAc (50 mL*2). The combined organic layers were washed with brine (50 mL*1), dried over anhydrous Na₂SO₄, filtered and concentrated under reduced pressure. The residue was purified by flash chromatography (ISCO®; 20 g SepaFlash® Silica Flash Column, petroleum ether/EtOAc with EtOAc from 0˜15%, 40 mL/min, 254 nm) to afford 3-[(5-bromo-2-chloro-4-pyridyl)oxy]butoxy-tert-butyl-dimethyl-silane (1.1 g, 49.2% yield, 85% purity) as a yellow solid.

¹H NMR (400 MHz, CDCl₃) δ ppm 8.37-8.32 (m, 1H), 6.91 (s, 1H), 4.80-4.71 (m, 1H), 3.80-3.69 (m, 2H), 2.07-1.98 (m, 1H), 1.88-1.79 (m, 1H), 1.43 (d, J=6.4 Hz, 3H), 0.90-0.87 (m, 9H), 0.02 (d, J=14.4 Hz, 6H).

Step 3: a mixture of 3-[(5-bromo-2-chloro-4-pyridyl)oxy]butoxy-tert-butyl-dimethyl-silane (1.1 g, 2.79 mmol, 1.0 eq), 1-methyl-4-[[4-(4,4,5,5-tetramethyl-1,3,2-dioxaborolan-2-yl)phenyl]methyl]piperazine (1.06 g, 3.34 mmol, 1.2 eq), Pd(dppf)Cl₂ (204 mg, 0.279 mmol, 0.1 eq), K₂CO₃ (1.16 g, 8.36 mmol, 3.0 eq) in dioxane (15.0 mL) and H₂O (3.0 mL) was degassed and purged with N₂ for 3 times, and then the mixture was stirred at 90° C. for 12 hours under N₂ atmosphere. The reaction mixture was diluted with H₂O (50 mL) and extracted with EtOAc (100 mL*2). The combined organic layers were washed with brine (100 mL*1), dried over anhydrous Na₂SO₄, filtered and concentrated under reduced pressure. The residue was purified by flash chromatography (ISCO®; 20 g SepaFlash® Silica Flash Column, EtOAc/MeOH with MeOH from 0˜15%, 40 mL/min, 254 nm) to afford tert-butyl-[3-[[2-chloro-5-[4-[(4-methylpiperazin-1-yl)methyl]phenyl]-4-pyridyl]oxy]butoxy]-dimethyl-silane (960 mg, 63.5% yield, 93% purity) as a yellow solid.

1-1 NMR (400 MHz, CDCl₃) δ ppm 8.18 (s, 1H), 7.44-7.40 (m, 2H), 7.39-7.35 (m, 2H), 6.98 (s, 1H), 4.79-4.71 (m, 1H), 3.65 (t, J=5.6 Hz, 2H), 3.59 (s, 2H), 3.50 (s, 1H), 2.77-2.50 (m, 7H), 2.42 (br s, 3H), 1.99-1.91 (m, 1H), 1.79-1.71 (m, 1H), 1.37 (d, J=6.0 Hz, 3H), 0.89 (s, 9H), 0.01 (d, J=5.6 Hz, 6H).

LCMS (ESI) [M+H]⁺ m/z: calcd 504.3, found 504.1.

Step 4: tert-butyl-[3-[[2-chloro-5-[4-[(4-methylpiperazin-1-yl)methyl]phenyl]-4-pyridyl]oxy]butoxy]-dimethyl-silane (300 mg, 0.595 mmol, 1.0 eq), 4-(4-aminopyrimidin-2-yl)-2-methyl-1-(2-trimethylsilylethoxymethyl)pyrazol-3-one (192 mg, 0.595 mmol, 1.0 eq), XantPhos (104 mg, 0.179 mmol, 0.3 eq), Cs₂CO₃ (582 mg, 1.79 mmol, 3.0 eq) and Pd₂(dba)₃ (82 mg, 0.0893 mmol, 0.15 eq) were taken up into a microwave tube in dioxane (10.0 mL). The sealed tube was heated at 130° C. for 2 hours under microwave. The reaction mixture was filtered and concentrated under reduced pressure. The residue was purified by flash chromatography (ISCO®; 4 g SepaFlash® Silica Flash Column, EtOAc/MeOH with MeOH from 0˜15%, 40 mL/min, 254 nm) to afford 4-[4-[[4-[3-[tert-butyl(dimethyl)silyl]oxy-1-methyl-propoxy]-5-[4-[(4-methylpiperazin-1-yl)methyl]phenyl]-2-pyridyl]amino]pyrimidin-2-yl]-2-methyl-1-(2-trimethylsilylethoxymethyl)pyrazol-3-one (350 mg, 69.5% yield, 93% purity) as a yellow solid.

LCMS (ESI) [M+H]⁺ m/z: calcd 789.5, found 789.4.

Step 5: to a solution of 4-[4-[[4-[3-[tert-butyl(dimethyl)silyl]oxy-1-methyl-propoxy]-5-[4-[(4-methylpiperazin-1-yl)methyl]phenyl]-2-pyridyl]amino]pyrimidin-2-yl]-2-methyl-1-(2-trimethylsilylethoxymethyl)pyrazol-3-one (200 mg, 0.253 mmol, 1.0 eq) in THE (5.0 mL) was added 1M TBAF/THF (0.5 mL, 0.5 mmol, 2.0 eq). The mixture was stirred at 70° C. for 1 hour. The reaction mixture was concentrated under reduced pressure. The crude product was purified by flash chromatography (Column: SepaFlash® Sphercial C18, 40 g, 40-60 μm, 120 Å; MeCN/water (0.5% NH₃—H₂O) with MeCN from 0-30%, 100 mL/min, 254 nm) to afford 4-[4-[[4-(3-hydroxy-1-methyl-propoxy)-5-[4-[(4-methylpiperazin-1-yl)methyl]phenyl]-2-pyridyl]amino]pyrimidin-2-yl]-2-methyl-pyrazol-3-ol (130 mg, 71.5% yield, 76% purity) as a yellow solid.

LCMS (ESI) [M+H]⁺ m/z: calcd 545.3, found 545.1.

Step 6: to a solution of 4-[4-[[4-(3-hydroxy-1-methyl-propoxy)-5-[4-[(4-methylpiperazin-1-yl)methyl]phenyl]-2-pyridyl]amino]pyrimidin-2-yl]-2-methyl-pyrazol-3-ol (130 mg. 0.239 mmol, 1.0 eq) in toluene (10.0 mL) was added 2-(tributyl-5-phosphanylidene)acetonitrile (288 mg, 1.19 mmol, 5.0 eq). The mixture was stirred at 130° C. for 12 hours under N₂. The reaction mixture was concentrated under reduced pressure. The residue was purified by flash chromatography (ISCO®: 20 g SepaFlash® Silica Flash Column, EtOAc/MeOH with MeOH from 0˜20%, 40 mL/min, 254 nm) to afford a crude product which was further purified by preparative 1-1PLC (column: Phenomenex Gemini-NX 80*40 mm*3 μm; mobile phase: [water (10 mM NH₄HCO₃)-ACN]; B %: 32%-62%, 7.8 min) to afford 5,10-dimethyl-13-[4-[(4-methylpiperazin-1-yl)methyl]phenyl]-7,11-dioxa-4,5,15,17,21,22-hexazatetracyclo[16.3.1.112,16.02,6]tricosa-1(22),2(6),3,12(23),13,15,18,20-octaene (compound (20)) (49.0 mg, 38.6% yield) as a white solid.

¹H NMR (400 MHz, CD₃OD) δ ppm 8.77 (s, 1H), 8.23 (d, J=5.6 Hz, 1H), 8.03 (d, J=8.8 Hz, 2H), 7.50 (d, J=8.0 Hz, 2H), 7.38 (d, J=8.0 Hz, 2H), 6.68 (d, J=5.6 Hz, 1H), 5.18-5.09 (n, 1H), 4.76-4.68 (m, 1H), 4.24-4.17 (m, 1H), 3.79 (s, 3H), 3.62 (s, 2H), 2.97-2.52 (m, 8H), 2.46 (s, 3H), 2.32-2.22 (m, 2H), 1.43 (d, J=6.4 Hz, 3H).

LCMS (ESI) [M+H]⁺ m/z: calcd 527.3, found 527.1.

The regio-chemistry was confirmed by HSQC (the chemical shift of —(CH₂—O— is 71.932 ppm).

### Example 2: Preparation of Compound (48)

### Synthesis of inhibitors: (10S)-5,10-dimethyl-13-[1-methyl-4-(2,2,2-trifluoroethoxy)pyrrol-3-yl]-7,11-dioxa-4,5,15,17,21,22-hexazatetracyclo[16.3.1.1^{12,16}.0^{2,6}]tricosa-1(22),2(6),3,12(23),13,15,18,20-octaene (compound (48))

### (I) Synthesis of Intermediate B (INT-B)

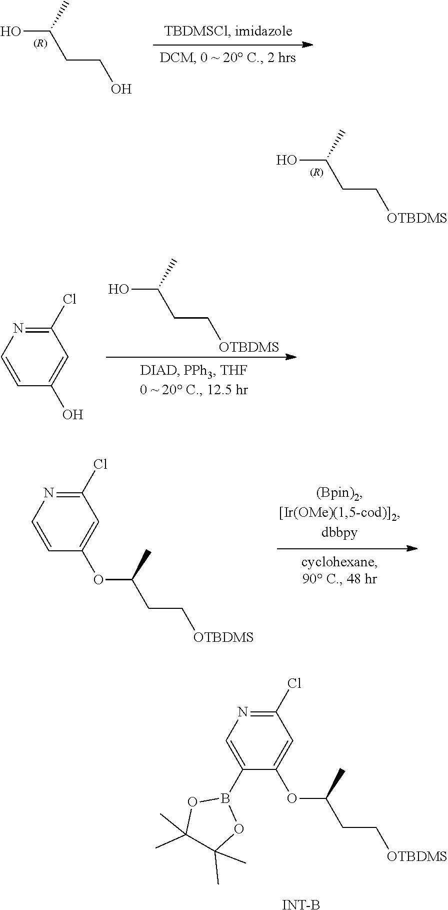

Step 1: to a solution of (3R)-butane-1,3-diol (20 g, 0.222 mol) in DCM (200 ml) was added imidazole (20 g, 0.294 mol) and tert-butyl-chloro-dimethyl-silane (34 g, 0.226 mol) at 0° C. The mixture was stirred at 20° C. for 12 hours. The reaction mixture was washed with brine (200 mL*2), dried over Na₂SO₄, filtered and concentrated under reduced pressure. The residue was purified by flash chromatography (ISCO®; 220 g SepaFlash® Silica Flash Column, petroleum ether/EtOAc with EtOAc from 0˜30%, flow rate: 100 mL/min, PMA) to give (2R)-4-[tert-butyl(dimethyl)silyl]oxybutan-2-ol (39 g, 86%) as colorless oil.

¹H NMR (400 MHz, CDCl₃) δ ppm 4.04-3.90 (m, 1H), 3.89-3.86 (m, 1H), 3.84-3.80 (m, 1H), 1.69-1.62 (m, 2H), 1.19 (d, J=6.4 Hz, 2H), 0.91 (s, 9H), 0.08 (s, 6H).

Step 2: a mixture of 2-chloropyridin-4-ol (10 g, 0.0772 mol), (2R)-4-[tert-butyl(dimethyl)silyl]oxybutan-2-ol (19 g, 0.0930 mmol) and PPh₃ (30 g, 0.114 mol) in THF (200 mL) was added DIAD (23 mL, 0.118 mol) dropwise at 0° C., and then the mixture was stirred at 20° C. for 2 hours under nitrogen atmosphere. The reaction mixture was concentrated under reduced pressure and the residue was purified by flash chromatography (ISCO®; 330 g SepaFlash® Silica Flash Column, petroleum ether/EtOAc with EtOAc from 0˜15%, flow rate: 100 mL/min, 254 nm) to give tert-butyl-[(3S)-3-[(2-chloro-4-pyridyl)oxy]butoxy]-dimethyl-silane (25 g, 88.2% yield, 86% purity) as a yellow oil.

¹H NMR (400 MHz, CDCl₃) δ ppm 8.14 (d, J=5.6 Hz, 1H), 6.84 (d, J=2.0 Hz, 1H), 6.74 (dd, J=5.6 Hz, 2.0 Hz, 2H), 4.72-4.64 (m, 1), 3.72-3.65 (m, 1H), 1.97-1.91 (m, 1H), 1.78-1.72 (m, 1H), 1.34 (d, J=6.4 Hz, 3H), 0.87 (s, 9H), 0.02 (s, 3H), −0.01 (s, 3H).

LCMS (ESI) [M+H]⁺ m/z: calcd 316.1, found 315.9.

Step 3: a mixture of tert-butyl-[(3S)-3-[(2-chloro-4-pyridyl)oxy]butoxy]-dimethyl-silane (13 g, 41.1 mmol), (Bpin)₂ (18 g, 70.9 mmol), 4-tert-butyl-2-(4-tert-butyl-2-pyridyl)pyridine (1 g, 3.73 mmol) and (1Z,5Z)-cycloocta-1,5-diene; 2,4-dimethyl-BLAH-bicyclo[1.1.0]butane (1 g, 1.51 mmol) in hexane (250 mL) was degassed and purged with nitrogen for 3 times, and then the mixture was stirred at 90° C. for 48 hours under nitrogen atmosphere. The reaction mixture was concentrated under reduced pressure. The residue was purified by flash chromatography (ISCO®; 330 g SepaFlash® Silica Flash Column, petroleum ether/EtOAc with EtOAc from 0˜24%, flow rate: 100 mL/min, 254 nm) to afford tert-butyl-[(3S)-3-[[2-chloro-5-(4,4,5,5-tetramethyl-1,3,2-dioxaborolan-2-yl)-4-pyridyl]oxy]butoxy]-dimethyl-silane (10 g, 44.0% yield) as a yellow oil. The regio-chemistry was confirmed by HSQC.

¹H NMR (400 MHz, CDCl₃) δ ppm 8.40 (s, 1H), 6.82 (s, 1H), 4.68-4.63 (m, 1H), 3.83-3.78 (m, 1H), 3.74-3.70 (m, 1H), 1.99-1.93 (m, 1H), 1.83-1.80 (m, 1H), 1.61 (d, J=6.0 Hz, 3H), 1.33 (s, 12H), 0.87 (s, 9H), 0.01 (s, 3H), −0.02 (s, 3H).

LCMS (ESI) [M+H]⁺ m/z: calcd 442.2, found 442.1 (the boronic acid was observed on LCMS with a different retention time).

### (II) Synthesis of Compound (48)

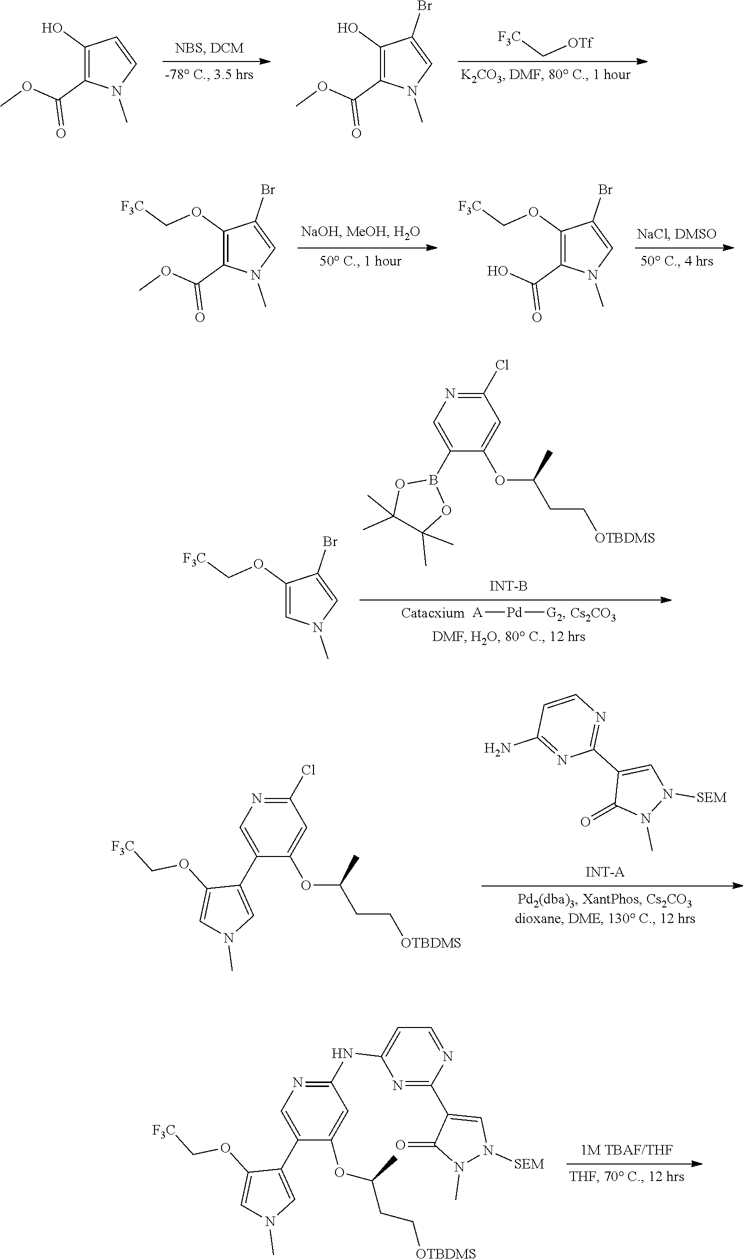

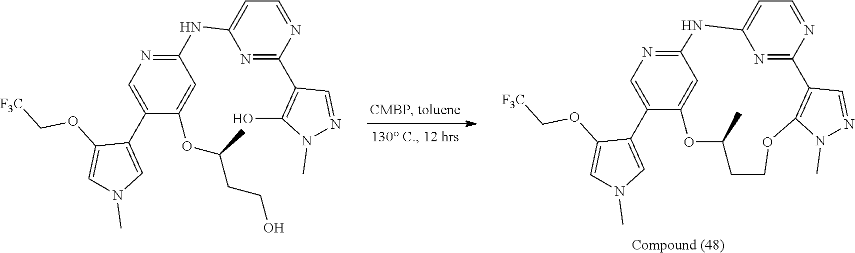

Step 1: to a solution of methyl 3-hydroxy-1-methyl-pyrrole-2-carboxylate (1.5 g, 9.67 mmol, 1.0 eq) in DCM (20.0 mL) was added NBS (2.06 g, 11.60 mmol, 1.2 eq). The mixture was stirred at −78° C. for 3.5 hours. The reaction mixture was quenched by addition saturated Na₂SO₃ aqueous solution (30 mL) at −78° C., and then diluted with H₂O (10 mL) and extracted with DCM (30 mL*2). The combined organic layers were washed with saturated Na₂SO₃ aqueous solution (40 mL*3) and brine (40 mL*2), dried over Na₂SO₄, filtered and concentrated under reduced pressure to afford methyl 4-bromo-3-hydroxy-1-methyl-pyrrole-2-carboxylate (1.7 g, crude) as a white solid. LCMS [M+H]⁺ m/z: calcd 233.9, found 233.8.

Step 2: to a solution of methyl 4-bromo-3-hydroxy-1-methyl-pyrrole-2-carboxylate (1.7 g, 7.26 mmol, 1.0 eq) and K₂CO₃ (3.1 g, 22.43 mmol, 3.1 eq) in DMF (20.0 mL) was added 2,2,2-trifluoroethyl trifluoromethanesulfonate (3.4 g, 14.65 mmol, 2.0 eq). The mixture was stirred at 80° C. for 1 hour. The reaction mixture was diluted with H₂O (50 mL) and extracted with EtOAc (50 mL*2). The combined organic layers were washed with brine (60 mL*5), dried over Na₂SO₄, filtered and concentrated under reduced pressure. The residue was purified by flash chromatography (ISCO®; 20 g SepaFlash® Silica Flash Column, petroleum ether/EtOAc with EtOAc from 0˜20%, flow rate: 80 mL/min, 254 nm) to afford methyl 4-bromo-1-methyl-3-(2,2,2-trifluoroethoxy)pyrrole-2-carboxylate (1.7 g, 68.8% yield, 93% purity) as a white solid.

¹H NMR (400 MHz, chloroform-d) δ ppm 6.68 (s, 1H), 4.37 (q, J=8.4 Hz, 2H), 3.86 (d, J=8.5 Hz, 6H). LCMS [M+H]⁺ m/z: calcd 315.9, found 317.8.

Step 3: to a solution of methyl 4-bromo-1-methyl-3-(2,2,2-trifluoroethoxy)pyrrole-2-carboxylate (1.7 g, 5.38 mmol, 1.0 eq) in MeOH (20.0 mL) was added NaOH (2.2 g, 55.00 mmol, 10.2 eq) in H₂O (4.0 ml). The mixture was stirred at 50° C. for 1 hour. The reaction mixture was concentrated under reduced pressure. The residue was diluted with H₂O (10 mL) and adjusted with 4N HCl to pH<5. Then the mixture was filtered and the filter cake was washed with H₂O (10 mL*3). The filter cake was concentrated under reduced pressure to afford 4-bromo-1-methyl-3-(2,2,2-trifluoroethoxy)pyrrole-2-carboxylic acid (1.7 g, crude) as a white solid.

¹H NMR (400 MHz, DMSO-d) δ ppm 12.84 (br s, 1H), 7.19 (s, 1:H), 4.53 (q, J=9.0 Hz, 2H), 3.77 (s, 3H). LCMS [M+H]⁺ m/z: calcd 301.9, found 301.9.

Step 4: to a solution of 4-bromo-1-methyl-3-(2,2,2-trifluoroethoxy)pyrrole-2-carboxylic acid (1.7 g, 5.63 mmol, 1.0 eq) in DMSO (20.0 mL was added NaCl (660 mg, 11.29 mmol, 2.0 eq). The mixture was stirred at 140° C. for 4 hours. The reaction mixture was diluted with H₂O (40 mL) and extracted with EtOAc (50 mL*2). The combined organic layers were washed with brine (60 mL*5), dried over Na₂SO₄, filtered and concentrated under reduced pressure. The residue was purified by flash chromatography (ISCO®; 20 g SepaFlash® Silica Flash Column, petroleum ether/EtOAc with EtOAc from 0-14%, flow rate: 80 mL/min, 254 nm) to afford 3-bromo-1-methyl-4-(2,2,2-trifluoroethoxy)pyrrole (1.2 g, 64.4% yield, 78% purity) as a yellow oil.

¹H NMR (400 MHz, chloroform-d) δ ppm 6.44 (d, J=2.6 Hz, 1H), 6.31 (d, J=2.6 Hz, 1H), 4.25 (q, J=8.4 Hz, 2H), 3.56 (s, 3H). LCMS [M+H]⁺ m/z: calcd 257.9, found 259.8.

Step 5: a mixture of 3-bromo-1-methyl-4-(2,2,2-trifluoroethoxy)pyrrole (1.2 g, 4.65 mmol, 1.0 eq), tert-butyl-[(3S)-3-[[2-chloro-5-(4,4,5,5-tetramethyl-1,3,2-dioxaborolan-2-yl)-4-pyridyl]oxy]butoxy]-dimethyl-silane (2.9 g, 6.56 mmol, 1.4 eq), Cs₂CO₃ (4.6 g, 14.12 mmol, 3.0 eq), [2-(2-aminophenyl)phenyl]-chloro-palladium; bis(1-adamantyl)-butyl-phosphane (310 mg, 0.463 mmol, 0.1 eq) in DMF (20.0 mL) and H₂O (2.0 mL) was degassed and purged with N₂ for 3 times, and then the mixture was stirred at 80° C. for 12 hours under N₂ atmosphere. The reaction mixture was filtered and the filter cake was washed with EtOAc (15 mL*4). The combined filtrate was extracted with EtOAc (30 mL*2). The combined organic layers were washed with brine (50 mL*5), dried over Na₂SO₄, filtered and concentrated under reduced pressure. The residue was purified by flash chromatography (ISCO®; 20 g SepaFlash® Silica Flash Column, petroleum ether/EtOAc with EtOAc from 0˜17% flow rate: 80 mL/min, 254 nm) to afford tert-butyl-[(3S)-3-[[2-chloro-5-[1-methyl-4-(2,2,2-trifluoroethoxy)pyrrol-3-yl]-4-pyridyl]oxy]butoxy]-dimethyl-silane (1 g, 41.0% yield, 94% purity) as a yellow oil.

LCMS [M+H]⁺ m/z: calcd 493.1, found 493.1.

Step 6: a mixture of tert-butyl-[(3S)-3-[[2-chloro-5-[1-methyl-4-(2,2,2-trifluoroethoxy)pyrrol-3-yl]-4-pyridyl]oxy]butoxy]-dimethyl-silane (1 g, 2.03 mmol, 1.0 eq), 4-(4-aminopyrimidin-2-yl)-2-methyl-1-(2-trimethylsilylethoxymethyl)pyrazol-3-one (700 mg, 2.18 mmol, 1.0 eq), Pd₂(dba)₃ (190 mg, 0.207 mmol, 0.1 eq), Xantphos (120 mg, 0.207 mmol, 0.1 eq) and C₈₂CO₃ (2 g, 6.14 mmol, 3.0 eq) in dioxane (20.0 mL) and DME (4.0 mL) was degassed and purged with N₂ for 3 times, and then the mixture was stirred at 130° C. for 12 hours under N₂ atmosphere. The reaction mixture was filtered and the filter cake was washed with DCM (15 mL*4). The combined filtrate was concentrated under reduced pressure. The residue was purified by flash silica gel chromatography (ISCO®; 20 g SepaFlash® Silica Flash Column, MeOH (0.05% N₁H₂O)/DCM with MeOH (0.05% NH₃H₂O) from 0˜17%, flow rate: 80 mL/min, 254 nm) to afford 4-[4-[[4-[(1S)-3-[tert-butyl(dimethyl)silyl]oxy-1-methyl-propoxy]-5-[1-methyl-4-(2,2,2-trifluoroethoxy)pyrrol-3-yl]-2-pyridyl]amino]pyrimidin-2-yl]-2-methyl-1-(2-trimethylsilylethoxymethyl)pyrazol-3-one (700 mg, crude) as a brown oil.

LCMS [M+H]⁺ m/z: calcd 7783, found 778.4.

Step 7: to a solution of 4-[4-[[4[(1S)-3-[tert-butyl(dimethyl)silyl]oxy-1-methyl-propoxy]-5-[1-methyl-4-(2,2,2-trifluoroethoxy)pyrrol-3-yl]-2-pyridyl]amino]pyrimidin-2-yl]-2-methyl-1-(2-trimethylsilylethoxymethyl)pyrazol-3-one (700 mg, 0.899 mmol, 1.0 eq) in THF (5.0 mL) was added 2.7 mL 1 M TBAF/THF. The mixture was stirred at 70° C. for 12 hours. The reaction mixture concentrated under reduced pressure. The residue was purified by flash silica gel chromatography (Biotage®, Column: SepaFlash® Sphercial C18, 60 g, 40-60 μm, 120 Å; MeCN/water (0.05% NH₃—H₂O) with MeCN from 0-47%, 50 mL/min, 254 nm) to afford 4-[4-[[4-[(1S)-3-hydroxy-1-methyl-propoxy]-5-[1-methyl-4-(2,2,2-trifluoroethoxy)pyrrol-3-yl]-2-pyridyl]amino]pyrimidin-2-yl]-2-methyl-pyrazol-3-ol (830 mg, crude) as a brown solid.

LCMS [M+H]⁺ m/z: calcd 534.2, found 534.1.

Step 8: a mixture of 4-[4-[[4-[(1S)-3-hydroxy-1-methyl-propoxy]-5-[1-methyl-4-(2,2,2-trifluoroethoxy)pyrrol-3-yl]-2-pyridyl]amino]pyrimidin-2-yl]-2-methyl-pyrazol-3-ol (830 mg, 1.56 mmol, 1.0 eq), 2-(tributyl-λ⁵-phosphanylidene)acetonitrile (1.9 g, 7.87 mmol, 5.0 eq) in toluene (20.0 mL) was degassed and purged with N₂ for 3 times, and then the mixture was stirred at 130° C. for 12 hours under N₂ atmosphere. The reaction mixture was concentrated under reduced pressure. The residue was purified by flash chromatography (ISCO©; 20 g SepaFlash® Silica Flash Column, DCM/EtOAc with EtOAc from 0˜100%, then DCM/MeOH (0.05% NH₃H₂O) with MeOH (0.05% NH₃H₂O) from 0˜20%, flow rate: 80 ml/min, 254 nm) to give a crude product. The crude product was purified by preparative HPLC (column: 2_Phenomenex Gemini C18 75*40 mm*3 um; mobile phase: [water (ammonia hydroxide v/v)-ACN]; B %: 40%-70%, 9.5 min. Column Temp: 30° C.) to afford (10S)-5,10-dimethyl-13-[1-methyl-4-(2,2,2-trifluoroethoxy)pyrrol-3-yl]-7,11-dioxa-4,5,15,17,21,22-hexazatetracyclo[16.3.1.1^{12,16}.0^{2,6}]tricosa-1(22),2(6),3,12(23),13,15,18,20-octaene (127.8 mg, 15.7% yield, 99% purity) as a white solid.

¹H NMR (400 MHz, methanol-d₄) δ ppm 8.57 (s, 1H), 8.48 (s, 1H), 8.13 (d, J=6.0 Hz, 1H), 7.97 (s, 1H), 6.92 (d, J=2.5 Hz, 1H), 6.58 (d, J=5.8 Hz, 1H), 6.44 (d, J=2.5 Hz, 1H), 5.07-4.99 (m, 1H), 4.63 (dt, J=3.0, 9.5 Hz, 1H), 4.32 (q, J=8.7 Hz, 2H), 4.10 (td, J=4.5, 9.3 Hz, 1H), 3.75 (s, 3H), 3.59 (s, 3H), 2.29-2.17 (m, 2H), 1.48 (d, J=6.3 Hz, 3H)

¹⁹F NMR (377 MHz, methanol-d₄) δ ppm −75.77 (s, 1F)

LCMS [M+H]⁺ m/z: calcd 516.1, found 516.1.

The regio-chemistry was confirmed by HM BC (the chemical shift of −CH₂—O— is 71.853 ppm).

### Example 3: Preparation of Compound (28) and (25)

### Synthesis of inhibitor: 2-[4-[(10S)-5,10-dimethyl-7,11-dioxa-4,5,15,17,21,22-hexazatetracyclo[16.3.1.1^{12,16}.0^{2,6}]tricosa-1(22),2(6),3,12(23),13,15,18,20-octaen-13-yl]-3-ethoxy-pyrazol-1-yl]cyclopropanecarbonitrile (compound (28)) and 2-[4-[(10S)-5,10-dimethyl-7,11-dioxa-4,5,15,17,21,22-hexazatetracyclo[16.3.1.1^{12,16}.0^{2,6}]tricosa-1(22),2(6),3,12(23),13,15,18,20-octaen-13-yl]-3-ethoxy-pyrazol-1-yl]cyclopropanecarbonitrile (compound (25))

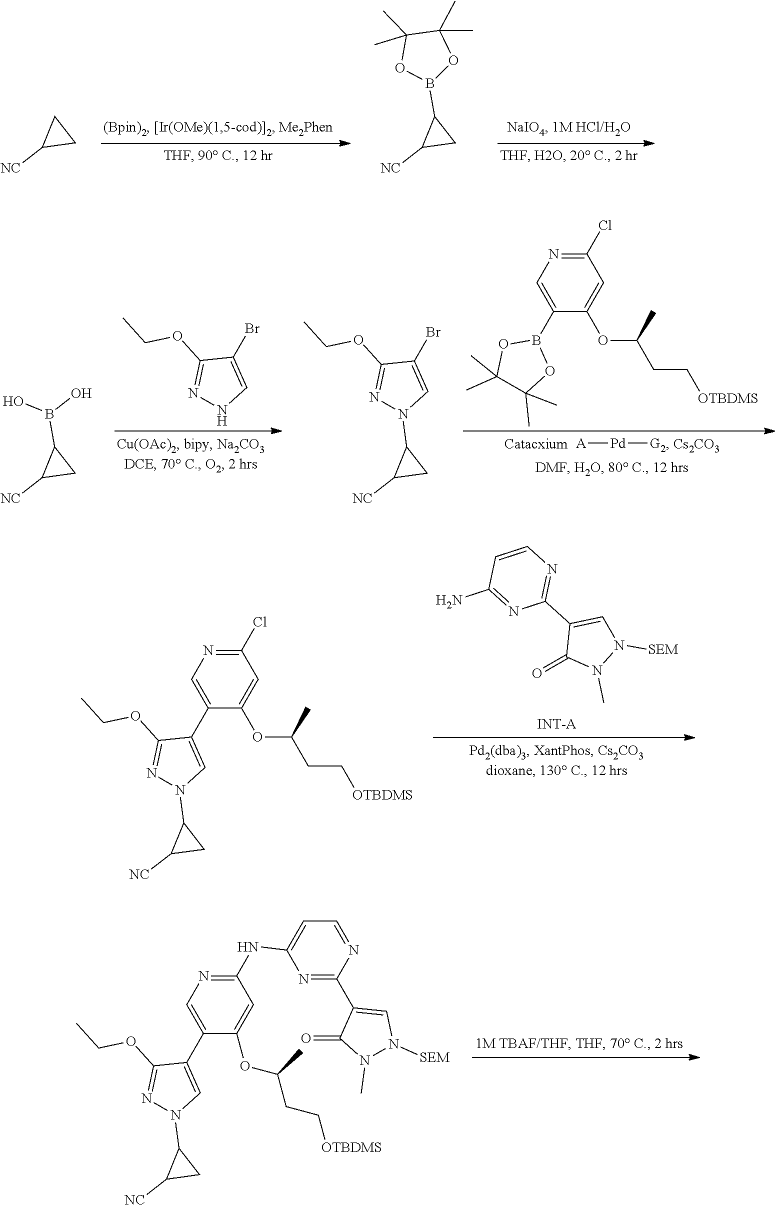

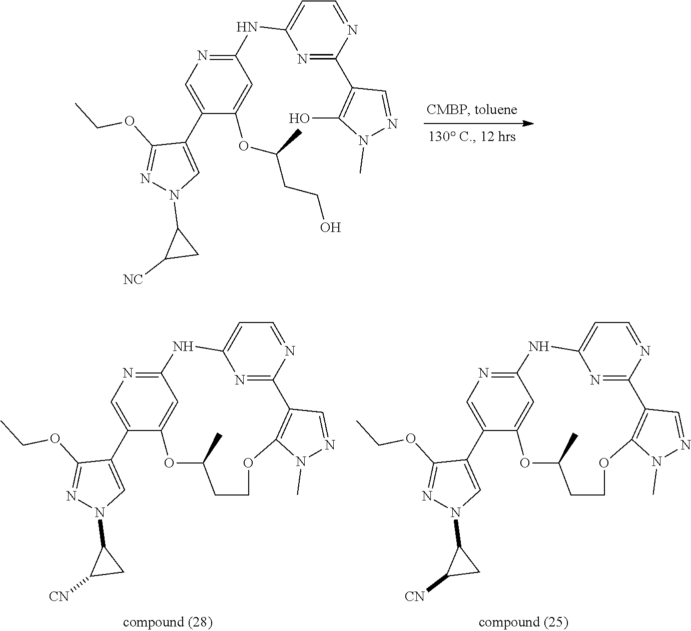

Step 1: a mixture of cyclopropanecarbonitrile (10.0 g, 149 mmol, 1.0 eq), Pin₂B₂ (34.0 g, 134 mmol, 0.9 eq), 2,9-dimethyl-1,10-phenanthroline (1.0 g, 4.80 mmol, 0.03 eq) and (1,5-Cyclooctadiene)(methoxy)iridium(I) Dimer (1.0 g, 1.51 mmol, 0.02 eq) in THF (150.0 mL) was degassed and purged with N₂ for 3 times, and then the mixture was stirred at 90° C. for 12 hours under N₂ atmosphere. The reaction mixture was concentrated under reduced pressure. The residue was purified by flash silica gel chromatography (ISCO®; 120 g SepaFlash® Silica Flash Column, Petroleum ether/EtOAc with EtOAc from 0˜15%, flow rate=100 mL/min, KMnO₄) to afford 2-(4,4,5,5-tetramethyl-1,3,2-dioxaborolan-2-yl)cyclopropanecarbonitrile (3.3 g, 11.4% yield) as a white solid.

¹H NMR (400 MHz, chloroform-d) δ ppm 1.49 (td, J=5.4, 8.2 Hz, 1H), 1.30 (td, J=4.6, 10.4 Hz, 1H), 1.23 (s, 12H), 1.09 (dt, J=4.0, 7.8 Hz, 1H), 0.63 (ddd, J=5.8, 7.6, 10.2 Hz, 1H)

Step 2: to a mixture of 2-(4,4,5,5-tetramethyl-1,3,2-dioxaborolan-2-yl)cyclopropanecarbonitrile (1.0 g, 5.18 mmol, 1.0 eq) in THF (8.0 mL) and 1.120 (2.0 mL) were added NaIO₄ (1.66 g, 7.76 mmol, 1.5 eq) and 1 M HCl/H₂O (6.2 mL, 1.2 eq). The mixture was stirred at 20° C. for 2 hours. The reaction mixture was diluted with water (30 mL) and extracted with EtOAc (50 mL*2). The combined organic layers were washed with saturated aqueous solution Na₂S₂O₃ (30 mL*2) and brine (30 mL*2), dried over Na₂SO₄, filtered and concentrated under reduced pressure to afford (2-cyanocyclopropyl)boronic acid (600 mg, crude) as a yellow solid.

¹H NMR (400 MHz, methanol-d₄) δ ppm 1.52 (br s, 1H), 1.27-1.17 (m, 1H), 1.04 (dt, J=3.6, 7.8 Hz, 1H), 0.70 (br s, 1H)

Step 3: a mixture of 4-bromo-3-ethoxy-1H-pyrazole (400 mg, 2.09 mmol, 1.0 eq), (2-cyanocyclopropyl)boronic acid (600 mg, 5.41 mmol, 2.6 eq), Cu(OAc)₂ (380 mg, 2.09 mmol, 1.0 eq), Na₂CO₃ (440 mg, 4.15 mmol, 2.0 eq) and 2-(2-pyridyl)pyridine (340 mg, 2.18 mmol, 1.0 eq) in DCE (10.0 mL) was stirred at 70° C. for 2 hours under O₂ atmosphere. The reaction mixture was filtered and the filter cake was washed with EtOAc (100 mL). The combined organic layers was washed with brine (50 mL*2), dried over Na₂SO₄, filtered and concentrated under reduced pressure. The residue was purified by flash silica gel chromatography (ISCO©;20 g SepaFlash® Silica Flash Column, Petroleum ether/EtOAc with EtOAc from 0˜24%, flow rate 80 mL/min, 254 nm) to afford 2-(4-bromo-3-ethoxy-pyrazol-1-yl)cyclopropanecarbonitrile (400 mg, 65.6% yield, 88% purity) as a yellow solid.

LCMS (ESI) [M+H]⁺ m/z: calcd 256.1, found 258.1.

¹H NMR (400 MHz, methanol-d₄) δ ppm 7.66 (s, 1H), 4.21 (q, J=7.0 Hz, 2H), 4.16 (dt, J=2.4, 5.4 Hz, 1H), 2.15 (ddd, J=3.2, 6.6, 10.0 Hz, 1H), 1.91 (ddd, J=5.0, 6.2, 10.0 Hz, 1H), 1.65 (td, J=6.4, 8.2 Hz, 1H), 1.36 (t, J=7.0 Hz, 3H)

The regio-chemistry was confirmed by NOE.

Step 4: a mixture of 2-(4-bromo-3-ethoxy-pyrazol-1-yl)cyclopropanecarbonitrile (600 mg, 2.34 mmol, 1.0 eq), tert-butyl-[(3S)-3-[[2-chloro-5-(4,4,5,5-tetramethyl-1,3,2-dioxaborolan-2-yl)-4-pyridyl]oxy]butoxy]-dimethyl-silane (1.35 g, 3.05 mmol, 1.3 eq), [2-(2-aminophenyl)phenyl]-chloro-palladium; bis(1-adamantyl)-butyl-phosphane (160 mg, 0.239 mmol, 0.1 eq) and Cs₂CO₃ (2.3 g, 7.06 mmol, 3.0 eq) in DMF (20.0 mL) and H₂O (2.0 mL) was degassed and purged with N₂ for 3 times, and then the mixture was stirred at 80° C. for 12 hours under N₂ atmosphere. The reaction mixture was filtered and the filter cake was washed with EtOAc (50 mL). The combined organic layer was washed with brine (30 mL*4), dried over Na₂SO₄, filtered and concentrated under reduced pressure. The residue was purified by flash silica gel chromatography (ISCO®; 40 g SepaFlash® Silica Flash Column, Petroleum ether/EtOAc with EtOAc from 0˜20%, flow rate=100 mL/min, 254 nm) to afford 2-[4-[4-[(1S)-3-[tert-butyl(dimethyl)silyl]oxy-1-methyl-propoxy]-6-chloro-3-pyridyl]-3-ethoxy-pyrazol-1-yl]cyclopropanecarbonitrile (300 mg, 21.6% yield, 83% purity) as a yellow oil.

LCMS (ESI) [M+H]⁺ m/z: calcd 491.2, found 491.1.

¹H NMR (400 MHz, chloroform-c) δ ppm 8.85 (s, 1H), 7.84 (s, 1H), 6.95 (s, 1H), 4.86-4.77 (m, 1H), 4.27 (q, J=7.0 Hz, 2H), 3.77-3.71 (m, 2H), 2.08-1.97 (m, 4H), 1.67-1.62 (m, 21H), 1.46 (dd, J=2.2, 6.2 Hz, 3), 1.42-1.39 (m, 3H), 0.91 (d, J 1.6 Hz, 9H), 0.04 (dd, J=2.8, 7.8 Hz, 6H).

Step 5: a mixture of 2-[4-[4-[(1S)-3-[tert-butyl(dimethyl)silyl]oxy-1-methyl-propoxy]-6-chloro-3-pyridyl]-3-ethoxy-pyrazol-1-yl]cyclopropanecarbonitrile (300 mg, 0.611 mmol, 1.0 eq), 4-(4-aminopyrimidine-2-yl)-2-methyl-1-(2-trimethylsilylethoxymethyl)pyrazol-3-one (200 mg, 0.622 mmol, 1.0 eq), Xantphos (100 mg, 0.173 mmol, 0.3 eq), Pd₂(dba)₃ (120 mg, 0.131 mmol, 0.2 eq) and Cs₂CO₃ (600 mg, 1.84 mmol, 3.0 eq) in dioxane (20.0 mL) and DME (4.0 mL) was degassed and purged with N₂ for 3 times, and then the mixture was stirred at 130° C. for 12 hours under N₂ atmosphere. The reaction mixture was filtered and the filter cake was washed with DCM (100 mL). The combined filtrate was concentrated under reduced pressure. The residue was purified by flash silica gel chromatography (ISCO®; 20 g SepaFlash® Silica Flash Column, Petroleum ether/EtOAc with EtOAc from 0˜100% to DCM/MeOH (0.05% NH₃—H₂O) with MeOH from 0˜15%, flow rate=80 mL/min, 254 nm) to afford 2-[4-[4-[(1S)-3-[tert-butyl(dimethyl)silyl]oxy-1-methyl-propoxy]-6-[[2-[2-methyl-3-oxo-1-(2-trimethylsilylethoxymethyl)pyrazol-4-yl]pyrimidin-4-yl]amino]-3-pyridyl]-3-ethoxy-pyrazol-1-yl]cyclopropanecarbonitrile (400 mg, 75.0% yield, 89% purity) as a yellow solid.

LCMS (ESI) [M+H]⁺ m/z: calcd 776.4, found 776.4.

Step 6: to a mixture of 2-[4-[4-[(1S)-3-[tert-butyl(dimethyl)silyl]oxy-1-methyl-propoxy]-6-[[2-[2-methyl-3-oxo-1-(2-trimethylsilylethoxymethyl)pyrazol-4-yl]pyrimidin-4-yl]amino]-3-pyridyl]-3-ethoxy-pyrazol-1-yl]cyclopropanecarbonitrile (400 mg, 0.515 mmol, 1.0 eq) in THF (10.0 mL) was added 1 m TBAF/THF (1.5 mL, 2.9 eq) and the mixture was stirred at 70° C. for 2 hours. The reaction mixture was concentrated under reduced pressure. The residue was purified by flash chromatography (Biotage®, Column: SepaFlash® Sphercial C18, 60 g, 40-60 μm, 120 Å; MeCN/water (0.05% NH₃—H₂O) with MeCN from 0˜38%, 50 ml/min, 254 am) to afford 2-[3-ethoxy-4-[4-[(1S)-3-hydroxy-1-methyl-propoxy]-6-[[2-(5-hydroxy-1-methyl-pyrazol-4-yl)pyrimidin-4-yl]amino]-3-pyridyl]pyrazol-1-yl]cyclopropanecarbonitrile (200 mg, 64.2% yield, 88% purity) as a yellow solid.

LCMS [M+H]⁺ m/z: calcd 532.2, found 532.3.

Step 7: a mixture of 2-[3-ethoxy-4-[4-[(1S)-3-hydroxy-1-methyl-propoxy]-6-[[2-(5-hydroxy-1-methyl-pyrazol-4-yl)pyrimidin-4-yl]amino]-3-pyridyl]pyrazol-1-yl]cyclopropanecarbonitrile (200 mg, 0.376 mmol, 1.0 eq) and 2-(tributyl-phosphanylidene)acetonitrile (450 mg, 1.86 mmol, 5.0 eq) in toluene (20.0 mL) was degassed and purged with N₂ for 3 times, and then the mixture was stirred at 130° C. for 12 hours under N₂ atmosphere. The reaction mixture concentrated under reduced pressure. The residue was purified by flash silica gel chromatography (ISCO®; 20 g SepaFlash® Silica Flash Column, Petroleum ether/EtOAc with EtOAc from 0˜100% to DCM/MeOH (0.05% NH₃·H₂O) with MeOH from 0˜15%, flow rate=80 mL/min, 254 nm) to give a crude product. The crude product was purified by prep-HPLC (Column: Welch Xtimate C:18 100*25 mm*3 um; Mobile phase: [water(FA)-ACN];B %: 16%-46%,8 min, Column Temp:30° C.) and to afford 2-[4-[(10S)-5,10-dimethyl-7,11-dioxa-4,5,15,17,21,22-hexazatetracyclo[16.3.1.1^{12,16}.0^{2,6}]tricosa-1(22),2(6),3,12(23),13,15,18,20-octaen-13-yl]-3-ethoxy-pyrazol-1-yl]cyclopropanecarbonitrile (44.3 mg, 22.6% yield) as a white solid and 2-[4-[(10S)-5,10-dimethyl-7,11-dioxa-4,5,15,17,21,22-hexazatetracyclo[16.3.1.1^{12,16}.0^{2,6}]tricosa-1(22),2(6),3,12(23),13,15,18,20-octaen-13-yl]-3-ethoxy-pyrazol-1-yl]cyclopropanecarbonitrile (7.2 mg, 3.6% yield) as a white solid. 2D NMR determined the major isomer to be trans-substituted cyclopropanes (relative configuration), namely compound (28).

LCMS [M+H]⁺ m/z: calcd 514.2, found 514.1.

Compound (28): ¹H NMR (400 MHz, methanol-d₄) δ ppm 8.74 (s, 1H), 8.63 (d, J=2.4 Hz, 1H), 8.24 (d, J=6.0 Hz, 1H), 8.03 (s, 1H), 8.02 (d, J=4.4 Hz, 1H), 6.70 (d, J=6.0 Hz, 1H), 5.19 (br d, J=3.8 Hz, 1H), 4.78-4.72 (m, 1H), 4.34-4.23 (m, 4H), 3.83 (s, 3H), 2.45-2.28 (m, 2H), 2.21 (ddd, J=3.4, 6.4, 9.8 Hz, 1H), 2.01-1.93 (m, 1H), 1.73-1.67 (m, 1H), 1.60 (d, J=6.2 Hz, 3H), 1.43 (t, J=7.0 Hz, 3H).

Compound (25): ¹H NMR (400 MHz, methanol-d₄) δ ppm 8.69 (d, J=2.0 Hz, 1H), 8.67 (br d, J=3.6 Hz, 1H), 8.19 (d, J=6.2 Hz, 1H), 8.04 (s, 1H), 8.02 (s, 1H), 6.68 (d, J=6.0 Hz, 1H), 5.16-5.09 (m, 1H), 4.68 (dt, J=2.8, 9.8 Hz, 1$H), 4.37 (q, J=7.0 Hz, 2H), 4.26-4.19 (m, 1H), 4.07-4.00 (m, 1H), 3.81 (s, 3H), 2.43-2.22 (m, 2H), 2.15 (td, J=6.8, 8.8 Hz, 1H), 2.08-2.00 (m, 1H), 1.78-1.70 (m, 1H), 1.58 (d, J=6.2 Hz, 3H), 1.45 (t, J=7.0 Hz, 3H).

### Example 4: Preparation of Compound (58)

### Synthesis of inhibitor: 2-[4-[(10S)-5,10-dimethyl-7,11-dioxa-4,5,15,17,21,22-hexazatetracyclo[16.3.1.1^{12,16}.0^{2,6}]tricosa-1(22),2(6),3,12(23),13,15,18,20-octaen-13-yl]-5-ethoxy-pyrazol-1-yl]-N,N-dimethyl-ethanamine (compound (58))

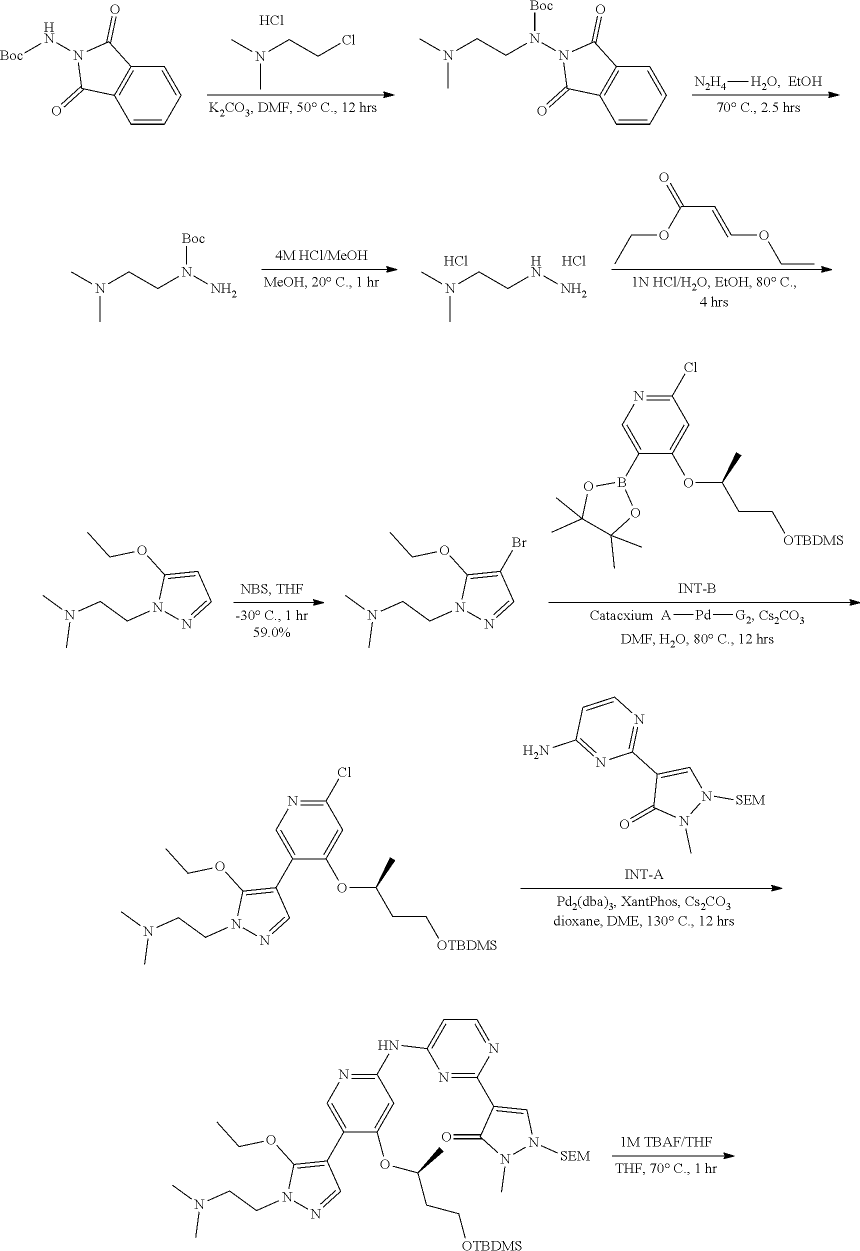

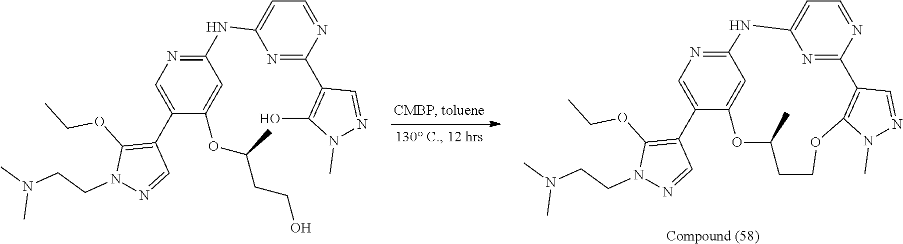

Step 1: a mixture of tert-butyl N-(1,3-dioxoisoindolin-2-yl)carbamate (5 g, 19.06 mmol, 1.0 eq), 2-chloro-N,N-dimethyl-ethanamine; hydrochloride (5.5 g, 38.18 mmol, 2.0 eq) and K₂CO₃ (8 g, 57.88 mmol, 3.0 eq) in DMF (80.0 mL) was stirred at 50° C. for 12 hours. The reaction mixture was quenched by addition saturated Na₂CO₃ aqueous solution (50 mL), and then extracted with EtOAc (25 mL*3). The combined organic layers were washed with brine (40 mL*3), dried over Na₂SO₄, filtered and concentrated under reduced pressure to give tert-butyl N-[2-(dimethylamino)ethyl]-N-(1,3-dioxoisoindolin-2-yl)carbamate (4 g, 47.8% yield, 76% purity) as a yellow oil. LC MS [M+H]⁺ m/z: calcd 334.2, found 334.2.

Step 2: a mixture of tert-butyl N-[2-(dimethylamino)ethyl]-N-(1,3-dioxoisoindolin-2-yl)carbamate (4 g, 12.00 mmol, 1.0 eq) and N₂H₄—H₂O (6.46 g, 126.46 mmol, 98% purity, 10.5 eq) in EtOH (100.0 mL) was stirred at 70° C. for 2.5 hours. The reaction mixture was filtered and concentrated under reduced pressure to give tert-butyl N-amino-N-[2-(dimethylamino)ethyl]carbamate (2 g, 82.0% yield) as a yellow oil.

¹H NMR (400 MHz, chloroform-cd 6 ppm 3.83 (br s, 2H), 3.48 (t, J=6.7 Hz, 2H), 2.48 (br t, J=6.5 Hz, 2H), 2.26 (s, 6H), 1.46 (s, 9H).

LCMS [M+H]⁺ m/z: calcd 204.2, found 204.3.

Step 3: a mixture of tert-butyl N-amino-N-[2-(dimethylamino)ethyl]carbamate (2 g, 9.84 mmol, 1.0 eq) and (50.0 mL) 4 M HCl/MeOH in MeOH (10.0 mL) was stirred at 20° C. for 1 hour. The reaction mixture was concentrated under reduced pressure to give 2-hydrazino-N,N-dimethyl-ethanamine; dihydrochloride (2 g, crude) as a yellow solid.

¹H NMR (400 MHz, methanol-d₄) δ ppm 3.41-3.36 (m, 2H), 3.36-3.34 (n, 2H), 2.94 (s, 6H)

Step 4: a mixture of 2-hydrazino-N,N-dimethyl-ethanamine; dihydrochloride (1.9 g, 10.79 mmol, 1.0 eq),ethyl (E)-3-ethoxyprop-2-enoate (4.0 mL, 27.69 mmol, 2.6 eq) and (30.0 mL) 1 M HCl/H₂O (2.8 eq) in EtOH (50.0 mL) was stirred at 80° C. for 4 hours. The mixture was adjusted to pH>10 with 1N NaOH aqueous solution and extracted with EtOAc (20 mL*3). The combined organic layers were washed with brine (30 mL*3), dried over Na₂SO₄, filtered and concentrated under reduced pressure. The residue was purified by flash silica gel chromatography (ISCO®; 20 g SepaFlash® Silica Flash Column, MeOH (0.05 v % TEA)/DCM with MeOH (0.05 v % TEA) from 0˜20%, flow rate: 80 mL/min, 254 nm) to afford 2-(5-ethoxypyrazol-1-yl)-N,N-dimethyl-ethanamine (0.63 g, 31.9% yield) as a yellow oil.

¹H NMR (400 MHz, chloroform-d) δ ppm 7.31 (d, J=1.8 Hz, 1H), 5.47 (d, J=1.6 Hz, 1H), 4.17-4.08 (m, 4H), 2.89 (t, J=6.8 Hz, 2H), 2.38 (s, 6H), 1.42 (t, J=7.1 Hz, 3H)

LCMS [M+H]⁺ m/z: calcd 184.1, found 184.0.

Step 5: to a mixture of 2-(5-ethoxypyrazol-1-yl)-N,N-dimethyl-ethanamine (0.63 g, 3.44 mmol, 1.0 eq) in THF (15.0 nmL) was added NBS (700 mg, 3.93 mmol, 1.1 eq) at −30° C. and the mixture was stirred at −30° C. for 1 hour. The reaction mixture was quenched by saturated Na₂SO₃ aqueous solution (20 mL) at 20° C., and extracted with EtOAc (15 mL*3). The combined organic layers were washed with brine (30 mL*3), dried over Na₂SO₄, filtered and concentrated under reduced pressure to give 2-(4-bromo-5-ethoxy-pyrazol-1-yl)-N,N-dimethyl-ethanamine (700 mg, 59.0% yield, 76% purity) as a yellow oil.

¹H NMR (400 MHz, chloroform-d) δ ppm 7.31 (s, 1H), 4.39 (q, J=7.0 Hz, 21), 4.13 (t, J=6.9 Hz, 2H), 2.82 (t, J=6.9 Hz, 2H), 2.36 (s, 6H), 1.42 (t, J=7.0 Hz, 3H)

LCMS [M+H]⁺ m/z: calcd 262.0, found 262.0.

Step 6: 2-(4-bromo-5-ethoxy-pyrazol-1-yl)-N,N-dimethyl-ethanamine (700 mg, 2.67 mmol, 1.0 eq), tert-butyl-[(3S)-3-[[2-chloro-5-(4,4,5,5-tetramethyl-1,3,2-dioxaborolan-2-yl)-4-pyridyl]oxy]butoxy]-dimethyl-silane (3 g, 6.78 mmol, 2.5 eq), [2-(2-aminophenyl)phenyl]-chloro-palladium; bis(1-adamantyl)-butyl-phosphane (180 mg, 0.269 mmol, 0.1 eq) and Cs₂CO₃ (2.6 g, 7.98 mmol, 3.0 eq) in DMF (15.0 mL) and H₂O (1.5 mL) was de-gassed and then heated to 80° C. for 12 hours under N₂. The reaction mixture was filtered. The filtrate was diluted with saturated Na₂CO₃ aqueous solution (20 mL) and extracted with EtOAc (30 mL*2). The combined organic layers were washed with brine (60 mL*3), dried over Na₂SO₄, filtered and concentrated under reduced pressure. The residue was purified by flash silica gel chromatography (ISCO®; 20 g SepaFlash® Silica Flash Column, MeOH (0.05 v % TEA)/DCM with MeOH (0.05 v % TEA) from 0˜15%, flow rate: 80 mL/min, 254 nm) to give 2-[4-[4-[(1S)-3-[tert-butyl(dimethyl)silyl]oxy-1-methyl-propoxy]-6-chloro-3-pyridyl]-5-ethoxy-pyrazol-1-yl]-N,N-dimethyl-ethanamine (1.1 g, 34.0% yield, 41% purity) as a yellow oil.

LCMS [M+H]⁺ m/z: calcd 497.3, found 497.2.

Step 7: 2-[4-[4-[(1S)-3-[tert-butyl(dimethyl)silyl]oxy-1-methyl-propoxy]-6-chloro-3-pyridyl]-5-ethoxy-pyrazol-1-yl]-N,N-dimethyl-ethanamine (1.1 g, 2.21 mmol, 1.0 eq), 4-(4-aminopyrimidin-2-yl)-2-methyl-1-(2-trimethylsilylethoxymethyl)pyrazol-3-one (560 mg, 1.74 mmol, 0.8 eq), Xantphos (130 mg, 0.225 mmol, 0.1 eq), Cs₂CO₃ (2 g, 6.14 mmol, 2.8 eq) and Pd₂(dba)₃ (202 mg, 0.221 mmol, 0.1 eq) in dioxane (25.0 mL) and DME (2.5 mL) was de-gassed and then heated to 130° C. for 12 hours under N₂. The reaction mixture was concentrated under reduced pressure. The residue was purified by flash silica gel chromatography (ISCO®; 20 g SepaFlash® Silica Flash Column, MeOH (0.05 v % NH₃·H₂O)/DCM with MeOH (0.05 v % NH₃·H₂O) from 0˜12%, flow rate: 80 mL/min, 254 nm) to give 4-[4-[[4-[(1S)-3-[tert-butyl(dimethyl)silyl]oxy-1-methyl-propoxy]-5-[1-[2-(dimethylamino)ethyl]-5-ethoxy-pyrazol-4-yl]-2-pyridyl]amino]pyrimidin-2-yl]-2-methyl-1-(2-trimethylsilylethoxymethyl)pyrazol-3-one (900 mg, 24.4% yield, 47% purity) as a yellow oil.

LCMS [M+H]⁺ m/z: calcd 782.4, found 782.5.

Step 8: a mixture of 4-[4-[[4-[(1S)-3-[tert-butyl(dimethyl)silyl]oxy-1-methyl-propoxy]-5-[1-[2-(dimethylamino)ethyl]-5-ethoxy-pyrazol-4-yl]-2-pyridyl]amino]pyrimidin-2-yl]-2-methyl-1-(2-trimethylsilylethoxymethyl)pyrazol-3-one (900 mg, 1.15 mmol, 1.0 eq) and (4.0 mL) 1 M TBAF/THF in THF (15.0 mL) was stirred at 70° C. for 1 hour. The reaction mixture was concentrated under reduced pressure. The residue was purified by flash silica gel chromatography (Biotage®, Column: SepaFlash® Sphercial C18, 60 g, 40-60 μm, 120 Å; MeCN/water (0.05% NH₃—H₂O) with MeCN from 0˜31%, 50 ml/min, 254 nm) to afford 4-[4-[[5-[1-[2-(dimethylamino)ethyl]-5-ethoxy-pyrazol-4-yl]-4-[(1S)-3-hydroxy-1-methyl-propoxy]-2-pyridyl]amino]pyrimidin-2-yl]-2-methyl-pyrazol-3-ol (700 mg, crude) as a yellow oil.

LCMS [M+H]⁺ m/z: calcd 538.3, found 538.3.

Step 9: a mixture of 4-[4-[[5-[1-[2-(dimethylamino)ethyl]-5-ethoxy-pyrazol-4-yl]-4-[(1S)-3-hydroxy-1-methyl-propoxy]-2-pyridyl]amino]pyrimidin-2-yl]-2-methyl-pyrazol-3-ol (700 mg, 1.30 mmol, 1.0 eq) and 2-(tributyl-λ⁵-phosphanylidene)acetonitrile (1.57 g, 6.51 mmol, 5.0 eq) in Tol. (30.0 mL) was stirred at 130° C. for 12 hours under N₂. The reaction mixture was concentrated under reduced pressure. The residue was purified by flash chromatography (ISCO®; 12 g SepaFlash® Silica Flash Column, DCM/MeOH (0.05% NH₃·H₂O) with MeOH (0.05% NH₃·H₂O) from 0˜20%, 40 mL/min, 254 nm) to give the crude product and the product was purified by preparative HPLC(Column: 2_Phenomenex Gemini C18 75*40 mm*3 um; Mobile phase: [water(NH₄HCO₃)-ACN]; B %: 30%-60%, 7.8 min, Column Temp. 30° C.) to afford 2-[4-[(10S)-5,10-dimethyl-7,11l-dioxa-4,5,15,17,21,22-hexazatetracyclo[16.3.1.1^{12,16}.0^{2,6}]tricosa-1(22),2(6),3,12(23),13,15,18,20-octaen-13-yl]-5-ethoxy-pyrazol-1-yl]-N,N-dimethyl-ethanamine (54.6 mg, 7.9% yield, 97.35% purity) as a off-white solid.

¹H NMR (400 MHz, methanol-d₄) δ ppm 8.69 (s, 1H), 8.19-8.16 (m, 2H), 7.99 (s, 1H), 7.58 (s, 1H), 6.63 (d, J=5.8 Hz, 1H), 5.13-5.05 (m, 1H), 4.65 (dt, J=3.4, 9.5 Hz, 1H), 4.22-4.13 (m, 3H), 4.05-3.95 (m, 2H), 3.77 (s, 3H), 2.79 (t, J=6.8 Hz, 2H), 2.31 (s, 6H), 2.29-2.20 (m, 2H), 1.47 (d, J=6.3 Hz, 3H), 1.30 (t, J=7.0 Hz, 3H)

LCMS [M+H]⁺ m/z: calcd 520.3, found 520.2.

### Example 5: Preparation of Compound (96)

### Synthesis of inhibitor: (10S)-13-[3-ethoxy-1-(1-methyl-4-piperidyl)pyrazol-4-yl]-5,10-dimethyl-7,11-dioxa-4,5,15,17,21,22-hexazatetracyclo[16.3.1.112,16.02,6]tricosa-1(22),2(6),3,12(23),13,15,18,20-octaene (compound (96))

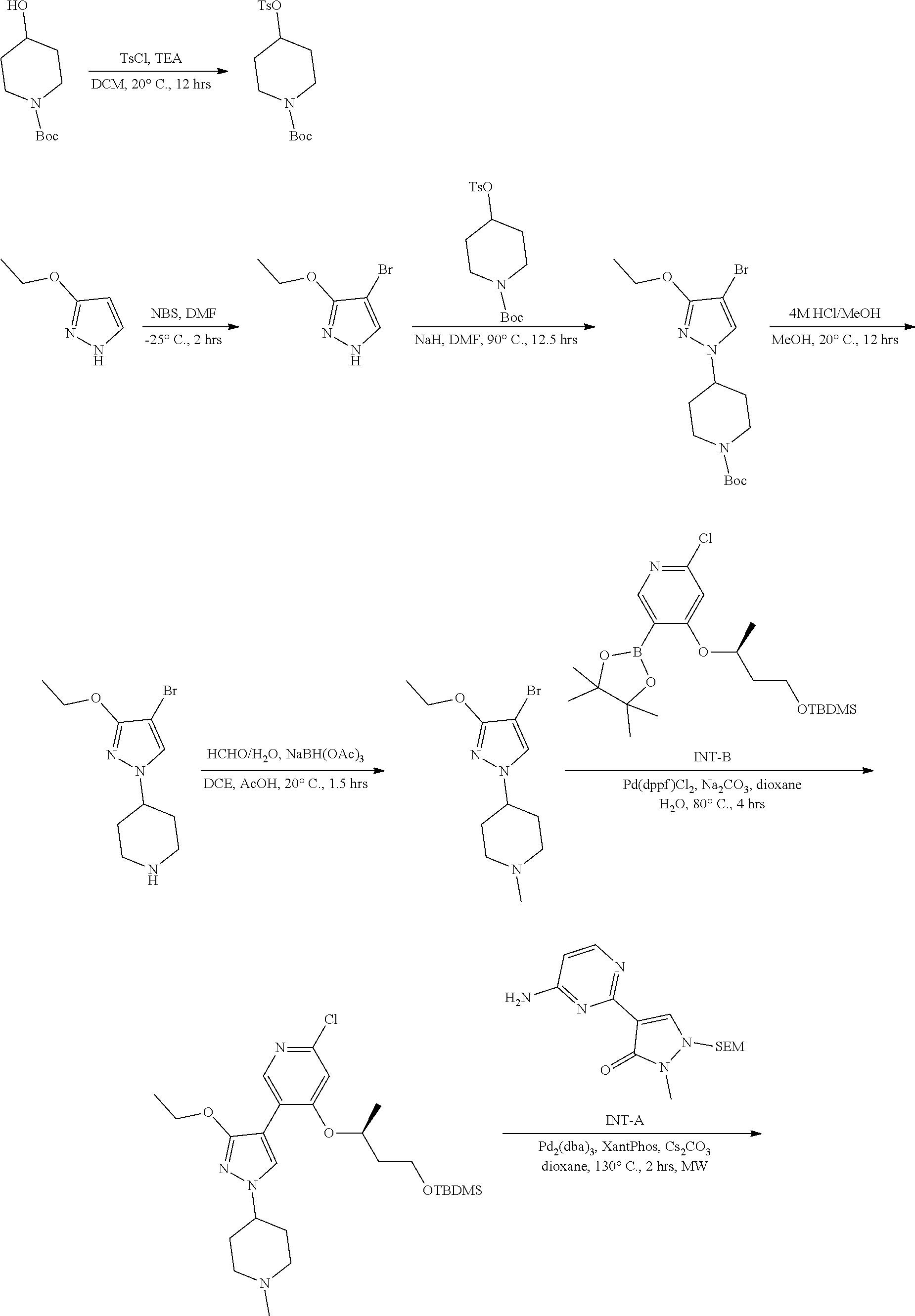

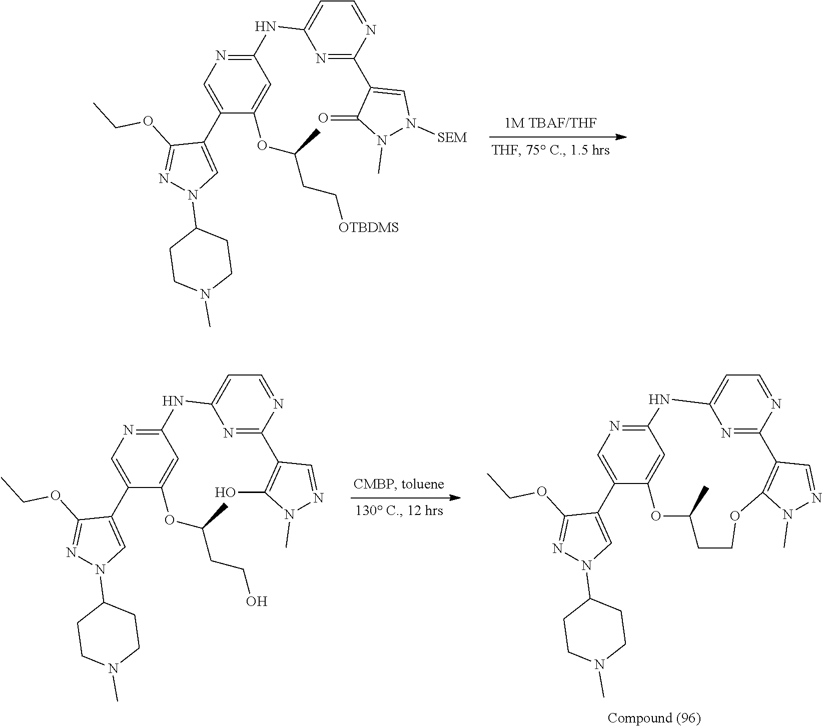

Step 1: to a solution of tert-butyl 4-hydroxypiperidine-1-carboxylate (5 g, 24.8 mmol, 1.0 eq) in DCM (50.0 mL) were added Et₃N (99.4 mmol, 13.8 mL, 4.0 ecg) and 4-methylbenzenesulfonyl chloride (9.47 g, 49.7 mmol, 2.0 eq). The mixture was stirred at 20° C. for 12 hours. The reaction mixture was concentrated. The residue was diluted with H₂O (100 mL) and extracted with EtOAc (150 mL*2). The combined organic layers were washed with brine (150 mL*1), dried over anhydrous Na₂SO₄, filtered and concentrated under reduced pressure. The residue was purified by flash chromatography (ISCO®; 80 g SepaFlash® Silica Flash Column, Petroleum ether gradient/EtOAc with EtOAc from 0˜50%, 100 mL/min, 254 nm) to afford tert-butyl 4-(p-tolylsulfonyloxy)piperidine-1-carboxylate (6.9 g, 77.3% yield, 99% purity) as an off white solid.

¹H NMR (400 MHz, CDCl₃) δ ppm 7.80 (d, J=8.4 Hz, 2H), 7.35 (d, J=8.0 Hz, 2H), 4.68 (tt, J=3.6, 7.2 Hz, 1H), 3.63-3.56 (m, 2H), 3.29-3.21 (m, 21H), 2.46 (s, 3H), 1.81-1.66 (m, 4H), 1.46-1.42 (m, 9H).

LCMS (ESI) [M+H]⁺ m/z: calcd 378.1, found 378.0.

Step 2: to a solution of 3-ethoxy-1H-pyrazole (1 g, 8.92 mmol, 1.0 eq) in DMF (20.0 mL) was added NBS (2.38 g, 13.4 mmol, 1.5 eq). The mixture was stirred at −25° C. for 2 hours. The reaction mixture was diluted with saturated aqueous Na₂CO₃ solution (50.0 mL) and extracted with EtOAc (80 mL*2). The combined organic layers were washed with brine (80 mL*1), dried over anhydrous Na₂SO₄, filtered and concentrated under reduced pressure. The residue was purified by flash chromatography (ISCO®; 20 g SepaFlash® Silica Flash Column, petroleum ether/EtOAc with EtOAc from 0˜20%, 40 mL/min, 254 nm) to afford 4-bromo-3-ethoxy-1:H-pyrazole (1.5 g, 88.1% yield, 100% purity) as a white solid.

¹H NMR (400 MHz, MeOD) δ ppm 7.52 (s, 1H), 4.21 (q, J=7.2 Hz, 2H), 1.37 (t, J=7.2 Hz, 3H).

LCMS (ESI) [M+H]⁺ m/z: calcd 191.0, found 190.8.

Step 3: to a solution of 4-bromo-3-ethoxy-1H-pyrazole (850 mg, 4.45 mmol, 1.0 eq) in DMF (10.0 mL) was added NaH (534 mg, 13.4 mmol, 60 wt % in mineral oil, 3.0 eq) at 20° C. for 30 mins. Then tert-butyl 4-(p-tolylsulfonyloxy)piperidine-1-carboxylate (2.05 g, 5.77 mmol, 1.30 eq) was added and the mixture was stirred at 90° C. for 12 hours. The reaction mixture was quenched by addition H₂O (50 mL) at 0° C., and then extracted with EtOAc (60 mL*2). The combined organic layers were washed with brine (50 mL*2), dried over anhydrous Na₂SO₄, filtered and concentrated under reduced pressure. The residue was purified by flash chromatography (ISCO®; 12 g SepaFlash® Silica Flash Column, Petroleum ether gradient/EtOAc with EtOAc from 0˜5%, 40 mL/min, 254 nm). The crude product was purified by prep-HPLC (column: Welch Xtimate C18 100*40 mm*3 μm; mobile phase: [water (TFA)-ACN]; B %: 40%-70%,10 min) to afford tert-butyl 4-(4-bromo-3-ethoxy-pyrazol-1-yl)piperidine-1-carboxylate (380 mg, 21.5% yield, 94% purity) as colorless oil.

¹H NMR (400 MHz, MeOD) δ ppm 7.55 (s, 1H), 4.25-4.08 (m, 5H), 3.02-2.81 (m, 2H), 2.04-1.95 (m, 2H), 1.82 (dq, J=4.4, 12.0 Hz, 21H), 1.47 (s, 9H), 1.35 (t, J=7.2 Hz, 3H).

LCMS (ESI) [M+H]⁺ m/z: calcd 374.1, found 375.9.

Step 4: to a solution of tert-butyl 4-(4-bromo-3-ethoxy-pyrazol-1-yl)piperidine-1-carboxylate (380 mg, 1.02 mmol, 1.0 eq) in MeOH (5.0 mL) was added 4M HCl/MeOH (5.0 mL, 20 mmol, 19.70 eq). The mixture was stirred at 20° C. for 12 hours. The reaction mixture was concentrated. The residue was diluted with MeOH (10 mL) and added saturated Na₂CO₃ aqueous solution to adjust pH˜8, concentrated under reduced pressure to afford 4-(4-bromo-3-ethoxy-pyrazol-1-yl)piperidine (270 mg, crude) as yellow oil.

LCMS (ESI) [M+H]⁺ m/z: calcd 274.0, found 275.8.

Step 5: to a solution of 4-(4-bromo-3-ethoxy-pyrazol-1-yl)piperidine (260 mg, 0.948 mmol, 1.0 eq) and formaldehyde (154 mg, 1.90 mmol, 37% purity, 2.0 eq) in DCE (10.0 mL) was added CH₃COOH (285 mg, 4.74 mmol, 5.0 eq). The mixture was stirred at 20° C. for 30 minutes. Then NaBH(OAc)₃ (1.0 g, 4.74 mmol, 5.0 eq) was added and the mixture was stirred at 20° C. for 1 hour. The reaction mixture was diluted with H₂O (10 ml) and extracted with EtOAc (10 mL*2). The combined organic layers were washed with brine (10 mL*1), dried over anhydrous Na₂SO₄, filtered and concentrated under reduced pressure. The residue was purified by flash chromatography (ISCO®; 4 g SepaFlash® Silica Flash Column, Petroleum ether/EtOAc with EtOAc from 0˜100%, then EtOAc/MeOH with MeOH from 0˜20%, 18 mL/min, 254 nm) to afford 4-(4-bromo-3-ethoxy-pyrazol-1-yl)-1-methyl-piperidine (260 mg, 95.1% yield, 100% purity) as yellow oil.

¹H NMR (400 MHz, CD₃OD) δ ppm 7.55 (s, 1H), 4.20 (q, J=7.2 Hz, 2H), 3.97 (tt, J=5.2, 10.4 Hz, 1H), 2.98 (br d, J=12.0 Hz, 2H), 2.33 (s, 3H), 2.23 (dt, J=3.6, 11.6 Hz, 2H), 2.06-1.96 (m, 4H), 1.35 (t, J=7.2 Hz, 3H).

LCMS (ESI) [M+H]⁺ m/z: calcd 288.1, found 287.9.

Step 6: a mixture of 4-(4-bromo-3-ethoxy-pyrazol-1-yl)-1-methyl-piperidine (250 mg, 0.868 mmol, 1.0 eq), tert-butyl-[(3S)-3-[[2-chloro-5-(4,4,5,5-tetramethyl-1,3,2-dioxaborolan-2-yl)-4-pyridyl]oxy]butoxy]-dimethyl-silane (498 mg, 1.13 mmol, 1.3 eq), Pd(dppf)Cl₂ (127 mg, 0.174 mmol, 0.2 eq), Na₂CO₃ (184 mg, 1.74 mmol, 2.0 eq) in dioxane (5.0 mL) and H₂O (1.0 mL) was degassed and purged with N₂ for 3 times, and then the mixture was stirred at 80° C. for 4 hours under N₂ atmosphere. The reaction mixture was diluted with H₂O (20 mL) and extracted with EtOAc (50 mL*2). The combined organic layers were washed with brine (50 mL*1), dried over anhydrous Na₂SO₄, filtered and concentrated under reduced pressure. The residue was purified by flash chromatography (ISCO®; 4 g SepaFlash® Silica Flash Column, Petroleum ether/EtOAc with EtOAc from 0˜100%, then EtOAc/MeOH with MeOH from 0˜20%, 18 mL/min, 254 nm) to afford tert-butyl-[(3S)-3-[[2-chloro-5-[3-ethoxy-1-(1-methyl-4-piperidyl)pyrazol-4-yl]-4-pyridyl]oxy]butoxy]-dimethyl-silane (140 mg, 19.4% yield, 63% purity) as yellow oil.

LCMS (ESI) [M+H]⁺ m/z: calcd 523.3, found 523.3.

Step 7: tert-butyl-[(3S)-3-[[2-chloro-5-[3-ethoxy-1-(1-methyl-4-piperidyl)pyrazol-4-yl]-4-pyridyl]oxy]butoxy]-dimethyl-silane (90 mg, 0.172 mmol, 1.0 eq), 4-(4-aminopyrimidin-2-yl)-2-methyl-1-(2-trimethylsilylethoxymethyl)pyrazol-3-one (61 mg, 0.189 mmol, 1.1 eq), Pd₂(dba)₃ (32 mg, 0.0344 mmol, 0.2 eq), Cs₂CO₃ (168 mg, 0.516 mmol, 3.0 eq) and XantPhos (40 mg, 0.0688 mmol, 0.4 eq) were taken up into a microwave tube in dioxane (2.0 mL). The sealed tube was heated at 130° C. for 2 hours under microwave. The reaction mixture was filtered and concentrated under reduced pressure. The residue was purified by flash chromatography (ISCO®; 4 g SepaFlash® Silica Flash Column, Petroleum ether/EtOAc with EtOAc from 0˜100%, EtOAc/MeOH with MeOH from 0˜20%, 18 mL/min, 254 nm) to afford 4-[4-[[4-[(1S)-3-[tert-butyl(dimethyl)silyl]oxy-1l-methyl-propoxy]-5-[3-ethoxy-1-(1-methyl-4-piperidyl)pyrazol-4-yl]-2-pyridyl]amino]pyrimidin-2-yl]-2-methyl-1-(2-trimethylsilylethoxymethyl)pyrazol-3-one (80 mg, 28.8% yield, 50% purity) as a light yellow solid.

LCMS (ESI) [M+H]⁺ m/z: calcd 808.5, found 808.7.

Step 8: to a solution of 4-[4-[[4-[(1S)-3-[tert-butyl(dimethyl)silyl]oxy-1-methyl-propoxy]-5-[3-ethoxy-1-(1-methyl-4-piperidyl)pyrazol-4-yl]-2-pyridyl]amino]pyrimidin-2-yl]-2-methyl-1-(2-trimethylsilylethoxymethyl)pyrazol-3-one (80 mg, 0.0496 mmol, 50% purity, 1.0 eq) in THF (5.0 mL) was added 1M TBAF/THF (0.1 mL, 0.1 mmol, 2.0 eq). The mixture was stirred at 75° C. for 1.5 hours. The reaction mixture was concentrated under reduced pressure. The crude product was purified by reversed-phase HPLC (Column: SepaFlash® Sphercial C18, 40 g, 40-60 μm, 120 Å; MeCN/water (0.5% NH₃—H₂O) with MeCN from 0˜30%, 50 m L/min, 254 nm) to afford 4-[4-[[5-[3-ethoxy-1-(1-methyl-4-piperidyl)pyrazol-4-yl]-4-[(1S)-3-hydroxy-1-methyl-propoxy]-2-pyridyl]amino]pyrimidin-2-yl]-2-methyl-pyrazol-3-ol (25 mg, 83.3% yield, 93% purity) as a yellow solid.

LCMS (ESI) [M+H]+ m/z: calcd 564.3, found 564.3.

Step 9: to a solution of 4-[4-[[5-[3-ethoxy-1-(1-methyl-4-piperidyl)pyrazol-4-yl]-4-[(1S)-3-hydroxy-1-methyl-propoxy]-2-pyridyl]amino]pyrimidin-2-yl]-2-methyl-pyrazol-3-o1 (25 mg, 0.0444 mmol, 1.0 eq) in toluene (15.0 mL) was added 2-(tributyl-λ⁵-phosphanylidene)acetonitrile (54 mg 0.222 mmol, 5.0 eq). The mixture was stirred at 130° C. for 12 hours under N₂. The reaction mixture was concentrated under reduced pressure. The residue was purified by flash chromatography (ISCO®; 4 g SepaFlash® Silica Flash Column, DCM/EtOAc with EtOAc from 0˜100%, DCM/MeOH with MeOH from 0˜20%, 18 mL/min, 254 nm). The crude product was further purified by preparative HPLC (column: 2_Phenomenex Gemini C18 75*40 mm*3 μm; mobile phase: [water(NH₄HCO₃)-ACN]; B %: 37%-67%, 9.5 min) to afford (10S)-13-[3-ethoxy-1-(1-methyl-4-piperidyl)pyrazol-4-yl]-5,10-dimethyl-7,11-dioxa-4,5,15,17,21,22-hexazatetracyclo[16.3.1.112,16.02,6]tricosa-1(22),2(6),3,12(23),13,15,18,20-octaene (4.3 mg, 17.7% yield, 99% purity) as a white solid.

¹H NMR (400 MHz, CD₃OD) δ ppm 8.73 (s, 1H), 8.65 (s, 1H), 8.22 (d, J=6.0 Hz, 1H), 8.02 (s, 1H), 7.94 (s, 1H), 6.68 (d, J=6.0 Hz, 1H), 5.22-5.12 (m, 1H), 4.77-4.72 (m, 1H), 4.31 (q, J=7.2 Hz, 2H), 4.27-4.21 (m, 1H), 4.11-4.00 (m, 1H), 3.82 (s, 3H), 3.05 (br d, J=12.0 Hz, 2H), 2.42-2.28 (m, 7H), 2.16-2.02 (m, 4H), 1.58 (d, J=6.4 Hz, 31H), 1.43 (t, J=7.2 Hz, 3H).

LCMS (ESI) [M+Na]⁺ m/z: calcd 568.3, found 568.2.

### Example 6: Preparation of Compound (105)

### Synthesis of inhibitor: (10S)-13-(3-ethoxy-1-methyl-pyrazol-4-yl)-5,10-dimethyl-7,11-dioxa-4,5,15,17,21,22-hexazatetracyclo[16.3.1.112,16.02,6]tricosa-1(22),2(6),3,12(23),13,15,18,20-octaene (compound (105))

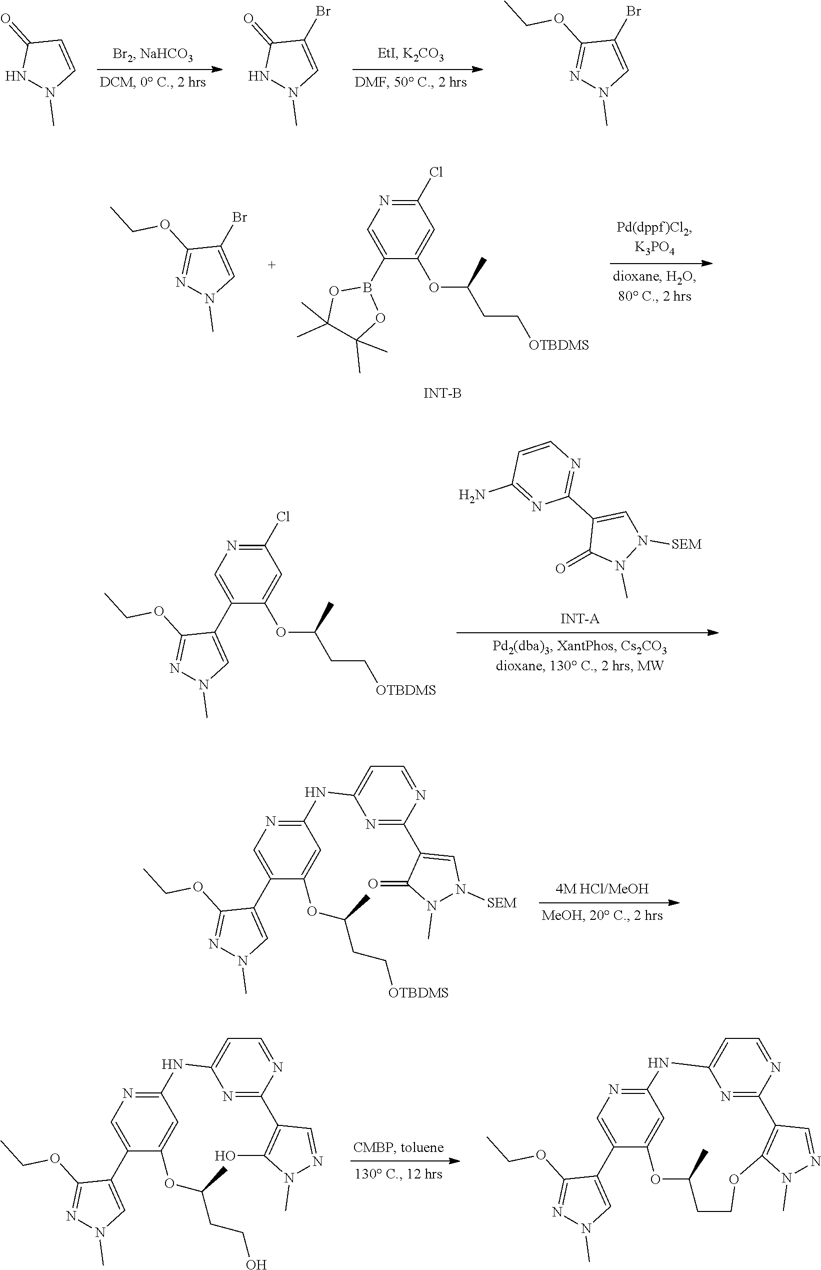

Step 1: to a solution of 2-methyl-1H-pyrazol-5-one (2 g, 20.4 mmol, 1 eq) in DCM (100 mL) was added NaHCO₃(2.06 g, 24.5 mmol, 1.2 eq), Br₂ (1.20 mL, 23.2 mmol, 1.14 eq). The mixture was stirred at 0° C. for 2 hours. The resulting mixture was quenched by addition of saturated Na₂SO₃ aqueous solution (50 mL) and extracted with DCM (50 mL*3). The combined organic layer was washed with brine (100 ml), dried over anhydrous Na₂SO₄, filtered and concentrated under reduced pressure to afford 4-bromo-2-methyl-1H-pyrazol-5-one (2 g, crude) as light-yellow solid.

LCMS (ESI) [M+H]⁺ m/z calcd 178.9, found 178.7.

Step 2: to a solution of 4-bromo-2-methyl-1H-pyrazol-5-one (2 g, 11.3 mmol, 1 eq) in DMF (20 mL) was added K₂CO₃ (4.0 g, 28.9 mmol, 2.56 eq). The mixture was stirred at 50° C. for 1 hour, iodoethane (2.20 g, 14.1 mmol, 1.25 eq) was added and the mixture was stirred at 50° C. for 1 hour. The resulting mixture was quenched by addition of water (30 mL) and extracted with EtOAc (30 mL*3). The combined organic layer was washed with brine (30 mL), dried over anhydrous Na₂SO₄, filtered and concentrated under reduced pressure to afford 4-bromo-3-ethoxy-1-methyl-pyrazole (2.0 g, crude) as yellow oil.

LCMS (ESI) [M+H]⁺ m/z calcd 205.0, found 204.9.

Step 3: to a solution of tert-butyl-[(3S)-3-[[2-chloro-5-(4,4,5,5-tetramethyl-1,3,2-dioxaborolan-2-yl)-4-pyridyl]oxy]butoxy]-dimethyl-silane (3.5 g, 7.92 mmol, 1.62 eq), 4-bromo-3-ethoxy-1-methyl-pyrazole (1 g, 4.88 mmol, 1 eq) in dioxane (50 mL)/H₂O (10 mL) was added Pd(dppf)Cl₂(400 mg, 0.547 mmol, 0.11 eq), K3PO4 (3.50 g, 16.5 mmol, 3.38 eq). The mixture was stirred at 80° C. for 2 hours under nitrogen. The resulting mixture was quenched by addition of water (100 mL) and extracted with EtOAc (100 mL*3). The combined organic layer was washed with saturated NH₄Cl aqueous solution (100 mL*2), brine (100 mL), dried over anhydrous Na₂SO₄, filtered and concentrated under reduced pressure to afford tert-butyl-[(3S)-3-[[2-chloro-5-(3-ethoxy-1-methyl-pyrazol-4-yl)-4-pyridyl]oxy]butoxy]-dimethyl-silane (800 mg, 37.3% yield) as yellow oil.

LCMS (ESI) [M+H]⁺ m/z calcd 440.2, found 440.2.

Step 4: to a solution of tert-butyl-[(3S)-3-[[2-chloro-5-(3-ethoxy-1-methyl-pyrazol-4-yl)-4-pyridyl]oxy]butoxy]-dimethyl-silane (450 mg, 1.02 mmol, 1 eq), 4-(4-aminopyrmidin-2-yl)-2-methyl-1-(2-trimethylsilylethoxymethyl)pyrazol-3-one (430 mg, 1.34 mmol, 1.31 eq) in dioxane (15 mL) was added Pd₂(dba)₃ (130 mg, 0.142 mmol, 0.14 eq), Cs₂CO₃ (1.01 g, 3.11 mmol, 3.04 eq) and Xantphos (178 mg, 0.308 mmol, 0.30 eq). The mixture was stirred at 130° C. for 2 hours under microwave. The resulting mixture was filtered, washed with DCM/MeOH (20 mL). The filtrate was concentrated under reduced pressure. The residue (combined with ES17560-230-P1) was purified by flash chromatography (ISCO®; 40 g SepaFlash® Silica Flash Column, DCM/MeOH with MeOH from 015%, flow rate@30 mL/min, 254 nm) to afford 4-[4-[[4-[(1S)-3-[tert-butyl(dimethyl)silyl]oxy-1-methyl-propoxy]-5-(3-ethoxy-1-methyl-pyrazol-4-yl)-2-pyridyl]amino]pyrimidin-2-yl]-2-methyl-1-(2-trimethylsilylethoxymethyl)pyrazol-3-one (700 mg, 65.4% yield) as yellow solid.

LCMS (ESI) [M+H]⁺ m/z calcd 725.4, found 725.4.

Step 5: to a solution of 4-[4-[[4-[(1S)-3-[tert-butyl(dimethyl)silyl]oxy-1-methyl-propoxy]-5-(3-ethoxy-1-methyl-pyrazol-4-yl)-2-pyridyl]amino]pyrimidin-2-yl]-2-methyl-1-(2-trimethylsilylethoxymethyl)pyrazol-3-one (700 mg, 0.965 mmol, 1 eq) in MeOH (5 mL) was added 4M HCl/MeOH (5 mL, 20 mmol). The mixture was stirred at 40° C. for 2 hours. The resulting mixture was concentrated under reduced pressure. The residue was diluted with H₂O (5 mL) and adjusted to pH=9 with saturated Na₂CO₃ aqueous solution, then extracted with DCM/i-PrOH (60 mL*3, v/v=20/1). The combined organic layer was concentrated under reduced pressure to afford 4-[4-[[5-(3-ethoxy-1-methyl-pyrazol-4-yl)-4-[(1S)-3-hydroxy-1-methyl-propoxy]-2-pyridyl]amino]pyrimidin-2-yl]-2-methyl-1H-pyrazol-3-one (360 mg, crude) as yellow solid.

LCMS (ESI) [M+H]⁺ m/z calcd 481.2, found 481.1.

Step 6: to a solution of 4-[4-[[5-(3-ethoxy-1-methyl-pyrazol-4-yl)-4-[(1S)-3-hydroxy-1-methyl-propoxy]-2-pyridyl]amino]pyrimidin-2-yl]-2-methyl-1H-pyrazol-3-one (360 mg, 0.749 mmol, 1 eq) in toluene (50 mL) was added CMBP (900 mg, 3.73 mmol, 4.98 eq). The mixture was stirred at 130° C. for 12 hours. The resulting mixture was concentrated under reduced pressure. The residue was purified by flash chromatography (ISCO®; 12 g SepaFlash® Silica Flash Column, DCM/EtOAc with EtOAc from 0-100%, then DCM/MeOH with MeOH From 0˜18%, flow rate@50 mL/min, 254 nm) to afford crude product (200 mg, yellow solid). The crude product was purified by preparative HPLC (Instrument: Gilson GX-281 Liquid Handler, Gilson 322 Pump, Gilson 156 UV Detector; Column: 2_Phenomenex Gemini C18 75*40 mm*3 um; Mobile phase A: H₂O with 0.05% NH₃—H₂O (v %);Mobile phase B: MeCN; Gradient: B from 35% to 68% in 7.8 min, hold 100% B for 2 min; Flow Rate: 25 mL/min; Column Temperature: 30° C.; Wavelength: 220 nm, 254 nm) to afford (10S)-13-(3-ethoxy-1-methyl-pyrazol-4-yl)-5,10-dimethyl-7,11-dioxa-4,5,15,17,21,22-hexazatetracyclo[16.3.1.112,16.02,6]tricosa-1(22),2(6),3,12(23),13,15,18,20-octaene (123 mg, 35.5% yield).

¹H NMR (400 MHz, methanol-d₄) δ ppm 8.67-8.71 (m, 1H), 8.65 (s, 1H), 8.18-8.23 (m, 1H), 8.02 (s, 1H), 7.83 (s, 1H), 6.64-6.69 (m, 1H), 5.11-5.20 (m, 1H), 4.73 (br d, J=3.0 Hz, 1H), 4.30 (q, J=7.0 Hz, 2H), 4.17-4.25 (m, 1H), 3.80 (d, J=12.0 Hz, 6H), 2.24-2.41 (n, 2H), 1.58 (d, J=6.3 Hz, 3H), 1.44 (t, J=7.0 Hz, 3H).

LCMS (ESI) [M+H]⁺ m/z calcd 463.2, found 463.2.

### Example 7: Preparation of Compound (151)

### Synthesis of inhibitor: (10S)-13-chloro-5,10-dimethyl-7,11-dioxa-4,5,15,17,21,22-hexazatetracyclo[16.3.1.112,16.02,6]tricosa-1(22),2(6),3,12(23),13,15,18,20-octaene (compound (151))

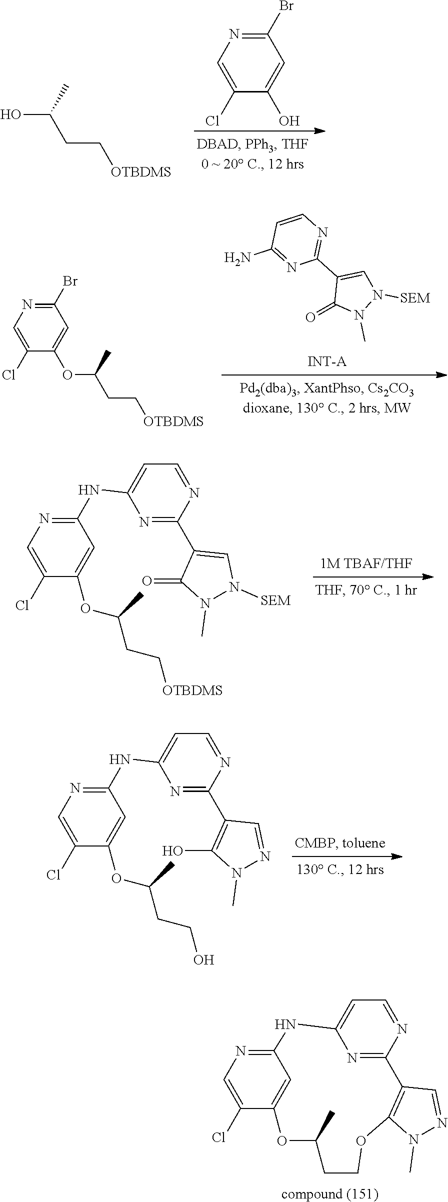

Step 1: a mixture of 2-bromo-5-chloro-pyridin-4-ol (1 g, 4.80 mmol, 1 eq), PPh₃ (3.77 g, 14.4 mmol, 3 eq) and THE (20 mL) was cooled to 0° C. and then di-tert-butyl azodicarboxylate (3.31 g, 14.4 mmol, 3 eq) in THF (5 mL) was added at 0° C. The mixture was stirred at 0° C. for 1 hour and then (2R)-4-[tert-butyl(dimethyl)silyl]oxybutan-2-ol (1.20 g, 5.87 mmol, 1.22 eq) was added at 0° C. The mixture was stirred at 20° C. for 11 hours. The resulting mixture was quenched by addition of water (100 mL) and extracted with EtOAc (100 mL*3). The combined organic layer was washed with saturated NH₄Cl aqueous solution (100 mL*2), brine (100 mL), dried over anhydrous Na₂SO₄, filtered and concentrated under reduced. The residue was purified by flash chromatography (ISCO®; 24 g AgelaFlash® Silica Flash Column, petroleum ether/EtOAc with EtOAc from 0˜10%, Flow Rate: 30 m L/min) to afford [(3S)-3-[(2-bromo-5-chloro-4-pyridyl)oxy]butoxy]-tert-butyl-dimethyl-silane (1.11 g, 58.6% yield) as yellow oil.

¹H NMR (400 MHz, chloroform-d) δ ppm 8.13-8.19 (m, 1H), 7.04 (s, 1H), 4.71 (sxt, J=6.2 Hz, 1H), 3.64-3.75 (m, 2H), 1.92-2.03 (n, 1H), 1.73-1.82 (n, 1H), 1.58 (d, J=4.5 Hz, 1H), 1.38 (d, 1=6.0 Hz, 3H), 0.80-0.86 (M, 9H), −0.02 (d, J=13.8 Hz, 6H).

Step 2: a mixture of [(3S)-3-[(2-bromo-5-chloro-4-pyridyl)oxy]butoxy]-tert-butyl-dimethyl-silane (1 g, 2.53 mmol, 1 eq), 4-(4-iminopyrimidin-2-yl)-2-methyl-1-(2-trimethylsilylethoxymethyl)pyrazol-3-one (900 mg, 2.80 mmol, 1.11 eq), Pd₂(dba)₃ (260 mg, 0.284 mmol, 1.12e-1 eq), XantPhos (300 mg, 0.518 mmol, 0.205 eq), Cs₂CO₃ (1.66 g, 5.09 mmol, 2.01 eq) and dioxane (15 mL) was stirred at 130° C. for 2 hours under microwave. The mixture was filtered and concentrated under reduced pressure. The residue was purified by flash chromatography (ISCO®; 24 g AgelaFlash® Silica Flash Column, DCM/MeOH with MeOH from 0˜20%, Flow Rate: 30 mL/min) to afford a crude product which was purified by flash chromatography (Column: SepaFlash® Sphercial C18, 25 g, 40-60 μm, 120 Å; MeCN/water (0.5% NH₃—H₂O) with MeCN from 0˜89%, 25 mL/min, 220 nm) to give 4-[4-[[4-[(1S)-3-[tert-butyl(dimethyl)silyl]oxy-1-methyl-propoxy]-5-chloro-2-pyridyl]amino]pyrimidin-2-yl]-2-methyl-1-(2-trimethylsilylethoxymethyl)pyrazol-3-one (320 mg, 18.9% yield, 95% purity) as yellow oil.

LCMS (ESI) [M+H]⁺ m/z: calcd 635.3, found 635.3.

Step 3: a mixture of 4-[4-[[4-[(1S)-3-[tert-butyl(dimethyl)silyl]oxy-1-methyl-propoxy]-5-chloro-2-pyridyl]amino]pyrimidin-2-yl]-2-methyl-1-(2-trimethylsilylethoxymethyl)pyrazol-3-one (320 mg, 0.504 mmol, 1 eq), 4-[4-[[4-[(1S)-3-[tert-butyl(dimethyl)silyl]oxy-1-methyl-propoxy]-5-chloro-2-pyridyl]amino]pyrimidin-2-yl]-2-methyl-1H-pyrazol-3-one (320 mg, 0.634 mmol, 1 eq) and 1M TBAF/THF (2.0 mL, 2.0 mmol, 2.12 eq) and THF (4 mL) was stirred at 70° C. for 1 hour. The mixture was concentrated under reduced pressure to give a residue which was purified by flash chromatography (Column: SepaFlash® Sphercial C18, 25 g, 40-60 μm, 120 Å; MeCN/water (0.5% NH₃—H₂O) with MeCN from 0˜45%, 25 mL/min, 220 nm) to give 4-[4-[[5-chloro-4-[(1S)-3-hydroxy-1-methyl-propoxy]-2-pyridyl]amino]pyrimidin-2-yl]-2-methyl-pyrazol-3-ol (500 mg, crude) as yellow oil. LC MS (ESI) [M+H]⁺ m/z: calcd 391.1, found 391.0.

Step 4: a mixture of 4-[4-[[5-chloro-4-[(1S)-3-hydroxy-1-methyl-propoxy]-2-pyridyl]amino]pyrimidin-2-yl]-2-methyl-pyrazol-3-ol (400 mg, 1.02 mmol, 1 eq), 2-(tributyl-λ⁵-phosphanylidene)acetonitrile (1.24 g, 5.14 mmol, 5.02 eq) and toluene (20 mL) was stirred at 130° C. for 12 hours. The mixture was filtered and concentrated under reduced pressure. The residue was purified by flash chromatography (ISCO®; 24 g AgelaFlash® Silica Flash Column, DCM/MeOH with MeOH from 0˜10%, Flow Rate: 30 mL/min, 254 nm) to afford (10S)-13-chloro-5,10-dimethyl-7,11-dioxa-4,5,15,17,21,22-hexazatetracyclo[16.3.1.112,16.02,6]tricosa-1(22),2(6),3,12(23),13,15,18,20-octaene (700 mg, crude) as yellow solid. 20 mg of this crude product was purified by preparative HPLC (Instrument: Gilson GX-281 Liquid Handler, Gilson 322 Pump, Gilson 156 UV Detector; Column: Waters Xbridge 150×25 mm×5 μm; Mobile phase A: H₂O with 0.05% NH₃—H₂O (v %); Mobile phase B: MeCN; Gradient: B from 52% to 82% in 9.5 min, hold 100% B3 for 2.5 min; Flow Rate: 25 mL/min; Column Temperature: 30° C.; Wavelength: 220 nm, 254 nm) to afford (10S)-13-chloro-5,10-dimethyl-7,11-dioxa-4,5,15,17,21,22-hexazatetracyclo[16.3.1.112,16.02,6]tricosa-1(22),2(6),3,12(23),13,15,18,20-octaene (5 mg) as white solid.

¹H NMR (400 MHz, methanol-di) 5 ppm 8.83 (s, 1H), 8.25 (d, J=6.4 Hz, 1H), 8.17 (s, 1H), 8.10 (s, 1H), 6.82 (d, J=6.4 Hz, 1H), 5.13-5.19 (m, 1H), 4.68-4.74 (m, 1H), 4.32-4.41 (m, 2H), 3.88 (s, 3H), 2.33-2.41 (m, 2H), 1.59 (d, J=6.3 Hz, 3H).

LCMS (ESI) [M+H]⁺ m/z: calcd 373.1, found 373.1.

### Example 8: Preparation of Compound (127)

### Synthesis of inhibitor: (10S)-5,10-dimethyl-13-[1-methyl-5-[(4-methylpiperazin-1-yl)methyl]pyrrol-3-yl]-7,11-dioxa-4,5,15,17,21,22-hexazatetracyclo[16.3.1.1^{12,16}.0^{2,6}]tricosa-1(22),2(6),3,12(23),13,15,18,20-octaene (compound (127))

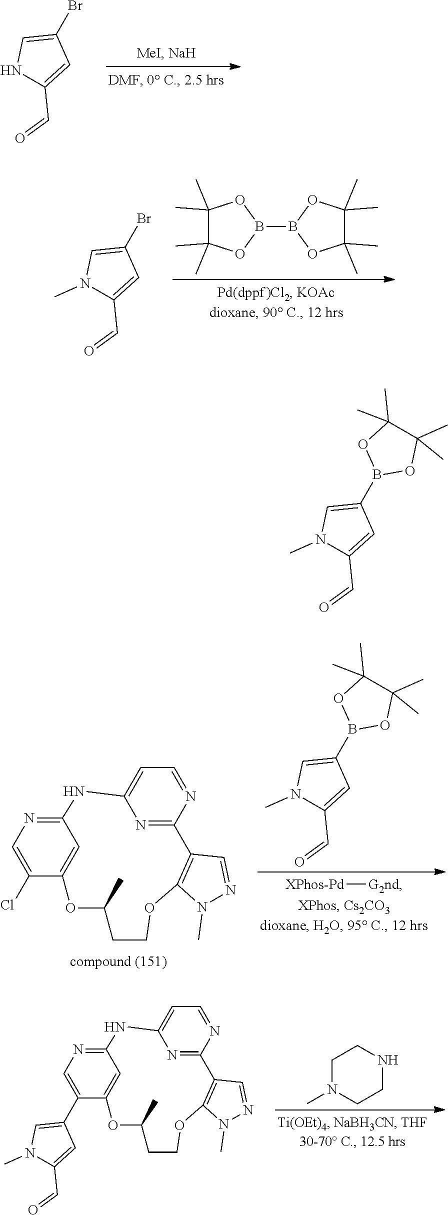

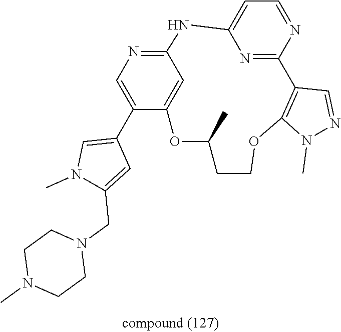 [SMILES: C[C@H]1CCOc2c(cnn2C)-c2nccc(n2)Nc2cc(c(-c3cc(CN4CCN(C)CC4)n(C)c3)cn2)O1]

Step 1: to a solution of 4-bromo-1H-pyrrole-2-carbaldehyde (1 g, 5.75 mmol, 1.0 eq) in DMF (20.0 mL) was added NaH (500 mg, 12.5 mmol, 60 wt % purity in mineral oil, 2.2 eq) at 0° C. After addition, the mixture was stirred at 0° C. for 30 minutes, and then Mel (1.82 g, 12.9 mmol, 2.2 eq) was added dropwise at 0° C. The resulting mixture was stirred at 20° C. for 1 hour. The reaction mixture was poured into ice-water (3 mL) and stirred for 3 minutes. The aqueous phase was extracted with EtOAc (20 mL*2) and the combined organic phase was washed with brine (15 mL*3), dried with anhydrous Na₂SO₄, concentrated under reduced pressure. The residue was purified by flash chromatography (ISCO®; 20 g SepaFlash® Silica Flash Column, petroleum ether/EtOAc with EtOAc from 0˜15%, flow rate: 80 mL/min, 254 nm) to afford compound 4-bromo-1-methyl-pyrrole-2-carbaldehyde (1 g, 74.3% yield, 80% purity) as a light yellow oil.

LCMS [M+H]⁺ m/z: calcd 187.9, found 189.7.

Step 2: 4-Bromo-1-methyl-pyrrole-2-carbaldehyde (300 mg, 1.60 mmol, 1.0 eq), (Bpin)₂ (810 mg, 3.19 mmol, 2.0 eq), Pd(dppf)Cl₂-DCM (130 mg, 0.159 mmol, 0.1 eq) and KOAc (314 mg, 3.20 mmol, 2.0 eq) in dioxane (10.0 mL) was de-gassed and then heated to 100° C. for 12 hours under N₂. The reaction mixture was filtered and the filter cake was washed with EtOAc (10 mL*2), then the reaction mixture was concentrated under reduced pressure. The residue was purified by flash chromatography (ISCO®; 12 g SepaFlash® Silica Flash Column, petroleum ether/EtOAc with EtOAc from 0˜20%, flow rate: 80 ml/min, 254 nm) to afford compound 1-methyl-4-(4,4,5,5-tetramethyl-1,3,2-dioxaborolan-2-yl)pyrrole-2-carbaldehyde (190 mg, 42.0% yield, 83% purity) as a light yellow solid.

LCMS [M+H]⁺ m/z: calcd 236.1, found 236.0.

Step 3: compound (151) (50 mg, 0.134 mmol, 1.0 eq), 1-methyl-4-(4,4,5,5-tetramethyl-1,3,2-dioxaborolan-2-yl)pyrrole-2-carbaldehyde (90 mg, 0.382 mmol, 2.9 eq), XPhos-Pd-G2 (11 mg, 0.013 mmol, 0.1 eq), XPhos (7 trig, 0.014 mmol, 1.0.1 eq) and Cs₂CO₃ (90 mg, 0.276 mmol, 2.0 eq) in dioxane (5.0 mL) and H₂O (1.0 mL) was de-gassed and then heated to 95° C. for 12 hours under N₂. The mixture was filtered and the filter cake was washed with DCM (10 mL*2), then the combined filtrate was concentrated under reduced pressure. The residue was purified by flash silica gel chromatography (ISCO®; 4 g SepaFlash® Silica Flash Column, MeOH (0.05% TEA)/DCM with MeOH (0.05% TEA) from 0˜20%, flow rate: 30 mL/min, 254 nm) to afford compound 4[(10S)-5,10-dimethyl-7,11-dioxa-4,5,15,17,21,22-hexazatetracyclo[16.3.1.^{12,16}.0^{2,6}]tricosa-1(22),2(6),3,12(23),13,15,18,20-octaen-13-yl]-1-methyl-pyrrole-2-carbaldehyde (89 mg, crude) as a light yellow solid.

LCMS [M+H]⁺ m/z: calcd 446.2, found 446.0.

Step 4: a mixture of 4-[10S)-5,10-dimethyl-7,11-dioxa-4,5,15,17,21,22-hexazatetracyclo-[16.3.1.1^{12,16}.0^{2,6}]-tricosa-1(22),2(6),3,12(23),13,15,18,20-octaen-13-yl]-1-methyl-pyrrole-2-carbaldehyde (89 rug, 0.199 mmol, 1.0 eq), 1-methylpiperazine (105 mg, 1.05 mmol, 5.3 eq) and Ti(OEt)₄ (240 m g, 1.05 mmol, 5.3 eq) in THE (10.0 mL) was stirred at 70° C. for 12 hours. Then NaBH₃CN (73 mg, 1.16 mmol, 5.8 eq) was added and the mixture was stirred at 30° C. for 30 minutes. The reaction mixture was quenched by addition H₂O (0.2 mL) and saturated Na₂CO₃ aqueous solution (0.2 mL), then silica gel was added and the mixture was dried over Na₂SO₄. The mixture was stirred at 20° C. for 15 minutes. The mixture was filtered and the filter cake was washed with DCM/MeOH (15 mL*5, v/v: 10/1) and the combined filtrate was dried over Na₂SO₄ and concentrated under reduced pressure. The crude product was purified by preparative HPLC (column: Phenomenex Gemini-NX 80*40 mm*3 μm; mobile phase: [water (10 mM NH₄HCO₃)-ACN]; B %: 30%-60%, 9.5 min. Temp: 30° C.) to afford (10S)-5,10-dimethyl-13-[1-methyl-5-[(4-methylpiperazin-1-yl)methyl]pyrrol-3-yl]-7,11-dioxa-4,5,15,17,21,22-hexazatetracyclo[163.1.1^{12,16}.0^{2,6}]tricosa-1(22),2(6),3,12(23),13,15,18,20-octaene (44.1 mg, 39.4% yield, 95% purity) as a white solid.

¹H NMR (400 MHz, methanol-d₄) δ ppm 8.67 (s, 1H), 8.24 (s, 1H), 8.20 (d, J=6.0 Hz, 1H), 8.02 (s, 1:H), 7.19 (d, J=1.8 Hz, 1H), 6.66 (d, J=6.0 Hz, 1H), 6.41 (d, J=1.8 Hz, 1H), 5.20-5.10 (m, 1H), 4.79-4.68 (m, 1H), 4.25-4.14 (m, 1H), 3.80 (s, 3H), 3.68 (s, 3H), 3.51 (s, 2H), 2.54 (br s, 7H), 2.33 (br d, P=4.5 Hz, 2H), 2.31 (s, 3H), 2.29-2.15 (m, 1H), 1.57 (d, J=6.3 Hz, 3H).

LCMS [M+H]⁺ m/z: calcd 530.3, found 530.1.

### Example 9: In Vitro Assays

The biological activity of compounds described herein can be studied according to standard methods known in the art. Methods can be used to study inhibition of EGFR, including mutant forms of EGFR comprising L858R, T790M, C797S, and/or Del19 mutations, or any combination thereof (e.g., L858R single, double, or triple mutants). Exemplary, non-limiting methods are described herein.

### Kinase Assays

Assays using an in vitro kinase assay kit (HTRF KinEASE-TK kit) can be used to study the inhibitory activity of compounds described herein with respect to EGFR mutants such as ECFR^{L858R}, EGFR^{L858R/T790M}, and EGFR^{L858R/T790M/C797S}.

### Ba/F3 Viability Assays

Inhibition of cell proliferation can be studied using Ba/F3 viability assays, including the Promega CellTiter-Glo cell viability assay. This assay can be used to study the effect of compounds described herein in the following assays: (1) Ba/F3 Parental; (2) Ba/F3 EGFR-Del19/T790M; (3); Ba/F3 EGFR-Del19/C797S; and (4) Ba/F3 EGFR-Del119/T790M/C797S.

### P-EGFR Signaling Assays

Phosphorylation of EGFR can be studied using multiplex immunoassay kits such as Phospho-EGFR (Tyr1068) Total EGFR MULTI-SPOT® 96 HB 4-Spot Custom EGFR Duplex ANALYTES assay.

One hundred thirty six compounds were studied using the kinase (EGFR) and BA/F3 assays described herein, with more than 60% of the tested compounds providing IC₅₀<50 nM values in all six assays. Accordingly, compounds of the invention are a new general class of kinase inhibitors, including potent inhibitors of EGFR mutants.

Exemplary kinase inhibition (Kinase) and anti-proliferation activity (Ba/F3) data are shown in Table 1 for certain compounds of the invention and are categorized according to the below legend.

TABLE 1
In vitro Assay Data

| | KINASE | KINASE | KINASE | BAF3 | BAF3 | BAF3 |
|---|---|---|---|---|---|---|
| compound | LTC | LT | L | DTC | DT | DC |
| (15) | A | A | A | A | A | A |
| (16) | A | A | A | A | A | A |
| (18) | A | A | A | A | A | A |
| (20) | A | A | A | A | A | A |
| (29) | A | A | A | A | A | A |
| (30) | A | A | A | A | A | A |
| (32) | A | A | A | A | A | A |
| (33) | A | A | A | A | B | B |
| (34) | A | A | A | A | B | A |
| (35) | C | A | D | C | C | D |
| (36) | A | A | A | A | A | A |
| (37) | A | A | A | A | A | A |
| (40) | A | A | A | A | A | A |
| (42) | A | A | A | A | A | A |
| (43) | A | A | A | A | A | A |
| (48) | A | A | A | A | A | A |
| (56) | A | A | A | A | A | A |
| (62) | A | A | A | A | A | A |
| (70) | A | A | A | A | A | A |
| (74) | C | C | D | C | C | D |
| (75) | A | A | B | A | B | C |
| (77) | A | A | A | A | B | C |
| (84) | A | A | A | A | A | A |
| (85) | A | A | A | A | A | A |
| (89) | C | A | D | D | D | D |
| (92) | A | A | A | A | A | A |
| (103) | A | A | C | C | C | C |
| (115) | A | A | A | A | A | A |
| (123) | A | A | A | A | A | A |
| (124) | A | A | A | A | A | A |
| (127) | A | A | A | A | A | A |
| (128) | A | A | A | A | B | B |
| (133) | A | A | A | A | A | A |
| (137) | A | A | A | A | A | A |
| (138) | A | A | A | A | B | B |
| (141) | A | A | A | A | A | A |
| (144) | A | A | A | A | A | A |
| (145) | A | A | C | C | C | C |
| (146) | A | A | A | A | A | A |
| (147) | A | A | A | A | A | A |
| (148) | A | A | A | A | A | A |
| (149) | A | A | A | A | B | A |
| (150) | A | A | A | A | A | A |
| (151) | A | A | B | C | C | C |
| (152) | A | A | A | A | A | A |
| (153) | A | A | C | C | C | C |
| (159) | A | A | A | A | A | A |
| (160) | A | A | A | A | A | A |
| (169) | A | A | A | A | A | A |

Legend:
A = IC₅₀ < 50 nM
B = 50 nM ≤ IC₅₀ < 100 nM
C = 100 nM ≤ IC₅₀ < 1000 nM
D = IC₅₀ ≥ 1000 nM

From the ongoing description one skilled in the art can easily ascertain the essential characteristics of this invention, and without departing from the spirit and scope thereof, can make various changes and modifications of the invention to adapt it to various usages and conditions.

All references, patents or applications, U.S. or foreign, cited in the application are hereby incorporated by reference as if written herein in their entireties. Where any inconsistencies arise, material literally disclosed herein controls.

What is claimed is:

1. A compound of Formula I: 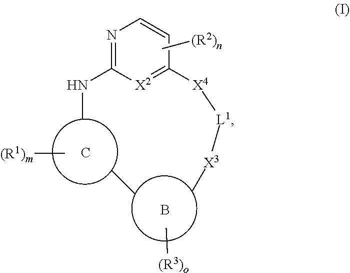
or a pharmaceutically acceptable salt thereof, wherein
X² is independently N or CR⁵;
each of X³ and X⁴ is independently a covalent bond, O, S, NR⁶, C(O)NR⁶, NR⁶C(O), NR⁶C(O)NR⁶, or (C(R⁷)₂)_{q};
L¹ is independently a covalent bond, C₁₋₆ heteroalkylene, C₁₋₆ alkylene, C₂₋₆ alkenylene, C₂₋₆ alkynylene, C₃₋₆ cycloalkylene, 3- to 10-membered heterocyclylene, phenylene, or 5- to 10-membered heteroarylene;
each R¹ and R² is independently 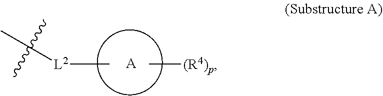
OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹(O)R¹⁰, NR¹¹CO₂R¹⁰, NR¹¹C(O)NR₈R₉, or (CH₂)ᵣR¹², or two R¹ or two R², together to which the atoms they are attached form a 5- to 10-membered ring;
L² is independently a covalent bond, O, NR^{L}, C(O), C(O)NR^{L}, NR^{L}C(O), CR^{L}₂;
R^{L} is independently H or C₁₋₆ alkyl;
A is independently phenyl, naphthyl, 5- to 13-membered heteroaryl, C₃-C₁₀ cycloaliphatic, or 3- to 10-membered heterocyclyl;
B is independently phenyl, naphthyl, 5- to 13-membered heteroaryl, C₃-C₁₀ cycloaliphatic, or 3- to 10-membered heterocyclyl;
C is independently 5- or 6-membered heteroaryl;
each R³ is independently OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, or (CH₂)ᵣR¹²;
each R⁴ is independently H, OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, NR¹¹(CH₂)ₛNR⁸R⁹, (CH₂)ₜNR⁸R⁹, (CH₂)ₜOH, (CH₂)ₜOCH₃, O(CH₂)ₜOH, O(CH₂)ₜOCH₃, O(CH₂)ᵣR¹², or (CH₂)ᵣR¹²; or R⁴ and R⁶ or R⁴ and R⁷, together with the atoms to which they are attached, form a 5- to 6-membered ring;
each R⁵ is independently H, OH, CN, halogen, C₁₋₆ aliphatic, C_{J}-6 alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹CC(O)NR⁸R⁹, or (CH₂)ᵣR¹²;
each R⁶ is independently H, a N-protecting group, or C₁₋₆ alkyl; or R⁶ and R⁴, together with the atoms to which they are attached, form a 5- to 6-membered ring;
each R⁷ is independently H or C₁₋₆ alkyl; or two R⁷ on the same carbon combine to from an oxo (═O) group; or R⁷ and R⁴, together with the atoms to which they are attached, form a 5- to 6-membered ring;
each R⁸, R⁹, and R¹¹ is independently H or C₁₋₆ alkyl; or R⁸ and R⁹, together with the nitrogen atom to which they are attached, form a 3- to 10-membered heterocyclyl, or R⁸ and R¹¹, together with the atoms to which they are attached, form a 3- to 10-membered heterocyclyl;
each R¹⁰ is independently C₁₋₆ aliphatic, C₃-C₁₀ cycloaliphatic, 3- to 10-membered heterocyclyl, phenyl, naphthyl, or a 5- to 12-membered heteroaryl, or R¹⁰ and R¹¹, together with the atoms to which they are attached, form a 3- to 10-membered heterocyclyl;
each R¹² is independently C₃-C₁₀ cycloaliphatic, 3- to 10-membered heterocyclyl, phenyl, naphthyl, or a 5- to 12-membered heteroaryl;
each m, n, and o, is independently 0, 1, or 2;
each p is independently 0, 1, 2; 3, or 4;
each q is independently 1 or 2;
each r is independently an integer of 0-4;
each s is independently an integer of 2-6; and
each t is independently an integer of 1-6.

2. The compound of claim 1, wherein at least one m or n is not 0.

3. The compound of claim 1 or 2, wherein one of R¹ and R² is present and is Substructure A or halogen.

4. The compound of any one of claims 1-3, wherein one of R¹ and R² is present and is Substructure A.

5. The compound of any one of claims 1-4, wherein no more than one Substructure A is present.

6. The compound of any one of claims 1-5, wherein C is 5- or 6-membered N-containing heteroaryl.

7. The compound of claim 6, wherein C is pyridyl, pyrimidyl, pyrazolyl, pyrrolyl, thiazolyl, oxazolyl, or imidazolyl.

8. The compound of any one of claims 1-7, having a structure according to Formula (I-A), 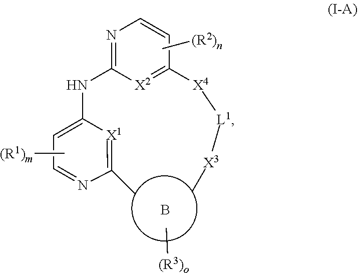
or a pharmaceutically acceptable salt thereof, wherein
X¹ is N or CR⁵.

9. The compound of any one of claims 1-7, having a structure according to Formula (I-B) 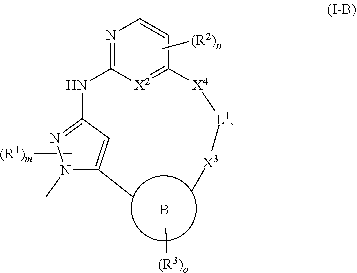
or a pharmaceutically acceptable salt thereof, wherein
m is 0 or 1.

10. The compound of any one of claims 1-7, having a structure according to Formula (I-C), 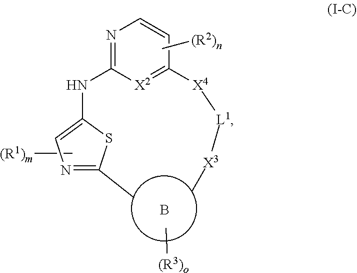
or a pharmaceutically acceptable salt thereof, wherein
m is 0 or 1.

11. The compound of any one of claims 1-8, having a structure according to Formula (II), 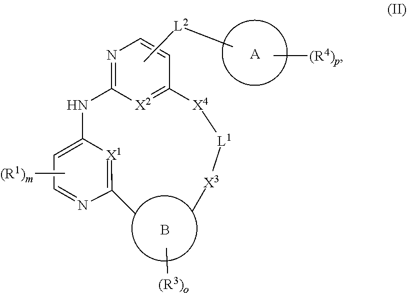
or a pharmaceutically acceptable salt thereof, wherein
each R¹ is independently OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, or R¹².

12. The compound of claim 11, having a structure according to Formula (II-A), 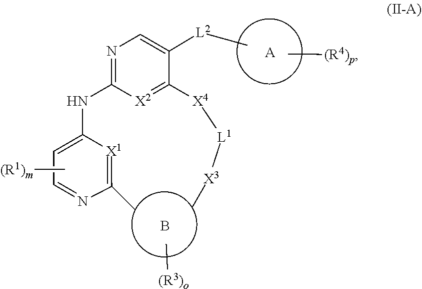
or a pharmaceutically acceptable salt thereof.

13. The compound of claim 11 or 12, wherein m is 0.

14. The compound of any one of claims 1-8, having a structure according to Formula (III), 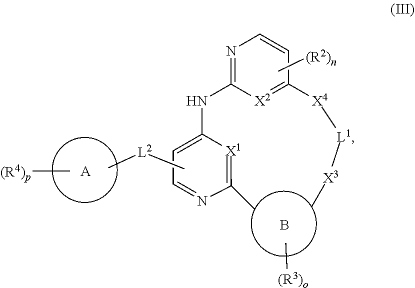
or a pharmaceutically acceptable salt thereof, wherein
each R² is independently OH, CN, halogen, C₁₋₆ aliphatic, C₁₋₆ alkoxy, NR⁸R⁹, C(O)R¹⁰, CO₂R¹⁰, C(O)NR⁸R⁹, NR¹¹C(O)R¹⁰, NR¹¹CO₂R¹⁰, NR¹¹C(O)NR⁸R⁹, or R¹².

15. The compound of claim 14, having a structure according to Formula (III-A), 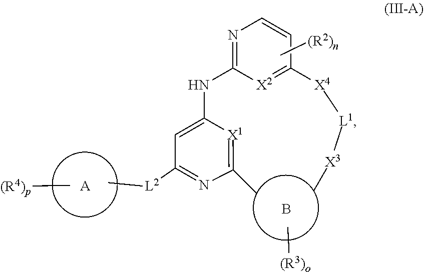
or a pharmaceutically acceptable salt thereof.

16. The compound of claim 14 or 15, wherein n is 0.

17. The compound of any one of claims 8-16, wherein each of X¹ and X² is independently N or CH.

18. The compound of any one of claims 8-17, wherein X¹ is N.

19. The compound of any one of claims 1-18, wherein X² is CH.

20. The compound of any one of claims 1-19, wherein X³ is O.

21. The compound of any one of claims 1-15 and 17-20, wherein X⁴ is NR⁶, R² is Substructure A, and wherein
R⁴ and R⁶, together with the atoms to which they are attached, form a 5 membered ring.

22. The compound of any one of claims 1-20, wherein X⁴ is O.

23. The compound of any one of claims 1-19, wherein each X³ and X⁴ is independently a covalent bond, O, S, NR⁶, C(O), CH₂, CHCH₃, or C(CH₃)₂.

24. The compound of any one of claims 1-23, wherein X² is CH, X is O, and X⁴ is O.

25. The compound of claim 24, wherein X¹ is N.

26. The compound of any one of claims 1-25, wherein L¹ is unsubstituted C₁₋₆ alkylene or C₁₋₆ alkylene comprising 1 or 2 oxo (═O) substituents.

27. The compound of claim 26, wherein L¹ is an unsubstituted linear C₄₋₆ alkylene or an unsubstituted branched C₄₋₆ alkylene.

28. The compound of claim 26 or 27, wherein L¹ is 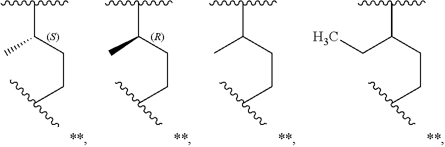
wherein * denotes the point of covalent attachment to X⁴, and ** denotes the point of covalent attachment to X³.

29. The compound of any one of claims 1-25, wherein L¹ is unsubstituted C₁₋₆ heteroalkylene or C₁₋₆ heteroalkylene comprising 1 or 2 oxo (═O) substituents.

30. The compound of claim 29, wherein the C₁₋₆ heteroalkylene comprises 1, 2, or 3 heteroatoms that are independently oxygen or nitrogen.

31. The compound of claim 29 or 30, wherein the C₁₋₆ heteroalkylene is —O(CH₂)ᵤ—, —(CH₂)ᵤ—, —O(CH₂)ᵤ—, —OCH₂OCH₂CH₂OCH₂—, —CH₂OCH₂CH₂O—, —OCH₂CH₂OCH₂—, —NH(CH₂)ᵤ—, —(CH₂)ᵤNH—, or —NH(CH₂)ᵤNH—, and wherein u is an integer of 1-4.

32. The compound of any one of claims 1-31, wherein B is phenyl or 5- to 6-membered heteroaryl.

33. The compound of claim 32, wherein B is phenyl, pyridyl, pyrimidyl, pyrazolyl, pyrrolyl, thiazolyl, oxazolyl, or imidazolyl.

34. The compound of claim 32 or 33, wherein R³ is methyl, halogen, or CN, and o is 0 or 1.

35. The compound of any one of claims 32-34, wherein B is 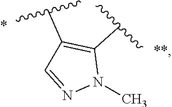 [SMILES: *c1cnn(C)c1*]
wherein * denotes the point of covalent attachment to C, and ** denotes the point of covalent attachment to X³.

36. The compound of any one of claims 1-35, wherein A is phenyl or 5- to 6-membered heteroaryl.

37. The compound of claim 36, wherein A is phenyl, pyridyl, pyrimidyl, pyrazolyl, pyrrolyl, thiazolyl, oxazolyl, or imidazolyl.

38. The compound of any one of claims 1-8, having a structure according to Formula (IV), 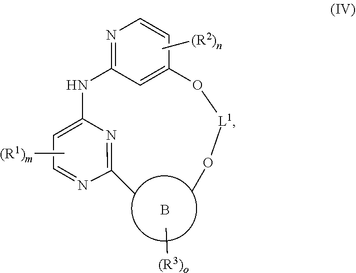
or a pharmaceutically acceptable salt thereof, wherein
L¹ is unsubstituted linear or branched C₂₋₆ alkylene;
B is phenyl or 5- to 6-membered heteroaryl;
R³ is methyl, halogen, or CN;
o is 0 or 1; and
one of R¹ and R² is present as Substructure A.

39. The compound of claim 38, having a structure according to Formula (V), 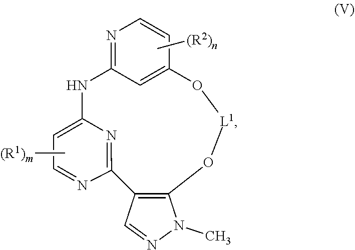
or a pharmaceutically acceptable salt thereof, wherein
L¹ is —(CH₂)₃— or —CH(CFI₃)CH₂CH₂.

40. The compound of claim 39, having a structure according to Formula (VI-1) or Formula (VI-2), 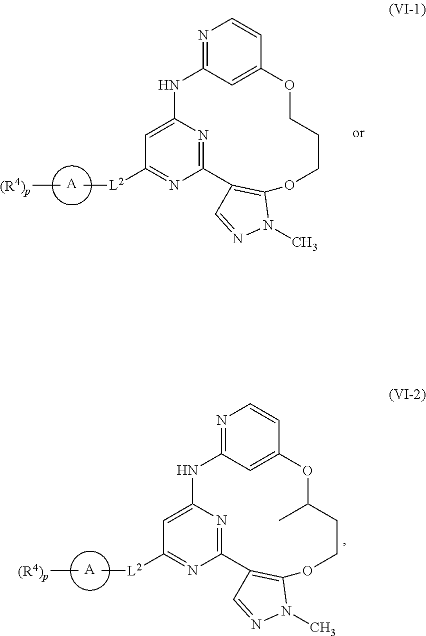
or a pharmaceutically acceptable salt thereof.

41. The compound of claim 40, having a structure according to Formula (VI-3) or Formula (VI-4), 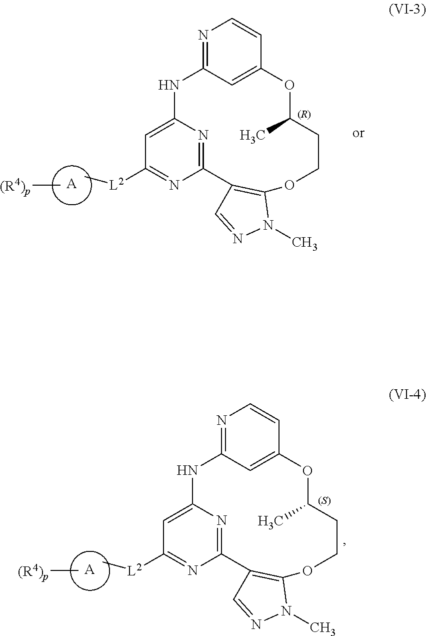
or a pharmaceutically acceptable salt thereof.

42. The compound of claim 39, having a structure according to Formula (VII-1) or Formula (VII-2), 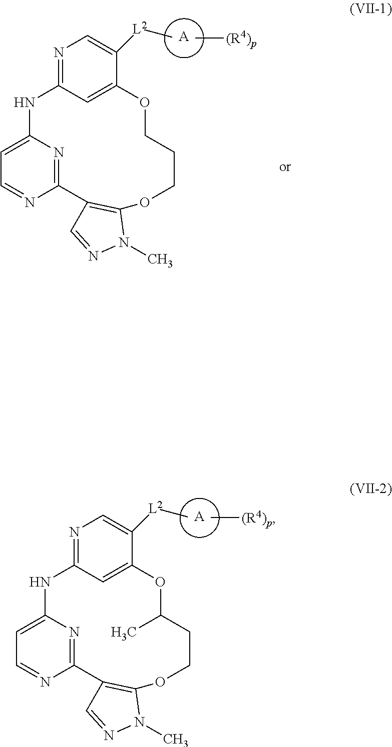
or a pharmaceutically acceptable salt thereof.

43. The compound of claim 42, having a structure according to Formula (VI-3) or Formula (VII-4), 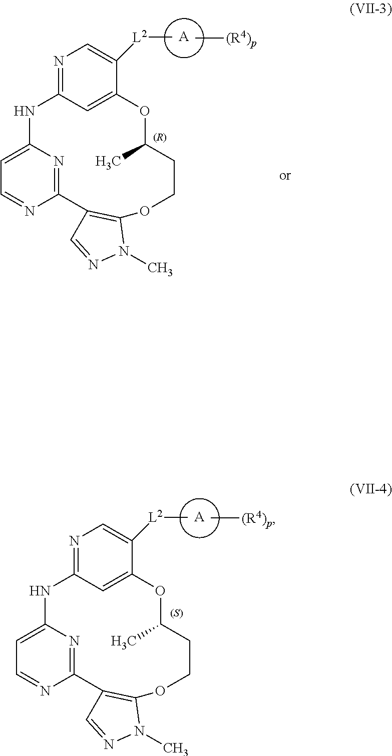
or a pharmaceutically acceptable salt thereof.

44. The compound of any one of claims 38-43, wherein A is phenyl or 5- to 6-membered heteroaryl.

45. The compound of any one of claims 1-44, wherein L² is a covalent bond.

46. The compound of any one of claims 1-44, wherein 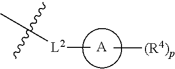
(Substructure A) is selected from the group consisting of:

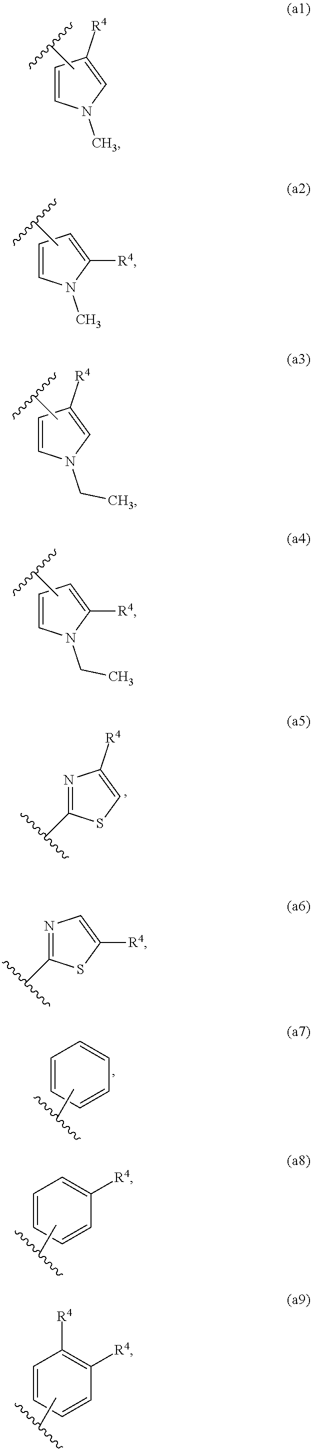

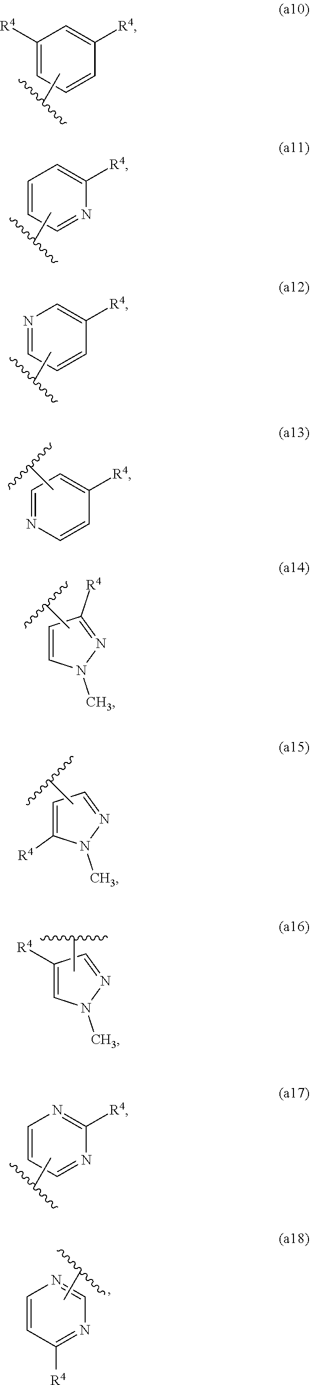

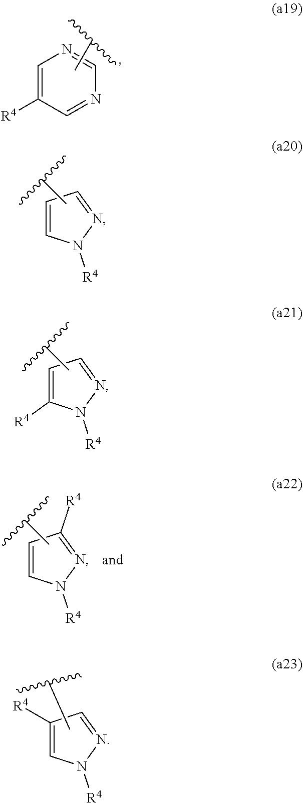

47. The compound of any one of claims 1-46, comprising one or more R⁴ groups selected from: —C≡N; —C≡CH; a saturated linear or branched C₁₋₆ aliphatic or C₁₋₆ alkoxy comprising 0-4 fluoro substituents; NR¹¹(CH₂)ₛNR⁸R⁹; (CH₂)ₜNR⁸R⁹; O(CH₂)OCH₃; O(CH₂)ᵣR¹²; and (CH₂)R¹².

48. The compound of claim 47, wherein R¹² is selected from the group consisting of: a C₃₋₆ cycloalkyl; a 3-9 membered heterocyclyl comprising 1-3 heteroatoms selected from O, N, and S; and 5- to 6-membered heteroaryl.

49. The compound of claim 47 or 48, wherein R¹² is selected from cyclopropyl, cyclobutyl, cyclopentyl, cyclohexyl, oxetanyl, tetrahydrofuryl, tetrahydropyanyl, azetidine, pyrroldinyl, piperidinyl, piperazinyl, and morpholino.

50. The compound of any one of claims 47-49, wherein R¹² is substituted with 0-4 R¹⁴, wherein each R¹⁴ is independently selected from —CN, oxo (═O), halogen, —OH, —NH₂, monoalkylamino, dialkylamino, unsubstituted C₃₋₆ cycloalkyl, or unsubstituted 3- to 4-membered heterocyclyl.

51. The compound of claim 50, wherein each R¹⁴ is independently selected from —CN, —F, —OH, —NH₂, —NHCH₃, —N(CH₃)₂, —NHCH₂CH₃, —N(CH₂CH₃)₂, —CH₃, —CH₂F, —CHF₂, —CF₃, —CH₂CH₃, —CH₂CH₂F, —CH₂CHF₂, —CH₂CF₃, —CH₂CH₂CH₃, —CHC₂CH₂F, —CH₂CH₂CHF₂, —CH₂CH₂CF₃, —CH₂CH₂OCH₃, —COCH₃, —COCH₂CH₃, —CH₂COCH₃, —CH₂COCH₂CH₃, cyclopropyl, cyclobutyl, oxetanyl, and azetidinyl.

52. The compound of claim 47, comprising:
an R⁴ group selected from: —CN, —CH₃, —CH₂F, —CHF₂, —CF₃, —CH₂CH₃, —CH₂CFH₂, —CH₂CHF₂, —CH₂CF₃, —CH(CH₃)₂, —C(CHF₃)₃, —C≡CH, 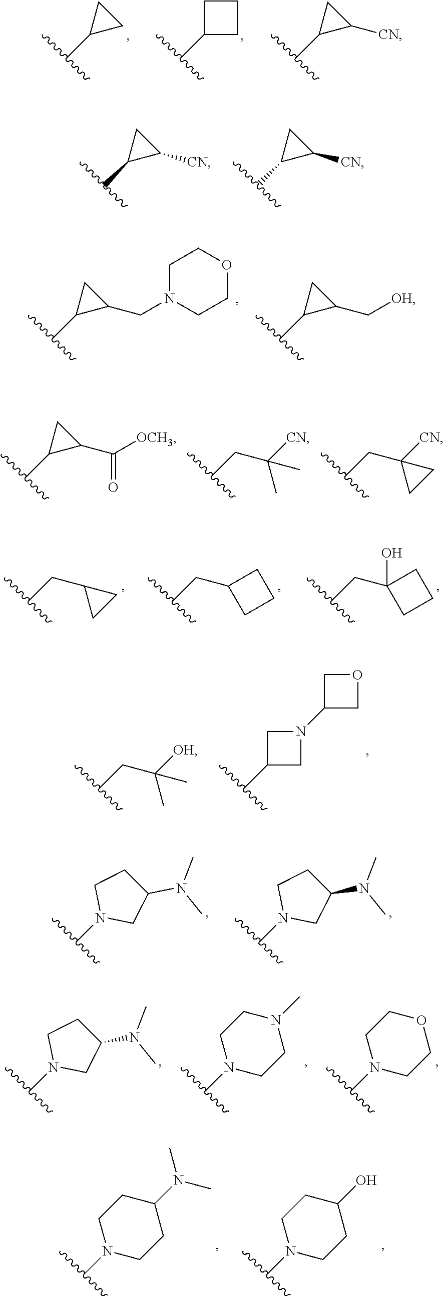 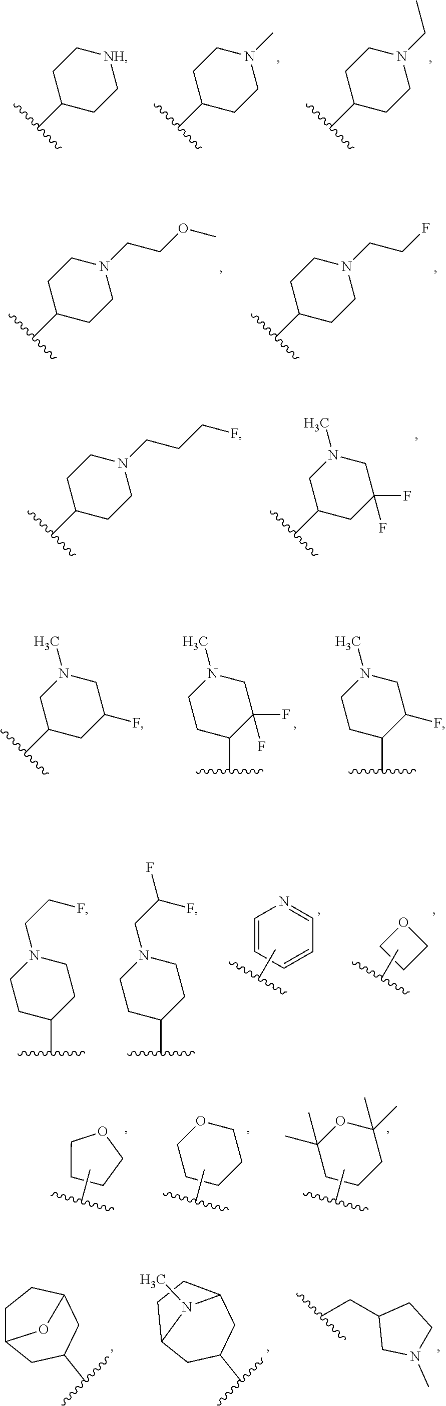 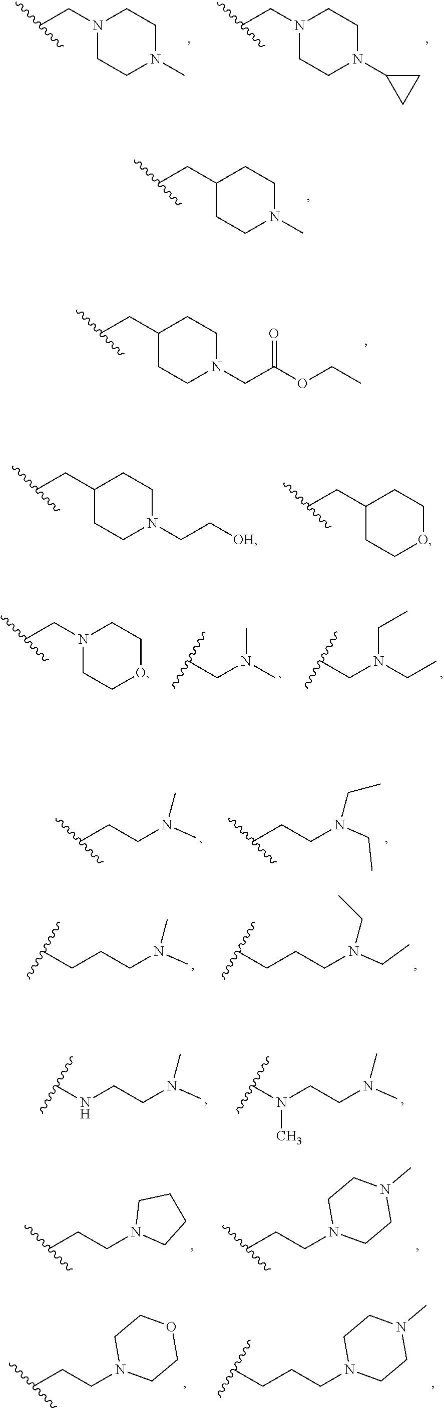
and/or
an R⁴ group selected from —CH₂OCH₃, —OCH₃, —OCH₂F, —OCHF₂, —OCF₃, —OCH₂CH₃, —OCH₂CH₂F, —OCH₂CHF₂, —OCH₂CF₃, —OCH₂CH₂OCH₃, 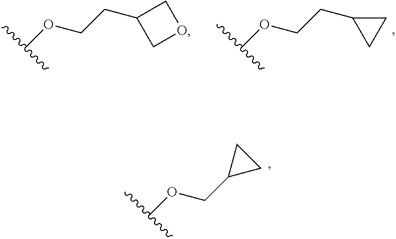 [SMILES: *OCCC1COC1 ; *OCCC1CC1 ; *OCC1CC1]
—CO₂CH₃, and CH₃.

53. The compound of any one of claims 1-46, wherein R⁴ is selected from unsubstituted C₁₋₆ alkyl, CO₂(unsubstituted C₁₋₆ alkyl), O-(unsubstituted C₁₋₆ alkyl), O—(C₁₋₆ haloalkyl), NH(CH₂)ₛNMe₂, (CH₂)ₜNMe2, or 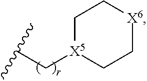
wherein
X⁵ is independently CH or N;
X⁶ is independently O, CHR¹³, or NR¹³;
X¹³ is independently H, C₁₋₆ alkyl, or C₃₋₆ cycloalkyl;
r is 0 or 1;
s is an integer of 2-4; and
t is an integer of 1-6.

54. The compound of claim 53, wherein one R⁴ is 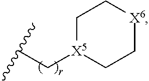
and, if present, a second R⁴ is selected from unsubstituted C₁₋₆ alkyl, CO₂(unsubstituted C₁₋₆ alkyl), O-(unsubstituted C₁₋₆ alkyl), O—(C₁₋₆ haloalkyl), NH(CH₂)₅NMe₂, and (CH₂)ₜNMe₂.

55. The compound of any one of claims 1-46, wherein 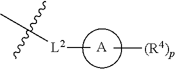
(Substructure A) is 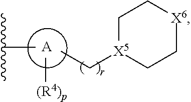
wherein
A is phenyl or 5- to 6-membered heteroaryl;
X⁵ is independently CH or N;
X⁶ is independently O, CHR³, or NR³;
R¹³ is independently H, unsubstituted C₁₋₆ alkyl, or unsubstituted C₃₋₆ cycloalkyl;
r is 0 or 1;
R⁴ is selected from unsubstituted C₁₋₆ alkyl, CO₂(unsubstituted C₁₋₆ alkyl), O-(unsubstituted C₁₋₆ alkyl), O—(C₁₋₆ haloalkyl), or NH(CH₂)ₛNMe₂;
p is 0 or 1; and
s is an integer of 2-6.

56. The compound of claim 55, wherein 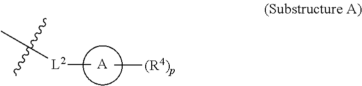
is 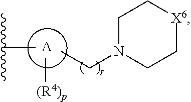
wherein X⁶ is O, NCH₃, or N(cyclopropyl).

57. The compound of any one of claims 53-56, wherein r is 0.

58. The compound of any one of claims 53-56, wherein r is 1.

59. The compound of any one of claims 46-58, comprising an R⁴ group that is —CO₂CH₃, —OCH₂CF₃, —CH₃, —CH₂CH₃, —OCH₃, —OCH₂CH₃, —NHCH₂CH₂N(CH₃)₂, or —CH₂N(CH₃)₂.

60. The compound of any one of claims 53-59, wherein A is phenyl, pyridyl, pyrimidyl, pyrazolyl, pyrrolyl, thiazolyl, oxazolyl, or imidazolyl.

61. The compound of claim 1, wherein said compound has a structure according to Formula (VIII), 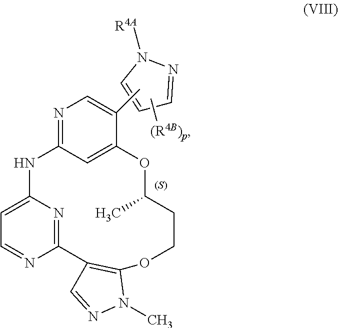
or a pharmaceutically acceptable salt thereof, wherein
R^{4A} is a first R⁴ group,
R^{4B} is a second R⁴ group, and
p is 0 or 1.

62. The compound of claim 1, wherein said compound has a structure according to Formula (IX), 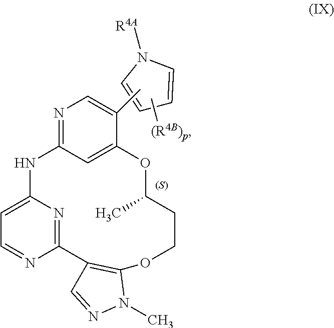
or a pharmaceutically acceptable salt thereof, wherein
R^{4A} is a first R⁴ group,
R^{4B} is a second R⁴ group, and
p is 0 or 1.

63. The compound of claim 1, wherein said compound has a structure according to Formula (X), 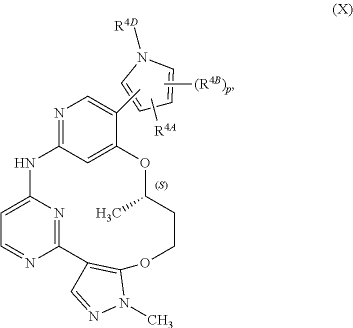
or a pharmaceutically acceptable salt thereof, wherein
R^{4A} is a first R⁴ group,
R^{4B} is a second R⁴ group,
p is 0 or 1, and
R^{4D} is a R⁴ group that is unsubstituted C₁₋₆ alkyl.

64. The compound of claim 1, wherein said compound has a structure according to Formula (XI), 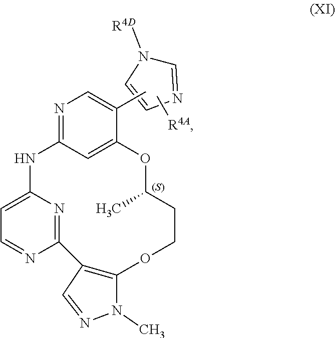
or a pharmaceutically acceptable salt thereof, wherein
R^{4A} is a first R⁴ group, and
R^{4D} is a R⁴ group that is unsubstituted C₁₋₆ alkyl.

65. The compound of claim 1, wherein said compound has a structure according to Formula (XII), 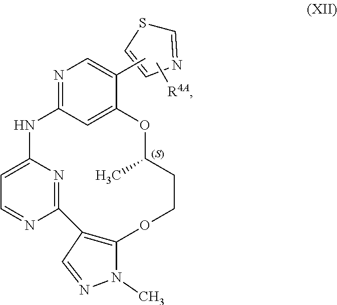
or a pharmaceutically acceptable salt thereof, wherein
R^{4A} is a first R⁴ group.

66. The compound of claim 1, wherein said compound has a structure according to Formula (XIII), 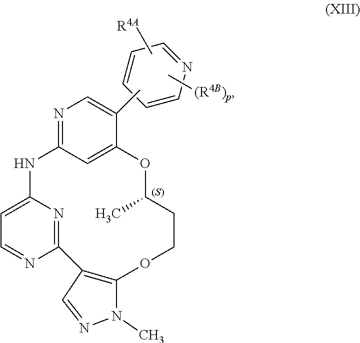
or a pharmaceutically acceptable salt thereof, wherein
R^{4A} is a first R⁴ group,
R^{4B} is a second R⁴ group, and
p is 0 or 1.

67. The compound of claim 1, wherein said compound has a structure according to Formula (XIV), 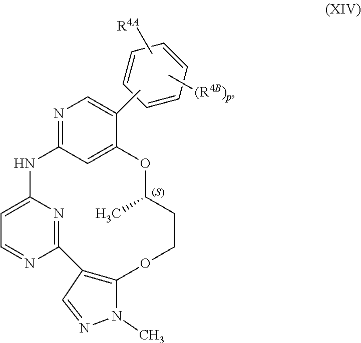
or a pharmaceutically acceptable salt thereof, wherein
R^{4A} is a first R⁴ group,
R^{4B} is a second R⁴ group, and
p is 0 or 1.

68. The compound of claim 1, wherein said compound has a structure according to Formula (XV), 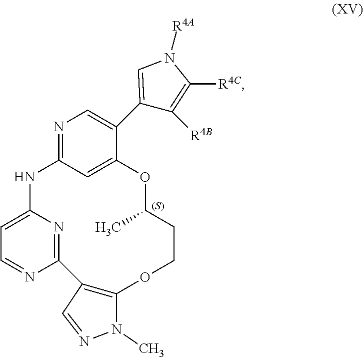
or a pharmaceutically acceptable salt thereof, wherein
R^{4A} is a first R⁴ group,
R^{4B} is a second R⁴ group, and
R^{4C} is a third R⁴ group.

69. The compound of claim 1, wherein said compound has a structure according to Formula (XVI), 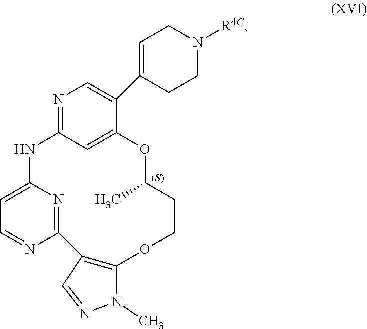
or a pharmaceutically acceptable salt thereof, wherein
R^{4C} is a first R⁴ group.

70. The compound of claim 1, wherein said compound has a structure according to Formula (XVII), 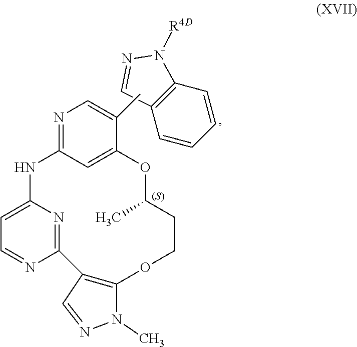
or a pharmaceutically acceptable salt thereof, wherein
R^{4D} is a R⁴ group that is unsubstituted C₁₋₆ alkyl.

71. The compound of claim 1, wherein said compound has a structure according to Formula (XVIII), 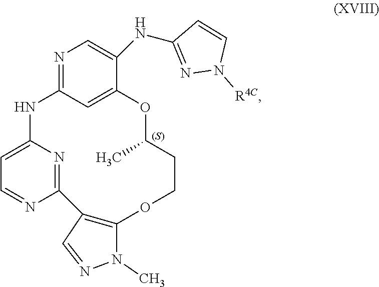
or a pharmaceutically acceptable salt thereof, wherein
R^{4C} is a first R⁴ group.

72. The compound of claim 1, wherein said compound has a structure according to Formula (XIX), 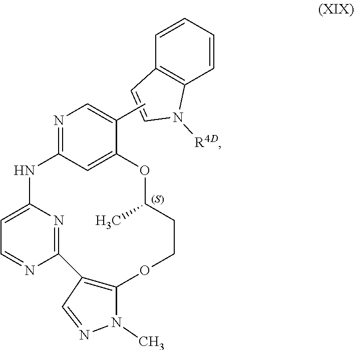
or a pharmaceutically acceptable salt thereof, wherein
R^{4D} is a R⁴ group that is unsubstituted C₁₋₆ alkyl.

73. The compound of claim 1, wherein said compound has a structure according to Formula (XX), 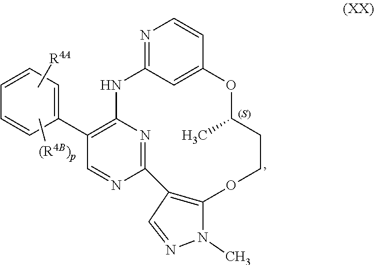
or a pharmaceutically acceptable salt thereof, wherein
R^{4A} is a first R⁴ group,
R^{4B} is a second R⁴ group, and
p is 0 or 1.

74. The compound of claim 1, wherein said compound has a structure according to Formula (XXI), 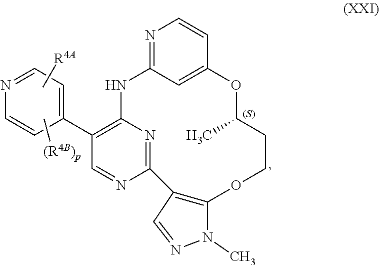
or a pharmaceutically acceptable salt thereof, wherein
R^{4A} is a first R⁴ group,
R^{4B} is a second R⁴ group, and
p is 0 or 1.

75. The compound of claim 1, wherein said compound has a structure according to Formula (XXI) 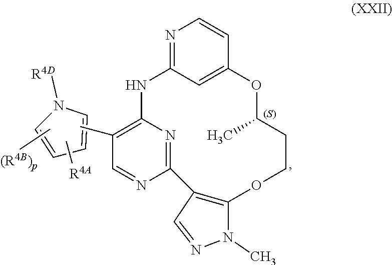
or a pharmaceutically acceptable salt thereof, wherein
R^{4A} is a first R⁴ group,
R^{4B} is a second R⁴ group,
p is 0 or 1, and
R^{4D} is a R⁴ group that is unsubstituted C₁₋₆ alkyl.

76. The compound of claim 1, wherein said compound has a structure according to Formula (XXIII), 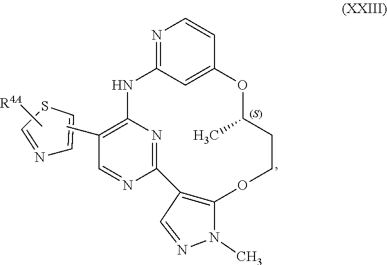
or a pharmaceutically acceptable salt thereof, wherein
R^{4A} is a first R⁴ group.

77. The compound of any one of claims 61-76, wherein each R^{4A}, R^{4B}, and R^{4C}, when present, is independently a R⁴ group selected from: —C≡N; —C≡CH; a saturated linear or branched C₁₋₆ aliphatic or C₁₋₆ alkoxy comprising 0-4 fluoro substituents; NR¹¹(CH₂)NR⁸R⁹; (CH₂)ₜNR⁸R⁹; O(CH₂)ₜOCH₃; O(CH₂)ᵣR¹²; and (CH₂)ᵣR¹².

78. The compound of claim 77, wherein R¹² is selected from the group consisting of:
a C₃₋₆ cycloalkyl; a 3-9 membered heterocyclyl comprising 1-3 heteroatoms selected from O, N, and S; and 5- to 6-membered heteroaryl.

79. The compound of claim 77 or 78, wherein R¹² is selected from cyclopropyl, cyclobutyl, cyclopentyl, cyclohexyl, oxetanyl, tetrahydrofuryl, tetrahydropyanyl, azetidine, pyrroldinyl, piperidinyl, piperazinyl, and morpholino.

80. The compound of any one of claims 77-79, wherein R¹² is substituted with 0-4 R¹⁴, wherein each R⁴ is independently selected from —CN, oxo (═O), halogen, —OH, —NH₂, monoalkylamino, dialkylamino, unsubstituted C₃₋₆ cycloalkyl, or unsubstituted 3- to 4-membered heterocyclyl.

81. The compound of claim 80, wherein each R¹⁴ is independently selected from —CN, —F, —OH, —NH₂, —NHCH₃, —N(CH₃)₂, —NHCH₂CH₃, —N(CH₂CH₃)₂, —CH₃, —CH₂F, —CHF₂, —CF₃, —CH₂CH₃, —CH₂CH₂F, —CH₂CHF₂, —CH₂CF₃, —CH₂CH₂CH₃, —CH₂CH₂CH₂F, —CH₂CH₂CHF₂, —CH₂CH₂CF₃, —CH₂CH₂OCH₃, —COCH₃, —COCH₂CH₃, —CH₂COCH₃, —CH₂COCH₂CH₃, cyclopropyl, cyclobutyl, oxetanyl, and azetidinyl.

82. The compound of any one of claims 61-81, comprising:
an R^{4A} and/or a R^{4C} group, when present, is selected
from: —CN, —CH₃, —CH₂F, —CHF₂,
—CF₃, —CH₂CH₃, —CH₂CF H₂, —CH₂CHF₂, —CH₂CF₃, —CH(CH₃)₂, —C(CH₃)₃, —C≡CH, 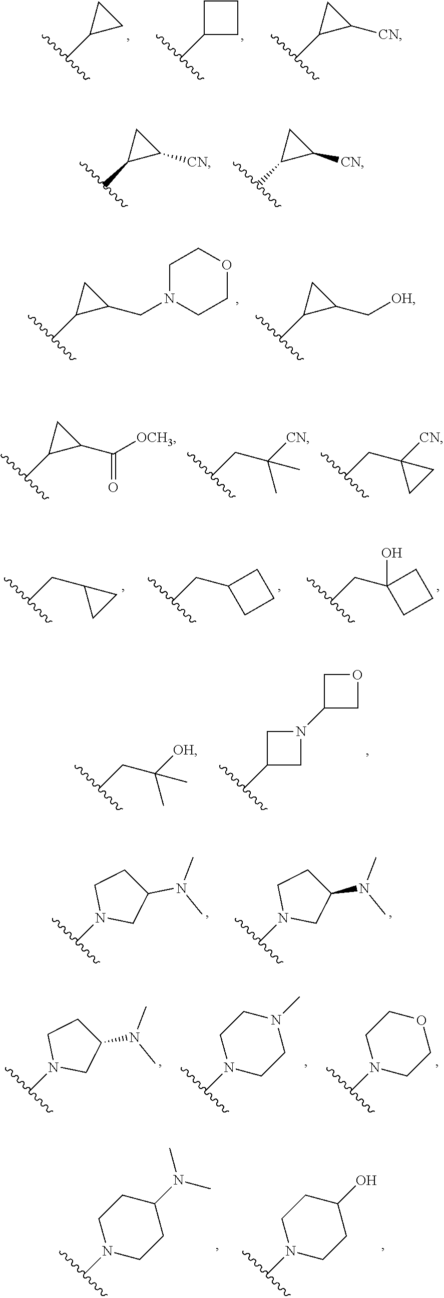 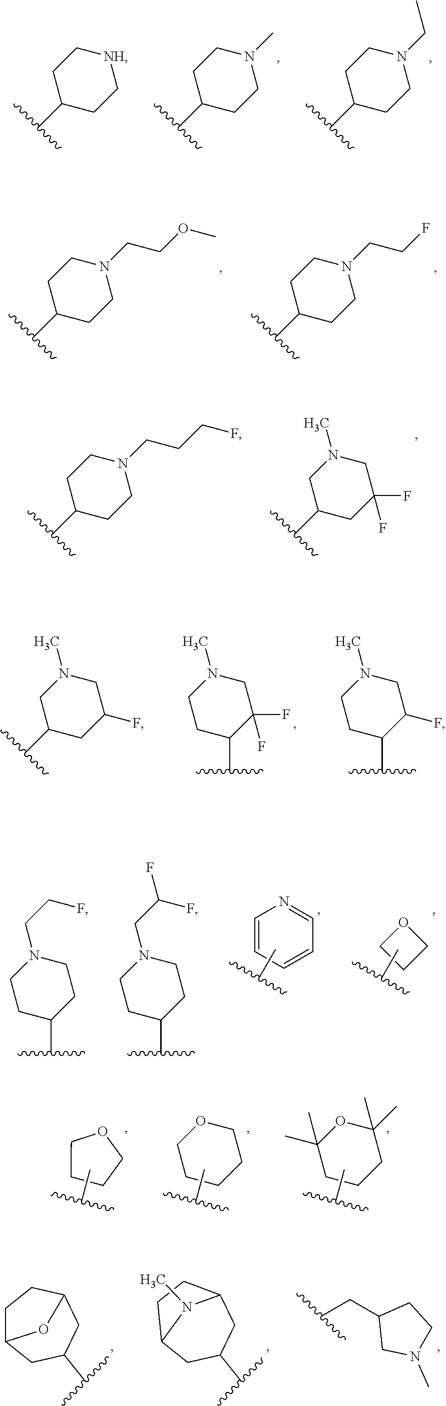 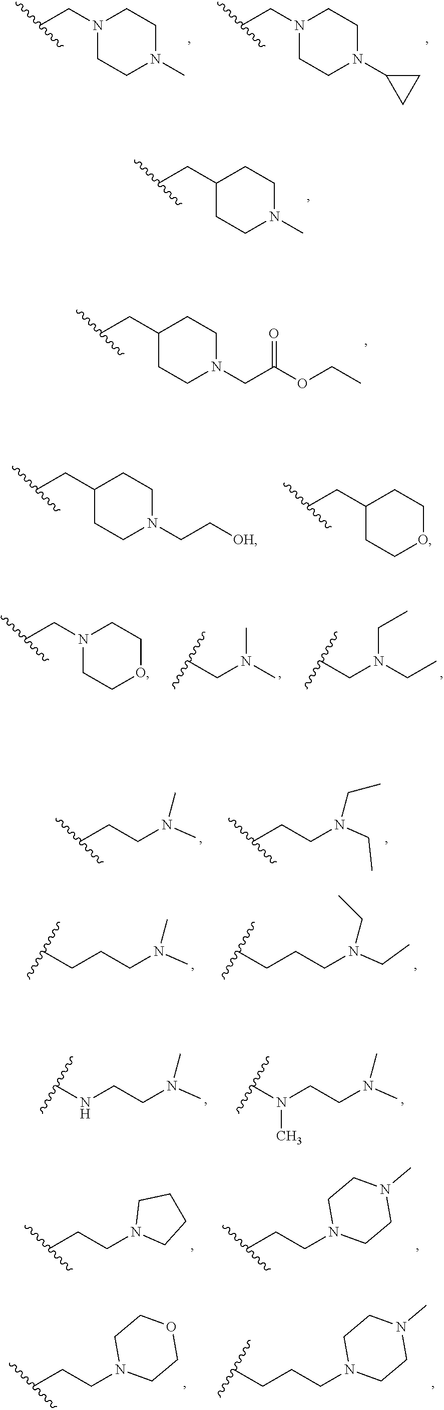
and/or
an R^{4B} group, when present, is selected from —CH₂OCH₃, —OCH₃, —OCH₂F, —OCHF₂, —OCF₃, —OCH₂CH₃, —OCH₂CH₂F, —OCH₂CHF₂, —OCH₂CF₃, —OCH₂CH₂CH₃, —OCH₂CH(CH₃)₂, —OCH₂CH₂OCH₃, 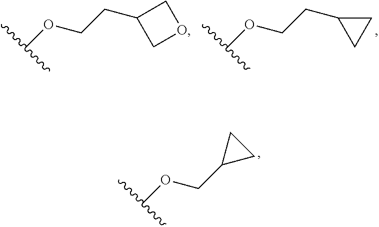 [SMILES: *OCCC1COC1 ; *OCCC1CC1 ; *OCC1CC1]
—CO₂CH₃, and CH₃.

83. The compound of claim 1, selected from the group consisting of Compounds (1)-(169), or a pharmaceutically acceptable salt thereof.

84. A pharmaceutical composition comprising a compound according to any one of claims 1-83, or a pharmaceutically acceptable salt thereof.

85. A method of treating cancer comprising administering to a human in need thereof an effective amount of a compound according to any one of claims 1-83 or a pharmaceutically acceptable salt thereof in a pharmaceutical composition.

86. The method of claim 85, wherein said cancer is a lung cancer.

87. The method of claim 85 or 86, wherein said cancer is non-small cell lung cancer.

88. The method of any one of claims 85-87, wherein said cancer is an EGFR-driven cancer.

89. The method of any one of claims 85-88, wherein said cancer is characterized by an EGFR mutation.
